SEQUENCE LISTING (742 sequences)

SEQ ID NO: 1 - protein, 1071 aa, Bacillus sp.
MRELDYRIGLDIGTNSIGWGVIELSWNKDREQYEKTRIVDKGVRMFDKAEIPKTGASLAEPRRIARSSRRRLNRKSQRKKDIRNLLVQHEIISQKELTSLYPLSKSSMDIWDIRLDGLDRLLDRFEWARLLIHLAQRRGFKSNRKSELKDVETGKVLSSIQVNEKRLSLYRTVGEMWMKNADCSKYGKRRNSPNEYVFSVSRADLEKEIVTLFEAQRKFHSSYASVDLQKTYIQIWAHQLPFASGNAIVNKVGYCSLLKGKEKRVPKATYTFQYFNTLDQINRTRLGPNFQPFTKEQRDIILDKMFQRTDYYKKKTIPEVTYYDIRKWLALDETIQFKGLTYDPNEELKKIEMKPFINLKPFYEIKKVVTNYAKKTNEVFSALDYDTVAYALTVYKTDKDIRSYLKRSNNLSKRCYDDQLIEELLTLSYTKFGHLSFKAINHVLPIMQEGRTYQEAIHQLGYDTTNLKKENRSMFLPIIPDEITNPIVKRALTQARKVVNAIIRRYGSPNSVHIELARELSKSHDERKKIMTAHDENYKKNKGAISILIENGILNPTGYDIVRYKLWKEQGERCAYSLKKIPANTFFNELKKERSGPPVLEVDHILPYSQSFIDSYHNKVLVYGDENQKKGNRIPYTFFSEEDKEWESFESYVRSNSFFSKKKRGYLLKKAYLPRESNLIKERHLNDTRYASSYLKNFIEKNLKFKEAVGITRKKYVQTVNGVITAHLRKRWGLEKDRQETYLHHAMDAIIVACTDHHMVTKVTEYYQIKEGNKSIKKPYFPLPWMGFREEILSHLESQPIARKISEELKIGYQSSDYILVSRMPKRSVTGSAHDQTVMKKGGIDKKGKTIIIKRVHLKDIKFDENGDFKMVGKEQDLATYEAIKQRYLEYRKKSKKAFETPLYKPSKKGKGNLIKKIKVEVQTKSFVREINGGVAQNGDLVRVDLFEKDNRYYMVPIYVVDTVRSELPNKAVTSSKGYEQWLSIDNSFTFKFSLYPYDLVRLVKGDEDRFLYFSTLDINSDRLNFKDVNKPSKQAEYRYSLKTIENLEKYEIGVLGDLRLVRQETRKIFK

SEQ ID NO: 2 - RNA, 34 nt, Bacillus sp.
  source 1..34: mol_type = other RNA
gtcatagttccattaaagccattgctgttttatg

SEQ ID NO: 3 - RNA, 148 nt, Bacillus sp.
  source 1..148: mol_type = other RNA
catgatacagcaatggctttgatgtttctatgataagggcttcggcccgtggcgttggggatcgcctgcccattttaatgggcttctccccatctatttaatgagaaatttacaaccttggctattcttaaatagctaaggttttttt

SEQ ID NO: 4 - RNA, 215 nt, synthetic construct
  misc_difference 1..29: note = a, c, u, g, unknown or other
  misc_feature 1..215: note = source = /note="Description of Artificial Sequence: Synthetic polynucleotide"
  source 1..215: mol_type = other RNA
nnnnnnnnnnnnnnnnnnnnnnnnnnnnngtcatagttccattaaagccattgctgttttatgaaagcatgatacagcaatggctttgatgtttctatgataagggcttcggcccgtggcgttggggatcgcctgcccattttaatgggcttctccccatctatttaatgagaaatttacaaccttggctattcttaaatagctaaggttttttt

SEQ ID NO: 5 - RNA, 216 nt, synthetic construct
  misc_feature 1..216: note = source = /note="Description of Artificial Sequence: Synthetic polynucleotide"
  source 1..216: mol_type = other RNA
gagcggacagcagcttcctatatctcgtacgtcatagttccattaaagccattgctgttttatgaaagcatgatacagcaatggctttgatgtttctatgataagggcttcggcccgtggcgttggggatcgcctgcccattttaatgggcttctccccatctatttaatgagaaatttacaaccttggctattcttaaatagctaaggttttttt

SEQ ID NO: 6 - RNA, 216 nt, synthetic construct
  misc_feature 1..216: note = source = /note="Description of Artificial Sequence: Synthetic polynucleotide"
  source 1..216: mol_type = other RNA
ccatgatatagacgttgtggctgttgtagtgtcatagttccattaaagccattgctgttttatgaaagcatgatacagcaatggctttgatgtttctatgataagggcttcggcccgtggcgttggggatcgcctgcccattttaatgggcttctccccatctatttaatgagaaatttacaaccttggctattcttaaatagctaaggttttttt

SEQ ID NO: 7-8 - skipped (2 entries)

SEQ ID NO: 9 - protein, 1403 aa, Chryseobacterium sp.
MVERILGLDLGVSSIGWALVEEDVENPENNKIIKLGVRVNPLTIDEKTNFEKGKSITTNAGRTSARSARRNLQRFKLRRKSLWEVLINYKIIKNDTVLAEIGKNSTFQTQYLRAKAAKEKIELDELARVLFLINKKRGYKSSRKVQSDDEGKAIDGMSVAKELYEKNYTPGQYVYDLLKNGKKQIPDFYRSDLQNELDRVWEFQKQYHPDILTDEYKKELEGKGQRATAAIFLNRYKIYTADNKGTREEKRFQAYQWRVEALSQPLDIKEVAYVITEINNNLNNSSGYLGAISDRSKKLYFNGQTIGEYLYEELSLNPHARLKNQVFYRQDYQDEFEKIWETQAKFHSVLTTELKEDVRDIIIFYQRKLKSQKGLISFCEFESKEIVIEENGKKKRKKVGLKVVPKSSPLFQEFKIWQVLNNIEIKNEDGRRYLDQEEKELLFNELNCKGNLTSQKCLEFLGFKSKETKINFKVIEGNRTNEKLYDAFLKILELKGYNVFELLKIKEDRDEAKLSELKASADEIKRMVKEIFNANLINTSILDFNAELEGKDFENQASYQFWHLIYSYEGDNSPSGNEKLYELLENKFGFKKEHSKILSTIVFPQEYGSLSAKAMRRIYPYIKDNKYSEACLLAGYNHSKQSLTKEELENRKLKEQLEVLPKNSLRNPVVEKILNQMINLVNAIISEHGKLDGVRIELARELKKSAQEREDMTKSINEATILHQKYATVLRQEFGVVNPSRNDIIRYKLYMELAGNGYKDLYTNVKIEKENIFTDKYDIDHIIPQSRFFDDSFSNKVLVPRQANLDKGNLTGYDFMSNKGAEREEHFLNVIKDLLENGSISKAKYEKLKKKGIEIGDGFIERDLRDTQYIARKAKEILFEITNSVISTSGRITDKLREDWDLVNTMKELNLDKYRRLGLTEIVINSKGEEKEVIPDWTKRNDHRHHAMDALTVAFTTRNHIQYLNYLNARKDEKHKEHQNIYAIENIITEIIEKKNGSKSRRFKTPMNRFRHEAKQHLKEVLVSHKTKNKVVTSNINKTKKKRGIHKKAELTPRGQLHKETVYGSKKFLQSREEKVSAKFDYETILMVVNPIHRNALLNRLKEYGNDPKKAFSGKNAVNKTPVYLSDSKIEVLPEKVTLSWFETGYTIRKAVTPDNFKDYKNLEKITDMGIKKILKERLDQFKGNAKEAFSNLDKEPIWLNEEKGIAIKTVTITGVSNAESLHFKKDHLGKEILDENGNKIPVDFVSTGNNHHLAIYLNEEEKLDDKMVTFYEAVLRVNQGLPVIDKNYNREKGYKFLMTLKQNEMFVFPNEEFDPSEIDLLDEKNLEQISQNLFRVQKISKVGYGNSFIRDFVFRHHLETTVEDRKELRNTTYIQLKSLEGLRNIVKVRLNHLGKIVQIGEY

SEQ ID NO: 10 - RNA, 24 nt, Chryseobacterium sp.
  source 1..24: mol_type = other RNA
gttgtgaaatgctttcaaaactta

SEQ ID NO: 11 - RNA, 78 nt, Chryseobacterium sp.
  source 1..78: mol_type = other RNA
ataaattttgaaagcacttcacaataaggattattccgttgtgaaaacattcaaggcggggcaactcgcctttttttt

SEQ ID NO: 12 - RNA, 135 nt, synthetic construct
  misc_difference 1..29: note = a, c, u, g, unknown or other
  misc_feature 1..135: note = source = /note="Description of Artificial Sequence: Synthetic polynucleotide"
  source 1..135: mol_type = other RNA
nnnnnnnnnnnnnnnnnnnnnnnnnnnnngttgtgaaatgctttcaaaacttaaaagataaattttgaaagcacttcacaataaggattattccgttgtgaaaacattcaaggcggggcaactcgcctttttttt

SEQ ID NO: 13 - RNA, 136 nt, synthetic construct
  misc_feature 1..136: note = source = /note="Description of Artificial Sequence: Synthetic polynucleotide"
  source 1..136: mol_type = other RNA
gagcggacagcagcttcctatatctcgtacgttgtgaaatgctttcaaaacttaaaagataaattttgaaagcacttcacaataaggattattccgttgtgaaaacattcaaggcggggcaactcgcctttttttt

SEQ ID NO: 14 - RNA, 136 nt, synthetic construct
  misc_feature 1..136: note = source = /note="Description of Artificial Sequence: Synthetic polynucleotide"
  source 1..136: mol_type = other RNA
ccatgatatagacgttgtggctgttgtagtgttgtgaaatgctttcaaaacttaaaagataaattttgaaagcacttcacaataaggattattccgttgtgaaaacattcaaggcggggcaactcgcctttttttt

SEQ ID NO: 15 - skipped

SEQ ID NO: 16 - protein, 1404 aa, Sphingobacterium sp.
MMKRILGLDLGTSSIGWAYVLEAQDENEISQIKKLGVRVNPLTTDEQLNFEKGKPITTNAGRTLARSARRNLQRYKLRRENLIEVLKREKWIDDKSILAENGNKSTFETYALRAAAASKEIKLEELARVLLMINKKRGYKSSRKVNKEDDGQLVDGMEVAKKLYHENLTPGEFVYRLLKEGKKKIPDFYRSDLQRELDLIWDFQFKYYPDLLTQDLKEELKGKGLRATSAIFWSKYKFNTAENKGTREEKKIQAYQWRKDGLTKQLEQEEVAYIIAEINGQLANSSGYLGAISDRSKELYFNKQTVGQFLFNQLQHNTHARVKNQVFYRQDYLDEFEKIWSVQSQYHQGLTEQLKEEIRDIIIFYQRKLKSQKGLISFCEFEQHEVVINGKNKIVGLRVAPKSSPIFQEFKIWQQINNVKLRNKITKEVYNLAEEQKRSLFETLNLKGKLSAYQVLNLIEVKPKEWELNYTELEGNSTNLALYNAYLDILDIEGYDVRSELKIKLNKDEITLSDLDIPVSEIKGMIRRIFQHLGINTSILDFNAQMQGDDFEKQLSYQLWHLLYSYEEDNSKTGMERLYNQLNLKFGFSLDQAKIFGKIALQEDYGNLSTKAIRKIYPYIQDVEYSVACKQAGYNHSKSSLTKEQLTNRVLKDHLDILPKNSLRNPVVEKILNQMVNVVNTLIETENDKLIKEGKNADFRFDEIRIELARELKKNAKEREELTKAINTSKSEHEKIIKILQTEDGIKNPTRNDIIRFKLYQELKNNGYKNLYTNEYIQRKDLFTNVYDIDHIIPQSRLFDDSFSNKVLVPRNINIEKGNQTAFDYVHAKFGEDGIEAYEARVERLFNLKEEGVSRSKYKKLLMRGLDIGEGFIERDLRDSQYIAKKAKAMLFEIAPSVISTSGSVTDRLREDWGLVSVMKELNLPKFKAVGLTEYLETKDGNRKEVIKDWSKRNDHRHHAMDALTVAFTKHSHIHYLNHLNARKNEKSEFFSTIKAIEIKETYVERDDLGNRKRLFKEPIPHFRSIAKEHLESVLVSHKAKNKVVTKNRNKIDGKKQAQEVLTPRGQLHKESIYGKILQYASKEEKISAKFDAATIAMVSNPRYRSALLDRLSAYGNDPKKAFTGKNSPNKSPIYLDQEKQLAVPEKVKLVWLEEDYTIRKEIGPDLKIEKVIDQGVKRILEQRLTEYGGDSKKAFSDLDNNPIWLNKEKGIAIKRVTISGVKNAEALHIKKDHLGQVLKDKDGKPMPVDFVSTGNNHHVAIYEDDNGALQERIVPFYEAVARVNNGFPIIDKHYNQHLGWKFLFSMKQNEMFLFPSEEFDPKQINLLDRSNYRLISKNLFRVQKFGELSKSGFWFRHHLETSVDVNKSLRSISYFDFYSKDFMKNIAKVRINHLGEIIHVGEY

SEQ ID NO: 17 - RNA, 18 nt, Sphingobacterium sp.
  source 1..18: mol_type = other RNA
gttgtgaattgctttcat

SEQ ID NO: 18 - RNA, 72 nt, Sphingobacterium sp.
  source 1..72: mol_type = other RNA
atgaaagcaattcacaataaggattatttccgttgtgtaaacatttagcgcctcgtctatctacggggcatt

SEQ ID NO: 19 - RNA, 123 nt, synthetic construct
  misc_difference 1..29: note = a, c, u, g, unknown or other
  misc_feature 1..123: note = source = /note="Description of Artificial Sequence: Synthetic polynucleotide"
  source 1..123: mol_type = other RNA
nnnnnnnnnnnnnnnnnnnnnnnnnnnnngttgtgaattgctttcataaagatgaaagcaattcacaataaggattatttccgttgtgtaaacatttagcgcctcgtctatctacggggcatt

SEQ ID NO: 20 - RNA, 124 nt, synthetic construct
  misc_feature 1..124: note = source = /note="Description of Artificial Sequence: Synthetic polynucleotide"
  source 1..124: mol_type = other RNA
gagcggacagcagcttcctatatctcgtacgttgtgaattgctttcataaagatgaaagcaattcacaataaggattatttccgttgtgtaaacatttagcgcctcgtctatctacggggcatt

SEQ ID NO: 21 - RNA, 124 nt, synthetic construct
  misc_feature 1..124: note = source = /note="Description of Artificial Sequence: Synthetic polynucleotide"
  source 1..124: mol_type = other RNA
ccatgatatagacgttgtggctgttgtagtgttgtgaattgctttcataaagatgaaagcaattcacaataaggattatttccgttgtgtaaacatttagcgcctcgtctatctacggggcatt

SEQ ID NO: 22 - skipped

SEQ ID NO: 23 - protein, 1453 aa, Chryseobacterium sp.
MMIKNILGLDLGTNSIGWALIKQDFENKYGEILGMGSRIIPMDQKVLGEFGSGNSVSQTADRTKYRGVRRLRERFLLRRERLHRVLNVLNFLPIHYASQIDFEKRFGKFREEIEPKLVYNNDGFIFKNSFEEMLSDFRKHQPQILENDKKIPYDWTIYYLRKKALTQKIEKEELAWILLNFNQKRGYYQLRGEDIEEEKDKTFVRLKVEKIVDSGDNVKGKILYDVYFDNGWKYDKQIVKTEDWIERVKEFIVTESFLKNGDIKRTYKAVDSEKDWIAIKTKTEQEIEKSHKTVGTYIYETILHNPKQKIKGKLVRTIERKFYKDELRQILEKQKEFHPELQNDDLYSDCVRELYRNNEAHQLTLSKKDFVHLFIEDIIFYQRPLRSQKSSISNCTLEYRRYKDKHGVEHIQYLKAIPKSNPYYQEFRIWQWMYNLNIYKRDDDTNVTKEFLNSTEDFENLFDFLNNRKEVEQKALLKHFKLNEKTHRWNFVENKKYPCNETKTMISSRLDKVENISENFLTSEIEHKIWHIIYSVNDKIEYEKALKSFANKNNLDEVSFFEAFKKFPPFKNEYGSFSEKAIKKLLPLMRVGKYWDEDEIVKNSDTYFKNIEDLLGNIARKEENISDDDRKKYNKTVNLKLREELEIFQDAEIASFQKLRLHIAQYLVYGRHSEASIIGKWNSADDLEEFLKEFKQHSLRNPIVEQVITETLRVVKDIWMKYGNGAKDFFNEIHIELGREMKLPAEDRKKLTSQISENENTNLRIKALLAEMMNDSSVENVRPFSPMQQEILKIYEDGVLNSGIEIEDEYLKISKTAQPSSSDLKRYKLWLEQKYKSPYTGQIIPLNKLFTPEYEIEHIIPKSRYFDDSFSNKIICESAVNKLKDNYIGLGFIKQFAGAIVELGFGKNVKVFEIEEYEDFVKKHYANNRGKRNKLLLEEIPEKMIERQLNDTRHISKYISSVLSNIVRVEDGTDEGVNSKNIVPGNGKITIQLKQDWGLNDVWNDLILPRFERMNQLTNSTDFTAWNKNHQKYLPTVPIEFSKGFSKKRIDHRHHALDALVIACTTKDHVNLLNNQSAKSDTKRYDLKKKLMKFEKVVYNHTQTGEKIERDIPKQFLKPWETFTIDAKNRLETIIVSFKQNLRVINKATNHYEKYVEKEGVKMKERVEQTGTNWAIRKSLHEETVSGKITLSWVETSKGEFITATRKPLDSSFTFEKINKITDTGIQKILNNYLEAKDNNPELAFSAEGIEDLNKNIEKYNDGKSHQPIIKIRLYEKGKGRFVLGQTGNKINKYVQGSPNLFFAIYKDENGKKIFESIRLDIVIERLKQGLQAIPETNQNGVSLYQSLSPLDLVYIPTEYELESPHILDFSKLNKTQITRLYNTNDFSGVTAYFSQNSFAKHIYPKEMDLSWNEKKQKLSGSFDSKTASYNNTSIKDIFIKVKVDRLGNISKA

SEQ ID NO: 24 - RNA, 29 nt, Chryseobacterium sp.
  source 1..29: mol_type = other RNA
gttgtgaattgctttcaaaaattattatc

SEQ ID NO: 25 - RNA, 83 nt, Chryseobacterium sp.
  source 1..83: mol_type = other RNA
taataattttgaaagcaattcacaataaggattattccgttgtgaaaacattcaaggcggggcaactcgccttttttcgtttt

SEQ ID NO: 26 - RNA, 145 nt, synthetic construct
  misc_difference 1..29: note = a, c, u, g, unknown or other
  misc_feature 1..145: note = source = /note="Description of Artificial Sequence: Synthetic polynucleotide"
  source 1..145: mol_type = other RNA
nnnnnnnnnnnnnnnnnnnnnnnnnnnnngttgtgaattgctttcaaaaattattatcaaagtaataattttgaaagcaattcacaataaggattattccgttgtgaaaacattcaaggcggggcaactcgccttttttcgtttt

SEQ ID NO: 27 - RNA, 146 nt, synthetic construct
  misc_feature 1..146: note = source = /note="Description of Artificial Sequence: Synthetic polynucleotide"
  source 1..146: mol_type = other RNA
gagcggacagcagcttcctatatctcgtacgttgtgaattgctttcaaaaattattatcaaagtaataattttgaaagcaattcacaataaggattattccgttgtgaaaacattcaaggcggggcaactcgccttttttcgtttt

SEQ ID NO: 28 - RNA, 146 nt, synthetic construct
  misc_feature 1..146: note = source = /note="Description of Artificial Sequence: Synthetic polynucleotide"
  source 1..146: mol_type = other RNA
ccatgatatagacgttgtggctgttgtagtgttgtgaattgctttcaaaaattattatcaaagtaataattttgaaagcaattcacaataaggattattccgttgtgaaaacattcaaggcggggcaactcgccttttttcgtttt

SEQ ID NO: 29 - skipped

SEQ ID NO: 30 - protein, 1093 aa, Brucella sp.
MKTLGIDLGTASIGWAILDEASIIASGVRIFSQSEMAGRDPQSKASLAVARRDARGARRRRDRYLKRRRRLLDLLTEHGLAPADEKSRKALVREYEDGKDGDLSNSVYALRARALDEALTPYEIGRAIFQLNQRRGFKSNRKTDSNDPEQGKIATAIHVLDAKMDEDKARTFGEWLHMRRLKGLSVRARMTADGDSYDFYPSRAALEREFDRLMASQKRFHPDLLNSSVIDDIRKVVFHQRPLKPVQPGKCSYNHRESRLPKAHPLFQKFRLLKEVNELEIVGEDQRYVKLTPAQRDVLTLALRTGLTKQGRLPFSKLRSILKLGKEVRFNKEKDNRTDLEGDVIHFRVSRPDCFGNRWAAMPVEEQAAVTEKLRTEPDYSALLDWLKNEAGLDEAHARAVADTPVPDGFGRMGPSALSALADAMEHEIDAQGFVITEAEAAKRVYGRTNSEADPGRKGVDQLPKYQEVLQRHIPPGTGEPDDPYDEYMGRITNPTVHIALNQLRRLVNALIRKYGKPNKIAIEVGRELKLNEKQRNEVNREIGQNTRAAMARGQQLVEIFKQPNTGYNRLRLELWEDLNREQPLKRLCTYCGKAIAAHMLFNGETDIDHILPYSKTLDDSKANRLLCCTPCNREKKNYAPANVLQWRDHYGEILARATALPKNKQWRFAEDAMTRYEAEGGFLARQLTDMQYISRLALTYLAHLYDYEEPDLDGVYKRHDRVRALPGRMTEMLRRQWALNELLHGHNLAGGDGAKEKNRLDHRHHAIDAIVIACTSQSLINRLSTAAGEAEERGAARVVERIDPPWPSFREDVREAVNAIVVSHKPDHGTASRSGYDKGRGQTAGKLHNDTAYGETGEKDHNGNNLVVRRIAISDIKRSADIMKIRTNAHGHSELRDRLYEATRDLEGKAFEQAVTAFVKHDAKFKGIRHVRVTEVQNPVWITHGGGKYKKGYLPGGNDRFDVWELPDGKWDAEVVTTFDAHRPDFTPRMRIEHHNARKIMSLKKGDMIAYDDPDSGKRVIAIVRKFDQRNKQLYLDPHNEAGNLDQREKEKTYKPLRPMPNPLKKYRPRQVRVDEIGQVFDPGPWWEKRSD

SEQ ID NO: 31 - RNA, 27 nt, Brucella sp.
  source 1..27: mol_type = other RNA
gttgcggttggcctgcgatttctgaac

SEQ ID NO: 32 - RNA, 81 nt, Brucella sp.
  source 1..81: mol_type = other RNA
gttcagaaatcgcagtccagccgttaacaagctgagatatgcaccaaataaggcgctcgcttcggcgggcgctttttcgtt

SEQ ID NO: 33 - RNA, 141 nt, synthetic construct
  misc_difference 1..29: note = a, c, u, g, unknown or other
  misc_feature 1..141: note = source = /note="Description of Artificial Sequence: Synthetic polynucleotide"
  source 1..141: mol_type = other RNA
nnnnnnnnnnnnnnnnnnnnnnnnnnnnngttgcggttggcctgcgatttctgaacaaaggttcagaaatcgcagtccagccgttaacaagctgagatatgcaccaaataaggcgctcgcttcggcgggcgctttttcgtt

SEQ ID NO: 34 - RNA, 142 nt, synthetic construct
  misc_feature 1..142: note = source = /note="Description of Artificial Sequence: Synthetic polynucleotide"
  source 1..142: mol_type = other RNA
gagcggacagcagcttcctatatctcgtacgttgcggttggcctgcgatttctgaacaaaggttcagaaatcgcagtccagccgttaacaagctgagatatgcaccaaataaggcgctcgcttcggcgggcgctttttcgtt

SEQ ID NO: 35 - RNA, 142 nt, synthetic construct
  misc_feature 1..142: note = source = /note="Description of Artificial Sequence: Synthetic polynucleotide"
  source 1..142: mol_type = other RNA
ccatgatatagacgttgtggctgttgtagtgttgcggttggcctgcgatttctgaacaaaggttcagaaatcgcagtccagccgttaacaagctgagatatgcaccaaataaggcgctcgcttcggcgggcgctttttcgtt

SEQ ID NO: 36-37 - skipped (2 entries)

SEQ ID NO: 38 - protein, 1054 aa, Staphylococcus sp.
MKEKYILGLDLGITSVGYGIINFETKKIIDAGVRLFPEANVDNNEGRRSKRGSRRLKRRRIHRLERVKLLLTEYDLINKEQIPTSNNPYQIRVKGLSEILSKDELAIALLHLAKRRGIHNINVSSEDEDASNELSTKEQINRNNKLLKDKYVCEVQLQRLKEGQIRGEKNRFKTTDILKEIDQLLKVQKDYHNLDIDFINQYKEIVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPQFTEFKLYHDLKSIVFDKSILENEAILDQIAEILTIYQDEQSIKEELNKLPEILNEQDKAEIAKLIGYNGTHRLSLKCIHLINEELWQTSRNQMEIFNYLNIKPNKVDLSEQNKIPKDMVNDFILSPVVKRTFIQSINVINKVIEKYGIPEDIIIELARENNSDDRKKFINNLQKKNEATRKRINEIIGQTGNQNAKRIVEKIRLHDQQEGKCLYSLESIALMDLLNNPQNYEVDHIIPRSVAFDNSIHNKVLVKQIENSKKGNRTPYQYLNSSDAKLSYNQFKQHILNLSKSKDRISKKKKDYLLEERDINKFEVQKEFINRNLVDTRYATRELTSYLKAYFSANNMDVKVKTINGSFTNHLRKVWRFDKYRNHGYKHHAEDALIIANADFLFKENKKLQNANKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 39 - RNA, 28 nt, Staphylococcus sp.
  source 1..28: mol_type = other RNA
gttttagtactctgtaattttaggtatg

SEQ ID NO: 40 - RNA, 85 nt, Staphylococcus sp.
  source 1..85: mol_type = other RNA
tcatacctaaaattacagaatctactgaaacaagactatatgtcgtgtttatcccactaatttattagtgggatttttttgtttt

SEQ ID NO: 41 - RNA, 146 nt, synthetic construct
  misc_difference 1..29: note = a, c, u, g, unknown or other
  misc_feature 1..146: note = source = /note="Description of Artificial Sequence: Synthetic polynucleotide"
  source 1..146: mol_type = other RNA
nnnnnnnnnnnnnnnnnnnnnnnnnnnnngttttagtactctgtaattttaggtatgaaagtcatacctaaaattacagaatctactgaaacaagactatatgtcgtgtttatcccactaatttattagtgggatttttttgtttt

SEQ ID NO: 42 - RNA, 147 nt, synthetic construct
  misc_feature 1..147: note = source = /note="Description of Artificial Sequence: Synthetic polynucleotide"
  source 1..147: mol_type = other RNA
gagcggacagcagcttcctatatctcgtacgttttagtactctgtaattttaggtatgaaagtcatacctaaaattacagaatctactgaaacaagactatatgtcgtgtttatcccactaatttattagtgggatttttttgtttt

SEQ ID NO: 43 - RNA, 147 nt, synthetic construct
  misc_feature 1..147: note = source = /note="Description of Artificial Sequence: Synthetic polynucleotide"
  source 1..147: mol_type = other RNA
ccatgatatagacgttgtggctgttgtagtgttttagtactctgtaattttaggtatgaaagtcatacctaaaattacagaatctactgaaacaagactatatgtcgtgtttatcccactaatttattagtgggatttttttgtttt

SEQ ID NO: 44-45 - skipped (2 entries)

SEQ ID NO: 46 - protein, 1076 aa, Bacillus sp.
MSELDYRIGLDIGTNSIGWGVIELFWNKDRERYEKVRIVDKGVRMFDKAEIPKTGASLAEPRRIARSSRRRLNRKSQRKKEIRNLLVQHGVITQEELDLLYPLSKKSMDIWDIRLDGLDRLLNHLEWTRLLIHLAQRRGFKSNRKSELKDAETGKVLSSIQVNEKRLSLYRTVGEMWIKDAEFSKYDRRRNSPNEYVFSVSRADLEKEIVTLFEAQRKFQSSYSSKNLQETYLQIWAHQLPFASGNAILNKVGYCSLLKGKERRIPKATYTFQYFSALDQVNRTRLGPDFQPFTQEQKEIILDKMFQRTDYYKKKTIPEVSYYDIRKWLELDETIQFKGLNYDPNEELKKIEKKPFINLKAFYEIKKVVANYAERTNEAFSTLDYDAIAYALTVYKTDKDIRSYLKKSNNLSKRCYDDQLIEELFTLSYTKFGHLSFKAINRVLPIMQEGRTYQEAIQQLGYDTTNLKKENRSMFLPLIPDEITNPIVKRALTQARKVVNAIIRRYGSPNSVHIELARELSKSHDERKKIMTAHDENYKKNKGAISILIENGILNPTGYDIVRYKLWKEQGERCAYSLKEIPPDTFFNELKKERNGSSILEVDHILPYSQSFIDSYHNKVLVYSDENRNKGNRIPYTYFLETNKDWEAFERYVRSNKLFSKKKREYLLKKAYLPRESELIKERHLNDTRYASTFLKNFIEQNLQFKEVEVNLRKKRVQTVNGVITAHLRKRWGLEKNRQETYLHHAMDAIIVACTDHHMVTRITEYYQIKESNKSVKKPYFPMPWEGFRDELLSHLASQPIAKKISEELKAGYQSSDYIFVSRMPKRSVTGAAHNQMIRRKGGIDKKGKTIIIKRVRLKDIKFDENGDFKMVGKEQDLATYEAIKQRYLDHGKNLKKAFETPLYKPSKKGTGNLIKRVKIEGQTKAFVREVNGGVAQNSDLVRVDLFEKDDKYYMVPIYVPDTVCSELPKKVVKSGKGYEQWLTLDNSFTFKFSLYPYDLVRLVKGDEDRFLYFGTLDIDSDRLNFKDVNKPSKQNEYRYSLKTIEDLEKYEVGVLGDLRLVRKETRRNFHEIKIN

SEQ ID NO: 47 - RNA, 20 nt, Bacillus sp.
  source 1..20: mol_type = other RNA
gtcatagttccattaaagcc

SEQ ID NO: 48 - RNA, 84 nt, Bacillus sp.
  source 1..84: mol_type = other RNA
ggctttgatgtttctatgataagggtttcggcccgtggcgtcggggatcgcctgcccattccgatgggcttctccccatttatt

SEQ ID NO: 49 - RNA, 137 nt, synthetic construct
  misc_difference 1..29: note = a, c, u, g, unknown or other
  misc_feature 1..137: note = source = /note="Description of Artificial Sequence: Synthetic polynucleotide"
  source 1..137: mol_type = other RNA
nnnnnnnnnnnnnnnnnnnnnnnnnnnnngtcatagttccattaaagccaaagggctttgatgtttctatgataagggtttcggcccgtggcgtcggggatcgcctgcccattccgatgggcttctccccatttatt

SEQ ID NO: 50 - RNA, 138 nt, synthetic construct
  misc_feature 1..138: note = source = /note="Description of Artificial Sequence: Synthetic polynucleotide"
  source 1..138: mol_type = other RNA
gagcggacagcagcttcctatatctcgtacgtcatagttccattaaagccaaagggctttgatgtttctatgataagggtttcggcccgtggcgtcggggatcgcctgcccattccgatgggcttctccccatttatt

SEQ ID NO: 51 - RNA, 138 nt, synthetic construct
  misc_feature 1..138: note = source = /note="Description of Artificial Sequence: Synthetic polynucleotide"
  source 1..138: mol_type = other RNA
ccatgatatagacgttgtggctgttgtagtgtcatagttccattaaagccaaagggctttgatgtttctatgataagggtttcggcccgtggcgtcggggatcgcctgcccattccgatgggcttctccccatttatt

SEQ ID NO: 52-53 - skipped (2 entries)

SEQ ID NO: 54 - protein, 1108 aa, Brevibacillus sp.
MAIRSIKLKLKTRTGPEAQNLRKGIWRTHRLLNEGVAYYMKMLLLFRQESTGGQTKKELQEELVRHIREQQQKNRADKNTQALPLDKAFAALRQLYELLVPSSIGQSGDAQIISRKFLSPLVDPNSEGGKGTSKAGAKPTWQKKKEANDPTWEQDYEKWKKRREEDPTASVITTLEEYGIRPIFPLYTNTVADIAWLPLQSNQFVRTWDRDMLQQAIERLLSWESWNKRVQEEYSKLQEKMTQLNEQLEGGQEWISLLEQYEEQREQELIENMTAANDKYRITKRQMKGWNELYEQWSTVLPNASHEQYREALKRVQQRLRGRFGDAHFFQYLMKEEHHLIWKGNPQRIHYFVARNELKKRLEEAKQNATMTLPDARKHPLWVRFDARGGNLQDYYLTAEADNPRSRRFVTFSQLIWPNESGWMEKQDVEVELALSKQFYQQVTLQKNDKGKQEIEFKDKGSGSTFSGHLGGAKLQLERGDLEKEEKDFEGGEIGSVYLNIVIDFEPLQEVKNGRLQSPYGQVLQLVRRPNEFPKVTTYKSEELVEWMKASQNHSSGVESLESGFRVMSIDLGLRTAAATSIFSVEESNDANAAGFSYWIEGTPLVAVHKRSYMLKLPGEQVEKQVREKRDERQDQQRRVRFQIRILSQVIRMAKKQNRERADELDHLSQALEKQKSLLDQTDRTFWNGIVCDLTDALREKEGGWEQAVVQIHRKAEEHVGKVVQAWRKRFDADERKGIAGLSMWSIEELDSLRKLLISWSRRTRNPQEINRFEQGHTSHQRLLTHIQNVKEDRLKQLSHAIVMTALGYVYDEKKLEWFAKYPACQVILFENLSQYRSHMDRSTKENSTLMKWAHRSIPKYVHMQAEPYGIQIGDVRAEYSSRFHAKTGTPGIRCKMVKGQELQGKRFENLQKRLVSEQFLTEEQVKQLRPGDIVPDDSGEWFMTLSDGSEGKEVVFLQADINAAQNLQKRFWQRYNELFKVSCRVLIRGEEEYLIPKAKSVQAKLGKGLFVKKTDTVMKDVYVWDSQAKLKGKTTFTEESESPEQLEDFQEIIEEAEEAKGTYRTLFRDPSGVFFPEFVWNTQKDFWSEVKRRLYGKLRERFLMKTR

SEQ ID NO: 55 - RNA, 22 nt, Brevibacillus sp.
  source 1..22: mol_type = other RNA
tggaaagcttcgaggttagcac

SEQ ID NO: 56 - RNA, 105 nt, Brevibacillus sp.
  source 1..105: mol_type = other RNA
cgacggttagaggccgtatgtcgatttgctttaatttcgtgcgtgtgcattgtcgtcctccattacagggcggctaccacgaatagtcacgaagttccactgagt

SEQ ID NO: 57 - RNA, 160 nt, synthetic construct
  misc_difference 132..160: note = a, c, u, g, unknown or other
  misc_feature 1..160: note = source = /note="Description of Artificial Sequence: Synthetic polynucleotide"
  source 1..160: mol_type = other RNA
cgacggttagaggccgtatgtcgatttgctttaatttcgtgcgtgtgcattgtcgtcctccattacagggcggctaccacgaatagtcacgaagttccactgagtaaagtggaaagcttcgaggttagcacnnnnnnnnnnnnnnnnnnnnnnnnnnnnn

SEQ ID NO: 58 - RNA, 161 nt, synthetic construct
  misc_feature 1..161: note = source = /note="Description of Artificial Sequence: Synthetic polynucleotide"
  source 1..161: mol_type = other RNA
cgacggttagaggccgtatgtcgatttgctttaatttcgtgcgtgtgcattgtcgtcctccattacagggcggctaccacgaatagtcacgaagttccactgagtaaagtggaaagcttcgaggttagcacgtacgagatataggaagctgctgtccgctc

SEQ ID NO: 59 - RNA, 161 nt, synthetic construct
  misc_feature 1..161: note = source = /note="Description of Artificial Sequence: Synthetic polynucleotide"
  source 1..161: mol_type = other RNA
cgacggttagaggccgtatgtcgatttgctttaatttcgtgcgtgtgcattgtcgtcctccattacagggcggctaccacgaatagtcacgaagttccactgagtaaagtggaaagcttcgaggttagcacactacaacagccacaacgtctatatcatgg

SEQ ID NO: 60 - skipped

SEQ ID NO: 61 - protein, 1150 aa, Enterococcus sp.
MYSIGLDLGISSVGWSVIDERTGNVIDLGIRLFSAKNSEKNLERRTNRGGRRLIRRKTNRLKDAKKILAAVGFYEDKSLKNSCPYQLRVKGLTEPLSKGEIYKVTLHILKKRGISYLDEDDTEAAKESQDYKEQVRENAQLLTKYTPGQIQLQRLKENNRVKTGINAQGNYQLNVFKVSAYANELATILKTQQAFYPNELTDDWIALFVQPGIAEEAGLIYRKRPYYHGPGNEANNSPYGRWSDFKKTGQPATNIFDKLIGKDFQGELRASGLSLSAQQYNLLNDLTNLKIDGEIPLSPEQKEYILAELMTKEFTRFGVNDVVKLLGVKKERLSGWRLDKKGKPEIHTLKGYRNWRKIFAESGIDLATLPTETIDCLAKVLTLNTEREGIENTLAFELSELAESVKLLVLDRYKELSQSVSTQAWHRFSLKTLHLLIPELMNATSEQNTLLEQFQLKSDVRKRYSEYKKLPTKDVLTEIYNPTVNKTVSQAFKVIDALLVKYGKEQIRYITIEMPRDDNEEDEKKRIKELHAKNSQRKNDSQSYFMQKSGWSQEKFQTTIQKNRRFLAKLLYYYEQDGICAYTGLSISPELLVSDSTEIDHIIPISISLDDSINNKVLVLSKANQVKGQQTPYDAWMDGSFKKINGKFSNWDDYQKWVESCHFSHKKENNLLETRNIFDSEQVEKFLARNLNDTRYASRLVLNTLQSFFANQETKVRVVNGSFTHTLRKKWGADLDKTRETHHHHAVDATLCAVTPFVKVSRYHYAVKEETGEKVMREIDFETGEIVDEMSYREFKKSKKYERKTYQVKWPNFREQLKPVNLHPRIKFSHQVDRKANRKLSDATIYSVREKTEVKTLKSGKQKITTDEYTIGKIKDIYTVDGWEAFKKKQDKLLMKDLDEKTYERLLSIAETTPDFQEVEEKNGKVKRVKRSPFAVYCEENDIPAIRKYAKKNNGPLIRSLKYYDGKLNKHINITKDSQGRPVEKTKNGRKVTLQSLKPYRYDIYQDLETKAYYTVQLYYSDLRFVEGKYGITEKEYMKKVAEQTKGQVVRFCFSLQKNDGLEIEWKDSQCYDVRFYNFQSANSINFKGLEQEMMPAENQFKQKPYNNGAINLNIAKYGKEGKKLRKFNTDILGKKHYLYYEKEPKNIIK

SEQ ID NO: 62 - RNA, 19 nt, Enterococcus sp.
  source 1..19: mol_type = other RNA
gtttttgtactctcaataa

SEQ ID NO: 63 - RNA, 73 nt, Enterococcus sp.
  source 1..73: mol_type = other RNA
ttattgagaatctacaaaaataaggcattttgccgaatttaccgccctacatatgtagggcggtttttttatt

SEQ ID NO: 64 - RNA, 125 nt, synthetic construct
  misc_difference 1..29: note = a, c, u, g, unknown or other
  misc_feature 1..125: note = source = /note="Description of Artificial Sequence: Synthetic polynucleotide"
  source 1..125: mol_type = other RNA
nnnnnnnnnnnnnnnnnnnnnnnnnnnnngtttttgtactctcaataaaaagttattgagaatctacaaaaataaggcattttgccgaatttaccgccctacatatgtagggcggtttttttatt

SEQ ID NO: 65 - RNA, 126 nt, synthetic construct
  misc_feature 1..126: note = source = /note="Description of Artificial Sequence: Synthetic polynucleotide"
  source 1..126: mol_type = other RNA
gagcggacagcagcttcctatatctcgtacgtttttgtactctcaataaaaagttattgagaatctacaaaaataaggcattttgccgaatttaccgccctacatatgtagggcggtttttttatt

SEQ ID NO: 66 - RNA, 126 nt, synthetic construct
  misc_feature 1..126: note = source = /note="Description of Artificial Sequence: Synthetic polynucleotide"
  source 1..126: mol_type = other RNA
ccatgatatagacgttgtggctgttgtagtgtttttgtactctcaataaaaagttattgagaatctacaaaaataaggcattttgccgaatttaccgccctacatatgtagggcggtttttttatt

SEQ ID NO: 67-68 - skipped (2 entries)

SEQ ID NO: 69 - protein, 1408 aa, Sphingobacterium sp.
MKRILGLDLGTSSIGWAYVLEAQDENEISKIEKLGVRVNPLTTDEQLNFEKGKPITTNAGRTLARSARRNLQRFKLRRNNLIEVLKREKWIDDKSILAENGNKSTFETYALRAAAASKEIKLEELARVLLMINKKRGYKSSRKVIKEDDGQLVDGMEVAKKLYHENLTPGEFVCRLLKEGKKKLPDFYRSDLQRELDLIWDFQFKYYPELLTRDFKEELKGKGLRATSAIFWSKYKFNTAENKGTKEEKKIQAYQWRKDGLTKQLAKEEVAYIIAEINGQLANSSGYLGAISDRSKELYFNKQTVGQFLFNQLQQNTHARLKNQVFYRQDYLDEFEKIWCIQSQFHQALTEQLKEEIRDIIIFYQRKLKSQKGLISFCEFEQHEVVVNGKNKVVGLRVAPKSSPIFQEFKIWQQLNNVKLRNNITKEVYNLAEEQKKLLFETLNLKGKLSAYQVLSLIEGKPKEWELNYTELEGNSTNSALYNAYLNILDIEGYDVRSELKIKLNKDEITLSDLDIPVSEIKDMIRRIFQHLGINTSILDFDAQMQGDDFEKQLSYQLWHLLYSYEEDDSKTGMDRLYNQLNLKFGFSLDQAKFIGKIALQDDYGNLSTKAIRQIYPYIQDAEYSAACKLAGYNHSKSSLTKEQLTNRVLKDHLDILPKNSLRNPVVEKILNQMVNVVNTLIDTENDKLIKEGKNADFRFDEIRIELARELKKNAKEREELTKAINTSKEQHEKIIKILQTEDGIKNPTRNDIIRFKLYQELKNNGYKNLYTNEYIQRKDLFTNIYDIDHIVPQSRLFDDSFSNKVLVPRNINIEKGNQTAFDYVRTKFGEDGIEAYEARVERLFNLKEEGVSRSKYKKLLMRGSDIGEGFIERDLRDSQYIAKKAKAMLFEITPSVISTSGSVTDRLREDWGLVSVMKELNLPKFKAVGLTEYLETKDGNRKEVIKDWSKRNDHRHHAMDALTVAFTKHSHIQYLNHLNARKNEKSEFFSSIKAIEIKETYVERDDLGNRKRLFKEPIAHFRTIVKEHLERVLVSHKAKNKVVTKNRNKIDGKKEAQEVLTPRGQLHKETIYGKILQYASKEEKISAKFDAATIAMVSNPRYRSALLDRLIEYGNDPKKAFTGKNSPSKSPIYLDQQRQLAVPEKVKLVWLEADYTIRKEIGPDLKIEKVIDQGVKRILKQRLSVYDGDTKKAFSDLDNNPIWLNEEKGIAIKRVTISGVKNAEALHTKKDHLGQVLEDKDGNTMPVDFVSTGNNHHVAIYEDSEGNLYDRVVSFYEAVERINQNLPIVDRHYKQEEGWKFLFSMKQNELFVFPSINFDPKEIDLLDKKNYKEVSKHLFRMQKFSKVEYGNSAVRDYVFRHHLETSIIDTKELRDIAYKVFKSIGEFKSFVKIRTNHLGEIVHLGEY

SEQ ID NO: 70 - RNA, 16 nt, Sphingobacterium sp.
  source 1..16: mol_type = other RNA
gttgtgaattgctttc

SEQ ID NO: 71 - RNA, 72 nt, Sphingobacterium sp.
  source 1..72: mol_type = other RNA
gaaagcaattcacaataaggattatttccgttgtgtaaacatttagcgcctcgtctatctacggggcatttt

SEQ ID NO: 72 - RNA, 121 nt, synthetic construct
  misc_difference 1..29: note = a, c, u, g, unknown or other
  misc_feature 1..121: note = source = /note="Description of Artificial Sequence: Synthetic polynucleotide"
  source 1..121: mol_type = other RNA
nnnnnnnnnnnnnnnnnnnnnnnnnnnnngttgtgaattgctttcaaaggaaagcaattcacaataaggattatttccgttgtgtaaacatttagcgcctcgtctatctacggggcatttt

SEQ ID NO: 73 - RNA, 122 nt, synthetic construct
  misc_feature 1..122: note = source = /note="Description of Artificial Sequence: Synthetic polynucleotide"
  source 1..122: mol_type = other RNA
gagcggacagcagcttcctatatctcgtacgttgtgaattgctttcaaaggaaagcaattcacaataaggattatttccgttgtgtaaacatttagcgcctcgtctatctacggggcatttt

SEQ ID NO: 74 - RNA, 122 nt, synthetic construct
  misc_feature 1..122: note = source = /note="Description of Artificial Sequence: Synthetic polynucleotide"
  source 1..122: mol_type = other RNA
ccatgatatagacgttgtggctgttgtagtgttgtgaattgctttcaaaggaaagcaattcacaataaggattatttccgttgtgtaaacatttagcgcctcgtctatctacggggcatttt

SEQ ID NO: 75 - protein, 1429 aa, Chryseobacterium sp.
MMIKHILGLDLGTNSIGWALIKQNFENKYGEILGMGSRIIPMSQDILGEFGKGNSVSQTAARTDYRGIRRLRERFLLRRERLHRILNVLNFLPEHYASQIDFDKRFGKFKVETEPKLAWKNSDGKFSFLFQTSFNEMLEDFKAHGQDLKVPYDWTIYYLRKKALSQKIEKEELAWILLNFNQKRGYYQLRGEEEEENPNKLIEFYSLKIIDVLADEPQKGKSDIWYSLVLENGWIYRRSSKTSLLDWKDKIRDFIVTTDLNDNGSVKTDKEGNEKRSFRAPGENDWTLVKTKTEQEIDRSRKTVGTYIYETLLQNPKQKIKGKLVRTIERKFYKEELKQILEKQKEFHYELQSDDLYDDCIRELYRNNEAHQLTLSKKDFVHLFMEDIIFYQRPLKSQKSSISNCTLEFRKYKDENGVEHTQFLKAIPKSNPYYQEFRIWQWIFNLNIYKKDNDDNVTKEFLSTTEDFENLFEFLNNRKEIDQKALLKHFKLNEKTHRWKYVEDKKYPCNETKTMISERLKKVENISDDFLTRGMEQKIWHIIYSVNDKTEYEKALKSFAEKNNLDENSFFEAFRKFPPFKSEYGSFSEKAIKKLLPLMRLGKYWSYANIDLYSKNRIQKIITGEFDENIKDRVREKAIHLTAENDFQGLQLWLAQYIVYGRHSEATMIGKWNSADDLEEFLKEFKQHSLRNPIVEQVITETLRVVKDIWLKYGNGAKDFFNEVHIELGREMKQTKDERANATKTITENENTNLRIKALLAEMMNDHSVENVRPYSPMQQEILKIYEDGILKSDIEIDDDILKISKTAQPSSSDLKRYKLWLEQKYKSPYTGQIIPLNKLFTPEYEIEHIIPQSRYFDDSLSNKIICESAVNKLKDNYIGLGFIKQFGGTIIECGFGKRVKVFKAEEYEEFVKKHYANNRGKRNKLLLEDIPEKMIERQMNDTRHISKYISGILSHIVRVEDGTDEGVNSKNVIPGNGKITTQLRQDWGLNDVWNELVLPRFERMNQLTNSTDFTSWNENHQKYLPTVPVEFSKGFSKKRIDHRHHALDALVIACATKDHVNLLNNQSAKSDTKRYDLKKKLMKFEKTVYKDPQTEKRIEREVPKYFLKPWETFTVDAKNKLETIIVSFKQNLRVINKATNYYEKYVDKDGVKTKERVEQTGTNWAIRKPMHKETVSGKIDLPWVKVPKGKILTATRKSLDTSFDLKAIASITDTGIQKILKNYLEFKESPELAFSPEGIEDMNKNIKKYNGGKPHQPISKVRVFELGSKFQVGQTGNKKDKYVEAAKGTNLFFAIYEDRKGKRSYETIPLNEVIERQKQGLSVVDLKNINDFFLCPNDLVYIPSGDELENGGSIEIKNITKEKSERFYKVVSFSGSQIFFVRHDIAVSIVNKGEFSTLNKMERAIDGSMVKESCIKLKIDRLGNVLKA

SEQ ID NO: 76 - RNA, 19 nt, Chryseobacterium sp.
  source 1..19: mol_type = other RNA
gttgtgaattgctttcaaa

SEQ ID NO: 77 - RNA, 75 nt, Chryseobacterium sp.
  source 1..75: mol_type = other RNA
tttgaaaagcaattcacaataaggattattccgttgtgaaaacattcaaggcggggcaactcgccttttttcgtt

SEQ ID NO: 78 - RNA, 127 nt, synthetic construct
  misc_difference 1..29: note = a, c, u, g, unknown or other
  misc_feature 1..127: note = source = /note="Description of Artificial Sequence: Synthetic polynucleotide"
  source 1..127: mol_type = other RNA
nnnnnnnnnnnnnnnnnnnnnnnnnnnnngttgtgaattgctttcaaaaaagtttgaaaagcaattcacaataaggattattccgttgtgaaaacattcaaggcggggcaactcgccttttttcgtt

SEQ ID NO: 79 - RNA, 128 nt, synthetic construct
  misc_feature 1..128: note = source = /note="Description of Artificial Sequence: Synthetic polynucleotide"
  source 1..128: mol_type = other RNA
gagcggacagcagcttcctatatctcgtacgttgtgaattgctttcaaaaaagtttgaaaagcaattcacaataaggattattccgttgtgaaaacattcaaggcggggcaactcgccttttttcgtt

SEQ ID NO: 80 - RNA, 128 nt, synthetic construct
  misc_feature 1..128: note = source = /note="Description of Artificial Sequence: Synthetic polynucleotide"
  source 1..128: mol_type = other RNA
ccatgatatagacgttgtggctgttgtagtgttgtgaattgctttcaaaaaagtttgaaaagcaattcacaataaggattattccgttgtgaaaacattcaaggcggggcaactcgccttttttcgtt

SEQ ID NO: 81 - skipped

SEQ ID NO: 82 - protein, 1440 aa, Chryseobacterium sp.
MKNILGLDLGTNSIGWALVKQDFKNKQGEILGMGTRIIPMSQDILGDFGKGNSVSQTAERTKYRSARRLRERFLLRRERLHRVLNILNFLPEHYASQIDFEKRFGKFKVETEPKLAWKNIEGQFSFLFQNSFNEMLEDFKANAQDLRIPYDWTIYYLRKKALSQKIEKEELVWILLNFNQKRGYYQLRGEEEEENPNKLVELYSLKVVDVFVDEPQKGKSDIWYSLVLENGWIYRRASKISLFDWKDKIRDFIVTTDLNDDGSVKTDKDGNEKRSFRAPGENDWTLVKKKTEQEIEQSHKTVGMYIYETLRANPKQKIKGKLVRTIERKFYKEELRQILEKQKEFHQELQNDDLYNDCIRELYRNNEAHQLTLSKKDFVHLFIEDIVFYQRPLRSQKSSISNCTLEFRKYKDENGAEHTQYLKAIPKSNPYYQEFRLWQWIFNLNLYKKDNDENVIKEFLTTTQDVENLFEFLNNRKEIDQKALLKHFKLSEKTHRWNFVEDKKYPCNETKTMIATRLEKVENISDDFLTREIEQKIWHIIYSVNDKIEYEKALKSFALKHNLDENSFFEAFRKFPPFKSEYGSFSEKAIKKLLPLMRLGKYWDYANIDQFSKGRIQKIINGEYDENIKDRVREKAVHLTSENDFQGLQLWLAQYIVYGRHSEASIAGKWNSADDLEEFLKDFKHHSLRNPIVEQVITETLRVVKDIWLKYGKGAKDFFNEIHIELGREMKLPADDRKKLTNQITENENTNLRIKALLAEMMNDHGVENVRPFSPQQQEILKIYEDGVLNSDIEIEDEYLKISKTAQPSPSDLKRYKLWLEQKYKSPYTGQIIPLNKLFTPEYEIEHIIPQSRYFDDSFSNKIICESAVNKLKDNYIGLGFIKQFGGTIIELGFGKSVKVFDIDEYEDFVKKHYANNRSKRNKLLLEDIPEKMIERQINDTRYISKYISGILSNIVRLEDGSDEGINSKNIVPGNGKITTQLKKDWGLNDVWNDLILPRFKRMNQLTNSTDFIAWNENFQKFLPTVPIEYSKGFSKKRIDHRHHALDALVIACATKDHVNLLNNQSAKSETKRYDLKKKLMKFERVVYLHTQTGEKIEREVPKHFLKPWENFTVDVKHNLDTVIVSFKQNLRVINKATNYYEKYAENGGARNKARVEQKGINWAIRKPMHKDTVSGKVDLPWVKVPKGKILTATRKSLDTSFDLKSIGSITDTGIQKILKNYLMLKDGNPELAFSPEGIEDLNKSIEKYNDGKPHQPINKVRVFELGSKFQIGQAGNKKDKYVEAAKGTNLFFAVYEDKNGKRSYETIPLNEVIERQKQGLPVVDLRSTNDFYLCPNDLVYILSDDERGNMTNNDFENLSDEQVKRIYKFVSCTGGEGHFIPYANATEIIKNENGTNSKSERMQNFYDGSCMLDKNSKPIMIKENCVKLKVDRLGNISKI

SEQ ID NO: 83 - RNA, 20 nt, Chryseobacterium sp.
  source 1..20: mol_type = other RNA
gttgtgaattgctttcaaaa

SEQ ID NO: 84 - RNA, 79 nt, Chryseobacterium sp.
  source 1..79: mol_type = other RNA
ttttgaaagcaattcacaataaggattattccgttgtgaaaacatttagagcctcgactaccttcggggcatttttatt

SEQ ID NO: 85 - RNA, 132 nt, synthetic construct
  misc_difference 1..29: note = a, c, u, g, unknown or other
  misc_feature 1..132: note = source = /note="Description of Artificial Sequence: Synthetic polynucleotide"
  source 1..132: mol_type = other RNA
nnnnnnnnnnnnnnnnnnnnnnnnnnnnngttgtgaattgctttcaaaaaaagttttgaaagcaattcacaataaggattattccgttgtgaaaacatttagagcctcgactaccttcggggcatttttatt

SEQ ID NO: 86 - RNA, 133 nt, synthetic construct
  misc_feature 1..133: note = source = /note="Description of Artificial Sequence: Synthetic polynucleotide"
  source 1..133: mol_type = other RNA
gagcggacagcagcttcctatatctcgtacgttgtgaattgctttcaaaaaaagttttgaaagcaattcacaataaggattattccgttgtgaaaacatttagagcctcgactaccttcggggcatttttatt

SEQ ID NO: 87 - RNA, 133 nt, synthetic construct
  misc_feature 1..133: note = source = /note="Description of Artificial Sequence: Synthetic polynucleotide"
  source 1..133: mol_type = other RNA
ccatgatatagacgttgtggctgttgtagtgttgtgaattgctttcaaaaaaagttttgaaagcaattcacaataaggattattccgttgtgaaaacatttagagcctcgactaccttcggggcatttttatt

SEQ ID NO: 88 - skipped

SEQ ID NO: 89 - protein, 1071 aa, Bacillus sp.
MRELDYRIGLDIGTNSIGWGVIELSWNKDREQYEKTRIVDKGVRMFDKAEIPKTGASLAEPRRIARSSRRRLNRKSQRKKDIRNLLVQHEIISQKELTSLYPLSKSSMDIWDIRLDGLDRLLDRFEWARLLIHLAQRRGFKSNRKSELKDVETGKVLSSIQVNEKRLSLYRTVGEMWMKNADFSKYGKRRNSPNEYVFSVSRADLEKEIVTLFEAQRKFHSSYASVDLQKTYIQIWAHQLPFASGNAIVNKVGYCSLLKGKEKRVPKATYTFQYFNTLDQINRTRLGPNFQPFTKEQRDIILDKMFQRTDYYKKKTIPEVTYYDIRKWLALDETIQFKGLTYDPNEELKKIEMKPFINLKPFYEIKKVVTNYAKKTNEVFSALDYDTVAYALTVYKTDKDIRSYLKRSNNLSKRCYDDQLIEELLTLSYTKFGHLSFKAINHVLPIMQEGRTYQEAIHQLGYDTTNLKKENRSMFLPIIPDEITNPIVKRALTQARKVVNAIIRRYGSPNSVHIELARELSKSHDERKKIMTAHDENYKKNKGAISILIENGILNPTGYDIVRYKLWKEQGERCAYSLKKISANTFFNELKKERSGPPVLEVDHILPYSQSFIDSYHNKVLVYGDENQKKGNRIPYTFFSEEDKEWESFESYVRSNSFFSKKKRGYLLKKAYLPRESNLIKERHLNDTRYASSYLKNFIEKNLKFKEAVGITRKKYVQTVNGVITAHLRKRWGLEKDRQETYLHHAMDAIIVACTDHHMVTKVTEYYQIKEGNKSIKKPYFPLPWMGFREEILSHLESQPIARKISEELKIGYQSSDYILVSRMPKRSVTGSAHDQTVMKKGGIDKKGKTIIIKRVHLKDIKFDENGDFKMVGKEQDLATYEAIKQRYLEYRKESKKAFETPLYKPSKKGKGNLIKKIKVEVQTKSFVREINGGVAQNGDLVRVDLFEKDNRYYMVPIYVVDTVRSELPNKAVTSSKGYEQWLSIDNSFTFKFSLYPYDLVRLVKGDEDRFLYFSTLDINSDRLNFKDVNKPSKQAEYRYSLKTIENLEKYEIGVLGDLRLVRQETRKIFK

SEQ ID NO: 90 - RNA, 24 nt, Bacillus sp.
  source 1..24: mol_type = other RNA
gtcatagttccattaaagccattg

SEQ ID NO: 91 - RNA, 138 nt, Bacillus sp.
  source 1..138: mol_type = other RNA
caatggctttgatgtttctatgataagggcttcggcccgtggcgttggggatcgcctgcccattttaatgggcttctccccatctatttaatgagaattttacaaccttggctattcttaaatagctaaggttttttt

SEQ ID NO: 92 - RNA, 195 nt, synthetic construct
  misc_difference 1..29: note = a, c, u, g, unknown or other
  misc_feature 1..195: note = source = /note="Description of Artificial Sequence: Synthetic polynucleotide"
  source 1..195: mol_type = other RNA
nnnnnnnnnnnnnnnnnnnnnnnnnnnnngtcatagttccattaaagccattgaaagcaatggctttgatgtttctatgataagggcttcggcccgtggcgttggggatcgcctgcccattttaatgggcttctccccatctatttaatgagaattttacaaccttggctattcttaaatagctaaggttttttt

SEQ ID NO: 93 - RNA, 196 nt, synthetic construct
  misc_feature 1..196: note = source = /note="Description of Artificial Sequence: Synthetic polynucleotide"
  source 1..196: mol_type = other RNA
gagcggacagcagcttcctatatctcgtacgtcatagttccattaaagccattgaaagcaatggctttgatgtttctatgataagggcttcggcccgtggcgttggggatcgcctgcccattttaatgggcttctccccatctatttaatgagaattttacaaccttggctattcttaaatagctaaggttttttt

SEQ ID NO: 94 - RNA, 196 nt, synthetic construct
  misc_feature 1..196: note = source = /note="Description of Artificial Sequence: Synthetic polynucleotide"
  source 1..196: mol_type = other RNA
ccatgatatagacgttgtggctgttgtagtgtcatagttccattaaagccattgaaagcaatggctttgatgtttctatgataagggcttcggcccgtggcgttggggatcgcctgcccattttaatgggcttctccccatctatttaatgagaattttacaaccttggctattcttaaatagctaaggttttttt

SEQ ID NO: 95 - protein, 1091 aa, Rhizobium sp.
MDIETLFAFDIGTNSIGFCVFALDENGEPYRIIDIGTRIYADGRDPQSKASLAVARREARAMSRRRDRYVNRRKAVLRTLTEYGLMPAEASARQALIAETSDRNGAAGEASANPYALRAMALDEKLPLYWIGRVLFHLNQRRGFKSNRKTDRKDNEKGVIALGIGELRAAMHKTKARTYGEWLAARREDGHVTRLRAGSDAFEGDGYAFYPERSLLEAEFREIWARQSSFYPDVLTEARGDHLFQVMFYQRPLKKPRVGKCAFNPLEERLAKAHPLFQEFRLYKEVNELELVLPDHSHKKLDIEQRNALVILLRANREVTFKKLRTALKLAPDIVFNKESESREKLRGDEVHSVLADKKRFGPQWGALSRERQWQIIQTLKDEENPEKLFGWLKEEFGFEGEKADAIANAPLPEGYGRLGETALSSMLDEMKAAVIPEAEAAKNCGYDHSKLGEDREEGEAFLPPYQEILSRQIPPGTNDPADIYDIRMGRFTNPTVHIGLNQLRRVVNALIARHGKPQFVSLELARDLQLSEKQKAEANRAIAKNTREAAARSKKLGEMGQLDTGYNRLLLKLWEELNPSKPEDRVCIYSGKPIGIDMLFSAEVDVDHILPWSKTLDDSQANKLLCLKSANRQKRNRAPADVPEWRDRYEEILARAARLAPNKRRRFAPNAMAEFEAEGGFLARQLTDTQYLSRMAREYLSCLFPSAEIDRRGERKKKIRVIVSPGRLTEMLRRNWGLNNILPDHNLGEMTQAKNRKDHRHHAIDAAVVGVTTRSLLQRIATAAGRLDEADFENLVRKMVSENPPWPTFREELQAAVNGIVVSHKPDHGTVSRKGYAEGKGRTAGRLHNDTAYGFGTDASGNPVAVRRKLFTSLEAKDIPMIRDPELQAELYAAIDGLDDRRALQDALLSFRRTHPKFKGIRRVRMAETLSFIPIRDSQGNVYKGYKGDANYRYDVWETLDGKWHADVVTMFNAHQPDWRSPVHQEHPTARRVLRLQQNDMVAYEHPNDGYTIARVVKFNTAGIVYFASHRESGSLKARDADKQDPFKYFSKSAAGLKDIQCRQIRIDAAGRVFDPGPQDRASKSTRKTN

SEQ ID NO: 96 - RNA, 29 nt, Rhizobium sp.
  source 1..29: mol_type = other RNA
gttgcggctggaccgcgttttctgatctg

SEQ ID NO: 97 - RNA, 78 nt, Rhizobium sp.
  source 1..78: mol_type = other RNA
cagatcagaaaacgcggtctggccgttaacaagctagaagcaccaaataaggccgttccttcgggagcggctttttct

SEQ ID NO: 98 - RNA, 140 nt, synthetic construct
  misc_difference 1..29: note = a, c, u, g, unknown or other
  misc_feature 1..140: note = source = /note="Description of Artificial Sequence: Synthetic polynucleotide"
  source 1..140: mol_type = other RNA
nnnnnnnnnnnnnnnnnnnnnnnnnnnnngttgcggctggaccgcgttttctgatctgaaagcagatcagaaaacgcggtctggccgttaacaagctagaagcaccaaataaggccgttccttcgggagcggctttttct

SEQ ID NO: 99 - RNA, 141 nt, synthetic construct
  misc_feature 1..141: note = source = /note="Description of Artificial Sequence: Synthetic polynucleotide"
  source 1..141: mol_type = other RNA
gagcggacagcagcttcctatatctcgtacgttgcggctggaccgcgttttctgatctgaaagcagatcagaaaacgcggtctggccgttaacaagctagaagcaccaaataaggccgttccttcgggagcggctttttct

SEQ ID NO: 100 - RNA, 141 nt, synthetic construct
  misc_feature 1..141: note = source = /note="Description of Artificial Sequence: Synthetic polynucleotide"
  source 1..141: mol_type = other RNA
ccatgatatagacgttgtggctgttgtagtgttgcggctggaccgcgttttctgatctgaaagcagatcagaaaacgcggtctggccgttaacaagctagaagcaccaaataaggccgttccttcgggagcggctttttct

SEQ ID NO: 101-102 - skipped (2 entries)

SEQ ID NO: 103 - protein, 1401 aa, Myroides sp.
MKRILGIDLGTTSIGWAYVHEAENENEISSIEKLGVRVNPLTVDEQINFEKGKPITTNATRTLFRSARRNLQRFKLRRKHLIEILKHNNWISKQTLLTENGNHTTFETISLRDKAATQKVTLEELARVLLMINKKRGYKSSRKVNNTEEGQLIDGMEVAKKLYHEQLTPGQLSLQLIKQGVKRLPDYYRSDLNHELDLIWAFQQKFYPHILTSEFKELLKGKGLRATSNQFWTNYNFNTADNKGTREEKKLQNYQWRSDALIKQLDKEIVAYVISEINGQINNSSGYLGAISDRSKELYFNQLTVGQYLHRQLIANPHTRLKSQVFYRQDYLDEFEKIWETQSKFYPKELTTVLKNEIRDTVIFYQRKLKSQKGLISFCEFEQEQKIINGKTKTIGHRVIPKSSPLFQEFKIWQQLHNVVLRNKKTNVITPLAEEQKSHLFEELNLKGKLSSTQILKLIEDKPKDWELNYSELEGNNTNKALYNAYLDILDLEGYDIRDELKIKLNKDDIELSDLDVNASEIKEMIFSIFKHLGINTEILEFDATLKDKAFEKQASYQLWHLLYSYEEDNSPTGLDRLHNLLQKKFNFTLDQAKLVGNVLFQDDYGNLSSKAIKNIFPYITDNNYSTACELAGYKHSKHSLTKEENEKRELKSRLTILTKNSLRNPVVEKILNQMINVINTLIESENDKLVAQGKEPNFQFDEIRIELARELKKNAKEREELTKSMTQGKTNHEKIIKILQKEDGIKNPTRNDITRFKLYSELKNNGYKDLYTNEYIERKDIFSKEYDIEHIIPQSKLFDDSFSNKTLVRRNVNLKKGNQTAYDFILSEYGQEKANEFETRITNLYSLGKDEGISKSKYKKLLMQESEIGKGFIERDLRETQYIAKKAKSLLLEITRNVVSTSGGITDRLREDWGLVNVMKELNLKKFRDAGLTEFVEMKDGNKKEVIIDWTKRNDHRHHAMDALTIAFTKHNHIQYLNHLNARKNTKDELHTNIIAIEKKETTIITDERGNKKRIFKEPIPNFRNIAKSHLEAILVSHKAKNKVVTKNINKISGKKIGQQTLTPRGQLHKETVYGKIRQYVSKEEKVGPKFTKEVIELVSNPTYRDLLLKRLQENNNDPKKAFGGKNALSKSPIYINLEKNIIVPEVVKLVWLEDDYTIRKEISPELKIDKVVDEGVKRILQNRLALYNGDAKIAFSDLDNNPIWLSKDKGITIKRVTLSGVKNATALHTKKDHLGQPILDHKGQEIPVDFISTGNNHHVAIYEDKNGKLQESVVSHFEAVERVNQQLPIIDKTFNQHLGWKFLFTMKQNELFLFPSDDFNPNEIDLFDTRNYAVISKHLFRAQKLSTKDYSFRHHLETNVEDIKDLKTITWRREGLNGIQNIVKIRTNHLGEIVHIGEY

SEQ ID NO: 104 - RNA, 21 nt, Myroides sp.
  source 1..21: mol_type = other RNA
gttgtgaattgctttcaaaca

SEQ ID NO: 105 - RNA, 105 nt, Myroides sp.
  source 1..105: mol_type = other RNA
tgaaagcaattcacaataaggattattccgttgtgaaaacatctagtcctcgactaatttcggggaataaagccttaactctgttaaggctttttttagtttttt

SEQ ID NO: 106 - RNA, 159 nt, synthetic construct
  misc_difference 1..29: note = a, c, u, g, unknown or other
  misc_feature 1..159: note = source = /note="Description of Artificial Sequence: Synthetic polynucleotide"
  source 1..159: mol_type = other RNA
nnnnnnnnnnnnnnnnnnnnnnnnnnnnngttgtgaattgctttcaaacaaaagtgaaagcaattcacaataaggattattccgttgtgaaaacatctagtcctcgactaatttcggggaataaagccttaactctgttaaggctttttttagtttttt

SEQ ID NO: 107 - RNA, 160 nt, synthetic construct
  misc_feature 1..160: note = source = /note="Description of Artificial Sequence: Synthetic polynucleotide"
  source 1..160: mol_type = other RNA
gagcggacagcagcttcctatatctcgtacgttgtgaattgctttcaaacaaaagtgaaagcaattcacaataaggattattccgttgtgaaaacatctagtcctcgactaatttcggggaataaagccttaactctgttaaggctttttttagtttttt

SEQ ID NO: 108 - RNA, 160 nt, synthetic construct
  misc_feature 1..160: note = source = /note="Description of Artificial Sequence: Synthetic polynucleotide"
  source 1..160: mol_type = other RNA
ccatgatatagacgttgtggctgttgtagtgttgtgaattgctttcaaacaaaagtgaaagcaattcacaataaggattattccgttgtgaaaacatctagtcctcgactaatttcggggaataaagccttaactctgttaaggctttttttagtttttt

SEQ ID NO: 109 - skipped

SEQ ID NO: 110 - protein, 1432 aa, Chryseobacterium sp.
MAKNILGLDLGTNSIGWALINQDFENKQGKILGMGSRIIPMSQDILGDFGKGNSVSQTAARTGYRGVRRLRERFLLRRERLHRVLNIINFLPEHYASQIDFEKRFGKFKEETEPKLAYNKDGFVFKDSFEEMLADFKNYQPQLLENDKKIPYDWTIYYLRKKALSQKIEKEELAWILLNFNQKRGYYQLRGEDFEEEKDKMFVRLKVEKIIDSGDNVKGKILYDVYFENGWKYDKQIVKTEDWIERVKEFIVTESFLKNGDIKRTFKAVDSEKDWIAIKTKTEQEIDKSHKTVGVYIYETLLQNPKQKIKGKLVRTIERKFYKDELKQILEKQKEFHQELKNDDLYNDCVRELYRNNEAHQLTLSKKEFVHLLMEDIIFYQRPLRSQKSSISNCSLEFRKYKDENGVEHIQYLKAVPKSNPYYQEFRIWQWIFNLNIYKRDDEQNPVTTEFLNTTTDIENLFEFLNNRKEVDQKALLKHFKLNEKTHRWKYVEDKKYPCNETKSMISERLKKVENIANDFLTREIEQKIWHIIYSVNDKIEYEKALKSFAKKNNLDENSFFEAFKKFPPFKSEYGSFSEKAIKKLLPLMRLGKYWNCENISDDSKERIQKIINGEYDENIKDRVREKAIHLTSENNFQGLQLWLAQYVVYDRHSEASMIGKWNSADDLEEFLKEFKQHSLRNPIVEQVITETLRVVKDIWLKYGNGAKDFFNEIHIELGREMKQTKDERLDATKRITENENTNLRIKALLAEMMNDNSVENVRPYSPMQQEILKIYEDGVLNSGIEIEDEYLKISKTAQPSPSDLKRYKLWLEQRYKSPYTGQIIPLNKLFTPEYEIEHIIPQSRYFDDSFSNKIICESAVNKLKDNYIGLGFIKQFGGTIVECGLGKNVKVFEVNEYEDFVKKHYANNRGKRNKLLLEEIPEKMIERQLNDTRHISKYISGVLSNIVRVEDGSDEGVNSKNIVPGNGKITTQLKQDWGLNDVWNDLILPRFERMNQLTNSTDFTAWNENHQKFLPTVPIEFSKGFSKKRIDHRHHALDALVIACATKDHINLLNNQSAKSDTKRYDLKKKLMKFEKGVYNHPQTGERIQRDVPKQFLKPWESFTIDAKNNLDKIIISFKQNLRVINKATNYYEKYVEKNGIKTKERVEQTGTNWAIRKPMHKETVSGIVNLPWVKVPKGKILTATRKSLDTTFDLKSINSITDTGIQKILRNYLEFKGSPELAFSPEGIEDMNKNIEKYNDGKLHQPINKVRVFELGSKFQVGQTGNKKDKYVEAAKGTNLFFAVYEDKNGKRNYETIPLNIVIERQKQGLLACPESNEKGEKLLFQLSPNDFVYLTTEEENDNATPINFSLLSKEQINNLYKIVSFTGNRLYGIPICVATTIVNKAEYTQLNKIEFTKEKDLLLKLNVNRLGDVKTFTANDIRKIFNRQE

SEQ ID NO: 111 - RNA, 16 nt, Chryseobacterium sp.
  source 1..16: mol_type = other RNA
gttgtgaattgctttc

SEQ ID NO: 112 - RNA, 73 nt, Chryseobacterium sp.
  source 1..73: mol_type = other RNA
gaaagcaattcacaataaggattattccgttgtgaaaacatttagcgcctcgactatcttcggggcatttttt

SEQ ID NO: 113 - RNA, 122 nt, synthetic construct
  misc_difference 1..29: note = a, c, u, g, unknown or other
  misc_feature 1..122: note = source = /note="Description of Artificial Sequence: Synthetic polynucleotide"
  source 1..122: mol_type = other RNA
nnnnnnnnnnnnnnnnnnnnnnnnnnnnngttgtgaattgctttcaaaggaaagcaattcacaataaggattattccgttgtgaaaacatttagcgcctcgactatcttcggggcatttttt

SEQ ID NO: 114 - RNA, 123 nt, synthetic construct
  misc_feature 1..123: note = source = /note="Description of Artificial Sequence: Synthetic polynucleotide"
  source 1..123: mol_type = other RNA
gagcggacagcagcttcctatatctcgtacgttgtgaattgctttcaaaggaaagcaattcacaataaggattattccgttgtgaaaacatttagcgcctcgactatcttcggggcatttttt

SEQ ID NO: 115 - RNA, 123 nt, synthetic construct
  misc_feature 1..123: note = source = /note="Description of Artificial Sequence: Synthetic polynucleotide"
  source 1..123: mol_type = other RNA
ccatgatatagacgttgtggctgttgtagtgttgtgaattgctttcaaaggaaagcaattcacaataaggattattccgttgtgaaaacatttagcgcctcgactatcttcggggcatttttt

SEQ ID NO: 116 - skipped

SEQ ID NO: 117 - protein, 1092 aa, Bacillus sp.
MRYSIGLDIGTTSIGNAVINKDLQRFEHLGVRIFDAAENPKDGSSLSAPRRLARSSRRRLRRRKHRVERTKQLLINKGLLTKTQVKNLYNSKNINLDIWDIRVSGIDRKLFNNEFARVLIHFSKNRGFKSNRKSELKEDDNGAILSAVKENRELMDEKGYRTIAEMLVSDEKYEGTKRNKGGDYSHVVARSDIENEICLLFQKQREYGHPFATEENEEAFLSIWSSQRPFSTKDDIVKKIGNCTLEPKEKRAPKSTYTFERFRALDKLNRLRILSTTAPSRPLTNEERKSILSSLFSKKEVKYKELRKLLKLTDDQRFNEIYYSPDETIEKTENRTFLSLESQYKIKKIIEKTESKNMQSSYHPIDYDTIGYALTVFKDDKDIQHYLQNSYIDSKGKAIPNMANREYNLELIEELLGLSFAKFGHLSLKALNNILPYMEEGEPYHIACEMASYQFSQRLSKEKHRLLPPIPVDEIPNPVVVRALTQVRKVLNSIIKKYGPPSDIYIELAREMSKPFKERKSLEREFNENRQINEKAKAHISELYRIPNDPRPHDILKFKLWNEQNGICPYSLKPISIEYLFNIGYAEVDHIIPYSRSFDDSNGNKVLVLTRENQNKLNRTPYEWFGHEENRWEDFVSFIRTMKVGKKKKNMLLKKNFDEEQEEQILSRNLNDTRYITRYIKSFIEDNLEFRTEENKEQYVHTVNGAYTSLMRKRWGLNKDRRGNDLHHAVDAAIIAVSLPFKNKVNAYFKRQETGLSKLLNNKKDIFPEPWRNFIKELEARMIQDPEKMKRALESLELETYGEIFLNKLKPIFVSRMPKHSIKGQIHEETIRRVRGFTEEGFLVTVKKTRLDQIPFDKNGDFPMYGKETDIKTYMAIKQRYLEYGQDKQKAFAVPLRKPSKNPKNAPIVRSVKIEGKANRVVMLDDKAAADNASIVRTEVFRHKKTGEYYLTPVYVADILSNKIPDRLITIKKSYSDWDRITDEHEYLFSLYNNDLVKIILPKEKETKKYTGGNHLWQEGFFYFKGVDSSNAGIKIINHLNSFEARIGTKRLIAFEKYQVNPLGEINKVHGEKRPGELLNKEEIKENRKNIS

SEQ ID NO: 118 - RNA, 23 nt, Bacillus sp.
  source 1..23: mol_type = other RNA
gccataattcctctgtaaaactt

SEQ ID NO: 119 - RNA, 112 nt, Bacillus sp.
  source 1..112: mol_type = other RNA
aaggtttatagagttattatggtaaggcaatatgccgtggcgttggggatcgcctatgtccggttttaccggatctccctaaaggtgactaactttggttagtcaccttttt

SEQ ID NO: 120 - RNA, 168 nt, synthetic construct
  misc_difference 1..29: note = a, c, u, g, unknown or other
  misc_feature 1..168: note = source = /note="Description of Artificial Sequence: Synthetic polynucleotide"
  source 1..168: mol_type = other RNA
nnnnnnnnnnnnnnnnnnnnnnnnnnnnngccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 121 - RNA, 169 nt, synthetic construct
  misc_feature 1..169: note = source = /note="Description of Artificial Sequence: Synthetic polynucleotide"
  source 1..169: mol_type = other RNA
gagcggacagcagcttcctatatctcgtacgccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 122 - RNA, 169 nt, synthetic construct
  misc_feature 1..169: note = source = /note="Description of Artificial Sequence: Synthetic polynucleotide"
  source 1..169: mol_type = other RNA
ccatgatatagacgttgtggctgttgtagtgccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 123 - skipped

SEQ ID NO: 124 - DNA, 13 bp, synthetic construct
  misc_feature 1..13: note = source = /note="Description of Artificial Sequence: Synthetic oligonucleotide"
  source 1..13: mol_type = other DNA
gccgccrccatgg

SEQ ID NO: 125 - protein, 7 aa, synthetic construct
  REGION 1..7: note = source = /note="Description of Artificial Sequence: Synthetic peptide"
PKKKRKV

SEQ ID NO: 126 - protein, 22 aa, synthetic construct
  REGION 1..22: note = source = /note="Description of Artificial Sequence: Synthetic peptide"
DYKDHDGDYKDHDIDYKDDDDK

SEQ ID NO: 127 - protein, 16 aa, synthetic construct
  REGION 1..16: note = source = /note="Description of Artificial Sequence: Synthetic peptide"
KRPAATKKAGQAKKKK

SEQ ID NO: 128 - protein, 1092 aa, synthetic construct
  REGION 1..1092: note = source = /note="Description of Artificial Sequence: Synthetic polypeptide"
MRYSIGLAIGTTSIGNAVINKDLQRFEHLGVRIFDAAENPKDGSSLSAPRRLARSSRRRLRRRKHRVERTKQLLINKGLLTKTQVKNLYNSKNINLDIWDIRVSGIDRKLFNNEFARVLIHFSKNRGFKSNRKSELKEDDNGAILSAVKENRELMDEKGYRTIAEMLVSDEKYEGTKRNKGGDYSHVVARSDIENEICLLFQKQREYGHPFATEENEEAFLSIWSSQRPFSTKDDIVKKIGNCTLEPKEKRAPKSTYTFERFRALDKLNRLRILSTTAPSRPLTNEERKSILSSLFSKKEVKYKELRKLLKLTDDQRFNEIYYSPDETIEKTENRTFLSLESQYKIKKIIEKTESKNMQSSYHPIDYDTIGYALTVFKDDKDIQHYLQNSYIDSKGKAIPNMANREYNLELIEELLGLSFAKFGHLSLKALNNILPYMEEGEPYHIACEMASYQFSQRLSKEKHRLLPPIPVDEIPNPVVVRALTQVRKVLNSIIKKYGPPSDIYIELAREMSKPFKERKSLEREFNENRQINEKAKAHISELYRIPNDPRPHDILKFKLWNEQNGICPYSLKPISIEYLFNIGYAEVDHIIPYSRSFDDSNGNKVLVLTRENQNKLNRTPYEWFGHEENRWEDFVSFIRTMKVGKKKKNMLLKKNFDEEQEEQILSRNLNDTRYITRYIKSFIEDNLEFRTEENKEQYVHTVNGAYTSLMRKRWGLNKDRRGNDLHHAVDAAIIAVSLPFKNKVNAYFKRQETGLSKLLNNKKDIFPEPWRNFIKELEARMIQDPEKMKRALESLELETYGEIFLNKLKPIFVSRMPKHSIKGQIHEETIRRVRGFTEEGFLVTVKKTRLDQIPFDKNGDFPMYGKETDIKTYMAIKQRYLEYGQDKQKAFAVPLRKPSKNPKNAPIVRSVKIEGKANRVVMLDDKAAADNASIVRTEVFRHKKTGEYYLTPVYVADILSNKIPDRLITIKKSYSDWDRITDEHEYLFSLYNNDLVKIILPKEKETKKYTGGNHLWQEGFFYFKGVDSSNAGIKIINHLNSFEARIGTKRLIAFEKYQVNPLGEINKVHGEKRPGELLNKEEIKENRKNIS

SEQ ID NO: 129 - protein, 230 aa, unidentified
  REGION 1..230: note = source = /note="Description of Unknown: APG09980 deaminase sequence"
MAAGPAPEARSLMDEQTFLDNFNNLKYPRKTYLCYEVELLVGENHIPLDDYKGFVHNEGFDMGLERCHAELIFLERMASWNLDTELRYRITVFISWSPCPECADELVKFLRENRHVNLRIFAARIYDWYQGYEAGLRALKAAGAEVAMMTLHEFEYCWNNFVDHQQDEDTPFPPWDNLVARSEELSQRLEGILQPSVLVFCWPSQVSVTAAHSDIMSQASRAWEKRRDPP

SEQ ID NO: 130 - protein, 182 aa, unidentified
  REGION 1..182: note = source = /note="Description of Unknown: APG07386 CTD sequence"
MEATYPHIFYFHFKNLRKAYGRNETWLCFTMEIIKQHSTVFWETGVFRNQVYPESLCHAERCFLSWFCEDILSPNTDYRVTWYTSWSPCLDCAGEVAEFLARHSNVKLAIFAARLYYFWDPHYQQGLRSLSEKGASVEIMGYKDFKYCWENFVYNGDEPFKPWKGLKYNFLFLDSKLQEILQ

SEQ ID NO: 131 - protein, 201 aa, unidentified
  REGION 1..201: note = source = /note="Description of Unknown: APG05840 deaminase sequence"
MEASPASRPRPLMDPHMFTGNFTNNPRVFGLHQTYLCYEVKRQGPDGTRDLMNEQRDFLCNQAKNHFSGSEDHHAERCFLDRIPSWQLDPAQTYRVTCFISWSPCFSCAQEVAEFLHENPHVNLRIFAARIYDYLPRYEEGLQMLQNAGAQVSIMTSEEFGHCWDTFVDRQGHPFQPWEGLDEHSQALSGRLQAILQNQGN

SEQ ID NO: 132 - protein, 192 aa, unidentified
  REGION 1..192: note = source = /note="Description of Unknown: APG00868 deaminase sequence"
MEPWRPSPRNPMDRIDPNTFRFHFPNLLYASGRKLCYLCFQVETGDYFSCDDSDRGVFRNKVHPWARCHAEQCFLSWFRDQYPCRDEYYNVTWFLSWSPCPTCAEEVVEFLEEYRNLTLSIFTSRLYYFYHPNYQQGLRKLWDAGVQLDIMSCDDFEHCWDNFVDHKGMRFQRRNLLKDYDFLAAELQEILR

SEQ ID NO: 133 - protein, 32 aa, synthetic construct
  REGION 1..32: note = source = /note="Description of Artificial Sequence: Synthetic polypeptide"
SGGSSGGSSGSETPGTSESATPESSGGSSGGS

SEQ ID NO: 134 - DNA, 159 bp, synthetic construct
  misc_feature 1..159: note = source = /note="Description of Artificial Sequence: Synthetic polynucleotide"
  source 1..159: mol_type = other DNA
gggccattaaaacctctccagccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 135 - DNA, 159 bp, synthetic construct
  misc_feature 1..159: note = source = /note="Description of Artificial Sequence: Synthetic polynucleotide"
  source 1..159: mol_type = other DNA
catggcagtacattagagcagccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 136 - DNA, 164 bp, synthetic construct
  misc_feature 1..164: note = source = /note="Description of Artificial Sequence: Synthetic polynucleotide"
  source 1..164: mol_type = other DNA
agctgtttgggaggtcagaaatagggccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 137 - protein, 1151 aa, Brevibacillus sp.
MAKRPAATKKAGQAKKKKAIRSIKLKLKTRTGPEAQNLRKGIWRTHRLLNEGVAYYMKMLLLFRQESTGGQTKKELQEELVRHIREQQQKNRADKNTQALPLDKAFAALRQLYELLVPSSIGQSGDAQIISRKFLSPLVDPNSEGGKGTSKAGAKPTWQKKKEANDPTWEQDYEKWKKRREEDPTASVITTLEEYGIRPIFPLYTNTVADIAWLPLQSNQFVRTWDRDMLQQAIERLLSWESWNKRVQEEYSKLQEKMTQLNEQLEGGQEWISLLEQYEEQREQELIENMTAANDKYRITKRQMKGWNELYEQWSTVLPNASHEQYREALKRVQQRLRGRFGDAHFFQYLMKEEHHLIWKGNPQRIHYFVARNELKKRLEEAKQNATMTLPDARKHPLWVRFDARGGNLQDYYLTAEADNPRSRRFVTFSQLIWPNESGWMEKQDVEVELALSKQFYQQVTLQKNDKGKQEIEFKDKGSGSTFSGHLGGAKLQLERGDLEKEEKDFEGGEIGSVYLNIVIDFEPLQEVKNGRLQSPYGQVLQLVRRPNEFPKVTTYKSEELVEWIKSSTKDSAGVESLESGFRVMSIDLGLRTAAATSIFSVEESNDANAAGFSYWIEGTPLVAVHKRSYMLKLPGEQVEKQVREKRDERQDQQRRVRFQIRILSQVIRMAKKQNRERADELDHLSQALEKQKSLLDQTDRTFWNGIVCDLTDALREKEGGWEQAVVQIHRKAEEHVGKVVQAWRKRFDADERKGIAGLSMWSIEELDSLRKLLISWSRRTRNPREINCFEQGHTSHQRLLTHIQNVKEDRLKQLSHAIVMTALGYVYDEKKLEWFAKYPACQVILFENLSQYRSNMDRSTKENSTLMKWAHRSIPKYVHMQAEPYGIQIGDVRAEYSSRFHAKTGTPGIRCKMVSGHDLQGRRFENLQKRLISEQFLTEEQVKQLRPGDIVPDDSGEWFMTLSDGSEGKEVVFLQADINAAQNLQKRFWQRYNELFKVSCRVLIRGEEEYLIPKTKSVQAKLGKGLFVKKTDTVMKDVYVWDSQAKLKGKTTFTEESESPEQLEDFQEIIEEAEEAKGTYRTLFRDPSGVFFPEFVWSTQKDFWSEVKRRLYGKLRERFLMKTRPKKKRKVENLYFQSGSHHHHHHHHHH

SEQ ID NO: 138 - DNA, 11 bp, synthetic construct
  misc_feature 1..11: note = source = /note="Description of Artificial Sequence: Synthetic oligonucleotide"
  source 1..11: mol_type = other DNA
aaaggaatccc

SEQ ID NO: 139 - DNA, 22 bp, synthetic construct
  misc_feature 1..22: note = source = /note="Description of Artificial Sequence: Synthetic oligonucleotide"
  source 1..22: mol_type = other DNA
ttcgattccggagagggagcct

SEQ ID NO: 140 - DNA, 50 bp, synthetic construct
  misc_feature 1..50: note = source = /note="Description of Artificial Sequence: Synthetic oligonucleotide"
  source 1..50: mol_type = other DNA
aggagcggacagcagcttcctatatctcgtacagggaactcaacatggtt

SEQ ID NO: 141 - DNA, 50 bp, synthetic construct
  misc_feature 1..50: note = source = /note="Description of Artificial Sequence: Synthetic oligonucleotide"
  source 1..50: mol_type = other DNA
agccatgatatagacgttgtggctgttgtagtagggaactcaacatggtt

SEQ ID NO: 142 - DNA, 50 bp, synthetic construct
  misc_feature 1..50: note = source = /note="Description of Artificial Sequence: Synthetic oligonucleotide"
  source 1..50: mol_type = other DNA
aggagcggacagcagcttcctatatctcgtacaggaccgtcaacatggtt

SEQ ID NO: 143 - DNA, 50 bp, synthetic construct
  misc_feature 1..50: note = source = /note="Description of Artificial Sequence: Synthetic oligonucleotide"
  source 1..50: mol_type = other DNA
agccatgatatagacgttgtggctgttgtagtaggaccgtcaacatggtt

SEQ ID NO: 144 - RNA, 142 nt, synthetic construct
  misc_feature 1..142: note = source = /note="Description of Artificial Sequence: Synthetic polynucleotide"
  source 1..142: mol_type = other RNA
ggcgacggttagaggccgtatgtcgatttgctttaatttcgtgcgtgtgcattgtcgtcctccattacagggcggctaccacgaatagccacgaagtaaaagcttcgtggctagcacgtacgagatataggaagctgctgtc

SEQ ID NO: 145 - RNA, 137 nt, synthetic construct
  misc_feature 1..137: note = source = /note="Description of Artificial Sequence: Synthetic polynucleotide"
  source 1..137: mol_type = other RNA
ggcgacggttagaggccgtatgtcgatttgctttaatttcgtgcgtgtgcattgtcgtcctccattacagggcggctaccacgaatagccacgaagtaaaagcttcgtggctagcacactacaacagccacaacgtc

SEQ ID NO: 146-219 - skipped (74 entries)

SEQ ID NO: 220 - DNA, 500 bp, Homo sapiens
  source 1..500: mol_type = genomic DNA
aaaaatggacaattatgaggaggggagagtgcagacaggggaagcttcacctcctttacaattttgggagtccacacggcatggcatacaaattatttcattcccattgagaaataaaatccaattctccatcaccaagagagccttccgaaagaggcccccctgggcaaacggccaccgatggagaggtctgccagtcctcttctaccccacccacgcccccaccctaatcagaggccaaacccttcctggagcctgtgataaaagcaactgttagcttgcactagactagcttcaaagttgtattgaccctggtgtgttatgtctaagagtagatgccatatctcttttctggcctatgttattacctgtatggactttgcactggaatcagctatctgctcttacttatgcacacctggggcatagagccagccctgtatcgcttttcagccatctcactacagataactcccaagtcctgtctagctgccttcctt

SEQ ID NO: 221 - DNA, 20 bp, Homo sapiens
  source 1..20: mol_type = genomic DNA
tattgaccctggtgtgttat

SEQ ID NO: 222 - DNA, 20 bp, Homo sapiens
  source 1..20: mol_type = genomic DNA
gcttgcactagactagcttc

SEQ ID NO: 223 - DNA, 20 bp, Homo sapiens
  source 1..20: mol_type = genomic DNA
cttttatcacaggctccagg

SEQ ID NO: 224 - RNA, 135 nt, synthetic construct
  misc_feature 1..135: note = source = /note="Description of Artificial Sequence: Synthetic polynucleotide"
  source 1..135: mol_type = other RNA
cgacggttagaggccgtatgtcgatttgctttaatttcgtgcgtgtgcattgtcgtcctccattacagggcggctaccacgaatagccacgaagtaaaagcttcgtggctagcactattgaccctggtgtgttat

SEQ ID NO: 225 - DNA, 1977 bp, synthetic construct
  misc_feature 1..1977: note = source = /note="Description of Artificial Sequence: Synthetic polynucleotide"
  source 1..1977: mol_type = other DNA
caagcatgaggagggaagtaggcagaatcctctggagccaaagctctggatgtctctcccctctgaccatggagcccacccctgctccactgctccagggacagccctatgctgcaggcagctctgcccccactcagcatcccaggggctgatttctttggttttggatccagctggatgtctgcattgccgaggccaccagggctggctcagcaactgtcggggaatcaccagggtctgagaaatcttgtgcgcatgtgaggggctgtgggagcagagaaccactgggtgggaaattctaatccccaccctgctggaaactctctgggtggccccaacatgctaatcctccggcaaacctctgtttcctcctcaaaaggcaggaggtcggaaagaataaacaatgagagtcacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgcca

SEQ ID NO: 226 - DNA, 566 bp, synthetic construct
  misc_feature 1..566: note = source = /note="Description of Artificial Sequence: Synthetic polynucleotide"
  source 1..566: mol_type = other DNA
tcgtcggcagcgtcagatgtgtataagagacagtttgggttttgccagactccacagtgcatacgtgggctccaacaggtcctcttccctcccagtcactgactaaccccggaaccacacagcttcccgttctcagctccacaaacttggtgccaaattcttctcccctgggaagcatccctggacacttcccaaaggaccccagtcactccagcctgttggctgccgctcactttgatgtctgcaggccagatgagggctccagatggcacattgtcagagggacacactgtggcccctgtgcccagccctgggctctctgtacatgaagcaactccagtcccaaatatgtagctgtttgggaggtcagaaatagggggtccaggagcaaactccccccaccccctttccaaagcccattccctctttagccagagccggggtgtgcagacggcagtcactagggggcgctcggccaccacagggaagctgggtgaatggagcgagcagcgtcttcgagagtgaggacgtgctgtctcttatacacatctccgagcccacgagac

SEQ ID NO: 227 - DNA, 25 bp, synthetic construct
  misc_feature 1..25: note = source = /note="Description of Artificial Sequence: Synthetic oligonucleotide"
  source 1..25: mol_type = other DNA
gaaagggggtggggggagtttgctc

SEQ ID NO: 228 - DNA, 53 bp, synthetic construct
  misc_feature 1..53: note = source = /note="Description of Artificial Sequence: Synthetic oligonucleotide"
  source 1..53: mol_type = other DNA
tcgtcggcagcgtcagatgtgtataagagacagtttgggttttgccagactcc

SEQ ID NO: 229 - DNA, 54 bp, synthetic construct
  misc_feature 1..54: note = source = /note="Description of Artificial Sequence: Synthetic oligonucleotide"
  source 1..54: mol_type = other DNA
gtctcgtgggctcggagatgtgtataagagacagcctctgacaatgtgccatct

SEQ ID NO: 230 - DNA, 20 bp, synthetic construct
  misc_feature 1..20: note = source = /note="Description of Artificial Sequence: Synthetic oligonucleotide"
  source 1..20: mol_type = other DNA
cccgtataaagcatgagacc

SEQ ID NO: 231 - DNA, 21 bp, synthetic construct
  misc_feature 1..21: note = source = /note="Description of Artificial Sequence: Synthetic oligonucleotide"
  source 1..21: mol_type = other DNA
caagcatgaggagggaagtag

SEQ ID NO: 232 - DNA, 21 bp, synthetic construct
  misc_feature 1..21: note = source = /note="Description of Artificial Sequence: Synthetic oligonucleotide"
  source 1..21: mol_type = other DNA
tggcagacagggagaaataag

SEQ ID NO: 233 - RNA, 137 nt, synthetic construct
  misc_feature 1..137: note = source = /note="Description of Artificial Sequence: Synthetic polynucleotide"
  source 1..137: mol_type = other RNA
ggcgacggttagaggccgtatgtcgatttgctttaatttcgtgcgtgtgcattgtcgtcctccattacagggcggctaccacgaatagccacgaagtaaaagcttcgtggctagcacgaaagggggtggggggagtt

SEQ ID NO: 234 - RNA, 137 nt, synthetic construct
  misc_feature 1..137: note = source = /note="Description of Artificial Sequence: Synthetic polynucleotide"
  source 1..137: mol_type = other RNA
ggcgacggttagaggccgtatgtcgatttgctttaatttcgtgcgtgtgcattgtcgtcctccattacagggcggctaccacgaatagccacgaagtaaaagcttcgtggctagcaccccgtataaagcatgagacc

SEQ ID NO: 235 - protein, 1071 aa, Bacillus sp.
MRELDYRIGLDIGTNSIGWGVIELSWNKDRERYEKVRIVDQGVRMFDRAEMPKTGASLAEPRRIARSSRRRLNRKSQRKKNIRNLLVQHGVITQEELDSLYPLSKKSMDIWGIRLDGLDRLLNHFEWARLLIHLAQRRGFKSNRKSELKDTETGKVLSSIQLNEKRLSLYRTVGEMWMKDPDFSKYDRKRNSPNEYVFSVSRAELEKEIVTLFAAQRRFQSPYASKDLQETYLQIWTHQLPFASGNAILNKVGYCSLLKGKERRIPKATYTFQYFSALDQVNRTRLGPDFQPFTKEQREIILNNMFQRTDYYKKKTIPEVTYYDIRKWLELDETIQFKGLNYDPNEELKKIEKKPFINLKAFYEINKVVANYSERTNETFSTLDYDGIGYALTVYKTDKDIRSYLKSSHNLPKRCYDDQLIEELLSLSYTKFGHLSLKAINHVLSIMQKGNTYKEAVDQLGYDTSGLKKEKRSKFLPPISDEITNPIVKRALTQARKVVNAIIRRHGSPHSVHIELARELSKNHDERTKIVSAQDENYKKNKGAISILSEHGILNPTGYDIVRYKLWKEQGERCAYSLKEIPADTFFNELKKERNGAPILEVDHILPYSQSFIDSYHNKVLVYSDENRKKGNRIPYTYFLETNKDWEAFERYVRSNKFFSKKKREYLLKRAYLPRESELIKERHLNDTRYASTFLKNFIEQNLQFKEAEDNPRKRRVQTVNGVITAHFRKRWGLEKDRQETYLHHAMDAIIVACTDHHMVTRVTEYYQIKESNKSVKKPYFPMPWEGFRDELLSHLASQPIAKKISEELKAGYQSLDYIFVSRMPKRSITGAAHKQTIMRKGGIDKKGKTIIIERLHLKDIKFDENGDFKMVGKEQDMATYEAIKQRYLEHGKNSKKAFETPLYKPSKKGTGNLIKRVKVEGQAKSFVREVNGGVAQNGDLVRVDLFEKDDKYYMVPIYVPDTVCSELPKKVVASSKGYEQWLTLDNSFTFKFSLYPYDLVRLVKGDEDRFLYFGTLDIDSDRLNFKDVNKPSKKNEYRYSLKTIEDLEKYEVGVLGDLRLVRKETRRNFH

SEQ ID NO: 236 - RNA, 46 nt, Bacillus sp.
  source 1..46: mol_type = other RNA
gacagcagcttcctatatctcgtacgtcatagttccattaaagcca

SEQ ID NO: 237 - RNA, 85 nt, Bacillus sp.
  source 1..85: mol_type = other RNA
tggctttgatgtttctatgataagggtttcgacccgtggcgtcggggatcgcctgcccattgaaatgggcttctccccatttatt

SEQ ID NO: 238 - RNA, 32 nt, synthetic construct
  misc_feature 1..32: note = source = /note="Description of Artificial Sequence: Synthetic oligonucleotide"
  source 1..32: mol_type = other RNA
gacagcagcttcctatatctcgtacgtcatag

SEQ ID NO: 239 - RNA, 71 nt, synthetic construct
  misc_feature 1..71: note = source = /note="Description of Artificial Sequence: Synthetic oligonucleotide"
  source 1..71: mol_type = other RNA
ctatgataagggtttcgacccgtggcgtcggggatcgcctgcccattgaaatgggcttctccccatttatt

SEQ ID NO: 240 - RNA, 36 nt, synthetic construct
  misc_feature 1..36: note = source = /note="Description of Artificial Sequence: Synthetic oligonucleotide"
  source 1..36: mol_type = other RNA
gacagcagcttcctatatctcgtacgtcatagccat

SEQ ID NO: 241 - RNA, 75 nt, synthetic construct
  misc_feature 1..75: note = source = /note="Description of Artificial Sequence: Synthetic oligonucleotide"
  source 1..75: mol_type = other RNA
atgtctatgataagggtttcgacccgtggcgtcggggatcgcctgcccattgaaatgggcttctccccatttatt

SEQ ID NO: 242 - RNA, 40 nt, synthetic construct
  misc_feature 1..40: note = source = /note="Description of Artificial Sequence: Synthetic oligonucleotide"
  source 1..40: mol_type = other RNA
gacagcagcttcctatatctcgtacgtcatagttccatta

SEQ ID NO: 243 - RNA, 79 nt, synthetic construct
  misc_feature 1..79: note = source = /note="Description of Artificial Sequence: Synthetic oligonucleotide"
  source 1..79: mol_type = other RNA
tgatgtttctatgataagggtttcgacccgtggcgtcggggatcgcctgcccattgaaatgggcttctccccatttatt

SEQ ID NO: 244 - RNA, 46 nt, synthetic construct; identical to SEQ ID NO: 236
  misc_feature 1..46: note = source = /note="Description of Artificial Sequence: Synthetic oligonucleotide"
  source 1..46: mol_type = other RNA

SEQ ID NO: 245 - RNA, 78 nt, synthetic construct
  misc_feature 1..78: note = source = /note="Description of Artificial Sequence: Synthetic oligonucleotide"
  source 1..78: mol_type = other RNA
tggctttgatgtttctatgataagggtttcgacccgtggcgtcggggatcgcctgcccattgaaatgggcttctcccc

SEQ ID NO: 246 - RNA, 46 nt, synthetic construct; identical to SEQ ID NO: 236
  misc_feature 1..46: note = source = /note="Description of Artificial Sequence: Synthetic oligonucleotide"
  source 1..46: mol_type = other RNA

SEQ ID NO: 247 - RNA, 75 nt, synthetic construct
  misc_feature 1..75: note = source = /note="Description of Artificial Sequence: Synthetic oligonucleotide"
  source 1..75: mol_type = other RNA
tggctttgatgtttctatgataagggtttcgacccgtggcgtcggggaccgcccattgaaatgggcctccccttt

SEQ ID NO: 248 - RNA, 46 nt, synthetic construct; identical to SEQ ID NO: 236
  misc_feature 1..46: note = source = /note="Description of Artificial Sequence: Synthetic oligonucleotide"
  source 1..46: mol_type = other RNA

SEQ ID NO: 249 - RNA, 71 nt, synthetic construct
  misc_feature 1..71: note = source = /note="Description of Artificial Sequence: Synthetic oligonucleotide"
  source 1..71: mol_type = other RNA
tggctttgatgtttctatgataagggtttcgacccgtggcgtcgggaccgccattgaaatggcctcccttt

SEQ ID NO: 250 - RNA, 38 nt, synthetic construct
  misc_feature 1..38: note = source = /note="Description of Artificial Sequence: Synthetic oligonucleotide"
  source 1..38: mol_type = other RNA
gacagcagcttcctatatctcgtacgtcatagttccat

SEQ ID NO: 251 - RNA, 77 nt, synthetic construct
  misc_feature 1..77: note = source = /note="Description of Artificial Sequence: Synthetic oligonucleotide"
  source 1..77: mol_type = other RNA
atgtttctatgataagggtttcgacccgtggcgtcggggatcgcctgcccattgaaatgggcttctccccatttatt

SEQ ID NO: 252 - RNA, 38 nt, synthetic construct; identical to SEQ ID NO: 250
  misc_feature 1..38: note = source = /note="Description of Artificial Sequence: Synthetic oligonucleotide"
  source 1..38: mol_type = other RNA

SEQ ID NO: 253 - RNA, 70 nt, synthetic construct
  misc_feature 1..70: note = source = /note="Description of Artificial Sequence: Synthetic oligonucleotide"
  source 1..70: mol_type = other RNA
atgtttctatgataagggtttcgacccgtggcgtcggggatcgcctgcccattgaaatgggcttctcccc

SEQ ID NO: 254 - RNA, 46 nt, synthetic construct; identical to SEQ ID NO: 236
  misc_feature 1..46: note = source = /note="Description of Artificial Sequence: Synthetic oligonucleotide"
  source 1..46: mol_type = other RNA

SEQ ID NO: 255 - RNA, 76 nt, synthetic construct
  misc_feature 1..76: note = source = /note="Description of Artificial Sequence: Synthetic oligonucleotide"
  source 1..76: mol_type = other RNA
tggctttgatgtttctatgataagggtttcgacccgtggcgtcggggatcgcctgcccattgaaatgggcttctcc

SEQ ID NO: 256 - RNA, 39 nt, synthetic construct
  misc_feature 1..39: note = source = /note="Description of Artificial Sequence: Synthetic oligonucleotide"
  source 1..39: mol_type = other RNA
gcttcctatatctcgtacgtcatagttccattaaagcca

SEQ ID NO: 257 - RNA, 85 nt, synthetic construct; identical to SEQ ID NO: 237
  misc_feature 1..85: note = source = /note="Description of Artificial Sequence: Synthetic oligonucleotide"
  source 1..85: mol_type = other RNA

SEQ ID NO: 258 - RNA, 38 nt, synthetic construct; identical to SEQ ID NO: 250
  misc_feature 1..38: note = source = /note="Description of Artificial Sequence: Synthetic oligonucleotide"
  source 1..38: mol_type = other RNA

SEQ ID NO: 259 - RNA, 71 nt, synthetic construct; identical to SEQ ID NO: 249
  misc_feature 1..71: note = source = /note="Description of Artificial Sequence: Synthetic oligonucleotide"
  source 1..71: mol_type = other RNA

SEQ ID NO: 260 - DNA, 682 bp, synthetic construct
  misc_feature 1..682: note = source = /note="Description of Artificial Sequence: Synthetic polynucleotide"
  source 1..682: mol_type = other DNA
ttgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacctgaagaccaggctcgaaagccttcacaccctcagtaagagctttctcaatgtcgattgtgccacacaccctgtcctcagtagcacctagcggtaaatcaaccatgttgacggtcctgtacgagatataggaagctgctgtccgctcctcaatttgtcacggacttctgcactcattacttcttggtcatctggatctgaattgaacggatcaccagaaataactttgatttcaggaagtaaatctaccaaagatctaaccgtggtggacttcccggttcctctatcacccattatcatcacaggctaggtggaggctcagtgatgataagtctgcgatggtggatgcatgtgtcatggtcatagctgtttcctgtgtgaaattgttatccgctcagagggcacaatcctattccgcgctatccgacaatctccaagac

SEQ ID NO: 261 - protein, 82 aa, synthetic construct
  REGION 1..82: note = source = /note="Description of Artificial Sequence: Synthetic polypeptide"
TNLSDHEKETGKQLVIQESILMLPEEVEEVIGNKPESDILVHTAYDESTDENVMLLTSDAPEYKPWALVIQDSNGENKIKML

SEQ ID NO: 262 - protein, 1072 aa, synthetic construct
  REGION 1..1072: note = source = /note="Description of Artificial Sequence: Synthetic polypeptide"
MSELDYRIGLAIGTNSIGWGVIELFWNKDRERYEKVRIVDKGVRMFDKAEIPNKGASLAEPRRIARSSRRRLNRKSQRKKEIRNLLVQHGMITQEELDLLYPLSKKSIDIWDIRLDGLDRLLNHLEWARLLIHLAQRRGFKSNRKSELKDAETGKVLSSIQVNEKRLFLYRTVGEMWIKDAEFSKYDRRRNSPNEYVFSVSRADLEKEIVTLFEAQRKFQSSYASKNLQETYLQIWAHQLPFASGNAILNKVGYCSLLKGKERRIPKATYTFQYFSALDQVNRTRLGPDFQPFTQEQKEIILDKMFQRTDYYKKKTIPEVSYYDIRKWLELDETIQFKGLNYDPNEELKKIEKKPFINLKAFYEIKKVVANYAERTNEAFSTLDYDAIAYALTVYKTDKDIRSYLKKSNNLSKRCYDDQLIEELFTLSYTKFGHLSFKAINHVLPIMQEGRTYQEAIHQLGYDTTNLKKENRSMFLPLIPDEITNPIVKRAITQARKVVNAIIRRYGSPNSVHIELARELSKSHDERKKIMTAHDENYKKNKGAISILIENGILNPTGYDIVRYKLWKEQGERCAYSLKEIPPDTFFNELKKERNGSPILEVDHILPYSQSFIDSYHNKVLVYSDENRNKGNRIPYTYFLETNKDWEAFERYVRSNKLFSKKKREYLLKKTYLPRESELIKERHLNDTRYASTFLKNFIEQNLQFKEVEVNLRKKRVQTVNGVITAHLRKRWGLEKNRQETYLHHAMDAIIVACTDHHMVTRITEYYQIKESNKSVKKPYFPMPWEGFRDELLSHLASQPIAKKISEELKAGYQSSDYIFVSRMPKRSVTGAAHDQTIRRKGGIDKKGKTIIIKRVRLKDIKFDENGDFKMVGKEQDLATYEAIKQRYLEHRKNSKKAFETPLYKPSKKGTGNLIKRVKIEGQTKAFVREVNGGVAQNSDLVRVDLFEKDDKYYMVPIYVPDTVCSELPKKVVKSGKGYEQWLTLDNSFTFKSSLYPYDLVRLVKGNEDRFLYFGTLDIDSDRLNFKDVNKPSKQNEYRYSLKTIENLEKYEVGVLGDLRLVKQETRRIFNR

SEQ ID NO: 263 - DNA, 130 bp, synthetic construct
  misc_feature 1..130: note = source = /note="Description of Artificial Sequence: Synthetic polynucleotide"
  source 1..130: mol_type = other DNA
catggcagtacattagagcagtcatagttccattaaagccaaaagtggctttgatgtttctatgataagggtttcgacccgtggcgtcggggatcgcctgcccattgaaatgggcttctccccatttatt

SEQ ID NO: 264 - DNA, 130 bp, synthetic construct
  misc_feature 1..130: note = source = /note="Description of Artificial Sequence: Synthetic polynucleotide"
  source 1..130: mol_type = other DNA
cacatctcgagcaagacgttgtcatagttccattaaagccaaaagtggctttgatgtttctatgataagggtttcgacccgtggcgtcggggatcgcctgcccattgaaatgggcttctccccatttatt

SEQ ID NO: 265 - DNA, 130 bp, synthetic construct
  misc_feature 1..130: note = source = /note="Description of Artificial Sequence: Synthetic polynucleotide"
  source 1..130: mol_type = other DNA
cttctatagcctccttccccgtcatagttccattaaagccaaaagtggctttgatgtttctatgataagggtttcgacccgtggcgtcggggatcgcctgcccattgaaatgggcttctccccatttatt

SEQ ID NO: 266 - DNA, 50 bp, synthetic construct
  misc_feature 1..50: note = source = /note="Description of Artificial Sequence: Synthetic oligonucleotide"
  source 1..50: mol_type = other DNA
caggtcgactctagaggatcccatgatatagacgttgtggctgttgtagt

SEQ ID NO: 267 - DNA, 53 bp, synthetic construct
  misc_difference 19..23: note = a, c, t, g, unknown or other
  misc_feature 1..53: note = source = /note="Description of Artificial Sequence: Synthetic oligonucleotide"
  source 1..53: mol_type = other DNA
aacgacggccagtgaattnnnnnactacaacagccacaacgtctatatcatgg

SEQ ID NO: 268 - protein, 1493 aa, Homo sapiens
MPNEGIPHSSQTQEQDCLQSQPVSNNEEMAIKQESGGDGEVEEYLSFRSVGDGLSTSAVGCASAAPRRGPALLHIDRHQIQAVEPSAQALELQGLGVDVYDQDVLEQGVLQQVDNAIHEASRASQLVDVEKEYRSVLDDLTSCTTSLRQINKIIEQLSPQAATSRDINRKLDSVKRQKYNKEQQLKKITAKQKHLQAILGGAEVKIELDHASLEEDAEPGPSSLGSMLMPVQETAWEELIRTGQMTPFGTQIPQKQEKKPRKIMLNEASGFEKYLADQAKLSFERKKQGCNKRAARKAPAPVTPPAPVQNKNKPNKKARVLSKKEERLKKHIKKLQKRALQFQGKVGLPKARRPWESDMRPEAEGDSEGEESEYFPTEEEEEEEDDEVEGAEADLSGDGTDYELKPLPKGGKRQKKVPVQEIDDDFFPSSGEEAEAASVGEGGGGGRKVGRYRDDGDEDYYKQRLRRWNKLRLQDKEKRLKLEDDSEESDAEFDEGFKVPGFLFKKLFKYQQTGVRWLWELHCQQAGGILGDEMGLGKTIQIIAFLAGLSYSKIRTRGSNYRFEGLGPTVIVCPTTVMHQWVKEFHTWWPPFRVAILHETGSYTHKKEKLIRDVAHCHGILITSYSYIRLMQDDISRYDWHYVILDEGHKIRNPNAAVTLACKQFRTPHRIILSGSPMQNNLRELWSLFDFIFPGKLGTLPVFMEQFSVPITMGGYSNASPVQVKTAYKCACVLRDTINPYLLRRMKSDVKMSLSLPDKNEQVLFCRLTDEQHKVYQNFVDSKEVYRILNGEMQIFSGLIALRKICNHPDLFSGGPKNLKGLPDDELEEDQFGYWKRSGKMIVVESLLKIWHKQGQRVLLFSQSRQMLDILEVFLRAQKYTYLKMDGTTTIASRQPLITRYNEDTSIFVFLLTTRVGGLGVNLTGANRVVIYDPDWNPSTDTQARERAWRIGQKKQVTVYRLLTAGTIEEKIYHRQIFKQFLTNRVLKDPKQRRFFKSNDLYELFTLTSPDASQSTETSAIFAGTGSDVQTPKCHLKRRIQPAFGADHDVPKRKKFPASNISVNDATSSEEKSEAKGAEVNAVTSNRSDPLKDDPHMSSNVTSNDRLGEETNAVSGPEELSVISGNGECSNSSGTGKTSMPSGDESIDEKLGLSYKRERPSQAQTEAFWENKQMENNFYKHKSKTKHHSVAEEETLEKHLRPKQKPKNSKHCRDAKFEGTRIPHLVKKRRYQKQDSENKSEAKEQSNDDYVLEKLFKKSVGVHSVMKHDAIMDGASPDYVLVEAEANRVAQDALKALRLSRQRCLGAVSGVPTWTGHRGISGAPAGKKSRFGKKRNSNFSVQHPSSTSPTEKCQDGIMKKEGKDNVPEHFSGRAEDADSSSGPLASSSLLAKMRARNHLILPERLESESGHLQEASALLPTTEHDDLLVEMRNFIAFQAHTDGQASTREILQEFESKLSASQSCVFRELLRNLCTFHRTSGGEGIWKLKPEYC

SEQ ID NO: 269 - RNA, 135 nt, synthetic construct
  misc_feature 1..135: note = source = /note="Description of Artificial Sequence: Synthetic polynucleotide"
  source 1..135: mol_type = other RNA
cgacggttagaggccgtatgtcgatttgctttaatttcgtgcgtgtgcattgtcgtcctccattacagggcggctaccacgaatagccacgaagtaaaagcttcgtggctagcacgcttgcactagactagcttc

SEQ ID NO: 270 - RNA, 135 nt, synthetic construct
  misc_feature 1..135: note = source = /note="Description of Artificial Sequence: Synthetic polynucleotide"
  source 1..135: mol_type = other RNA
cgacggttagaggccgtatgtcgatttgctttaatttcgtgcgtgtgcattgtcgtcctccattacagggcggctaccacgaatagccacgaagtaaaagcttcgtggctagcaccttttatcacaggctccagg

SEQ ID NO: 271 - RNA, 25 nt, synthetic construct
  misc_feature 1..25: note = source = /note="Description of Artificial Sequence: Synthetic oligonucleotide"
  source 1..25: mol_type = other RNA
gacagcagcttcctatatctcgtac

SEQ ID NO: 272 - RNA, 18 nt, synthetic construct
  misc_feature 1..18: note = source = /note="Description of Artificial Sequence: Synthetic oligonucleotide"
  source 1..18: mol_type = other RNA
gcttcctatatctcgtac

SEQ ID NO: 273 - RNA, 22 nt, Brevibacillus sp.
  source 1..22: mol_type = other RNA
tggaaagcttcgaggttagcac

SEQ ID NO: 274 - RNA, 95 nt, Brevibacillus sp.
  source 1..95: mol_type = other RNA
cgacggttagaggccgtatgtcgatttgctttaatttcgtgcgtgtgcattgtcgtcctccattacagggcggctaccacgaatagccacgaagt

SEQ ID NO: 275 - RNA, 151 nt, synthetic construct
  misc_difference 132..151: note = a, c, u, g, unknown or other
  misc_feature 1..151: note = source = /note="Description of Artificial Sequence: Synthetic polynucleotide"
  source 1..151: mol_type = other RNA
cgacggttagaggccgtatgtcgatttgctttaatttcgtgcgtgtgcattgtcgtcctccattacagggcggctaccacgaatagtcacgaagttccactgagtaaagtggaaagcttcgaggttagcacnnnnnnnnnnnnnnnnnnnn

SEQ ID NO: 276 - protein, 6 aa, synthetic construct
  REGION 1..6: note = source = /note="Description of Artificial Sequence: Synthetic 6xHis tag"
HHHHHH

SEQ ID NO: 277 - protein, 10 aa, synthetic construct
  REGION 1..10: note = source = /note="Description of Artificial Sequence: Synthetic 10xHis tag"
HHHHHHHHHH

SEQ ID NO: 278 - DNA, 25 bp, synthetic construct
  misc_feature 1..25: note = source = /note="Description of Artificial Sequence: Synthetic oligonucleotide"
  source 1..25: mol_type = other DNA
cccgtataaagcatgagaccgtgac

SEQ ID NO: 279 - DNA, 25 bp, synthetic construct
  misc_feature 1..25: note = source = /note="Description of Artificial Sequence: Synthetic oligonucleotide"
  source 1..25: mol_type = other DNA
ctcagactgtttgccccttactgct

SEQ ID NO: 280 - RNA, 135 nt, synthetic construct
  misc_feature 1..135: note = source = /note="Description of Artificial Sequence: Synthetic polynucleotide"
  source 1..135: mol_type = other RNA
cgacggttagaggccgtatgtcgatttgctttaatttcgtgcgtgtgcattgtcgtcctccattacagggcggctaccacgaatagccacgaagtaaaagcttcgtggctagcaccccgtataaagcatgagacc

SEQ ID NO: 281 - RNA, 135 nt, synthetic construct
  misc_feature 1..135: note = source = /note="Description of Artificial Sequence: Synthetic polynucleotide"
  source 1..135: mol_type = other RNA
cgacggttagaggccgtatgtcgatttgctttaatttcgtgcgtgtgcattgtcgtcctccattacagggcggctaccacgaatagccacgaagtaaaagcttcgtggctagcacctcagactgtttgcccctta

SEQ ID NO: 282 - RNA, 156 nt, synthetic construct
  misc_feature 1..156: note = source = /note="Description of Artificial Sequence: Synthetic polynucleotide"
  source 1..156: mol_type = other RNA
cgacggttagaggccgtatgtcgatttgctttaatttcgtgcgtgtgcattgtcgtcctccattacagggcggctaccacgaatagtcacgaagttccactgagtaaagtggaaagcttcgaggttagcaccccgtataaagcatgagaccgtgac

SEQ ID NO: 283 - RNA, 156 nt, synthetic construct
  misc_feature 1..156: note = source = /note="Description of Artificial Sequence: Synthetic polynucleotide"
  source 1..156: mol_type = other RNA
cgacggttagaggccgtatgtcgatttgctttaatttcgtgcgtgtgcattgtcgtcctccattacagggcggctaccacgaatagtcacgaagttccactgagtaaagtggaaagcttcgaggttagcacctcagactgtttgccccttactgct

SEQ ID NO: 284 - skipped

SEQ ID NO: 285 - RNA, 135 nt, synthetic construct
  misc_difference 116..135: note = a, c, u, g, unknown or other
  misc_feature 1..135: note = source = /note="Description of Artificial Sequence: Synthetic polynucleotide"
  source 1..135: mol_type = other RNA
cgacggttagaggccgtatgtcgatttgctttaatttcgtgcgtgtgcattgtcgtcctccattacagggcggctaccacgaatagccacgaagtaaaagcttcgtggctagcacnnnnnnnnnnnnnnnnnnnn

SEQ ID NO: 286 - RNA, 95 nt, synthetic construct; identical to SEQ ID NO: 274
  misc_feature 1..95: note = source = /note="Description of Artificial Sequence: Synthetic oligonucleotide"
  source 1..95: mol_type = other RNA

SEQ ID NO: 287 - RNA, 16 nt, synthetic construct
  misc_feature 1..16: note = source = /note="Description of Artificial Sequence: Synthetic oligonucleotide"
  source 1..16: mol_type = other RNA
gcttcgtggctagcac

SEQ ID NO: 288 - RNA, 151 nt, synthetic construct; identical to SEQ ID NO: 275
  misc_difference 132..151: note = a, c, u, g, unknown or other
  misc_feature 1..151: note = source = /note="Description of Artificial Sequence: Synthetic polynucleotide"
  source 1..151: mol_type = other RNA

SEQ ID NO: 289 - RNA, 135 nt, synthetic construct
  misc_difference 116..135: note = a, c, u, g, unknown or other
  misc_feature 1..135: note = source = /note="Description of Artificial Sequence: Synthetic polynucleotide"
  source 1..135: mol_type = other RNA
cgacggttagaggccgtatgtcgatttgctttaatttcgtgcgtgtgcattgtcgtcctccattacagggcggctaccacgaatagtcacgaagtaaaagcttcgaggttagcacnnnnnnnnnnnnnnnnnnnn

SEQ ID NO: 290 - RNA, 144 nt, synthetic construct
  misc_difference 125..144: note = a, c, u, g, unknown or other
  misc_feature 1..144: note = source = /note="Description of Artificial Sequence: Synthetic polynucleotide"
  source 1..144: mol_type = other RNA
cgacggttagaggccgtatgtcgatttgctttaatttcgtgcgtgtgcattgtcgtcctccattacagggcggctaccacgaatagccacgaagttccaaaatggaaagcttcgtggctagcacnnnnnnnnnnnnnnnnnnnn

SEQ ID NO: 291 - RNA, 135 nt, synthetic construct; identical to SEQ ID NO: 285
  misc_difference 116..135: note = a, c, u, g, unknown or other
  misc_feature 1..135: note = source = /note="Description of Artificial Sequence: Synthetic polynucleotide"
  source 1..135: mol_type = other RNA

SEQ ID NO: 292 - DNA, 30 bp, synthetic construct
  misc_feature 1..30: note = source = /note="Description of Artificial Sequence: Synthetic oligonucleotide"
  source 1..30: mol_type = other DNA
gtacgagatataggaagctgctgtccgctc

SEQ ID NO: 293 - DNA, 29 bp, synthetic construct
  misc_feature 1..29: note = source = /note="Description of Artificial Sequence: Synthetic oligonucleotide"
  source 1..29: mol_type = other DNA
actacaacagccacaacgtctatatcatg

SEQ ID NO: 294 - RNA, 149 nt, synthetic construct
  misc_feature 1..149: note = source = /note="Description of Artificial Sequence: Synthetic polynucleotide"
  source 1..149: mol_type = other RNA
cgacggttagaggccgtatgtcgatttgctttaatttcgtgcgtgtgcattgtcgtcctccattacagggcggctaccacgaatagtcacgaagttccactgagtaaagtggaaagcttcgaggttagcacactacaacagccacaacg

SEQ ID NO: 295 - RNA, 151 nt, synthetic construct
  misc_feature 1..151: note = source = /note="Description of Artificial Sequence: Synthetic polynucleotide"
  source 1..151: mol_type = other RNA
cgacggttagaggccgtatgtcgatttgctttaatttcgtgcgtgtgcattgtcgtcctccattacagggcggctaccacgaatagtcacgaagttccactgagtaaagtggaaagcttcgaggttagcacgtacgagatataggaagctg

SEQ ID NO: 296 - RNA, 151 nt, synthetic construct
  misc_feature 1..151: note = source = /note="Description of Artificial Sequence: Synthetic polynucleotide"
  source 1..151: mol_type = other RNA
cgacggttagaggccgtatgtcgatttgctttaatttcgtgcgtgtgcattgtcgtcctccattacagggcggctaccacgaatagtcacgaagttccactgagtaaagtggaaagcttcgaggttagcacactacaacagccacaacgtc

SEQ ID NO: 297 - RNA, 156 nt, synthetic construct
  misc_feature 1..156: note = source = /note="Description of Artificial Sequence: Synthetic polynucleotide"
  source 1..156: mol_type = other RNA
cgacggttagaggccgtatgtcgatttgctttaatttcgtgcgtgtgcattgtcgtcctccattacagggcggctaccacgaatagtcacgaagttccactgagtaaagtggaaagcttcgaggttagcacgtacgagatataggaagctgctgtc

SEQ ID NO: 298 - RNA, 156 nt, synthetic construct
  misc_feature 1..156: note = source = /note="Description of Artificial Sequence: Synthetic polynucleotide"
  source 1..156: mol_type = other RNA
cgacggttagaggccgtatgtcgatttgctttaatttcgtgcgtgtgcattgtcgtcctccattacagggcggctaccacgaatagtcacgaagttccactgagtaaagtggaaagcttcgaggttagcacactacaacagccacaacgtctatat

SEQ ID NO: 299 - RNA, 140 nt, synthetic construct
  misc_feature 1..140: note = source = /note="Description of Artificial Sequence: Synthetic polynucleotide"
  source 1..140: mol_type = other RNA
cgacggttagaggccgtatgtcgatttgctttaatttcgtgcgtgtgcattgtcgtcctccattacagggcggctaccacgaatagtcacgaagtaaaagcttcgaggttagcacactacaacagccacaacgtctatat

SEQ ID NO: 300 - RNA, 149 nt, synthetic construct
  misc_feature 1..149: note = source = /note="Description of Artificial Sequence: Synthetic polynucleotide"
  source 1..149: mol_type = other RNA
cgacggttagaggccgtatgtcgatttgctttaatttcgtgcgtgtgcattgtcgtcctccattacagggcggctaccacgaatagccacgaagttccaaaatggaaagcttcgtggctagcacactacaacagccacaacgtctatat

SEQ ID NO: 301 - RNA, 140 nt, synthetic construct
  misc_feature 1..140: note = source = /note="Description of Artificial Sequence: Synthetic polynucleotide"
  source 1..140: mol_type = other RNA
cgacggttagaggccgtatgtcgatttgctttaatttcgtgcgtgtgcattgtcgtcctccattacagggcggctaccacgaatagccacgaagtaaaagcttcgtggctagcacactacaacagccacaacgtctatat

SEQ ID NO: 302 - RNA, 135 nt, synthetic construct
  misc_feature 1..135: note = source = /note="Description of Artificial Sequence: Synthetic polynucleotide"
  source 1..135: mol_type = other RNA
cgacggttagaggccgtatgtcgatttgctttaatttcgtgcgtgtgcattgtcgtcctccattacagggcggctaccacgaatagccacgaagtaaaagcttcgtggctagcacactacaacagccacaacgtc

SEQ ID NO: 303 - RNA, 140 nt, synthetic construct
  misc_feature 1..140: note = source = /note="Description of Artificial Sequence: Synthetic polynucleotide"
  source 1..140: mol_type = other RNA
cgacggttagaggccgtatgtcgatttgctttaatttcgtgcgtgtgcattgtcgtcctccattacagggcggctaccacgaatagccacgaagtaaaagcttcgtggctagcacgtacgagatataggaagctgctgtc

SEQ ID NO: 304 - DNA, 3324 bp, synthetic construct
  misc_feature 1..3324: note = source = /note="Description of Artificial Sequence: Synthetic polynucleotide"
  source 1..3324: mol_type = other DNA
atggccattcggagcatcaagctcaagctgaagaccagaaccggtcctgaggctcagaacctgcggaagggcatctggagaacccaccggctgctgaacgagggcgtggcctactacatgaagatgctgctgctcttccggcaggaatccactggcggccagacaaagaaggaactgcaggaggaactggtgcggcacatccgggagcagcagcagaaaaacagagccgacaagaatacccaggccctgcctctggataaggccttcgccgctctgagacaactgtacgagctgctcgtgccatctagcatcggccagagcggcgatgcccagatcatctctagaaaattcctgtctcctttggtggaccctaacagcgagggcggcaagggcactagcaaggccggagccaagcccacatggcagaaaaagaaggaggccaatgaccctacatgggagcaagattacgagaagtggaaaaagcgtagagaggaagatcctaccgccagtgtgataacaaccctggaagaatatggaattagacctatctttccactgtacaccaacaccgtggccgatatcgcctggctgcctctgcagtctaaccagtttgtccggacatgggatcgggacatgctgcagcaggccatcgagaggctactctcttgggagtcttggaacaagcgggtgcaagaggaatacagcaagctgcaggagaagatgacgcaactgaacgagcaactggagggcggacaggagtggatcagcctgctggaacaatacgaggaacagagagagcaggagctgatcgaaaacatgaccgccgcaaacgataaataccgcatcaccaagcggcagatgaagggctggaatgagctgtatgagcagtggtctaccgtgctgcccaatgccagccacgagcagtacagagaggcactgaaaagagtgcaacagcggctgaggggcagattcggcgacgctcatttcttccagtatctgatgaaagaagaacaccacctgatctggaaggggaaccctcagagaatccactacttcgtggccagaaacgagctgaagaaaagactggaagaagccaagcagaacgccaccatgaccctgcctgatgccagaaagcaccctctgtgggtcagatttgacgccagaggcggcaacctgcaagactactacctgacggccgaagccgacaaccccagaagcagaaggttcgtgaccttcagccaactgatctggcctaacgagagcggctggatggaaaaacaggacgtggaagttgaactggccctgagcaagcagttctaccagcaagtgacactgcagaaaaacgacaaggggaagcaggaaatcgagttcaaagacaagggcagtggcagcaccttcagcggccacctgggcggggccaagctgcaactggagagaggagatctggagaaggaagaaaaagactttgaaggcggcgaaatcggcagcgtgtacctgaacatcgtgatcgacttcgagccactgcaggaggtgaagaacggccgactgcaatctccttacggccaggtgctgcaactggtcagaaggcctaatgagttccccaaggtgaccacctacaagtctgaggaactggtcgagtggatcaagagcagcaccaaggacagcgccggcgtggagtccttagagagcggttttagagtgatgagcatcgacctgggactgcggaccgccgccgcaacaagcatcttctctgtagaggaatccaacgacgccaacgccgcgggtttcagctactggatcgagggaactcctctggttgccgttcataagcggtcatatatgctgaaactgcccggagagcaagtcgaaaagcaggtgcgagagaagcgggacgagcggcaggatcagcagagaagagtgaggttccaaatcagaatcctgagccaggtgatccggatggccaagaagcagaaccgggagcgggctgatgagctggaccacctgtcccaggccctggagaagcaaaaatctctgctggaccagaccgatcggaccttttggaacggcatcgtgtgcgacctgacagacgctctgagagagaaagagggcggatgggaacaggccgtggtccagatccacaggaaggccgaggagcacgtgggcaaggtggtgcaagcctggcggaaacggttcgacgccgatgaacgcaagggcatcgccggcctgtctatgtggtctatcgaggagctggacagcctgcggaagctgctgatctcttggagcagaagaacaagaaaccccagagaaatcaactgcttcgagcagggccacaccagccaccagcggctgctgacacacatccagaacgtgaaggaggaccggctgaagcaactgagccacgccattgtgatgacagccttgggctacgtgtacgacgagaagaaattggaatggtttgccaagtaccctgcttgtcaggtgatcctgttcgagaacctgtcccagtaccggtccaacatggacagaagcaccaaagagaatagcaccctgatgaaatgggcccacaggagcatccctaagtacgtgcacatgcaggccgagccttacggcatccagatcggcgatgtgcgggccgagtactccagcagattccacgccaagacaggcacacctggcatccggtgcaagatggtgtccggacacgacctgcaaggcaggcgcttcgagaacctgcagaagcggttaatctctgaacagttcctgacagaggagcaagtgaagcagctcagacccggcgacatcgtgcccgacgactccggcgagtggttcatgaccctgagcgacggcagcgaaggcaaagaggttgtgttcctccaagccgacatcaacgccgcccaaaatctgcaaaagagattctggcagcggtacaacgagctgtttaaggtctcctgcagagtgctgatccgaggagaagaggaatacctgatccccaagacaaagtccgtgcaagccaagctgggcaaaggcctgttcgtgaaaaaaaccgacaccgtgatgaaggacgtgtacgtgtgggacagccaggctaagctgaagggcaaaaccacattcaccgaggagtccgaaagccctgagcaactggaggattttcaggagatcatcgaagaagccgaagaagctaagggcacatacagaacactgtttagagaccccagcggagtgttcttccctgagttcgtgtggtccacccagaaagatttctggtccgaggtgaagagacggctgtacggcaagctgagagagcggttcctgatgaagaccagg

SEQ ID NO: 305 - DNA, 3324 bp, synthetic construct
  misc_feature 1..3324: note = source = /note="Description of Artificial Sequence: Synthetic polynucleotide"
  source 1..3324: mol_type = other DNA
atggccatcagatctatcaagctgaagctcaagaccagaacaggtcctgaggcccagaacctgcggaagggcatttggcggacacaccgcctcctcaacgagggcgtcgcctattatatgaaaatgctgctgctgttcagacaggagagcacaggcggccagaccaagaaagagctgcaggaagaactggtccggcacatcagagagcagcagcagaagaatcgggccgacaagaacacccaggccctgcctctggacaaggcttttgccgctctgcggcagctctacgagctgctggtgccttcttctatcggccagagcggagacgcgcagatcatcagcagaaagttcctgtcccctctcgtggaccccaacagcgagggcggcaagggcacatctaaagctggcgccaagcctacatggcagaaaaagaaggaggccaacgaccccacctgggaacaggattacgagaaatggaagaagcggagagaggaggaccctaccgccagcgtgattaccaccctggaagaatacggcatcagaccaatcttcccactgtacaccaacaccgtggccgacatcgcctggctgcctctgcagtccaaccagttcgtgcggacatgggacagagacatgctgcagcaggctatcgagagactgctgagctgggaaagctggaacaagagagtgcaagaggaatacagcaagctgcaagagaagatgacacagctcaacgagcaactggagggcggccaggagtggatcagcctgctggaacagtacgaggagcaacgggagcaggagctgatcgagaacatgaccgccgctaacgacaaatatagaataacaaagcggcagatgaagggctggaacgagctgtacgagcagtggagcaccgtgctgcccaatgcctctcacgagcagtaccgggaagcccttaagcgggtccagcaaagactgcggggcagattcggcgacgctcatttcttccagtatctgatgaaggaagagcaccacctgatttggaagggcaacccccagagaatccactactttgtggccagaaacgagctgaagaaaagactggaagaggccaagcagaacgccactatgaccctgccagacgccagaaagcaccccctgtgggtgcggttcgacgccagaggcggaaatctgcaggactactacctgaccgccgaggccgataaccccagatctagaagattcgttacctttagccagttgatctggcctaacgagtccggctggatggaaaagcaggacgtggaagtggaactcgccctgagcaagcagttctaccagcaagtgaccctgcagaagaacgataaggggaaacaggagatcgagtttaaggacaagggctccggatctacgttcagtggccatctgggcggggctaagctgcaactggagcgaggcgacctggagaaagaagagaaggactttgagggcggagaaatcggaagcgtgtacctgaacatcgtgatcgacttcgagcccttgcaggaggtgaaaaacggcagactgcagagcccatacggccaggtgctgcagctcgttcggagacctaatgagttccctaaggtgaccacatacaagtctgaagaacttgttgagtggatgaaggccagccagaatcacagcagcggcgtggagtctctggagtcgggcttcagagtgatgagcatcgatctgggactgaggacagccgctgccaccagcattttctctgtggaagaaagcaacgatgccaacgccgctggcttcagctactggatcgagggcacccctctggtcgccgtgcacaagagaagctacatgctgaagctgccaggcgaacaagtggaaaaacaggtgcgggaaaagagagatgagagacaagaccagcagaggcgcgtcagatttcagatcagaatcctgagccaggtgatcaggatggccaagaaacaaaacagagaaagagctgacgaactggaccacctgagccaggcactggagaagcagaagtccctgctcgatcagaccgatagaaccttctggaacggcatcgtttgtgacctgaccgatgcgctgcgcgaaaaggagggaggctgggagcaagccgtggtccaaatccacagaaaggccgaggaacacgtgggcaaggtggtgcaagcctggcggaaaagatttgacgccgatgagcggaagggcatcgccggcctgagcatgtggtccatagaagagctggacagcctccggaagctgctcattagctggagcagaaggacaagaaaccctcaggagatcaacagattcgagcagggccacacctctcaccagcggctgctgacacatatccagaacgtgaaggaagatagactgaagcaactgagccacgccatcgtgatgaccgccctgggctacgtgtacgacgagaagaagctggagtggttcgccaaataccccgcctgccaggtgatcctgttcgagaatctgtctcagtacagaagccacatggacagatccacgaaggaaaatagcaccctgatgaaatgggcccacagaagcatccctaagtacgtccacatgcaggccgagccttacggaatccagatcggagatgtgagagccgaatatagcagcaggttccacgccaagacagggacacctggcatccgttgcaagatggtgaagggacaagaactgcaaggcaagcgattcgagaacctgcaaaagagactggtgtccgaacagtttctgaccgaggaacaggtgaagcagcttcggcctggagatatcgtgccagatgacagcggagagtggttcatgaccctgagtgatggcagcgaaggcaaggaagtggtgttcctgcaagccgacatcaacgccgcccagaaccttcagaaacgattctggcagagatacaacgaactgttcaaggtgtcatgcagagtgctgatcagaggcgaggaagagtacctgatccccaaggccaagagcgtgcaagccaaactcggcaagggactgttcgtgaagaaaaccgacaccgtgatgaaggacgtgtacgtgtgggatagccaggccaaactgaagggcaaaacaaccttcaccgaggaaagcgagagccctgagcaactggaggacttccaggagatcatcgaagaagccgaggaagccaagggcacctacagaacactgtttagagatcctagcggcgttttcttccccgagttcgtgtggaatacccagaaagacttctggtccgaggtgaagagaaggctgtacggcaagctgcgcgagagattcctgatgaagacccgg

SEQ ID NO: 306 - DNA, 203 bp, synthetic construct
  misc_feature 1..203: note = source = /note="Description of Artificial Sequence: Synthetic polynucleotide"
  source 1..203: mol_type = other DNA
tgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 307 - DNA, 304 bp, synthetic construct
  misc_feature 1..304: note = source = /note="Description of Artificial Sequence: Synthetic polynucleotide"
  source 1..304: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 308 - DNA, 318 bp, Homo sapiens
  source 1..318: mol_type = genomic DNA
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 309 - DNA, 3324 bp, synthetic construct
  misc_feature 1..3324: note = source = /note="Description of Artificial Sequence: Synthetic polynucleotide"
  source 1..3324: mol_type = other DNA
atggccatcagatctatcaagctgaagctcaagaccagaacaggtcctgaggcccagaacctgcggaagggcatttggcggacacaccgcctcctcaacgagggcgtcgcctattatatgaaaatgctgctgctgttcagacaggagagcacaggcggccagaccaagaaagagctgcaggaagaactggtccggcacatcagagagcagcagcagaagaatcgggccgacaagaacacccaggccctgcctctggacaaggcttttgccgctctgcggcagctctacgagctgctggtgccttcttctatcggccagagcggagacgcgcagatcatcagcagaaagttcctgtcccctctcgtggaccccaacagcgagggcggcaagggcacatctaaagctggcgccaagcctacatggcagaaaaagaaggaggccaacgaccccacctgggaacaggattacgagaaatggaagaagcggagagaggaggaccctaccgccagcgtgattaccaccctggaagaatacggcatcagaccaatcttcccactgtacaccaacaccgtggccgacatcgcctggctgcctctgcagtccaaccagttcgtgcggacatgggacagagacatgctgcagcaggctatcgagagactgctgagctgggaaagctggaacaagagagtgcaagaggaatacagcaagctgcaagagaagatgacacagctcaacgagcaactggagggcggccaggagtggatcagcctgctggaacagtacgaggagcaacgggagcaggagctgatcgagaacatgaccgccgctaacgacaaatatagaataacaaagcggcagatgaagggctggaacgagctgtacgagcagtggagcaccgtgctgcccaatgcctctcacgagcagtaccgggaagcccttaagcgggtccagcaaagactgcggggcagattcggcgacgctcatttcttccagtatctgatgaaggaagagcaccacctgatttggaagggcaacccccagagaatccactactttgtggccagaaacgagctgaagaaaagactggaagaggccaagcagaacgccactatgaccctgccagacgccagaaagcaccccctgtgggtgcggttcgacgccagaggcggaaatctgcaggactactacctgaccgccgaggccgataaccccagatctagaagattcgttacctttagccagttgatctggcctaacgagtccggctggatggaaaagcaggacgtggaagtggaactcgccctgagcaagcagttctaccagcaagtgaccctgcagaagaacgataaggggaaacaggagatcgagtttaaggacaagggctccggatctacgttcagtggccatctgggcggggctaagctgcaactggagcgaggcgacctggagaaagaagagaaggactttgagggcggagaaatcggaagcgtgtacctgaacatcgtgatcgacttcgagcccttgcaggaggtgaaaaacggcagactgcagagcccatacggccaggtgctgcagctcgttcggagacctaatgagttccctaaggtgaccacatacaagtctgaagaacttgttgagtggatgaaggccagccagaatcacagcagcggcgtggagtctctggagtcgggcttcagagtgatgagcatcgatctgggactgaggacagccgctgccaccagcattttctctgtggaagaaagcaacgatgccaacgccgctggcttcagctactggatcgagggcacccctctggtcgccgtgcacaagagaagctacatgctgaagctgccaggcgaacaagtggaaaaacaggtgcgggaaaagagagatgagagacaagaccagcagaggcgcgtcagatttcagatcagaatcctgagccaggtgatcaggatggccaagaaacaaaacagagaaagagctgacgaactggaccacctgagccaggcactggagaagcagaagtccctgctcgatcagaccgatagaaccttctggaacggcatcgtttgtgacctgaccgatgcgctgcgcgaaaaggagggaggctgggagcaagccgtggtccaaatccacagaaaggccgaggaacacgtgggcaaggtggtgcaagcctggcggaaaagatttgacgccgatgagcggaagggcatcgccggcctgagcatgtggtccatagaagagctggacagcctccggaagctgctcattagctggagcagaaggacaagaaaccctcaggagatcaacagattcgagcagggccacacctctcaccagcggctgctgacacatatccagaacgtgaaggaagatagactgaagcaactgagccacgccatcgtgatgaccgccctgggctacgtgtacgacgagaagaagctggagtggttcgccaaataccccgcctgccaggtgatcctgttcgagaatctgtctcagtacagaagccacatggacagatccacgaaggaaaatagccgcctgatgaaatgggcccacagaagcatccctaagtacgtccacatgcaggccgagccttacggaatccagatcggagatgtgagagccgaatatagcagcaggttccacgccaagacagggacacctggcatccgttgcaagatggtgaagggacaagaactgcaaggcaagcgattcgagaacctgcaaaagagactggtgtccgaacagtttctgaccgaggaacaggtgaagcagcttcggcctggagatatcgtgccagatgacagcggagagtggttcatgaccctgagtgatggcagcgaaggcaaggaagtggtgttcctgcaagccgacatcaacgccgcccagaaccttcagaaacgattctggcagagatacaacgaactgttcaaggtgtcatgcagagtgctgatcagaggcgaggaagagtacctgatccccaaggccaagagcgtgcaagccaaactcggcaagggactgttcgtgaagaaaaccgacaccgtgatgaaggacgtgtacgtgtgggatagccaggccaaactgaagggcaaaacaaccttcaccgaggaaagcgagagccctgagcaactggaggacttccaggagatcatcgaagaagccgaggaagccaagggcacctacagaacactgtttagagatcctagcggcgttttcttccccgagttcgtgtggaatacccagaaagacttctggtccgaggtgaagagaaggctgtacggcaagctgcgcgagagattcctgatgaagacccgg

SEQ ID NO: 310 - DNA, 21 bp, synthetic construct
  misc_feature 1..21: note = source = /note="Description of Artificial Sequence: Synthetic oligonucleotide"
  source 1..21: mol_type = other DNA
cccagccctaggttgtttatt

SEQ ID NO: 311 - DNA, 23 bp, synthetic construct
  misc_feature 1..23: note = source = /note="Description of Artificial Sequence: Synthetic oligonucleotide"
  source 1..23: mol_type = other DNA
ctggctacatcttccttgactac

SEQ ID NO: 312 - RNA, 140 nt, synthetic construct
  misc_feature 1..140: note = source = /note="Description of Artificial Sequence: Synthetic polynucleotide"
  source 1..140: mol_type = other RNA
cgacggttagaggccgtatgtcgatttgctttaatttcgtgcgtgtgcattgtcgtcctccattacagggcggctaccacgaatagccacgaagtaaaagcttcgtggctagcacccttggctagtcttgaattcctagg

SEQ ID NO: 313 - DNA, 25 bp, Homo sapiens
  source 1..25: mol_type = genomic DNA
ccaacagtcctacttccctgtttca

SEQ ID NO: 314 - DNA, 25 bp, synthetic construct
  misc_feature 1..25: note = source = /note="Description of Artificial Sequence: Synthetic oligonucleotide"
  source 1..25: mol_type = other DNA
gagatgtccccagtgaactccaaat

SEQ ID NO: 315 - DNA, 25 bp, synthetic construct
  misc_feature 1..25: note = source = /note="Description of Artificial Sequence: Synthetic oligonucleotide"
  source 1..25: mol_type = other DNA
cctgtcgttgcccctcccagatcat

SEQ ID NO: 316 - RNA, 49 nt, synthetic construct
  misc_feature 1..49: note = source = /note="Description of Artificial Sequence: Synthetic oligonucleotide"
  source 1..49: mol_type = other RNA
gtctgattgcctgtcgttgcccctcccagatcatggaggagttggcaga

SEQ ID NO: 317 - DNA, 41 bp, synthetic construct
  misc_feature 1..41: note = source = /note="Description of Artificial Sequence: Synthetic oligonucleotide"
  source 1..41: mol_type = other DNA
gtctgattgcctgtcgttgcccctcccaaggagttggcaga

SEQ ID NO: 318 - DNA, 69 bp, synthetic construct
  misc_feature 1..69: note = source = /note="Description of Artificial Sequence: Synthetic oligonucleotide"
  source 1..69: mol_type = other DNA
gtctgattgcctgtcgttgcccctaagtgtattaagcattgtctcagagattttggaggagttggcaga

SEQ ID NO: 319 - DNA, 39 bp, synthetic construct
  misc_feature 1..39: note = source = /note="Description of Artificial Sequence: Synthetic oligonucleotide"
  source 1..39: mol_type = other DNA
gtctgattgcctgtcgttgcccctggaggagttggcaga

SEQ ID NO: 320 - DNA, 47 bp, synthetic construct
  misc_feature 1..47: note = source = /note="Description of Artificial Sequence: Synthetic oligonucleotide"
  source 1..47: mol_type = other DNA
gtctgattgcctgtcgttgcccctcccagatcggaggagttggcaga

SEQ ID NO: 321 - DNA, 44 bp, synthetic construct
  misc_feature 1..44: note = source = /note="Description of Artificial Sequence: Synthetic oligonucleotide"
  source 1..44: mol_type = other DNA
gtctgattgcctgtcgttgcccctcccagataggagttggcaga

SEQ ID NO: 322 - DNA, 39 bp, synthetic construct
  misc_feature 1..39: note = source = /note="Description of Artificial Sequence: Synthetic oligonucleotide"
  source 1..39: mol_type = other DNA
gtctgattgcctgtcgttgcccatctgggagttggcaga

SEQ ID NO: 323 - DNA, 41 bp, synthetic construct
  misc_feature 1..41: note = source = /note="Description of Artificial Sequence: Synthetic oligonucleotide"
  source 1..41: mol_type = other DNA
gtctgattgcctgtcgttgcccctctggaggagttggcaga

SEQ ID NO: 324 - DNA, 23 bp, synthetic construct
  misc_feature 1..23: note = source = /note="Description of Artificial Sequence: Synthetic oligonucleotide"
  source 1..23: mol_type = other DNA
gtctgattggaggagttggcaga

SEQ ID NO: 325 - DNA, 40 bp, synthetic construct
  misc_feature 1..40: note = source = /note="Description of Artificial Sequence: Synthetic oligonucleotide"
  source 1..40: mol_type = other DNA
gtctgattgcctgtcgttgcccctcggaggagttggcaga

SEQ ID NO: 326 - DNA, 42 bp, synthetic construct
  misc_feature 1..42: note = source = /note="Description of Artificial Sequence: Synthetic oligonucleotide"
  source 1..42: mol_type = other DNA
gtctgattgcctgtcgttgcccctccaggaggagttggcaga

SEQ ID NO: 327 - DNA, 34 bp, synthetic construct
  misc_feature 1..34: note = source = /note="Description of Artificial Sequence: Synthetic oligonucleotide"
  source 1..34: mol_type = other DNA
gtctgattgcctgtcgttgcccggagttggcaga

SEQ ID NO: 328 - DNA, 42 bp, synthetic construct
  misc_feature 1..42: note = source = /note="Description of Artificial Sequence: Synthetic oligonucleotide"
  source 1..42: mol_type = other DNA
gtctgattgcctgtcgttgcccatcatggaggagttggcaga

SEQ ID NO: 329 - DNA, 43 bp, synthetic construct
  misc_feature 1..43: note = source = /note="Description of Artificial Sequence: Synthetic oligonucleotide"
  source 1..43: mol_type = other DNA
gtctgattgcctgtcgttgcccctccatggaggagttggcaga

SEQ ID NO: 330 - DNA, 33 bp, synthetic construct
  misc_feature 1..33: note = source = /note="Description of Artificial Sequence: Synthetic oligonucleotide"
  source 1..33: mol_type = other DNA
gtctgattgcctgtcgttgcccctcccagtact

SEQ ID NO: 331 - DNA, 44 bp, synthetic construct
  misc_feature 1..44: note = source = /note="Description of Artificial Sequence: Synthetic oligonucleotide"
  source 1..44: mol_type = other DNA
gtctgattgcctgtcgttgccccaatcttggaggagttggcaga

SEQ ID NO: 332 - DNA, 43 bp, synthetic construct
  misc_feature 1..43: note = source = /note="Description of Artificial Sequence: Synthetic oligonucleotide"
  source 1..43: mol_type = other DNA
gtctgattgcctgtcgttgccctgggatggaggagttggcaga

SEQ ID NO: 333 - DNA, 35 bp, synthetic construct
  misc_feature 1..35: note = source = /note="Description of Artificial Sequence: Synthetic oligonucleotide"
  source 1..35: mol_type = other DNA
gtctgattgcctgtcgttgcccctcagttggcaga

SEQ ID NO: 334 - DNA, 42 bp, synthetic construct
  misc_feature 1..42: note = source = /note="Description of Artificial Sequence: Synthetic oligonucleotide"
  source 1..42: mol_type = other DNA
gtctgattgcctgtcgttgcccctcatggaggagttggcaga

SEQ ID NO: 335 - DNA, 25 bp, Homo sapiens
  source 1..25: mol_type = genomic DNA
tctttggacagtgtccatactggtg

SEQ ID NO: 336 - DNA, 25 bp, Homo sapiens
  source 1..25: mol_type = genomic DNA
tggtgagcacacctcgggactgggc

SEQ ID NO: 337 - DNA, 25 bp, Homo sapiens
  source 1..25: mol_type = genomic DNA
ctgccacaataccttggcccttctc

SEQ ID NO: 338 - DNA, 25 bp, Homo sapiens
  source 1..25: mol_type = genomic DNA
gctgcgacccatacacccaaaggat

SEQ ID NO: 339 - DNA, 25 bp, Homo sapiens
  source 1..25: mol_type = genomic DNA
ttgtcgatggtcagcacagccttat

SEQ ID NO: 340 - DNA, 25 bp, Homo sapiens
  source 1..25: mol_type = genomic DNA
tgacttcatggtaactgggagaggg

SEQ ID NO: 341 - DNA, 25 bp, Homo sapiens
  source 1..25: mol_type = genomic DNA
tgtagtccagaaccgtgctggcagc

SEQ ID NO: 342 - DNA, 25 bp, Homo sapiens
  source 1..25: mol_type = genomic DNA
cacttcaggtcctgacagacactag

SEQ ID NO: 343 - DNA, 25 bp, Homo sapiens
  source 1..25: mol_type = genomic DNA
cacaggtcctcaagggcagaagagt

SEQ ID NO: 344 - DNA, 25 bp, Homo sapiens
  source 1..25: mol_type = genomic DNA
ccaagaaacggggctgaaagccggg

SEQ ID NO: 345 - DNA, 25 bp, Homo sapiens
  source 1..25: mol_type = genomic DNA
tccttcaccattatcaccacccagt

SEQ ID NO: 346 - DNA, 25 bp, Homo sapiens
  source 1..25: mol_type = genomic DNA
ggcttagggttaccgaagaggggcc

SEQ ID NO: 347 - DNA, 25 bp, Homo sapiens
  source 1..25: mol_type = genomic DNA
ggctcagggttactgaagaggggcc

SEQ ID NO: 348 - DNA, 20 bp, Homo sapiens
  source 1..20: mol_type = genomic DNA
cctaaaggaaatgactgcag

SEQ ID NO: 349 - DNA, 20 bp, Homo sapiens
  source 1..20: mol_type = genomic DNA
acttcgtctattcccagatc

SEQ ID NO: 350 - DNA, 20 bp, Homo sapiens
  source 1..20: mol_type = genomic DNA
tggactgcatcttagcccta

SEQ ID NO: 351 - DNA, 25 bp, Homo sapiens
  source 1..25: mol_type = genomic DNA
tgggtaggtaggctgggctgtgggt

SEQ ID NO: 352 - DNA, 25 bp, Homo sapiens
  source 1..25: mol_type = genomic DNA
ttgctcattgacctccactcagtgt

SEQ ID NO: 353 - DNA, 25 bp, Homo sapiens
  source 1..25: mol_type = genomic DNA
taacccccacccccactgccgggta

SEQ ID NO: 354 - DNA, 25 bp, Homo sapiens
  source 1..25: mol_type = genomic DNA
ttgctcgttgatctccactcagtgt

SEQ ID NO: 355 - DNA, 20 bp, Homo sapiens
  source 1..20: mol_type = genomic DNA
caccacggctgtcgacacca

SEQ ID NO: 356 - DNA, 20 bp, Homo sapiens
  source 1..20: mol_type = genomic DNA
caacagtggaggaaagcctt

SEQ ID NO: 357 - DNA, 25 bp, Homo sapiens
  source 1..25: mol_type = genomic DNA
ctatctggtcgtgggagtacgggat

SEQ ID NO: 358 - DNA, 20 bp, Homo sapiens
  source 1..20: mol_type = genomic DNA
aaataagcagcaggaagcag

SEQ ID NO: 359 - DNA, 20 bp, Homo sapiens
  source 1..20: mol_type = genomic DNA
ctcgatataggtggagtctt

SEQ ID NO: 360 - DNA, 25 bp, Homo sapiens
  source 1..25: mol_type = genomic DNA
tggttgtgcagccagatgcctagac

SEQ ID NO: 361 - DNA, 25 bp, Homo sapiens
  source 1..25: mol_type = genomic DNA
tcttgctcactttggaccttggtgg

SEQ ID NO: 362 - DNA, 25 bp, Homo sapiens
  source 1..25: mol_type = genomic DNA
ctggctgtaagtcgggctttcttca

SEQ ID NO: 363 - DNA, 25 bp, Homo sapiens
  source 1..25: mol_type = genomic DNA
ctgcaagcaacctccagtggtgacc

SEQ ID NO: 364 - DNA, 20 bp, Homo sapiens
  source 1..20: mol_type = genomic DNA
aggagctctcgagccagttt

SEQ ID NO: 365 - DNA, 20 bp, Homo sapiens
  source 1..20: mol_type = genomic DNA
gtcagaggaaggttgagccc

SEQ ID NO: 366 - DNA, 25 bp, Homo sapiens
  source 1..25: mol_type = genomic DNA
gggtctgtttgaacatggagaacac

SEQ ID NO: 367 - DNA, 25 bp, Homo sapiens
  source 1..25: mol_type = genomic DNA
gaaagatctagctcaccgtcctctt

SEQ ID NO: 368 - DNA, 25 bp, Homo sapiens
  source 1..25: mol_type = genomic DNA
tgcaagcccaccttggtccatgtgg

SEQ ID NO: 369 - DNA, 25 bp, Homo sapiens
  source 1..25: mol_type = genomic DNA
ttccctccatcatgtgtcccagggc

SEQ ID NO: 370 - DNA, 25 bp, Homo sapiens
  source 1..25: mol_type = genomic DNA
ggaagtggttggacaagtaagcgcc

SEQ ID NO: 371 - DNA, 25 bp, Homo sapiens
  source 1..25: mol_type = genomic DNA
cccgcctgcttacagatttcagttt

SEQ ID NO: 372 - DNA, 20 bp, Homo sapiens
  source 1..20: mol_type = genomic DNA
gagagtacagccacaccatt

SEQ ID NO: 373 - DNA, 25 bp, Homo sapiens
  source 1..25: mol_type = genomic DNA
tatcacttacggatcacagaggggg

SEQ ID NO: 374 - DNA, 25 bp, Homo sapiens
  source 1..25: mol_type = genomic DNA
gtgacagagctggtttcaaagttgc

SEQ ID NO: 375 - DNA, 25 bp, Homo sapiens
  source 1..25: mol_type = genomic DNA
cctgaagccatcaaggggctgcagc

SEQ ID NO: 376 - DNA, 25 bp, Homo sapiens
  source 1..25: mol_type = genomic DNA
tttactctcccacagtggcgctgct

SEQ ID NO: 377 - DNA, 25 bp, Homo sapiens
  source 1..25: mol_type = genomic DNA
ctccaattcaggacccacatgacgg

SEQ ID NO: 378 - DNA, 25 bp, Homo sapiens
  source 1..25: mol_type = genomic DNA
atctcctcctcatcctccagcctct

SEQ ID NO: 379 - DNA, 25 bp, Homo sapiens
  source 1..25: mol_type = genomic DNA
gggggctggtttgccatccaagggg

SEQ ID NO: 380 - DNA, 25 bp, Homo sapiens
  source 1..25: mol_type = genomic DNA
gccggacagggatgttcattgtgat

SEQ ID NO: 381 - DNA, 25 bp, Homo sapiens
  source 1..25: mol_type = genomic DNA
gccattttggctgagttggtccagt

SEQ ID NO: 382 - DNA, 25 bp, Homo sapiens
  source 1..25: mol_type = genomic DNA
gccactttggctgagttggtccggt

SEQ ID NO: 383 - DNA, 25 bp, Homo sapiens
  source 1..25: mol_type = genomic DNA
agctgctagtcccagctgaggacgt

SEQ ID NO: 384 - DNA, 25 bp, Homo sapiens
  source 1..25: mol_type = genomic DNA
ttggactcccttgatgcccaggaga

SEQ ID NO: 385 - DNA, 20 bp, Homo sapiens
  source 1..20: mol_type = genomic DNA
cctaccctgaaaatccgaaa

SEQ ID NO: 386 - DNA, 25 bp, Homo sapiens
  source 1..25: mol_type = genomic DNA
tagtagcacacactctgcaccttga

SEQ ID NO: 387 - DNA, 25 bp, Homo sapiens
  source 1..25: mol_type = genomic DNA
ccctcgttgacatcgatgcttgaga

SEQ ID NO: 388 - DNA, 25 bp, Homo sapiens
  source 1..25: mol_type = genomic DNA
gacagcggaaaccccggaaaagcag

SEQ ID NO: 389 - DNA, 25 bp, Homo sapiens
  source 1..25: mol_type = genomic DNA
acctgagtgttttagctacggtgag

SEQ ID NO: 390 - DNA, 20 bp, Homo sapiens
  source 1..20: mol_type = genomic DNA
gaaggtctcctccctggtca

SEQ ID NO: 391 - DNA, 25 bp, Homo sapiens
  source 1..25: mol_type = genomic DNA
ctgctgaatcagccggcagaaccag

SEQ ID NO: 392 - DNA, 20 bp, Homo sapiens
  source 1..20: mol_type = genomic DNA
tgtgcccgcactcgaaaaag

SEQ ID NO: 393 - DNA, 20 bp, Homo sapiens
  source 1..20: mol_type = genomic DNA
cttgggactgtaaaagctgt

SEQ ID NO: 394 - DNA, 20 bp, Homo sapiens
  source 1..20: mol_type = genomic DNA
gtaagtgatccatcctaggt

SEQ ID NO: 395 - DNA, 20 bp, Homo sapiens
  source 1..20: mol_type = genomic DNA
aacgaagccctccgtgagag

SEQ ID NO: 396 - DNA, 20 bp, Homo sapiens
  source 1..20: mol_type = genomic DNA
caagaatctgaccatcttgg

SEQ ID NO: 397 - DNA, 20 bp, Homo sapiens
  source 1..20: mol_type = genomic DNA
tcgtccgtgacctgggagcc

SEQ ID NO: 398 - DNA, 20 bp, Homo sapiens
  source 1..20: mol_type = genomic DNA
acagatgaacactggtcagc

SEQ ID NO: 399 - DNA, 20 bp, Homo sapiens
  source 1..20: mol_type = genomic DNA
tcagtgccaacctcctcaca

SEQ ID NO: 400 - DNA, 20 bp, Homo sapiens
  source 1..20: mol_type = genomic DNA
ttgatcttgtcgggagttcc

SEQ ID NO: 401 - DNA, 20 bp, synthetic construct
  misc_feature 1..20: note = source = /note="Description of Artificial Sequence: Synthetic oligonucleotide"
  source 1..20: mol_type = other DNA
aagactaacctggccaacat

SEQ ID NO: 402 - DNA, 20 bp, synthetic construct
  misc_feature 1..20: note = source = /note="Description of Artificial Sequence: Synthetic oligonucleotide"
  source 1..20: mol_type = other DNA
gtcttgaactccggacctca

SEQ ID NO: 403 - DNA, 20 bp, synthetic construct
  misc_feature 1..20: note = source = /note="Description of Artificial Sequence: Synthetic oligonucleotide"
  source 1..20: mol_type = other DNA
gccgggcgtggtgtcgcgcg

SEQ ID NO: 404 - DNA, 20 bp, synthetic construct
  misc_feature 1..20: note = source = /note="Description of Artificial Sequence: Synthetic oligonucleotide"
  source 1..20: mol_type = other DNA
tcctgccgcagcctctggag

SEQ ID NO: 405 - RNA, 135 nt, synthetic construct
  misc_feature 1..135: note = source = /note="Description of Artificial Sequence: Synthetic polynucleotide"
  source 1..135: mol_type = other RNA
cgacggttagaggccgtatgtcgatttgctttaatttcgtgcgtgtgcattgtcgtcctccattacagggcggctaccacgaatagccacgaagtaaaagcttcgtggctagcacaagactaacctggccaacat

SEQ ID NO: 406 - RNA, 135 nt, synthetic construct
  misc_feature 1..135: note = source = /note="Description of Artificial Sequence: Synthetic polynucleotide"
  source 1..135: mol_type = other RNA
cgacggttagaggccgtatgtcgatttgctttaatttcgtgcgtgtgcattgtcgtcctccattacagggcggctaccacgaatagccacgaagtaaaagcttcgtggctagcacgtcttgaactccggacctca

SEQ ID NO: 407 - RNA, 135 nt, synthetic construct
  misc_feature 1..135: note = source = /note="Description of Artificial Sequence: Synthetic polynucleotide"
  source 1..135: mol_type = other RNA
cgacggttagaggccgtatgtcgatttgctttaatttcgtgcgtgtgcattgtcgtcctccattacagggcggctaccacgaatagccacgaagtaaaagcttcgtggctagcacgccgggcgtggtgtcgcgcg

SEQ ID NO: 408 - RNA, 135 nt, synthetic construct
  misc_feature 1..135: note = source = /note="Description of Artificial Sequence: Synthetic polynucleotide"
  source 1..135: mol_type = other RNA
cgacggttagaggccgtatgtcgatttgctttaatttcgtgcgtgtgcattgtcgtcctccattacagggcggctaccacgaatagccacgaagtaaaagcttcgtggctagcactcctgccgcagcctctggag

SEQ ID NO: 409 - DNA, 3324 bp, synthetic construct; identical to SEQ ID NO: 304
  misc_feature 1..3324: note = source = /note="Description of Artificial Sequence: Synthetic polynucleotide"
  source 1..3324: mol_type = other DNA

SEQ ID NO: 410 - DNA, 3324 bp, synthetic construct; identical to SEQ ID NO: 305
  misc_feature 1..3324: note = source = /note="Description of Artificial Sequence: Synthetic polynucleotide"
  source 1..3324: mol_type = other DNA

SEQ ID NO: 411 - DNA, 3279 bp, synthetic construct
  misc_feature 1..3279: note = source = /note="Description of Artificial Sequence: Synthetic polynucleotide"
  source 1..3279: mol_type = other DNA
atgaaaaccctggggatcgatctgggaaccgccagcatcggctgggctatcctggacgaggccagcatcatcgcctccggcgtgcggatctttagccagagcgagatggctggcagagatccccagagcaaggccagcctggccgtcgcccggagggacgccagaggagctagaaggaggagagacagatacctgaaaagacggaggagactgctggacctgctgacagagcacggcctggctccagccgatgagaaaagccggaaggccctggtgcgggaatacgaggatggcaaagatggagatctgagcaacagcgtgtacgcccttagagctagagccttggatgaagccctgaccccttacgagatcgggcgggccatcttccaactgaaccagcggcggggctttaagtctaacagaaagaccgacagcaacgaccccgagcagggaaaaatcgccacagcaatccacgtgctggatgccaaaatggacgaggataaggcccggaccttcggagagtggctgcacatgagaagactcaaaggcctgagcgtgcgcgctcggatgaccgccgacggcgattcttacgatttctaccctagccgtgccgccctggaaagagagttcgacagactgatggccagccagaagagattccaccctgacctgctgaactcaagcgtgatcgacgacatcaggaaggtggtgtttcaccagcggcctctgaagcctgtgcaacctggcaagtgtagctacaaccacagagaatctcggctgcccaaggcacaccccttgtttcagaaattccggctgctgaaagaggtgaatgagctggaaatcgtcggcgaagatcagagatacgtgaagctgacccctgcccagcgcgatgtgctgacactggccctcagaacaggcctgaccaagcaaggtagactgccgttctctaagctgcggagcattctgaagctgggcaaggaggttcggtttaacaaggaaaaggacaaccggaccgacctggaaggcgatgtgatccacttcagagtgtccaggcccgactgcttcggcaatagatgggccgctatgcctgtggaagagcaagccgctgtgaccgagaagctgagaaccgagccagattacagcgccctgctcgattggctgaagaacgaggctggcttggatgaggctcatgcccgagccgtggccgacacacctgtgcccgacggcttcggcagaatgggacccagcgccttgtctgccctggctgatgctatggaacacgagattgatgcacagggcttcgtgatcaccgaggccgaggccgccaagagagtgtacggcagaaccaatagcgaggctgaccccggccggaaaggagtggaccaactgcctaagtaccaggaggtcctgcagaggcatattcctcctggaacaggcgaacctgatgacccctacgacgagtacatgggcagaatcaccaaccccaccgtgcacatcgccctgaaccaactgagaagactggtcaacgcactgatccggaagtacggcaagcccaacaaaatcgccatcgaagtgggccgggagctgaaactgaacgaaaagcagagaaacgaggtcaaccgagaaatcggccaaaataccagagccgctatggccagaggccagcaactggtggaaatcttcaagcagccaaacaccggatacaaccggctgagactggaactgtgggaagatctgaaccgggaacagcctctgaaacggctctgtacctactgcggcaaggccatcgctgcccacatgctgttcaacggcgagacagatatcgatcacatcctgccatattccaagacactggatgactccaaggccaacaggctgttgtgctgcacaccttgcaacagagagaagaagaactacgcccctgccaacgtgctgcagtggcgggaccactacggcgagatcctggccagagccaccgccctccctaagaacaagcagtggagattcgccgaggatgctatgaccagatacgaggccgagggcggtttcctggcccggcagctcacagatatgcagtacatcagcagactcgccctgacatacctggcccacctgtacgactacgaggaacccgacctggacggcgtgtacaagagacacgacagagtgagagccctccctgggcgcatgaccgagatgctgagacggcagtgggccctgaacgaactgctccacggccacaacctggctggtggcgacggcgccaaggaaaagaacagactggatcacagacaccacgccattgatgctatcgtgatcgcttgtaccagccagtctcttattaacagactgtccaccgccgccggcgaggccgaagaacggggcgccgccagagtggtcgagagaatcgaccctccttggcctagcttccgggaagatgtgagagaagctgttaatgccattgtggtttctcacaagcctgaccacggcaccgctagccggagcggctacgacaagggcagaggccagaccgccggcaagctgcacaacgacactgcctatggcgagacaggcgagaaagaccacaacggcaacaacctggtggtgcggcggatcgccatcagcgacatcaagcggagcgccgacatcatgaagatcagaacaaacgcccacggccacagcgagctgagagacagactgtacgaagccactcgggatctggaaggcaaggctttcgagcaggccgtgacagcttttgtgaagcacgacgccaagttcaaggggatcagacacgtgcgagtgaccgaagttcagaaccccgtctggatcacccacggcggaggcaaatacaagaagggctacctgcctggaggcaatgatcggttcgacgtgtgggagctgcctgatggtaaatgggacgccgaggtggtgacgacctttgatgcccatcggcctgacttcacccctagaatgcggatcgagcaccacaacgctcggaagatcatgtctctgaagaaaggcgacatgatcgcctacgacgatcctgacagcgggaagcgcgtgattgccatcgtgcggaaattcgaccagcggaacaagcaactgtacctggaccctcacaatgaggccggcaacctggaccagagagaaaaagaaaaaacatacaagcccctaagacctatgcccaaccctctgaaaaagtacagacccagacaggtgcgggtggacgagattggacaagtgttcgaccctggcccttggtgggagaagcgctctgat

SEQ ID NO: 412 - DNA, 3162 bp, synthetic construct
  misc_feature 1..3162: note = source = /note="Description of Artificial Sequence: Synthetic polynucleotide"
  source 1..3162: mol_type = other DNA
atgaaagagaagtatatcctgggcctagacctgggcattacatctgtgggatacggcatcatcaacttcgagacaaagaaaatcattgacgccggcgtgcggctgtttcctgaagccaacgtggacaacaatgagggaagacggagcaagagaggcagcagaaggctgaagagaagaagaattcacagactggaaagagtgaagctgctgctgaccgagtacgacctgattaacaaggaacagatccctacaagcaacaacccctaccagatcagagtgaaaggactgagcgagatactgagcaaggatgaactggccatcgctcttctgcacctggctaaaagacggggaatccacaacatcaacgtcagcagcgaagatgaggatgctagcaacgagctgtccaccaaggaacagattaaccggaacaacaagctgctcaaggacaaatacgtgtgcgaggtgcaactgcagcggctgaaggagggccagatccggggcgagaagaacagattcaagacaaccgacatcctgaaagaaatcgaccaactgctgaaggtgcaaaaagattaccacaacctggacatcgacttcatcaatcaatacaaggagatcgtggaaacacggagagagtacttcgagggccccggccagggctctccattcggctggaacggggatctgaagaaatggtatgagatgctgatgggccactgcacctacttcccccaggagctgagaagcgtgaagtacgcctacagcgccgacttgtttaacgccctcaatgatctgaacaatctgatcattcagagagacaactccgagaaactggaataccacgagaagtaccacataattgaaaatgtgttcaaacagaagaagaaacccaccctgaagcagattgctaaagagatcggcgtgaaccctgaggacatcaagggctacagaatcaccaagagcggcaccccacagttcaccgagttcaagttataccacgatctgaagagcatcgtctttgacaagtccatcctggaaaacgaggccatcctcgatcagatcgccgagatcctgaccatctaccaagatgaacagagcatcaaggaagagctgaacaaactgcctgagattctgaatgagcaggacaaggccgagattgccaaactcattggctacaacggtacacacagactgagcctgaagtgtatccacctgatcaatgaggaactgtggcagacaagtagaaaccagatggaaatcttcaactacctgaacatcaagcccaacaaggtggacctgtctgaacagaacaagatccccaaggacatggtgaacgacttcattctgagccctgtggtgaaaagaaccttcatccagagcatcaatgtgatcaataaggtgatcgagaagtacggcatcccagaggacatcatcattgagctggccagagagaacaacagcgacgaccggaagaagttcatcaacaacctgcagaagaagaacgaggccacaagaaagcggattaacgagatcatcggccagaccggcaaccagaatgccaaacgcatcgtggaaaagatcagactgcacgaccagcaggagggcaagtgcctgtacagcctggaaagcattgctctgatggacctgctgaacaatcctcagaattacgaggtggaccacatcatccccagaagcgtggccttcgacaacagcatccacaacaaggtgctggtgaagcagatcgagaatagcaagaagggcaaccggaccccttaccaatacctgaattctagcgatgccaagctgtcttacaaccagttcaagcaacacatcctgaacctgtccaaaagcaaggatagaatcagcaaaaagaagaaggactacctgctggaagagagagatatcaacaagttcgaggtgcaaaaggagttcatcaaccgaaacctggtggacaccagatacgccaccagagaactgacgtcttatctgaaggcctacttttctgccaacaacatggatgtgaaagtcaagaccatcaacggctccttcaccaaccacctgcggaaagtgtggcggttcgacaagtaccgcaaccacggctacaagcaccacgccgaggatgccctgatcatcgcaaacgccgatttcctgtttaaggaaaacaaaaagctgcagaacgctaacaagattctggaaaagcccacaatcgagaacaataccaagaaggtgaccgtggaaaaagaggaagattataataatgtgttcgagactcctaagctggtcgaggatatcaaacagtaccgggactacaagttcagccaccgggtggataagaagcctaacagacaactgatcaacgacactctgtattccacaagaatgaaggacgagcacgactatatcgttcagaccatcaccgacatctacggcaaggataacaccaatctgaaaaagcagttcaacaagaaccccgaaaaattcctgatgtaccagaacgatcctaagaccttcgagaagctgtccatcatcatgaaacagtacagcgacgagaagaaccctctggccaagtattatgaggaaaccggagagtatctgacaaagtactcaaagaagaataacggcccaatcgtgaagaagatcaagctgctgggcaacaaggtgggcaatcacctggatgtcaccaacaaatacgagaacagcacgaaaaagctggtgaagctgagtatcaagaactacagattcgacgtctacctgacagagaagggctacaaattcgtgaccatcgcttacttaaatgtgttcaagaaggacaactactactacattcctaaggacaagtaccaggagttgaaggaaaaaaagaagatcaaagataccgaccagtttatcgcttctttttacaagaatgacctgatcaagctgaacggcgacctttataagatcatcggcgtgaactccgatgacagaaatatcatcgagctggactactacgacataaaatacaaggactactgcgaaatcaacaacatcaagggagagcctagaatcaagaaaaccatcggaaaaaagacagagagcatcgagaagttcaccacagatgtgctgggaaatctgtacctgcattctaccgaaaaggcccctcaactgatcttcaagagagggctg

SEQ ID NO: 413 - DNA, 3213 bp, synthetic construct
  misc_feature 1..3213: note = source = /note="Description of Artificial Sequence: Synthetic polynucleotide"
  source 1..3213: mol_type = other DNA
atgagggagctggactacaggatcggcctggacatcggcaccaacagcatcggctggggcgtgatcgagctgagctggaacaaggacagggagcagtacgagaagaccaggatcgtggacaagggcgtgaggatgttcgacaaggccgagatccccaagaccggcgccagcctggccgagcccaggaggatcgccaggagcagcaggaggaggctgaacaggaagagccagaggaagaaggacatcaggaacctgctggtgcagcacgagatcatcagccagaaggagctgaccagcctgtaccccctgagcaagagcagcatggacatctgggacatcaggctggacggcctggacaggctgctggacaggttcgagtgggccaggctgctgatccacctggcccagaggaggggcttcaagagcaacaggaagagcgagctgaaggacgtggagaccggcaaggtgctgagcagcatccaggtgaacgagaagaggctgagcctgtacaggaccgtgggcgagatgtggatgaagaacgccgactgcagcaagtacggcaagaggaggaacagccccaacgagtacgtgttcagcgtgagcagggccgacctggagaaggagatcgtgaccctgttcgaggcccagaggaagttccacagcagctacgccagcgtggacctgcagaagacctacatccagatctgggcccaccagctgcccttcgccagcggcaacgccatcgtgaacaaggtgggctactgcagcctgctgaagggcaaggagaagagggtgcccaaggccacctacaccttccagtacttcaacaccctggaccagatcaacaggaccaggctgggccccaacttccagcccttcaccaaggagcagagggacatcatcctggacaagatgttccagaggaccgactactacaagaagaagaccatccccgaggtgacctactacgacatcaggaagtggctggccctggacgagaccatccagttcaagggcctgacctacgaccccaacgaggagctgaagaagatcgagatgaagcccttcatcaacctgaagcccttctacgagatcaagaaggtggtgaccaactacgccaagaagaccaacgaggtgttcagcgccctggactacgacaccgtggcctacgccctgaccgtgtacaagaccgacaaggacatcaggagctacctgaagaggagcaacaacctgagcaagaggtgctacgacgaccagctgatcgaggagctgctgaccctgagctacaccaagttcggccacctgagcttcaaggccatcaaccacgtgctgcccatcatgcaggagggcaggacctaccaggaggccatccaccagctgggctacgacaccaccaacctgaagaaggagaacaggagcatgttcctgcccatcatccccgacgagatcaccaaccccatcgtgaagagggccctgacccaggccaggaaggtggtgaacgccatcatcaggaggtacggcagccccaacagcgtgcacatcgagctggccagggagctgagcaagagccacgacgagaggaagaagatcatgaccgcccacgacgagaactacaagaagaacaagggcgccatcagcatcctgatcgagaacggcatcctgaaccccaccggctacgacatcgtgaggtacaagctgtggaaggagcagggcgagaggtgcgcctacagcctgaagaagatccccgccaacaccttcttcaacgagctgaagaaggagaggagcggcccccccgtgctggaggtggaccacatcctgccctacagccagagcttcatcgacagctaccacaacaaggtgctggtgtacggcgacgagaaccagaagaagggcaacaggatcccctacaccttcttcagcgaggaggacaaggagtgggagagcttcgagagctacgtgaggagcaacagcttcttcagcaagaagaagaggggctacctgctgaagaaggcctacctgcccagggagagcaacctgatcaaggagaggcacctgaacgacaccaggtacgccagcagctacctgaagaacttcatcgagaagaacctgaagttcaaggaggccgtgggcatcaccaggaagaagtacgtgcagaccgtgaacggcgtgatcaccgcccacctgaggaagaggtggggcctggagaaggacaggcaggagacctacctgcaccacgccatggacgccatcatcgtggcctgcaccgaccaccacatggtgaccaaggtgaccgagtactaccagatcaaggagggcaacaagagcatcaagaagccctacttccccctgccctggatgggcttcagggaggagatcctgagccacctggagagccagcccatcgccaggaagatcagcgaggagctgaagatcggctaccagagcagcgactacatcctggtgagcaggatgcccaagaggagcgtgaccggcagcgcccacgaccagaccgtgatgaagaagggcggcatcgacaagaagggcaagaccatcatcatcaagagggtgcacctgaaggacatcaagttcgacgagaacggcgacttcaagatggtgggcaaggagcaggacctggccacctacgaggccatcaagcagaggtacctggagtacaggaagaagagcaagaaggccttcgagacccccctgtacaagcccagcaagaagggcaagggcaacctgatcaagaagatcaaggtggaggtgcagaccaagagcttcgtgagggagatcaacggcggcgtggcccagaacggcgacctggtgagggtggacctgttcgagaaggacaacaggtactacatggtgcccatctacgtggtggacaccgtgaggagcgagctgcccaacaaggccgtgaccagcagcaagggctacgagcagtggctgagcatcgacaacagcttcaccttcaagttcagcctgtacccctacgacctggtgaggctggtgaagggcgacgaggacaggttcctgtacttcagcaccctggacatcaacagcgacaggctgaacttcaaggacgtgaacaagcccagcaagcaggccgagtacaggtacagcctgaagaccatcgagaacctggagaagtacgagatcggcgtgctgggcgacctgaggctggtgaggcaggagaccaggaagatcttcaag

SEQ ID NO: 414 - DNA, 3228 bp, synthetic construct
  misc_feature 1..3228: note = source = /note="Description of Artificial Sequence: Synthetic polynucleotide"
  source 1..3228: mol_type = other DNA
atgagcgagctggactacagaatcggcctggacataggaacaaacagcatcggctggggcgttatagaactgttttggaacaaggacagagaaagatacgagaaggtgaggatagtggacaagggcgtgcggatgtttgacaaggctgaaatccccaagaccggagcctctctggccgaacctagaagaatcgccagaagcagcagaaggagacttaatagaaagagccagagaaaaaaggagatcagaaacctgctggtgcaacacggcgtgatcacccaggaagagctggacctgctgtaccccctgtctaagaagagcatggacatttgggacatccgcctggacggcctcgacagactgctgaaccacctggaatggacacggctgctgattcacctggctcagaggcgtggatttaagtctaaccggaagtctgagctgaaagacgccgaaacaggcaaagttctgagcagcatccaggtgaacgagaaaagactgagcctgtatagaaccgtgggcgaaatgtggatcaaggacgccgaattctcaaaatacgatagacggagaaatagccctaacgaatacgtctttagcgtgtccagagccgacctggagaaggaaatcgttaccctcttcgaggcccagcggaaattccagagcagctacagcagcaaaaacctccaggagacctacctgcaaatctgggcccaccaactgcctttcgccagcggcaacgccatcttaaacaaggtgggctactgcagcctcctgaaaggcaaggagcggagaatccctaaggctacatacaccttccagtacttcagcgccctggaccaagtgaaccggaccagactcggtcctgacttccaaccttttacacaggagcagaaagaaatcatcctggacaagatgttccagcggaccgactactacaagaagaagaccattcccgaggtgtcctattatgatatcagaaagtggctggaactcgacgaaactatccaatttaagggcctgaactacgatcctaacgaggaactgaaaaagatcgagaagaagcctttcatcaacctgaaggccttctacgagatcaaaaaggttgttgccaactacgccgagagaaccaacgaggccttctccaccctggactacgacgccatcgcttacgctctgacagtgtacaagacagacaaggatattagaagttatctgaagaaaagcaacaacctcagcaaacggtgctacgacgaccaactgatcgaagaactcttcaccctgagctataccaagttcggccacctgagcttcaaggccatcaacagagtgctgcctatcatgcaggagggcagaacctaccaggaggccatccagcaactgggctacgacaccaccaacctgaagaaagagaatagaagcatgttcctgcctctgatccccgatgagatcaccaatcctatcgtgaagcgggccctgacccaggccagaaaagtggtgaacgccatcatccgccgctacggatctccaaactccgtgcacatcgagctggctagagagctgtcaaagagccacgatgagagaaagaagatcatgacagcccacgacgaaaactacaagaagaataagggcgccatcagcatcctgatcgagaacggcatcctgaaccccacaggctacgatatcgtgcggtacaaactgtggaaggagcagggcgagcggtgcgcctactccctgaaggaaattccacctgacacttttttcaacgagctgaaaaaagaacgcaacggcagtagcatcctggaagtggaccacattctcccctactcccagtcttttatcgatagctaccacaacaaagtgctggtctacagcgacgaaaatcggaacaagggcaacagaatcccttacacctacttcctggaaacaaacaaagattgggaagccttcgagagatacgtgcggagcaataagctcttcagcaagaaaaagagagagtacctgctgaagaaggcttacctgccacgggagtctgaactgatcaaggaaagacacctgaacgacaccagatacgccagcaccttcctgaagaacttcatcgagcagaacctgcagttcaaggaagtggaagtgaacctgagaaaaaagcgggtgcaaaccgtgaacggcgtgatcaccgcccacctcagaaagcggtggggcctggaaaagaaccggcaggagacctacctgcaccacgctatggacgctattatcgtggcttgtacagaccatcacatggtcaccagaatcacagagtactaccagatcaaggagagcaacaagtccgtgaagaaaccttatttccccatgccctgggagggcttcagagatgagctgctgagccatctggctagccagcctatcgccaagaagatttctgaagagctgaaagccggataccagagcagcgattacatcttcgtgagcagaatgcctaagcggtccgtgaccggcgccgcccataatcagatgatccggagaaagggcggaatcgacaaaaagggcaagacgatcatcattaagagagtgagactgaaagatatcaaattcgatgagaacggcgatttcaagatggtgggaaaagaacaagacctggccacatacgaggccattaaacagagatacctggatcacggcaagaacctcaaaaaggcctttgaaacccctctgtacaagccttctaaaaaaggcaccggaaacctgatcaagcgggtgaagatcgagggacagaccaaggcattcgtgagagaggtgaacggcggagtggcccaaaattctgacctcgtgcgggtggaccttttcgagaaggatgacaagtactacatggtgcccatctacgtgcctgatacagtgtgtagcgagctgcctaagaaagtggtcaaaagtggcaagggctatgagcagtggctgaccctggacaacagcttcaccttcaagttctccctgtacccttacgacttagtgagactggtgaagggcgacgaagatagattcctgtactttggcacactggatatcgacagcgaccggctgaacttcaaggacgtgaataaaccttctaagcagaacgagtacagatatagcctgaagaccatcgaggacctcgaaaagtacgaggtgggcgtgctgggcgacctgcggctggtgcgaaaagaaactcggcggaatttccacgagatcaaaattaac

SEQ ID NO: 415 - DNA, 3276 bp, synthetic construct
  misc_feature 1..3276: note = source = /note="Description of Artificial Sequence: Synthetic polynucleotide"
  source 1..3276: mol_type = other DNA
atgcggtacagcattggactggatatcggaaccacatctatcggaaatgccgtgatcaacaaggatctgcagaggtttgagcacctgggcgtgagaatcttcgatgccgcagagaacccgaaagacggctcttctctgagcgcccctagaagactggcccggagcagcagacggcggctgcggagaagaaagcaccgggtcgagcggaccaaacaactgctgatcaacaagggtctgctgaccaagacccaggtgaagaacctgtacaacagcaaaaacatcaacctggacatctgggacatccgggtgtcaggcatcgatagaaagctgttcaacaacgagttcgccagggtgctgatccacttcagcaaaaaccgaggattcaagtccaacaggaaaagcgagctgaaagaagatgacaatggcgctatcctgagcgccgtcaaggaaaacagagagctgatggacgagaagggctacagaacaatcgccgagatgctggtgtccgatgagaagtacgagggcaccaagagaaacaagggcggcgattacagccacgtggtggcccgaagcgacatcgagaacgaaatctgcctgctgttccagaagcagagggagtacggccaccccttcgccacagaggagaacgaggaagccttcctgagcatctggagcagccagagaccttttagcaccaaagacgacatcgtcaagaagatcggcaactgcaccctggaacctaaggaaaagagggcccctaagtccacttacaccttcgagcggttcagagccctggacaagctcaacagactgagaatcctgtctaccaccgccccttctcggcccctgactaacgaagaaagaaagagcatcctgagcagcctgttctccaagaaggaagtgaaatacaaggaactgcggaagctcctcaaactgacagacgaccagcggttcaacgagatctactactctccagatgagacaatcgagaaaaccgagaatagaacattcctgagccttgagtcccagtacaagatcaagaagatcatcgagaagaccgaaagcaagaatatgcagagcagctaccaccctattgactacgacaccatcggctatgccctgaccgtgttcaaagacgacaaggacatccaacactacctgcagaacagctacatcgacagcaagggcaaggccatccccaacatggccaacagagagtacaacctggaactgatcgaggagctgcttggcctgagctttgccaaattcggccacctgagcctcaaggctctgaacaacatcctgccttatatggaagagggcgagccctatcacatcgcttgtgaaatggctagctaccagttcagccagcggctgtccaaagaaaagcacagactgctgcctccaatcccagtggacgagatccccaaccccgtggtcgtgcgggccctgacgcaggtgcgcaaagtgctgaacagcatcatcaagaagtacggccctcctagcgacatctacatcgagctagccagagaaatgagcaagcccttcaaggagagaaaatctctggaaagagaattcaacgagaaccggcagatcaacgagaaggccaaggcccacatcagcgagctgtatagaatccccaatgaccccagaccccacgacatcctgaagttcaagctgtggaacgagcaaaacggcatctgcccctactccctgaagcctatttctatcgagtacctgttcaacatcgggtacgccgaggtggaccacattatcccatacagccggagcttcgacgacagcaacggaaacaaggtgctggtcctgaccagagaaaaccagaacaaactgaaccggaccccttatgagtggttcggccatgaggaaaaccggtgggaggatttcgtgtcgtttatcaggacaatgaaggtgggcaagaagaaaaaaaatatgctgctgaaaaagaatttcgacgaagagcaagaggagcagatcctgtctagaaatctgaatgacaccagatacatcacaagatacatcaagagcttcatcgaagataacctggagttccggacagaagaaaacaaggaacagtacgtgcacaccgtgaatggggcctatacaagcctgatgagaaagagatggggcctgaacaaggaccggagaggaaatgatctgcaccacgccgtggacgccgccatcattgccgtcagcctccctttcaagaacaaggtgaacgcctactttaagagacaggagaccggcctgagcaagctgctgaacaacaagaaggacatcttccccgagccttggcggaacttcatcaaggaactggaagccagaatgatccaggaccctgagaagatgaagagagccctggagagcctggagctggaaacatacggagaaatcttcctgaacaagctgaagcccatctttgtgtccagaatgcctaagcattctatcaaaggccagatccacgaggaaacaattagacgggtgcggggctttaccgaggaaggctttctggtgaccgtgaagaagaccagactggatcagatccctttcgacaagaacggcgacttccctatgtacggcaaggaaaccgacatcaagacctacatggccatcaagcagcgatacctggaatacggccaggacaaacaaaaggcttttgctgttcctctgagaaagcctagcaaaaaccccaaaaatgcccctatcgtgcggagcgtgaagatcgagggcaaggccaatagagtggtgatgctggacgataaggccgctgccgacaacgccagcattgtgcggaccgaagtgtttagacacaagaagaccggcgagtactacctgacacctgtgtacgtggccgatatcctgtccaataaaatcccagacagactgattaccatcaagaagagctacagcgactgggatagaatcacagacgaacacgagtatctgttttctctgtacaacaacgacctcgtgaaaattatcctgcccaaggagaaagagacaaagaagtacaccggaggcaatcacctctggcaggagggtttcttctacttcaagggagttgatagctccaacgccggcatcaagataatcaaccacctgaattctttcgaggccaggatcggcaccaagcggctgattgcatttgagaagtatcaggtgaaccctctgggcgaaatcaacaaagtgcatggcgagaaacggcctggcgagttgctgaataaagaagagatcaaagaaaatagaaagaatatctct

SEQ ID NO: 416 - DNA, 3450 bp, synthetic construct
  misc_feature 1..3450: note = source = /note="Description of Artificial Sequence: Synthetic polynucleotide"
  source 1..3450: mol_type = other DNA
atgtacagcatcggcctggacctggggatcagcagcgtgggctggagtgttatagatgagagaaccggcaacgtgatcgacctcggcatcagactgttttcagccaagaacagcgaaaagaatctggaacggcgcaccaacaggggcggacgacggctgatccggcgaaagaccaaccggctgaaggacgccaagaagatcctcgccgccgtgggcttctacgaggacaagagcctcaaaaactcttgcccttatcaactgagagtgaaaggcctgacagagcctctgtctaagggggaaatctacaaggtgaccctccatatcctgaagaagcggggcatcagctacctggatgaagatgatacagaggccgctaaggagagccaggactacaaagagcaagtgcgggaaaacgcccaactgctgacaaagtacacccctggacaaatccaactgcagcggctgaaagaaaacaacagagtgaagactggcatcaacgcccagggcaattaccaactgaacgtgttcaaggtgtccgcttacgccaacgagctggccactattctgaagacacagcaggccttctaccccaacgagctgaccgatgactggatcgccctgttcgtgcaacctggcatcgccgaggaggccggcctgatctatagaaaacggccttactaccacggccctggcaacgaagctaacaatagcccctacggcagatggagcgattttaagaaaacaggacagcctgctaccaacatcttcgacaagctgatcggcaaagacttccaaggagaactcagagccagcggactcagcctgagcgctcaacagtacaacctgctgaacgaccttacaaacctgaagatcgacggcgagattcccctgagccctgagcagaaggagtacatcctggccgagctgatgaccaaggagttcaccagattcggcgtgaacgacgtggtgaagctgctgggagtgaagaaggaacggctgagcggctggcggctggataagaagggcaagcctgagatccacaccctgaaaggctacagaaactggagaaagatcttcgccgagagcgggatcgacctcgctacactgcctacggagaccatcgactgcctggccaaggtgctgaccctgaacactgagagagaaggcatcgagaacacccttgcctttgagctgtctgaactggccgagagcgtcaagctgctggtgctggaccggtacaaggaactgagccagagcgtgagcacccaggcctggcacagattttctctgaaaaccctgcacctgctgatccctgagctgatgaacgccaccagcgagcagaacacccttctggaacagtttcaactgaagtccgatgtgcggaaaagatacagcgagtacaagaagctgcctacaaaggatgtcctgaccgaaatctacaaccctaccgtgaacaaaaccgttagccaagccttcaaggtgatcgatgccctgctcgtgaagtacggaaaagagcagatcagatacattaccatcgagatgcccagagatgacaacgaggaggatgagaagaaaagaattaaggaactgcacgccaaaaacagccagagaaaaaacgactcacagagctacttcatgcagaaaagcggatggtcccaagagaagttccagacaaccatccagaagaacagaagatttctggctaagctgctgtactactacgagcaggatggcatctgcgcctacaccggcctgtctatctctccagagctgctggtgtccgactccaccgagatcgaccacattatccctatcagcatcagcctggacgacagtatcaacaacaaggtgctggtgctgtccaaggccaaccaggtgaaaggccagcagaccccttacgacgcctggatggacggatctttcaagaagatcaacggcaagtttagcaactgggacgattaccagaagtgggtcgaaagctgtcacttcagccacaagaaagagaacaacctgctggaaaccagaaacatctttgactccgagcaagtcgaaaaatttctggctagaaacctgaacgacaccagatacgcttctagactggtgctgaacaccctgcagagcttcttcgctaaccaggagacaaaagtgcgggtggtgaatggcagcttcacccacaccctgcggaaaaagtggggcgccgacctcgacaagacaagagagacacaccaccaccatgccgttgatgccaccctgtgcgccgtgacccctttcgtgaaggtgtctcggtatcactacgcggtgaaagaagaaacaggcgagaaggtgatgagagagatcgacttcgagaccggcgagatcgtggacgaaatgtcttatagagaattcaagaagagcaagaagtacgagcggaagacgtaccaggtgaagtggcctaacttcagagagcaactgaagcccgtgaacctgcatcctagaatcaagttcagtcaccaggtggacagaaaagccaatagaaaactgagcgacgccacgatctatagcgtgcgggaaaagaccgaggtgaagaccctcaagtcaggaaaacagaaaataacaacagacgagtacaccatcggcaagatcaaggacatctacaccgtggatgggtgggaggccttcaagaaaaagcaggacaagctgctgatgaaagacctggacgagaagacctacgaaagactgctgtccatcgccgaaaccacccccgacttccaggaggtggaagagaagaacggcaaggtgaagcgggtgaagaggtccccattcgccgtgtactgcgaggaaaatgacatccccgctatccggaaatacgccaagaaaaacaacggccccctgatcagatctctgaagtattacgacggcaaactgaacaagcacatcaacattacaaaagatagtcaaggcagacctgtggaaaaaaccaagaatggcagaaaggtgacactgcagagcctgaaaccctacagatatgatatctaccaagacctggaaaccaaggcctactatacagtgcaactgtactacagcgacctgcggttcgtggaaggcaagtacggcataacagagaaagaatacatgaagaaggtggccgagcagaccaagggccaagtggttagattctgcttcagcctgcaaaagaacgatggactggaaatcgagtggaaggacagccaatgttacgacgtgcgattttacaacttccagtctgctaattctatcaacttcaagggcctggaacaggagatgatgccagccgaaaatcagttcaagcaaaagccatataacaacggggccatcaacctgaatatcgctaagtacggcaaggaaggtaagaaactgagaaagttcaacaccgacatcctgggcaaaaagcactacctgtactacgagaaggaacccaagaatatcatcaaa

SEQ ID NO: 417 - DNA, 4287 bp, synthetic construct
  misc_feature 1..4287: note = source = /note="Description of Artificial Sequence: Synthetic polynucleotide"
  source 1..4287: mol_type = other DNA
atgatgatcaaacacattctgggcctggatctgggcacgaacagcatcggatgggccctcatcaagcaaaatttcgagaacaaatatggagagatcctgggaatgggcagcagaatcatacctatgagccaagatattctcggcgagttcggcaagggaaatagcgtgtcacagaccgccgctagaaccgactaccggggcatccgccggctgagagaaaggtttctgctgcggagagaacggctgcacagaatcctgaacgtgctgaacttcctgcctgaacactacgccagccagatcgacttcgacaaacgcttcggcaagtttaaagtcgagacagaacctaagctggcctggaaaaacagcgacggcaaattctcattcctgttccagaccagcttcaacgagatgctggaggacttcaaggcccacggccaggacctgaaggtgccctatgactggaccatctattacctgcggaaaaaagctctgagccagaaaatcgaaaaagaggaactcgcctggattctgctgaacttcaaccagaagagaggctactaccaactgagaggagaagaggaagaagaaaatcccaataaactgatcgagttctacagcctgaaaatcatcgacgtgctggccgacgagcctcagaagggcaaatctgatatctggtacagccttgtccttgagaacggctggatctacagaagatcctccaaaacaagcctgctggactggaaggataagatcagagattttatcgtcaccaccgatcttaacgacaacggctccgtgaagacagacaaggagggaaatgaaaagcggagcttcagagcccctggcgagaacgactggacacttgttaagaccaaaaccgagcaggagatagaccgctccagaaagaccgtcggcacgtacatctacgagacactgctgcagaaccccaagcagaagatcaagggcaagctcgtgagaacaatcgagagaaaattctacaaggaggagctgaaacagatcctggaaaagcaaaaggaattccactacgagctgcagtccgacgatctgtacgacgactgcatccgggagctgtacagaaacaacgaggcccatcaactgaccctcagcaagaaagacttcgtgcacctgttcatggaagatataattttctatcaaagacctctgaagtcccagaaaagcagcatatctaactgcaccctggagttcagaaagtacaaagacgagaacggcgtggagcacacccagttcctgaaggccattcctaagagcaacccctattatcaggaattcagaatttggcagtggattttcaatctgaacatctacaagaaggacaacgatgacaacgtgaccaaggaattcctgagcaccaccgaagacttcgagaacctgttcgagtttctgaataatcggaaagagatcgatcagaaggctcttctgaaacacttcaagctcaatgagaaaactcacagatggaagtacgtggaagataagaagtatccttgcaacgagacaaaaaccatgatttccgagagattgaaaaaggtggaaaatatctccgacgactttctgacaagaggcatggaacagaagatctggcacatcatctactccgtgaatgacaaaaccgaatacgagaaagctctgaagagctttgccgaaaagaacaatctggatgagaatagcttcttcgaggcctttcggaaatttccacctttcaagtccgaatacggctctttctccgaaaaggccatcaagaagctgctcccactgatgagactgggcaagtattggagctacgccaacatcgacctctactcaaagaacagaatccagaaaatcatcaccggcgaattcgacgagaacatcaaggatagagtgcgggagaaagcaatccacctgaccgccgagaatgacttccagggcctgcaactgtggctggcccagtacatcgtgtacgggagacatagcgaggcaaccatgatcggcaagtggaactctgccgatgatctggaggaatttctgaaggagtttaagcagcacagcctgcggaacccaatcgtggagcaggtgattacagaaaccctgcgtgtggtgaaggacatttggctgaaatacggaaacggcgccaaggacttctttaacgaggtgcacatcgagctgggaagagagatgaagcagacaaaggacgaacgggctaatgccacaaagacaatcaccgaaaatgaaaacaccaacctgagaatcaaagccctgctggctgagatgatgaacgaccacagcgtggaaaacgtgcggccttattctcctatgcagcaggagatcctgaaaatctacgaggacggcatcctgaaatcggatatcgagatcgatgacgacatcctgaagatcagcaaaaccgcccaacctagcagcagcgatctgaaaagatacaaactgtggctggagcagaaatacaagtccccttacaccggacagatcatccccctgaataagctcttcacccctgagtacgaaatcgaacacatcatccctcagtccagatacttcgacgacagcctgagcaacaaaattatctgcgagagcgccgtgaacaagctgaaagataactacatcggcctgggtttcatcaagcaattcggcggcacaatcatcgagtgcggcttcggcaagagagtgaaggttttcaaggccgaggaatacgaggaattcgtgaagaagcactacgccaacaaccggggcaagagaaacaagctgctgctggaagacatcccagagaagatgatcgagcggcagatgaacgacacccggcatatctctaagtacatcagcggcattctgtcccacatcgtccgggtggaagacggcaccgacgagggcgttaacagcaaaaatgtgatccccggcaacggcaaaatcaccacacaactgagacaggactggggactgaatgatgtgtggaacgagcttgtgctgccgcgattcgagagaatgaaccaactgaccaacagcacagattttacaagctggaacgagaaccaccagaagtacctgcctacagtgcctgtggagtttagcaagggattctctaagaaaagaatcgaccaccggcaccacgccctggacgccctggtgatcgcctgcgccaccaaggatcatgtgaacctgctgaacaatcagagcgccaagagcgacacgaagcgatacgatctgaagaagaagctgatgaagttcgagaagacagtgtacaaggacccccagaccgagaagcgaatcgagcgagaagtgccaaagtactttctgaagccttgggagacttttaccgtggacgccaagaacaagctggaaaccatcatcgtgtccttcaaacagaacctcagagtcatcaacaaggccacaaactactacgagaagtacgtggacaaggacggcgtgaagacgaaggagcgggttgagcagactggaacaaactgggccatcagaaaacctatgcacaaggaaaccgtgtccggcaaaattgacctgccttgggtgaaagtgcccaagggcaagatcctgaccgccaccagaaagagcctggacaccagcttcgacctcaaggctatcgccagcatcacggacaccggcatccagaagatcctgaagaactacctggagttcaaggaatcccctgagctggccttcagccctgagggcatcgaggacatgaacaagaacatcaaaaagtacaacggcggaaagccccaccagcctatctctaaggtgcgcgtctttgagctgggcagcaagttccaagtgggccagaccggcaataagaaggacaagtacgtggaagccgccaagggaaccaacctgttcttcgccatctatgaggacagaaagggcaagcgctcctacgagaccatccctctgaacgaggtgatcgagagacagaagcagggtctgtctgtggtggacctgaagaacattaacgacttcttcctgtgtcctaacgacctggtgtacattcccagcggcgacgagttggaaaacggcggctccatagagatcaaaaacatcaccaaagaaaagtccgaaagattttataaggtggtctctttttctggcagccaaatcttcttcgtgcggcacgacattgccgtgtccatcgtgaacaagggggaattctccaccctaaacaagatggaacgcgccatcgatggctctatggtgaaggaatcttgtatcaagctgaagatcgacagactgggcaacgtgctgaaagcg

SEQ ID NO: 418 - DNA, 4296 bp, synthetic construct
  misc_feature 1..4296: note = source = /note="Description of Artificial Sequence: Synthetic polynucleotide"
  source 1..4296: mol_type = other DNA
atggccaagaatatcctcggcctggacctgggcaccaacagcatcggctgggcccttatcaaccaagacttcgagaacaagcagggtaagatcctcggaatgggcagccggatcattccaatgagccaagatatcctgggcgacttcggcaagggcaactctgtgtcccagaccgccgctagaaccggctaccggggcgtgcggagactcagagagcggtttctgctgagaagagagagactgcaccgggtgctgaatataatcaacttcctgcccgaacattacgcctctcagatcgactttgagaaaagattcggcaagttcaaggaagaaaccgagcctaagctggcctacaacaaagacggcttcgtgttcaaggatagctttgaggaaatgctggccgattttaagaactaccagcctcaactgctggaaaacgacaagaaaattccctacgactggaccatctactacctgagaaagaaggccctgtcccagaaaatcgagaaggaagagttggcctggattctgctcaatttcaaccagaagagaggctattaccaactgaggggcgaagacttcgaggaagagaaggacaaaatgttcgtccgcctgaaggtggagaagatcatcgatagcggcgacaacgtgaagggtaagattctgtacgacgtctacttcgagaacggctggaaatacgacaaacagatcgttaagaccgaggactggatcgaaagagtgaaggaattcatcgtcaccgaatctttcctgaagaatggcgacatcaagagaacatttaaggccgtggactccgagaaggattggatcgccatcaaaacaaaaacagaacaagagattgacaagtcccacaagaccgtgggcgtgtacatctacgaaacactgctccagaaccctaagcagaagattaagggcaagctggtgagaacaatcgagcgcaagttttacaaggacgaactgaagcagatcttggagaagcagaaagagttccaccaggagctgaagaacgatgacctgtataacgactgcgtgagagaactgtacagaaacaatgaagctcatcaactgaccctctctaagaaggagtttgtgcacctgctgatggaagatatcattttctaccaaagacccctgaggtcccagaagagcagcatcagcaattgcagcctggaattcagaaagtacaaggacgagaacggcgtggagcatatccaatatctgaaagccgtgcctaagagcaatccttactaccaggagtttcggatctggcagtggatctttaacctgaacatctacaagcgggacgatgagcaaaaccccgtgaccacagagttcctgaacaccaccaccgatattgaaaacctcttcgagttcctcaacaaccggaaggaagtggatcagaaagccctgctgaaacatttcaagctgaacgagaagacccacagatggaagtacgtggaggataagaaatacccctgcaacgaaaccaagagcatgatttccgagcggctgaagaaggtggagaacatagctaacgacttcctgaccagagagattgagcagaaaatctggcatatcatctattctgtgaacgacaaaatcgaatacgaaaaagccctgaagagttttgccaagaagaacaacctggacgagaactcctttttcgaggctttcaagaaattcccccccttcaaaagcgagtacggcagtttcagcgaaaaggccatcaagaagctgctgcctctgatgcggcttggcaagtactggaactgcgagaacatcagcgacgacagcaaggaaagaatccagaagattatcaacggcgagtacgacgagaatatcaaggatagagtgcgcgagaaagccattcacctgaccagcgaaaacaacttccagggcctgcagctctggctggcccagtacgtggtgtacgacagacattcggaagccagcatgatcggcaagtggaatagcgccgacgacctcgaagaattcctgaaggagttcaaacagcacagccttagaaaccctattgtggagcaggtgatcacagagacactgcgggtggtgaaggacatctggctgaagtacggcaatggagccaaagatttcttcaatgagattcacatcgaactgggaagagagatgaagcaaaccaaagacgagcgcctcgatgctacgaagcggatcaccgagaacgagaataccaatctgagaatcaaggctctgctggctgagatgatgaatgataacagcgtggaaaacgtccggccttacagccctatgcagcaggagatcctgaagatctacgaggatggcgtgctgaatagtggaattgagattgaagatgaatacctgaagatttctaagaccgcccagcctagcccttctgacctcaagcggtacaagctgtggctggaacagagatacaagagcccatacaccggccagatcattcccctgaacaagctgtttacccctgaatacgagattgagcacatcatccctcagagcagatacttcgacgacagcttcagcaacaagatcatctgtgaaagcgccgtgaacaagctgaaggacaactatatcggactgggcttcatcaagcagttcggcggaaccatcgtggagtgcggcctgggaaaaaacgtgaaggtgtttgaagtgaacgagtacgaggacttcgtgaagaaacactacgccaacaacagaggaaagaggaataagctgctcctggaggaaatccctgagaaaatgatcgagaggcaactgaacgacaccaggcatatcagcaagtacatctccggcgtgctgagcaacatcgtgcgggtggaagacggctctgacgagggagtgaacagcaaaaacatcgtgccaggcaatggcaagatcaccacccaactgaagcaggattggggactgaacgatgtgtggaacgacctgatcctgcctagattcgagagaatgaaccaactgaccaattcaacagacttcaccgcctggaacgaaaatcaccagaagtttctccctacagtgcccatcgaatttagcaagggatttagcaagaaaagaatcgaccacagacaccacgccctggacgccctcgtgatcgcctgtgccaccaaggaccacatcaatctgctgaacaatcagtctgccaaaagcgacaccaaacggtacgacctgaagaaaaagctcatgaaattcgagaagggcgtgtacaaccaccctcagaccggcgagagaatccagcgggacgtgccgaagcaatttctgaagccttgggagagcttcaccatcgacgctaagaacaacctggacaagatcatcatcagcttcaagcagaacttgagggtgatcaacaaagccacaaactactacgagaagtatgtggaaaagaacggcatcaagacaaaagagagagtcgaacagacaggcacaaactgggccatccggaagcccatgcacaaggaaaccgtgtctggcatcgttaacctgccttgggtgaaagtgcctaaaggcaaaatcctgaccgccacccggaaaagcctggacaccacatttgacctgaaaagcatcaactccataacagataccggcattcagaagatcttgagaaactacctggaattcaaaggcagtcctgaactggccttcagcccagagggcatcgaggacatgaacaagaacatcgagaaatacaacgatggcaagctgcaccaacctataaacaaagtgagagttttcgagctgggcagcaaattccaagtgggacagaccggcaacaagaaggacaagtacgtggaggccgccaagggcaccaacctgttcttcgccgtctacgaggacaagaacggcaaaagaaactacgaaacaatccctctgaacatagtgatcgagagacagaagcagggcctcctggcctgccccgagagtaatgaaaaaggcgagaagctgctgttccaactgagcccaaacgacttcgtgtatctcacgacagaggaagaaaatgataacgccacacctatcaatttctctctgctgagcaaggaacagattaacaacctgtacaaaatcgtgtcctttaccggcaacagactgtacggcatccctatctgcgtggccaccacaatcgtgaacaaagccgagtacacccaactgaacaagatcgagttcaccaaagagaaagatctgctgctgaagctcaatgtcaatagactgggcgacgtgaagacctttacagccaatgacatcagaaagattttcaaccggcaggag

SEQ ID NO: 419 - RNA, 137 nt, synthetic construct
  misc_feature 1..137: note = source = /note="Description of Artificial Sequence: Synthetic polynucleotide"
  source 1..137: mol_type = other RNA
aagtgggagagtactgaatgggcacgttgcggttggcctgcgatttctgaacaaaggttcagaaatcgcagtccagccgttaacaagctgagatatgcaccaaataaggcgctcgcttcggcgggcgctttttcgtt

SEQ ID NO: 420 - RNA, 137 nt, synthetic construct
  misc_feature 1..137: note = source = /note="Description of Artificial Sequence: Synthetic polynucleotide"
  source 1..137: mol_type = other RNA
accttcttcccatggcagtacattagttgcggttggcctgcgatttctgaacaaaggttcagaaatcgcagtccagccgttaacaagctgagatatgcaccaaataaggcgctcgcttcggcgggcgctttttcgtt

SEQ ID NO: 421 - RNA, 137 nt, synthetic construct
  misc_feature 1..137: note = source = /note="Description of Artificial Sequence: Synthetic polynucleotide"
  source 1..137: mol_type = other RNA
gcccacaactgcgataatgactctagttgcggttggcctgcgatttctgaacaaaggttcagaaatcgcagtccagccgttaacaagctgagatatgcaccaaataaggcgctcgcttcggcgggcgctttttcgtt

SEQ ID NO: 422 - RNA, 137 nt, synthetic construct
  misc_feature 1..137: note = source = /note="Description of Artificial Sequence: Synthetic polynucleotide"
  source 1..137: mol_type = other RNA
tgggggcgctcagtttccagaacctgttgcggttggcctgcgatttctgaacaaaggttcagaaatcgcagtccagccgttaacaagctgagatatgcaccaaataaggcgctcgcttcggcgggcgctttttcgtt

SEQ ID NO: 423 - RNA, 133 nt, synthetic construct
  misc_feature 1..133: note = source = /note="Description of Artificial Sequence: Synthetic polynucleotide"
  source 1..133: mol_type = other RNA
acagtgcgcatctccctggtcaccgttttagtactctgtaattttaggtatgaaagtcatacctaaaattacagaatctactgaaacaagactatatgtcgtgtttatcccactaatttattagtgggatttt

SEQ ID NO: 424 - RNA, 131 nt, synthetic construct
  misc_feature 1..131: note = source = /note="Description of Artificial Sequence: Synthetic polynucleotide"
  source 1..131: mol_type = other RNA
atttacagcctggcctttgggggttttagtactctgtaattttaggtatgaaagtcatacctaaaattacagaatctactgaaacaagactatatgtcgtgtttatcccactaatttattagtgggatttt

SEQ ID NO: 425 - RNA, 134 nt, synthetic construct
  misc_feature 1..134: note = source = /note="Description of Artificial Sequence: Synthetic polynucleotide"
  source 1..134: mol_type = other RNA
ccttggggcccagactgagcacgtggttttagtactctgtaattttaggtatgaaagtcatacctaaaattacagaatctactgaaacaagactatatgtcgtgtttatcccactaatttattagtgggatttt

SEQ ID NO: 426 - RNA, 134 nt, synthetic construct
  misc_feature 1..134: note = source = /note="Description of Artificial Sequence: Synthetic polynucleotide"
  source 1..134: mol_type = other RNA
cttctcctcaggagtcagatgcaccgttttagtactctgtaattttaggtatgaaagtcatacctaaaattacagaatctactgaaacaagactatatgtcgtgtttatcccactaatttattagtgggatttt

SEQ ID NO: 427 - RNA, 132 nt, synthetic construct
  misc_feature 1..132: note = source = /note="Description of Artificial Sequence: Synthetic polynucleotide"
  source 1..132: mol_type = other RNA
gatgatattttctttaatggtgcgttttagtactctgtaattttaggtatgaaagtcatacctaaaattacagaatctactgaaacaagactatatgtcgtgtttatcccactaatttattagtgggatttt

SEQ ID NO: 428 - RNA, 133 nt, synthetic construct
  misc_feature 1..133: note = source = /note="Description of Artificial Sequence: Synthetic polynucleotide"
  source 1..133: mol_type = other RNA
ggctgggccattaaaacctctccagttttagtactctgtaattttaggtatgaaagtcatacctaaaattacagaatctactgaaacaagactatatgtcgtgtttatcccactaatttattagtgggatttt

SEQ ID NO: 429 - RNA, 206 nt, synthetic construct
  misc_feature 1..206: note = source = /note="Description of Artificial Sequence: Synthetic polynucleotide"
  source 1..206: mol_type = other RNA
cacatctcgagcaagacgttgtcatagttccattaaagccattgctgttttatgaaagcatgatacagcaatggctttgatgtttctatgataagggcttcggcccgtggcgttggggatcgcctgcccattttaatgggcttctccccatctatttaatgagaaatttacaaccttggctattcttaaatagctaaggttttttt

SEQ ID NO: 430 - RNA, 206 nt, synthetic construct
  misc_feature 1..206: note = source = /note="Description of Artificial Sequence: Synthetic polynucleotide"
  source 1..206: mol_type = other RNA
catggcagtacattagagcagtcatagttccattaaagccattgctgttttatgaaagcatgatacagcaatggctttgatgtttctatgataagggcttcggcccgtggcgttggggatcgcctgcccattttaatgggcttctccccatctatttaatgagaaatttacaaccttggctattcttaaatagctaaggttttttt

SEQ ID NO: 431 - RNA, 206 nt, synthetic construct
  misc_feature 1..206: note = source = /note="Description of Artificial Sequence: Synthetic polynucleotide"
  source 1..206: mol_type = other RNA
cctgaatgctgtgcggctctgtcatagttccattaaagccattgctgttttatgaaagcatgatacagcaatggctttgatgtttctatgataagggcttcggcccgtggcgttggggatcgcctgcccattttaatgggcttctccccatctatttaatgagaaatttacaaccttggctattcttaaatagctaaggttttttt

SEQ ID NO: 432 - RNA, 206 nt, synthetic construct
  misc_feature 1..206: note = source = /note="Description of Artificial Sequence: Synthetic polynucleotide"
  source 1..206: mol_type = other RNA
cttctatagcctccttccccgtcatagttccattaaagccattgctgttttatgaaagcatgatacagcaatggctttgatgtttctatgataagggcttcggcccgtggcgttggggatcgcctgcccattttaatgggcttctccccatctatttaatgagaaatttacaaccttggctattcttaaatagctaaggttttttt

SEQ ID NO: 433 - RNA, 206 nt, synthetic construct
  misc_feature 1..206: note = source = /note="Description of Artificial Sequence: Synthetic polynucleotide"
  source 1..206: mol_type = other RNA
ggacagtgcgcatctccctggtcatagttccattaaagccattgctgttttatgaaagcatgatacagcaatggctttgatgtttctatgataagggcttcggcccgtggcgttggggatcgcctgcccattttaatgggcttctccccatctatttaatgagaaatttacaaccttggctattcttaaatagctaaggttttttt

SEQ ID NO: 434 - RNA, 133 nt, synthetic construct
  misc_feature 1..133: note = source = /note="Description of Artificial Sequence: Synthetic polynucleotide"
  source 1..133: mol_type = other RNA
aagacattacactcatattacacccgtcatagttccattaaagccaaagggctttgatgtttctatgataagggtttcggcccgtggcgtcggggatcgcctgcccattccgatgggcttctccccatttatt

SEQ ID NO: 435 - RNA, 133 nt, synthetic construct
  misc_feature 1..133: note = source = /note="Description of Artificial Sequence: Synthetic polynucleotide"
  source 1..133: mol_type = other RNA
agagccatcaccatcacatccctaagtcatagttccattaaagccaaagggctttgatgtttctatgataagggtttcggcccgtggcgtcggggatcgcctgcccattccgatgggcttctccccatttatt

SEQ ID NO: 436 - RNA, 133 nt, synthetic construct
  misc_feature 1..133: note = source = /note="Description of Artificial Sequence: Synthetic polynucleotide"
  source 1..133: mol_type = other RNA
atctggagggaacttacagcatatggtcatagttccattaaagccaaagggctttgatgtttctatgataagggtttcggcccgtggcgtcggggatcgcctgcccattccgatgggcttctccccatttatt

SEQ ID NO: 437 - RNA, 133 nt, synthetic construct
  misc_feature 1..133: note = source = /note="Description of Artificial Sequence: Synthetic polynucleotide"
  source 1..133: mol_type = other RNA
ccacccagcctgctctgccttgggggtcatagttccattaaagccaaagggctttgatgtttctatgataagggtttcggcccgtggcgtcggggatcgcctgcccattccgatgggcttctccccatttatt

SEQ ID NO: 438 - RNA, 133 nt, synthetic construct
  misc_feature 1..133: note = source = /note="Description of Artificial Sequence: Synthetic polynucleotide"
  source 1..133: mol_type = other RNA
gaacaactcaaatggaaatgaatatgtcatagttccattaaagccaaagggctttgatgtttctatgataagggtttcggcccgtggcgtcggggatcgcctgcccattccgatgggcttctccccatttatt

SEQ ID NO: 439 - RNA, 133 nt, synthetic construct
  misc_feature 1..133: note = source = /note="Description of Artificial Sequence: Synthetic polynucleotide"
  source 1..133: mol_type = other RNA
gcttctactcttggcttacaacccagtcatagttccattaaagccaaagggctttgatgtttctatgataagggtttcggcccgtggcgtcggggatcgcctgcccattccgatgggcttctccccatttatt

SEQ ID NO: 440 - RNA, 133 nt, synthetic construct
  misc_feature 1..133: note = source = /note="Description of Artificial Sequence: Synthetic polynucleotide"
  source 1..133: mol_type = other RNA
ggccaaaatccagctgccttccttggtcatagttccattaaagccaaagggctttgatgtttctatgataagggtttcggcccgtggcgtcggggatcgcctgcccattccgatgggcttctccccatttatt

SEQ ID NO: 441 - RNA, 133 nt, synthetic construct
  misc_feature 1..133: note = source = /note="Description of Artificial Sequence: Synthetic polynucleotide"
  source 1..133: mol_type = other RNA
taaaatgatttggaagagcagagacgtcatagttccattaaagccaaagggctttgatgtttctatgataagggtttcggcccgtggcgtcggggatcgcctgcccattccgatgggcttctccccatttatt

SEQ ID NO: 442 - RNA, 133 nt, synthetic construct
  misc_feature 1..133: note = source = /note="Description of Artificial Sequence: Synthetic polynucleotide"
  source 1..133: mol_type = other RNA
tcctgttccatcaccatcaaaaaaagtcatagttccattaaagccaaagggctttgatgtttctatgataagggtttcggcccgtggcgtcggggatcgcctgcccattccgatgggcttctccccatttatt

SEQ ID NO: 443 - RNA, 164 nt, synthetic construct
  misc_feature 1..164: note = source = /note="Description of Artificial Sequence: Synthetic polynucleotide"
  source 1..164: mol_type = other RNA
aagacattacactcatattacacccgccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 444 - RNA, 164 nt, synthetic construct
  misc_feature 1..164: note = source = /note="Description of Artificial Sequence: Synthetic polynucleotide"
  source 1..164: mol_type = other RNA
agctgtttgggaggtcagaaatagggccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 445 - RNA, 159 nt, synthetic construct
  misc_feature 1..159: note = source = /note="Description of Artificial Sequence: Synthetic polynucleotide"
  source 1..159: mol_type = other RNA
aggttttaatggcccagcctgccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 446 - RNA, 164 nt, synthetic construct
  misc_feature 1..164: note = source = /note="Description of Artificial Sequence: Synthetic polynucleotide"
  source 1..164: mol_type = other RNA
atgtactgccatgggaagaaggtgagccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 447 - RNA, 164 nt, synthetic construct
  misc_feature 1..164: note = source = /note="Description of Artificial Sequence: Synthetic polynucleotide"
  source 1..164: mol_type = other RNA
cagaagtcaggctggacactgaggagccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 448 - RNA, 164 nt, synthetic construct
  misc_feature 1..164: note = source = /note="Description of Artificial Sequence: Synthetic polynucleotide"
  source 1..164: mol_type = other RNA
cagggcctcgataatgagataatttgccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 449 - RNA, 164 nt, synthetic construct
  misc_feature 1..164: note = source = /note="Description of Artificial Sequence: Synthetic polynucleotide"
  source 1..164: mol_type = other RNA
catctactaatttcttcccactcccgccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 450 - RNA, 159 nt, synthetic construct
  misc_feature 1..159: note = source = /note="Description of Artificial Sequence: Synthetic polynucleotide"
  source 1..159: mol_type = other RNA
catggcagtacattagagcagccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 451 - RNA, 164 nt, synthetic construct
  misc_feature 1..164: note = source = /note="Description of Artificial Sequence: Synthetic polynucleotide"
  source 1..164: mol_type = other RNA
ccctggctgtgcacattccctcctggccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 452 - RNA, 164 nt, synthetic construct
  misc_feature 1..164: note = source = /note="Description of Artificial Sequence: Synthetic polynucleotide"
  source 1..164: mol_type = other RNA
ccgctgtttccattcctcatcattcgccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 453 - RNA, 164 nt, synthetic construct
  misc_feature 1..164: note = source = /note="Description of Artificial Sequence: Synthetic polynucleotide"
  source 1..164: mol_type = other RNA
cctagagcccaagagaacccactgagccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 454 - RNA, 164 nt, synthetic construct
  misc_feature 1..164: note = source = /note="Description of Artificial Sequence: Synthetic polynucleotide"
  source 1..164: mol_type = other RNA
cgcacagcattcaggtcgtagtcccgccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 455 - RNA, 164 nt, synthetic construct
  misc_feature 1..164: note = source = /note="Description of Artificial Sequence: Synthetic polynucleotide"
  source 1..164: mol_type = other RNA
cggcactatcattgactcagctgctgccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 456 - RNA, 164 nt, synthetic construct
  misc_feature 1..164: note = source = /note="Description of Artificial Sequence: Synthetic polynucleotide"
  source 1..164: mol_type = other RNA
ctcaacattccagggctcaagcgatgccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 457 - RNA, 164 nt, synthetic construct
  misc_feature 1..164: note = source = /note="Description of Artificial Sequence: Synthetic polynucleotide"
  source 1..164: mol_type = other RNA
gaatgctgtgcggctctgcttccaggccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 458 - RNA, 164 nt, synthetic construct
  misc_feature 1..164: note = source = /note="Description of Artificial Sequence: Synthetic polynucleotide"
  source 1..164: mol_type = other RNA
gctaccacctctgtgcccccccggcgccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 459 - RNA, 164 nt, synthetic construct
  misc_feature 1..164: note = source = /note="Description of Artificial Sequence: Synthetic polynucleotide"
  source 1..164: mol_type = other RNA
ggaggcctgcagctcctgcacctccgccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 460 - RNA, 159 nt, synthetic construct
  misc_feature 1..159: note = source = /note="Description of Artificial Sequence: Synthetic polynucleotide"
  source 1..159: mol_type = other RNA
gggccattaaaacctctccagccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 461 - RNA, 164 nt, synthetic construct
  misc_feature 1..164: note = source = /note="Description of Artificial Sequence: Synthetic polynucleotide"
  source 1..164: mol_type = other RNA
gggccattaaaacctctccaggggcgccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 462 - RNA, 164 nt, synthetic construct
  misc_feature 1..164: note = source = /note="Description of Artificial Sequence: Synthetic polynucleotide"
  source 1..164: mol_type = other RNA
gttttaatggcccagcctcacacccgccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 463 - RNA, 164 nt, synthetic construct
  misc_feature 1..164: note = source = /note="Description of Artificial Sequence: Synthetic polynucleotide"
  source 1..164: mol_type = other RNA
taaaatgatttggaagagcagagacgccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 464 - RNA, 164 nt, synthetic construct
  misc_feature 1..164: note = source = /note="Description of Artificial Sequence: Synthetic polynucleotide"
  source 1..164: mol_type = other RNA
tcctgttccatcaccatcaaaaaaagccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 465 - RNA, 164 nt, synthetic construct
  misc_feature 1..164: note = source = /note="Description of Artificial Sequence: Synthetic polynucleotide"
  source 1..164: mol_type = other RNA
tgtctcaaataataaaaaaaataaggccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 466 - RNA, 164 nt, synthetic construct
  misc_feature 1..164: note = source = /note="Description of Artificial Sequence: Synthetic polynucleotide"
  source 1..164: mol_type = other RNA
tgttggttacctccctgccaccaccgccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 467 - RNA, 164 nt, synthetic construct
  misc_feature 1..164: note = source = /note="Description of Artificial Sequence: Synthetic polynucleotide"
  source 1..164: mol_type = other RNA
tttaggttcgtatctgtaaaaccaagccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 468 - RNA, 118 nt, synthetic construct
  misc_feature 1..118: note = source = /note="Description of Artificial Sequence: Synthetic polynucleotide"
  source 1..118: mol_type = other RNA
aagtgggagagtactgaatgggcacgtttttgtactctcaataaaaagttattgagaatctacaaaaataaggcattttgccgaatttaccgccctacatatgtagggcggttttttt

SEQ ID NO: 469 - RNA, 113 nt, synthetic construct
  misc_feature 1..113: note = source = /note="Description of Artificial Sequence: Synthetic polynucleotide"
  source 1..113: mol_type = other RNA
accggaggacaaagtacaaagtttttgtactctcaataaaaagttattgagaatctacaaaaataaggcattttgccgaatttaccgccctacatatgtagggcggttttttt

SEQ ID NO: 470 - RNA, 113 nt, synthetic construct
  misc_feature 1..113: note = source = /note="Description of Artificial Sequence: Synthetic polynucleotide"
  source 1..113: mol_type = other RNA
ccacggctcctccgaagcgagtttttgtactctcaataaaaagttattgagaatctacaaaaataaggcattttgccgaatttaccgccctacatatgtagggcggttttttt

SEQ ID NO: 471 - RNA, 113 nt, synthetic construct
  misc_feature 1..113: note = source = /note="Description of Artificial Sequence: Synthetic polynucleotide"
  source 1..113: mol_type = other RNA
ccctgatcctcttgtcccacgtttttgtactctcaataaaaagttattgagaatctacaaaaataaggcattttgccgaatttaccgccctacatatgtagggcggttttttt

SEQ ID NO: 472 - RNA, 113 nt, synthetic construct
  misc_feature 1..113: note = source = /note="Description of Artificial Sequence: Synthetic polynucleotide"
  source 1..113: mol_type = other RNA
ctggagcaacaaatctgactgtttttgtactctcaataaaaagttattgagaatctacaaaaataaggcattttgccgaatttaccgccctacatatgtagggcggttttttt

SEQ ID NO: 473 - RNA, 113 nt, synthetic construct
  misc_feature 1..113: note = source = /note="Description of Artificial Sequence: Synthetic polynucleotide"
  source 1..113: mol_type = other RNA
gggggtcggggctcgcggcggtttttgtactctcaataaaaagttattgagaatctacaaaaataaggcattttgccgaatttaccgccctacatatgtagggcggttttttt

SEQ ID NO: 474 - RNA, 113 nt, synthetic construct
  misc_feature 1..113: note = source = /note="Description of Artificial Sequence: Synthetic polynucleotide"
  source 1..113: mol_type = other RNA
tggatttagagtctctcagcgtttttgtactctcaataaaaagttattgagaatctacaaaaataaggcattttgccgaatttaccgccctacatatgtagggcggttttttt

SEQ ID NO: 475 - RNA, 123 nt, synthetic construct
  misc_feature 1..123: note = source = /note="Description of Artificial Sequence: Synthetic polynucleotide"
  source 1..123: mol_type = other RNA
aagtgggagagtactgaatgggcacgttgtgaattgctttcaaaaaagtttgaaaagcaattcacaataaggattattccgttgtgaaaacattcaaggcggggcaactcgccttttttcgtt

SEQ ID NO: 476 - RNA, 123 nt, synthetic construct
  misc_feature 1..123: note = source = /note="Description of Artificial Sequence: Synthetic polynucleotide"
  source 1..123: mol_type = other RNA
aggtgattcacagagacataaagccgttgtgaattgctttcaaaaaagtttgaaaagcaattcacaataaggattattccgttgtgaaaacattcaaggcggggcaactcgccttttttcgtt

SEQ ID NO: 477 - RNA, 123 nt, synthetic construct
  misc_feature 1..123: note = source = /note="Description of Artificial Sequence: Synthetic polynucleotide"
  source 1..123: mol_type = other RNA
atttagctcttagtcctggcaagccgttgtgaattgctttcaaaaaagtttgaaaagcaattcacaataaggattattccgttgtgaaaacattcaaggcggggcaactcgccttttttcgtt

SEQ ID NO: 478 - RNA, 123 nt, synthetic construct
  misc_feature 1..123: note = source = /note="Description of Artificial Sequence: Synthetic polynucleotide"
  source 1..123: mol_type = other RNA
ctgggaggtcaagactgcaatgagagttgtgaattgctttcaaaaaagtttgaaaagcaattcacaataaggattattccgttgtgaaaacattcaaggcggggcaactcgccttttttcgtt

SEQ ID NO: 479 - RNA, 123 nt, synthetic construct
  misc_feature 1..123: note = source = /note="Description of Artificial Sequence: Synthetic polynucleotide"
  source 1..123: mol_type = other RNA
gcccacaactgcgataatgactctagttgtgaattgctttcaaaaaagtttgaaaagcaattcacaataaggattattccgttgtgaaaacattcaaggcggggcaactcgccttttttcgtt

SEQ ID NO: 480 - RNA, 118 nt, synthetic construct
  misc_feature 1..118: note = source = /note="Description of Artificial Sequence: Synthetic polynucleotide"
  source 1..118: mol_type = other RNA
aagcaactctcctgtctcagcctccgttgtgaattgctttcaaaggaaagcaattcacaataaggattattccgttgtgaaaacatttagcgcctcgactatcttcggggcatttttt

SEQ ID NO: 481 - RNA, 118 nt, synthetic construct
  misc_feature 1..118: note = source = /note="Description of Artificial Sequence: Synthetic polynucleotide"
  source 1..118: mol_type = other RNA
gggaagaaggtgattcacagagacagttgtgaattgctttcaaaggaaagcaattcacaataaggattattccgttgtgaaaacatttagcgcctcgactatcttcggggcatttttt

SEQ ID NO: 482 - DNA, 25 bp, synthetic construct
  misc_feature 1..25: note = source = /note="Description of Artificial Sequence: Synthetic oligonucleotide"
  source 1..25: mol_type = other DNA
aagtgggagagtactgaatgggcac

SEQ ID NO: 483 - DNA, 25 bp, synthetic construct
  misc_feature 1..25: note = source = /note="Description of Artificial Sequence: Synthetic oligonucleotide"
  source 1..25: mol_type = other DNA
accttcttcccatggcagtacatta

SEQ ID NO: 484 - DNA, 25 bp, synthetic construct
  misc_feature 1..25: note = source = /note="Description of Artificial Sequence: Synthetic oligonucleotide"
  source 1..25: mol_type = other DNA
gcccacaactgcgataatgactcta

SEQ ID NO: 485 - DNA, 25 bp, synthetic construct
  misc_feature 1..25: note = source = /note="Description of Artificial Sequence: Synthetic oligonucleotide"
  source 1..25: mol_type = other DNA
tgggggcgctcagtttccagaacct

SEQ ID NO: 486 - DNA, 24 bp, synthetic construct
  misc_feature 1..24: note = source = /note="Description of Artificial Sequence: Synthetic oligonucleotide"
  source 1..24: mol_type = other DNA
acagtgcgcatctccctggtcacc

SEQ ID NO: 487 - DNA, 22 bp, synthetic construct
  misc_feature 1..22: note = source = /note="Description of Artificial Sequence: Synthetic oligonucleotide"
  source 1..22: mol_type = other DNA
atttacagcctggcctttgggg

SEQ ID NO: 488 - DNA, 25 bp, synthetic construct
  misc_feature 1..25: note = source = /note="Description of Artificial Sequence: Synthetic oligonucleotide"
  source 1..25: mol_type = other DNA
ccttggggcccagactgagcacgtg

SEQ ID NO: 489 - DNA, 25 bp, synthetic construct
  misc_feature 1..25: note = source = /note="Description of Artificial Sequence: Synthetic oligonucleotide"
  source 1..25: mol_type = other DNA
cttctcctcaggagtcagatgcacc

SEQ ID NO: 490 - DNA, 23 bp, synthetic construct
  misc_feature 1..23: note = source = /note="Description of Artificial Sequence: Synthetic oligonucleotide"
  source 1..23: mol_type = other DNA
gatgatattttctttaatggtgc

SEQ ID NO: 491 - DNA, 24 bp, synthetic construct
  misc_feature 1..24: note = source = /note="Description of Artificial Sequence: Synthetic oligonucleotide"
  source 1..24: mol_type = other DNA
ggctgggccattaaaacctctcca

SEQ ID NO: 492 - DNA, 20 bp, synthetic construct
  misc_feature 1..20: note = source = /note="Description of Artificial Sequence: Synthetic oligonucleotide"
  source 1..20: mol_type = other DNA
cacatctcgagcaagacgtt

SEQ ID NO: 493 - DNA, 20 bp, synthetic construct
  misc_feature 1..20: note = source = /note="Description of Artificial Sequence: Synthetic oligonucleotide"
  source 1..20: mol_type = other DNA
catggcagtacattagagca

SEQ ID NO: 494 - DNA, 20 bp, synthetic construct
  misc_feature 1..20: note = source = /note="Description of Artificial Sequence: Synthetic oligonucleotide"
  source 1..20: mol_type = other DNA
cctgaatgctgtgcggctct

SEQ ID NO: 495 - DNA, 20 bp, synthetic construct
  misc_feature 1..20: note = source = /note="Description of Artificial Sequence: Synthetic oligonucleotide"
  source 1..20: mol_type = other DNA
cttctatagcctccttcccc

SEQ ID NO: 496 - DNA, 20 bp, synthetic construct
  misc_feature 1..20: note = source = /note="Description of Artificial Sequence: Synthetic oligonucleotide"
  source 1..20: mol_type = other DNA
ggacagtgcgcatctccctg

SEQ ID NO: 497 - DNA, 25 bp, synthetic construct
  misc_feature 1..25: note = source = /note="Description of Artificial Sequence: Synthetic oligonucleotide"
  source 1..25: mol_type = other DNA
aagacattacactcatattacaccc

SEQ ID NO: 498 - DNA, 25 bp, synthetic construct
  misc_feature 1..25: note = source = /note="Description of Artificial Sequence: Synthetic oligonucleotide"
  source 1..25: mol_type = other DNA
agagccatcaccatcacatccctaa

SEQ ID NO: 499 - DNA, 25 bp, synthetic construct
  misc_feature 1..25: note = source = /note="Description of Artificial Sequence: Synthetic oligonucleotide"
  source 1..25: mol_type = other DNA
atctggagggaacttacagcatatg

SEQ ID NO: 500 - DNA, 25 bp, synthetic construct
  misc_feature 1..25: note = source = /note="Description of Artificial Sequence: Synthetic oligonucleotide"
  source 1..25: mol_type = other DNA
ccacccagcctgctctgccttgggg

SEQ ID NO: 501 - DNA, 25 bp, synthetic construct
  misc_feature 1..25: note = source = /note="Description of Artificial Sequence: Synthetic oligonucleotide"
  source 1..25: mol_type = other DNA
gaacaactcaaatggaaatgaatat

SEQ ID NO: 502 - DNA, 25 bp, synthetic construct
  misc_feature 1..25: note = source = /note="Description of Artificial Sequence: Synthetic oligonucleotide"
  source 1..25: mol_type = other DNA
gcttctactcttggcttacaaccca

SEQ ID NO: 503 - DNA, 25 bp, synthetic construct
  misc_feature 1..25: note = source = /note="Description of Artificial Sequence: Synthetic oligonucleotide"
  source 1..25: mol_type = other DNA
ggccaaaatccagctgccttccttg

SEQ ID NO: 504 - DNA, 25 bp, synthetic construct
  misc_feature 1..25: note = source = /note="Description of Artificial Sequence: Synthetic oligonucleotide"
  source 1..25: mol_type = other DNA
taaaatgatttggaagagcagagac

SEQ ID NO: 505 - DNA, 25 bp, synthetic construct
  misc_feature 1..25: note = source = /note="Description of Artificial Sequence: Synthetic oligonucleotide"
  source 1..25: mol_type = other DNA
tcctgttccatcaccatcaaaaaaa

SEQ ID NO: 506 - DNA, 25 bp, synthetic construct
  misc_feature 1..25: note = source = /note="Description of Artificial Sequence: Synthetic oligonucleotide"
  source 1..25: mol_type = other DNA
agctgtttgggaggtcagaaatagg

SEQ ID NO: 507 - DNA, 20 bp, synthetic construct
  misc_feature 1..20: note = source = /note="Description of Artificial Sequence: Synthetic oligonucleotide"
  source 1..20: mol_type = other DNA
aggttttaatggcccagcct

SEQ ID NO: 508 - DNA, 25 bp, synthetic construct
  misc_feature 1..25: note = source = /note="Description of Artificial Sequence: Synthetic oligonucleotide"
  source 1..25: mol_type = other DNA
atgtactgccatgggaagaaggtga

SEQ ID NO: 509 - DNA, 25 bp, synthetic construct
  misc_feature 1..25: note = source = /note="Description of Artificial Sequence: Synthetic oligonucleotide"
  source 1..25: mol_type = other DNA
cagaagtcaggctggacactgagga

SEQ ID NO: 510 - DNA, 25 bp, synthetic construct
  misc_feature 1..25: note = source = /note="Description of Artificial Sequence: Synthetic oligonucleotide"
  source 1..25: mol_type = other DNA
cagggcctcgataatgagataattt

SEQ ID NO: 511 - DNA, 25 bp, synthetic construct
  misc_feature 1..25: note = source = /note="Description of Artificial Sequence: Synthetic oligonucleotide"
  source 1..25: mol_type = other DNA
catctactaatttcttcccactccc

SEQ ID NO: 512 - DNA, 25 bp, synthetic construct
  misc_feature 1..25: note = source = /note="Description of Artificial Sequence: Synthetic oligonucleotide"
  source 1..25: mol_type = other DNA
ccctggctgtgcacattccctcctg

SEQ ID NO: 513 - DNA, 25 bp, synthetic construct
  misc_feature 1..25: note = source = /note="Description of Artificial Sequence: Synthetic oligonucleotide"
  source 1..25: mol_type = other DNA
ccgctgtttccattcctcatcattc

SEQ ID NO: 514 - DNA, 25 bp, synthetic construct
  misc_feature 1..25: note = source = /note="Description of Artificial Sequence: Synthetic oligonucleotide"
  source 1..25: mol_type = other DNA
cctagagcccaagagaacccactga

SEQ ID NO: 515 - DNA, 25 bp, synthetic construct
  misc_feature 1..25: note = source = /note="Description of Artificial Sequence: Synthetic oligonucleotide"
  source 1..25: mol_type = other DNA
cgcacagcattcaggtcgtagtccc

SEQ ID NO: 516 - DNA, 25 bp, synthetic construct
  misc_feature 1..25: note = source = /note="Description of Artificial Sequence: Synthetic oligonucleotide"
  source 1..25: mol_type = other DNA
cggcactatcattgactcagctgct

SEQ ID NO: 517 - DNA, 25 bp, synthetic construct
  misc_feature 1..25: note = source = /note="Description of Artificial Sequence: Synthetic oligonucleotide"
  source 1..25: mol_type = other DNA
ctcaacattccagggctcaagcgat

SEQ ID NO: 518 - DNA, 25 bp, synthetic construct
  misc_feature 1..25: note = source = /note="Description of Artificial Sequence: Synthetic oligonucleotide"
  source 1..25: mol_type = other DNA
gaatgctgtgcggctctgcttccag

SEQ ID NO: 519 - DNA, 25 bp, synthetic construct
  misc_feature 1..25: note = source = /note="Description of Artificial Sequence: Synthetic oligonucleotide"
  source 1..25: mol_type = other DNA
gctaccacctctgtgcccccccggc

SEQ ID NO: 520 - DNA, 25 bp, synthetic construct
  misc_feature 1..25: note = source = /note="Description of Artificial Sequence: Synthetic oligonucleotide"
  source 1..25: mol_type = other DNA
ggaggcctgcagctcctgcacctcc

SEQ ID NO: 521 - DNA, 20 bp, synthetic construct
  misc_feature 1..20: note = source = /note="Description of Artificial Sequence: Synthetic oligonucleotide"
  source 1..20: mol_type = other DNA
gggccattaaaacctctcca

SEQ ID NO: 522 - DNA, 25 bp, synthetic construct
  misc_feature 1..25: note = source = /note="Description of Artificial Sequence: Synthetic oligonucleotide"
  source 1..25: mol_type = other DNA
gggccattaaaacctctccaggggc

SEQ ID NO: 523 - DNA, 25 bp, synthetic construct
  misc_feature 1..25: note = source = /note="Description of Artificial Sequence: Synthetic oligonucleotide"
  source 1..25: mol_type = other DNA
gttttaatggcccagcctcacaccc

SEQ ID NO: 524 - DNA, 25 bp, synthetic construct
  misc_feature 1..25: note = source = /note="Description of Artificial Sequence: Synthetic oligonucleotide"
  source 1..25: mol_type = other DNA
tgtctcaaataataaaaaaaataag

SEQ ID NO: 525 - DNA, 25 bp, synthetic construct
  misc_feature 1..25: note = source = /note="Description of Artificial Sequence: Synthetic oligonucleotide"
  source 1..25: mol_type = other DNA
tgttggttacctccctgccaccacc

SEQ ID NO: 526 - DNA, 25 bp, synthetic construct
  misc_feature 1..25: note = source = /note="Description of Artificial Sequence: Synthetic oligonucleotide"
  source 1..25: mol_type = other DNA
tttaggttcgtatctgtaaaaccaa

SEQ ID NO: 527 - DNA, 20 bp, synthetic construct
  misc_feature 1..20: note = source = /note="Description of Artificial Sequence: Synthetic oligonucleotide"
  source 1..20: mol_type = other DNA
accggaggacaaagtacaaa

SEQ ID NO: 528 - DNA, 20 bp, synthetic construct
  misc_feature 1..20: note = source = /note="Description of Artificial Sequence: Synthetic oligonucleotide"
  source 1..20: mol_type = other DNA
ccacggctcctccgaagcga

SEQ ID NO: 529 - DNA, 20 bp, synthetic construct
  misc_feature 1..20: note = source = /note="Description of Artificial Sequence: Synthetic oligonucleotide"
  source 1..20: mol_type = other DNA
ccctgatcctcttgtcccac

SEQ ID NO: 530 - DNA, 20 bp, synthetic construct
  misc_feature 1..20: note = source = /note="Description of Artificial Sequence: Synthetic oligonucleotide"
  source 1..20: mol_type = other DNA
ctggagcaacaaatctgact

SEQ ID NO: 531 - DNA, 20 bp, synthetic construct
  misc_feature 1..20: note = source = /note="Description of Artificial Sequence: Synthetic oligonucleotide"
  source 1..20: mol_type = other DNA
gggggtcggggctcgcggcg

SEQ ID NO: 532 - DNA, 20 bp, synthetic construct
  misc_feature 1..20: note = source = /note="Description of Artificial Sequence: Synthetic oligonucleotide"
  source 1..20: mol_type = other DNA
tggatttagagtctctcagc

SEQ ID NO: 533 - DNA, 25 bp, synthetic construct
  misc_feature 1..25: note = source = /note="Description of Artificial Sequence: Synthetic oligonucleotide"
  source 1..25: mol_type = other DNA
aggtgattcacagagacataaagcc

SEQ ID NO: 534 - DNA, 25 bp, synthetic construct
  misc_feature 1..25: note = source = /note="Description of Artificial Sequence: Synthetic oligonucleotide"
  source 1..25: mol_type = other DNA
atttagctcttagtcctggcaagcc

SEQ ID NO: 535 - DNA, 25 bp, synthetic construct
  misc_feature 1..25: note = source = /note="Description of Artificial Sequence: Synthetic oligonucleotide"
  source 1..25: mol_type = other DNA
ctgggaggtcaagactgcaatgaga

SEQ ID NO: 536 - DNA, 25 bp, synthetic construct
  misc_feature 1..25: note = source = /note="Description of Artificial Sequence: Synthetic oligonucleotide"
  source 1..25: mol_type = other DNA
aagcaactctcctgtctcagcctcc

SEQ ID NO: 537 - DNA, 25 bp, synthetic construct
  misc_feature 1..25: note = source = /note="Description of Artificial Sequence: Synthetic oligonucleotide"
  source 1..25: mol_type = other DNA
gggaagaaggtgattcacagagaca

SEQ ID NO: 538 - DNA, 25 bp, Homo sapiens
  source 1..25: mol_type = genomic DNA
gacaggctgtgtgtcagagttcgcc

SEQ ID NO: 539 - DNA, 25 bp, Homo sapiens
  source 1..25: mol_type = genomic DNA
agtggacaggctgtgtgtcagagtt

SEQ ID NO: 540 - DNA, 25 bp, Homo sapiens
  source 1..25: mol_type = genomic DNA
ctgcgattgcagaagatgacctggg

SEQ ID NO: 541 - DNA, 25 bp, Homo sapiens
  source 1..25: mol_type = genomic DNA
acgcgcggactgcgattgcagaaga

SEQ ID NO: 542 - DNA, 25 bp, Homo sapiens
  source 1..25: mol_type = genomic DNA
agtactcacgattcgggggtataca

SEQ ID NO: 543 - DNA, 25 bp, Homo sapiens
  source 1..25: mol_type = genomic DNA
agtactcacgattcgggggtataca

SEQ ID NO: 544 - DNA, 25 bp, Homo sapiens
  source 1..25: mol_type = genomic DNA
atcacgtgccttgtacactgtccca

SEQ ID NO: 545 - DNA, 25 bp, Homo sapiens
  source 1..25: mol_type = genomic DNA
gtggggatcacgtgccttgtacact

SEQ ID NO: 546 - DNA, 25 bp, Homo sapiens
  source 1..25: mol_type = genomic DNA
ctttgatgggttcatccgaccagtc

SEQ ID NO: 547 - DNA, 25 bp, Homo sapiens
  source 1..25: mol_type = genomic DNA
tgggttcatccgaccagtcccggca

SEQ ID NO: 548 - DNA, 25 bp, Homo sapiens
  source 1..25: mol_type = genomic DNA
cttggacctggttccatgtccccac

SEQ ID NO: 549 - DNA, 25 bp, Homo sapiens
  source 1..25: mol_type = genomic DNA
accaggaagtggccatgcgcaagct

SEQ ID NO: 550 - DNA, 25 bp, Homo sapiens
  source 1..25: mol_type = genomic DNA
cacagatcgaatagaagactccttc

SEQ ID NO: 551 - DNA, 25 bp, Homo sapiens
  source 1..25: mol_type = genomic DNA
cacagatcgaatagaagactccttc

SEQ ID NO: 552 - DNA, 25 bp, Homo sapiens
  source 1..25: mol_type = genomic DNA
ccctaactgagcagctcaggctgcc

SEQ ID NO: 553 - DNA, 25 bp, Homo sapiens
  source 1..25: mol_type = genomic DNA
caggctgccgtggctcacctgtcca

SEQ ID NO: 554 - DNA, 25 bp, Homo sapiens
  source 1..25: mol_type = genomic DNA
cttccatgaagataacattccccag

SEQ ID NO: 555 - DNA, 25 bp, Homo sapiens
  source 1..25: mol_type = genomic DNA
attccccagctggaagaagtttctc

SEQ ID NO: 556 - DNA, 25 bp, Homo sapiens
  source 1..25: mol_type = genomic DNA
ggtacaagctgcagtcatttccttt

SEQ ID NO: 557 - DNA, 25 bp, Homo sapiens
  source 1..25: mol_type = genomic DNA
gtcatttcctttagggaatcatctt

SEQ ID NO: 558 - DNA, 25 bp, Homo sapiens
  source 1..25: mol_type = genomic DNA
catcaccccctagactatagttagc

SEQ ID NO: 559 - DNA, 25 bp, Homo sapiens
  source 1..25: mol_type = genomic DNA
tcaccccctagactatagttagcca

SEQ ID NO: 560 - DNA, 25 bp, Homo sapiens
  source 1..25: mol_type = genomic DNA
acttcttggcaagtcggttaagatc

SEQ ID NO: 561 - DNA, 25 bp, Homo sapiens
  source 1..25: mol_type = genomic DNA
aagatctgggaatagacgaagtaaa

SEQ ID NO: 562 - DNA, 25 bp, Homo sapiens
  source 1..25: mol_type = genomic DNA
aaaatggctggtagtacatatggaa

SEQ ID NO: 563 - DNA, 25 bp, Homo sapiens
  source 1..25: mol_type = genomic DNA
tggctggtagtacatatggaacagg

SEQ ID NO: 564 - DNA, 25 bp, Homo sapiens
  source 1..25: mol_type = genomic DNA
caattgactgtcaccagcccctact

SEQ ID NO: 565 - DNA, 25 bp, Homo sapiens
  source 1..25: mol_type = genomic DNA
agcccctactagggctaagatgcag

SEQ ID NO: 566 - DNA, 25 bp, Homo sapiens
  source 1..25: mol_type = genomic DNA
catcagtcgagttccaaccttcgcc

SEQ ID NO: 567 - DNA, 25 bp, Homo sapiens
  source 1..25: mol_type = genomic DNA
atacactgtttctatggcttttctc

SEQ ID NO: 568 - DNA, 25 bp, Homo sapiens
  source 1..25: mol_type = genomic DNA
aggaagttgattctaccagatgatc

SEQ ID NO: 569 - DNA, 25 bp, Homo sapiens
  source 1..25: mol_type = genomic DNA
accgtggacaatattcgctccatct

SEQ ID NO: 570 - DNA, 25 bp, Homo sapiens
  source 1..25: mol_type = genomic DNA
ggaaatgctgttagtcgttatgtcg

SEQ ID NO: 571 - DNA, 25 bp, Homo sapiens
  source 1..25: mol_type = genomic DNA
taccatgaatagaacatttcctttc

SEQ ID NO: 572 - DNA, 25 bp, Homo sapiens
  source 1..25: mol_type = genomic DNA
gtgtcttactagccattttaatact

SEQ ID NO: 573 - DNA, 25 bp, Homo sapiens
  source 1..25: mol_type = genomic DNA
ggacctacacaagcagcatcttctt

SEQ ID NO: 574 - DNA, 25 bp, Homo sapiens
  source 1..25: mol_type = genomic DNA
tggtggacctacacaagcagcatct

SEQ ID NO: 575 - DNA, 25 bp, Homo sapiens
  source 1..25: mol_type = genomic DNA
aaagtgactctctaattttctattt

SEQ ID NO: 576 - DNA, 25 bp, Homo sapiens
  source 1..25: mol_type = genomic DNA
tttttggtaatagaacatctccaag

SEQ ID NO: 577 - DNA, 25 bp, Homo sapiens
  source 1..25: mol_type = genomic DNA
caccacggctgtcgacaccaatccc

SEQ ID NO: 578 - DNA, 25 bp, Homo sapiens
  source 1..25: mol_type = genomic DNA
tattccaccacggctgtcgacacca

SEQ ID NO: 579 - DNA, 25 bp, Homo sapiens
  source 1..25: mol_type = genomic DNA
actttgcaacagtggaggaaagcct

SEQ ID NO: 580 - DNA, 25 bp, Homo sapiens
  source 1..25: mol_type = genomic DNA
aacagtggaggaaagcctttggagt

SEQ ID NO: 581 - DNA, 25 bp, Homo sapiens
  source 1..25: mol_type = genomic DNA
aggtggctgcttctttggttgtgct

SEQ ID NO: 582 - DNA, 25 bp, Homo sapiens
  source 1..25: mol_type = genomic DNA
tggctccttggaaagtgagaattcc

SEQ ID NO: 583 - DNA, 25 bp, Homo sapiens
  source 1..25: mol_type = genomic DNA
tcaaataagcagcaggaagcagacc

SEQ ID NO: 584 - DNA, 25 bp, Homo sapiens
  source 1..25: mol_type = genomic DNA
aaataagcagcaggaagcagaccca

SEQ ID NO: 585 - DNA, 25 bp, Homo sapiens
  source 1..25: mol_type = genomic DNA
atgctggcattgttaaatccatccc

SEQ ID NO: 586 - DNA, 25 bp, Homo sapiens
  source 1..25: mol_type = genomic DNA
atccatcccaagggggtgagtgtgg

SEQ ID NO: 587 - DNA, 25 bp, Homo sapiens
  source 1..25: mol_type = genomic DNA
gataggtagtacatgcacagttgct

SEQ ID NO: 588 - DNA, 25 bp, Homo sapiens
  source 1..25: mol_type = genomic DNA
ccatcattagatgataggtagtaca

SEQ ID NO: 589 - DNA, 25 bp, Homo sapiens
  source 1..25: mol_type = genomic DNA
acaatttcaacacaagctaaactag

SEQ ID NO: 590 - DNA, 25 bp, Homo sapiens
  source 1..25: mol_type = genomic DNA
taaactagtaggatatagttcttca

SEQ ID NO: 591 - DNA, 25 bp, Homo sapiens
  source 1..25: mol_type = genomic DNA
aatttcaaaaaatgttggcctctct

SEQ ID NO: 592 - DNA, 25 bp, Homo sapiens
  source 1..25: mol_type = genomic DNA
tccaatagtcatttttacctacgat

SEQ ID NO: 593 - DNA, 25 bp, Homo sapiens
  source 1..25: mol_type = genomic DNA
tctgagtgtttccctccttcataaa

SEQ ID NO: 594 - DNA, 25 bp, Homo sapiens
  source 1..25: mol_type = genomic DNA
ccttcataaacaggccagataattt

SEQ ID NO: 595 - DNA, 25 bp, Homo sapiens
  source 1..25: mol_type = genomic DNA
cagttaacagttctttatttgaagt

SEQ ID NO: 596 - DNA, 25 bp, Homo sapiens
  source 1..25: mol_type = genomic DNA
tgtgtatttacaagaaagagcagat

SEQ ID NO: 597 - DNA, 25 bp, Homo sapiens
  source 1..25: mol_type = genomic DNA
agttctactgtgctcatagataata

SEQ ID NO: 598 - DNA, 25 bp, Homo sapiens
  source 1..25: mol_type = genomic DNA
acaatcttagatcttttattgtgcc

SEQ ID NO: 599 - DNA, 25 bp, Homo sapiens
  source 1..25: mol_type = genomic DNA
cataaacaatcttagatcttttatt

SEQ ID NO: 600 - DNA, 25 bp, Homo sapiens
  source 1..25: mol_type = genomic DNA
cttaattagcatatcctgtatatct

SEQ ID NO: 601 - DNA, 25 bp, Homo sapiens
  source 1..25: mol_type = genomic DNA
tcttcttaattagcatatcctgtat

SEQ ID NO: 602 - DNA, 25 bp, Homo sapiens
  source 1..25: mol_type = genomic DNA
tgctttcagagagattagaggcaga

SEQ ID NO: 603 - DNA, 25 bp, Homo sapiens
  source 1..25: mol_type = genomic DNA
cctactgctgctttcagagagatta

SEQ ID NO: 604 - DNA, 25 bp, Homo sapiens
  source 1..25: mol_type = genomic DNA
ggtggatgactcataccctccaatg

SEQ ID NO: 605 - DNA, 25 bp, Homo sapiens
  source 1..25: mol_type = genomic DNA
gttggctgatggtggatgactcata

SEQ ID NO: 606 - DNA, 25 bp, Homo sapiens
  source 1..25: mol_type = genomic DNA
ggtggatggatcataccctccaatg

SEQ ID NO: 607 - DNA, 25 bp, Homo sapiens
  source 1..25: mol_type = genomic DNA
gttggctgatggtggatggatcata

SEQ ID NO: 608 - DNA, 25 bp, Homo sapiens
  source 1..25: mol_type = genomic DNA
tcactatagatggctcatatggaaa

SEQ ID NO: 609 - DNA, 25 bp, Homo sapiens
  source 1..25: mol_type = genomic DNA
ataatcactatagatggctcatatg

SEQ ID NO: 610 - DNA, 25 bp, Homo sapiens
  source 1..25: mol_type = genomic DNA
aaagcattacattacgtaatcatat

SEQ ID NO: 611 - DNA, 25 bp, Homo sapiens
  source 1..25: mol_type = genomic DNA
cttcaactaaaatacagccaagttt

SEQ ID NO: 612 - DNA, 25 bp, Homo sapiens
  source 1..25: mol_type = genomic DNA
acagttggacagaagattattcttc

SEQ ID NO: 613 - DNA, 25 bp, Homo sapiens
  source 1..25: mol_type = genomic DNA
gcagactgacagttggacagaagat

SEQ ID NO: 614 - DNA, 25 bp, Homo sapiens
  source 1..25: mol_type = genomic DNA
gaataaggcttctagtctctttagt

SEQ ID NO: 615 - DNA, 25 bp, Homo sapiens
  source 1..25: mol_type = genomic DNA
ggcttctagtctctttagttgggcc

SEQ ID NO: 616 - DNA, 22 bp, Homo sapiens
  source 1..22: mol_type = genomic DNA
gcttacaatacgcaacttccac

SEQ ID NO: 617 - DNA, 25 bp, Homo sapiens
  source 1..25: mol_type = genomic DNA
ccttgataaacctagttccttttgt

SEQ ID NO: 618 - DNA, 25 bp, Homo sapiens
  source 1..25: mol_type = genomic DNA
tgtcgctgctaacagtatgttagct

SEQ ID NO: 619 - DNA, 25 bp, Homo sapiens
  source 1..25: mol_type = genomic DNA
acctcagaacaagatggcagaaagt

SEQ ID NO: 620 - DNA, 25 bp, Homo sapiens
  source 1..25: mol_type = genomic DNA
ctcagaacaagatggcagaaagtct

SEQ ID NO: 621 - DNA, 25 bp, Homo sapiens
  source 1..25: mol_type = genomic DNA
acaaatcaaagagaagctgcaagtc

SEQ ID NO: 622 - DNA, 25 bp, Homo sapiens
  source 1..25: mol_type = genomic DNA
agtcatggcaagtcctctgtttagt

SEQ ID NO: 623 - DNA, 25 bp, Homo sapiens
  source 1..25: mol_type = genomic DNA
atttcatctgaaacatacagaaaac

SEQ ID NO: 624 - DNA, 25 bp, Homo sapiens
  source 1..25: mol_type = genomic DNA
gctctcgagccagtttttctgatct

SEQ ID NO: 625 - DNA, 25 bp, Homo sapiens
  source 1..25: mol_type = genomic DNA
taaggagctctcgagccagtttttc

SEQ ID NO: 626 - DNA, 25 bp, Homo sapiens
  source 1..25: mol_type = genomic DNA
gtgagcccacaccattagtcagagg

SEQ ID NO: 627 - DNA, 25 bp, Homo sapiens
  source 1..25: mol_type = genomic DNA
cacaccattagtcagaggaaggttg

SEQ ID NO: 628 - DNA, 25 bp, Homo sapiens
  source 1..25: mol_type = genomic DNA
caagttctcagtaataagatcttgg

SEQ ID NO: 629 - DNA, 25 bp, Homo sapiens
  source 1..25: mol_type = genomic DNA
tggttggagagtacagccacaccat

SEQ ID NO: 630 - DNA, 20 bp, Homo sapiens
  source 1..20: mol_type = genomic DNA
taccagctctcctagataca

SEQ ID NO: 631 - DNA, 25 bp, Homo sapiens
  source 1..25: mol_type = genomic DNA
ctaccctgaaaatccgaaagtgttt

SEQ ID NO: 632 - DNA, 25 bp, Homo sapiens
  source 1..25: mol_type = genomic DNA
aattcaccgttgacattaacttcaa

SEQ ID NO: 633 - DNA, 25 bp, Homo sapiens
  source 1..25: mol_type = genomic DNA
gcagactctcttccgaggcaacagc

SEQ ID NO: 634 - DNA, 25 bp, Homo sapiens
  source 1..25: mol_type = genomic DNA
gcagactctcttccgaggcaacagc

SEQ ID NO: 635 - DNA, 25 bp, Homo sapiens
  source 1..25: mol_type = genomic DNA
agagcggtcccggaagctggcggag

SEQ ID NO: 636 - DNA, 25 bp, Homo sapiens
  source 1..25: mol_type = genomic DNA
tgattgagactagtgagagggtgca

SEQ ID NO: 637 - DNA, 25 bp, Homo sapiens
  source 1..25: mol_type = genomic DNA
ctcacgcattggctggctgcacaac

SEQ ID NO: 638 - DNA, 25 bp, Homo sapiens
  source 1..25: mol_type = genomic DNA
cacacatggactcacgcattggctg

SEQ ID NO: 639 - DNA, 25 bp, Homo sapiens
  source 1..25: mol_type = genomic DNA
tcctccatgtattccactcgaggga

SEQ ID NO: 640 - DNA, 25 bp, Homo sapiens
  source 1..25: mol_type = genomic DNA
tgtattccactcgagggatgggctg

SEQ ID NO: 641 - DNA, 25 bp, Homo sapiens
  source 1..25: mol_type = genomic DNA
cgccagtaagtctgccttgcttgtt

SEQ ID NO: 642 - DNA, 25 bp, Homo sapiens
  source 1..25: mol_type = genomic DNA
gacattgcctacaactaccgccagt

SEQ ID NO: 643 - DNA, 25 bp, Homo sapiens
  source 1..25: mol_type = genomic DNA
gccacccacccacccggctaggctg

SEQ ID NO: 644 - DNA, 25 bp, Homo sapiens
  source 1..25: mol_type = genomic DNA
ggacggggtggagctgaccagggag

SEQ ID NO: 645 - DNA, 25 bp, Homo sapiens
  source 1..25: mol_type = genomic DNA
gatcagctgatcacagcgctcctca

SEQ ID NO: 646 - DNA, 25 bp, Homo sapiens
  source 1..25: mol_type = genomic DNA
gaccggctcactggactacgagacc

SEQ ID NO: 647 - DNA, 25 bp, Homo sapiens
  source 1..25: mol_type = genomic DNA
accggctcactggactacgagaccg

SEQ ID NO: 648 - DNA, 25 bp, Homo sapiens
  source 1..25: mol_type = genomic DNA
aactactactcatggtgactcaagc

SEQ ID NO: 649 - DNA, 25 bp, Homo sapiens
  source 1..25: mol_type = genomic DNA
cctaaactactactcatggtgactc

SEQ ID NO: 650 - DNA, 25 bp, Homo sapiens
  source 1..25: mol_type = genomic DNA
tgcatctcctacaagtgagtctgcg

SEQ ID NO: 651 - DNA, 25 bp, Homo sapiens
  source 1..25: mol_type = genomic DNA
tgcatctcctacaagtgagtctgcg

SEQ ID NO: 652 - DNA, 25 bp, Homo sapiens
  source 1..25: mol_type = genomic DNA
tcaaccgtaagttaaaaagaaccac

SEQ ID NO: 653 - DNA, 25 bp, Homo sapiens
  source 1..25: mol_type = genomic DNA
actcttcatcaaccgtaagttaaaa

SEQ ID NO: 654 - DNA, 25 bp, Homo sapiens
  source 1..25: mol_type = genomic DNA
atactcatttatgattttgccaatt

SEQ ID NO: 655 - DNA, 25 bp, Homo sapiens
  source 1..25: mol_type = genomic DNA
ttcatttttattttcagtgaagaac

SEQ ID NO: 656 - DNA, 25 bp, Homo sapiens
  source 1..25: mol_type = genomic DNA
agccagcatgtgaatgagggaggaa

SEQ ID NO: 657 - DNA, 25 bp, Homo sapiens
  source 1..25: mol_type = genomic DNA
catctgggccattacagctaataag

SEQ ID NO: 658 - DNA, 25 bp, Homo sapiens
  source 1..25: mol_type = genomic DNA
ccctgggctcagcatggtcttcttg

SEQ ID NO: 659 - DNA, 25 bp, Homo sapiens
  source 1..25: mol_type = genomic DNA
ccctgggctcagcatggtcttcttg

SEQ ID NO: 660 - DNA, 25 bp, Homo sapiens
  source 1..25: mol_type = genomic DNA
ggggcagaacagcaggctcacctgc

SEQ ID NO: 661 - DNA, 25 bp, Homo sapiens
  source 1..25: mol_type = genomic DNA
acagtgcctcgaagctcctgcagca

SEQ ID NO: 662 - DNA, 25 bp, Homo sapiens
  source 1..25: mol_type = genomic DNA
gtgtgcccgcactcgaaaaagcagc

SEQ ID NO: 663 - DNA, 25 bp, Homo sapiens
  source 1..25: mol_type = genomic DNA
gcccgcactcgaaaaagcagccggg

SEQ ID NO: 664 - DNA, 25 bp, Homo sapiens
  source 1..25: mol_type = genomic DNA
gtttattttgtgtagatgtatacgt

SEQ ID NO: 665 - DNA, 25 bp, Homo sapiens
  source 1..25: mol_type = genomic DNA
gtttattttgtgtagatgtatacgt

SEQ ID NO: 666 - DNA, 25 bp, Homo sapiens
  source 1..25: mol_type = genomic DNA
ttacagtggatggagaagacatcat

SEQ ID NO: 667 - DNA, 25 bp, Homo sapiens
  source 1..25: mol_type = genomic DNA
taacacattattacagtggatggag

SEQ ID NO: 668 - DNA, 25 bp, Homo sapiens
  source 1..25: mol_type = genomic DNA
tattgacgttcccaaaaccatccag

SEQ ID NO: 669 - DNA, 25 bp, Homo sapiens
  source 1..25: mol_type = genomic DNA
gcggtctcagaggtcagagatggtc

SEQ ID NO: 670 - DNA, 25 bp, Homo sapiens
  source 1..25: mol_type = genomic DNA
agataaatcggtactgtgcttctgt

SEQ ID NO: 671 - DNA, 25 bp, Homo sapiens
  source 1..25: mol_type = genomic DNA
gtctggaccctccctgacctctgag

SEQ ID NO: 672 - DNA, 25 bp, Homo sapiens
  source 1..25: mol_type = genomic DNA
agttccctctgcgtgttctcataaa

SEQ ID NO: 673 - DNA, 25 bp, Homo sapiens
  source 1..25: mol_type = genomic DNA
cgtgttctcataaacaggcatgtgt

SEQ ID NO: 674 - DNA, 25 bp, Homo sapiens
  source 1..25: mol_type = genomic DNA
gagtgtattggtaagtgatccatcc

SEQ ID NO: 675 - DNA, 25 bp, Homo sapiens
  source 1..25: mol_type = genomic DNA
cgtggggagtgtattggtaagtgat

SEQ ID NO: 676 - DNA, 25 bp, Homo sapiens
  source 1..25: mol_type = genomic DNA
acttcaatctcactgtccaaggtga

SEQ ID NO: 677 - DNA, 25 bp, Homo sapiens
  source 1..25: mol_type = genomic DNA
cttcaatctcactgtccaaggtgag

SEQ ID NO: 678 - DNA, 25 bp, Homo sapiens
  source 1..25: mol_type = genomic DNA
gcaggcgctgctgggggcccgggct

SEQ ID NO: 679 - DNA, 25 bp, Homo sapiens
  source 1..25: mol_type = genomic DNA
ggcgctgctgggggcccgggctaag

SEQ ID NO: 680 - DNA, 25 bp, Homo sapiens
  source 1..25: mol_type = genomic DNA
agtttaacgaagccctccgtgagag

SEQ ID NO: 681 - DNA, 25 bp, Homo sapiens
  source 1..25: mol_type = genomic DNA
gtgaagtttaacgaagccctccgtg

SEQ ID NO: 682 - DNA, 25 bp, Homo sapiens
  source 1..25: mol_type = genomic DNA
aagctgctctgagggctcccaggtc

SEQ ID NO: 683 - DNA, 25 bp, Homo sapiens
  source 1..25: mol_type = genomic DNA
gtcacggacgagaactcccacagtc

SEQ ID NO: 684 - DNA, 25 bp, Homo sapiens
  source 1..25: mol_type = genomic DNA
tggggatgaggctggtgtactcgct

SEQ ID NO: 685 - DNA, 25 bp, Homo sapiens
  source 1..25: mol_type = genomic DNA
tgtactcgctcaggtccagcgtcat

SEQ ID NO: 686 - DNA, 25 bp, Homo sapiens
  source 1..25: mol_type = genomic DNA
acctcaccttgtgaaaaagttatat

SEQ ID NO: 687 - DNA, 25 bp, Homo sapiens
  source 1..25: mol_type = genomic DNA
gtaaatctgtgaaagagaatggatt

SEQ ID NO: 688 - DNA, 25 bp, Homo sapiens
  source 1..25: mol_type = genomic DNA
tgacagaaacactttttgacatagt

SEQ ID NO: 689 - DNA, 25 bp, Homo sapiens
  source 1..25: mol_type = genomic DNA
gaaacactttttgacatagtgtggt

SEQ ID NO: 690 - DNA, 25 bp, Homo sapiens
  source 1..25: mol_type = genomic DNA
acttcttggcaagtaggttaagatc

SEQ ID NO: 691 - DNA, 25 bp, Homo sapiens
  source 1..25: mol_type = genomic DNA
tggcaagtaggttaagatctgggaa

SEQ ID NO: 692 - DNA, 25 bp, Homo sapiens
  source 1..25: mol_type = genomic DNA
tcgctccatagatgcaacacccgat

SEQ ID NO: 693 - DNA, 25 bp, Homo sapiens
  source 1..25: mol_type = genomic DNA
cttacagctaatctctagaatttca

SEQ ID NO: 694 - DNA, 25 bp, Homo sapiens
  source 1..25: mol_type = genomic DNA
tcttttcttacagctaatctctaga

SEQ ID NO: 695 - DNA, 25 bp, Homo sapiens
  source 1..25: mol_type = genomic DNA
gacagcccactgcctataagtacaa

SEQ ID NO: 696 - DNA, 25 bp, Homo sapiens
  source 1..25: mol_type = genomic DNA
ggtccatccctacgctgaccagtgt

SEQ ID NO: 697 - DNA, 25 bp, Homo sapiens
  source 1..25: mol_type = genomic DNA
ccacttacatttgcaaatgctgatt

SEQ ID NO: 698 - DNA, 25 bp, Homo sapiens
  source 1..25: mol_type = genomic DNA
aaatgcttttgaagcaccacttaca

SEQ ID NO: 699 - DNA, 25 bp, Homo sapiens
  source 1..25: mol_type = genomic DNA
cccgcccggcacccgcgtctgcgcc

SEQ ID NO: 700 - DNA, 25 bp, Homo sapiens
  source 1..25: mol_type = genomic DNA
cccgcccggcacccgcgtctgcgcc

SEQ ID NO: 701 - DNA, 25 bp, Homo sapiens
  source 1..25: mol_type = genomic DNA
gctgccagaacatccttgggggcta

SEQ ID NO: 702 - DNA, 25 bp, Homo sapiens
  source 1..25: mol_type = genomic DNA
catggctgccagaacatccttgggg

SEQ ID NO: 703 - DNA, 25 bp, Homo sapiens
  source 1..25: mol_type = genomic DNA
atgattctgaattagctatatcgtc

SEQ ID NO: 704 - DNA, 25 bp, Homo sapiens
  source 1..25: mol_type = genomic DNA
gaattagctatatcgtcaaggcact

SEQ ID NO: 705 - DNA, 25 bp, Homo sapiens
  source 1..25: mol_type = genomic DNA
acctgtgaggaggttggcactgaaa

SEQ ID NO: 706 - DNA, 25 bp, Homo sapiens
  source 1..25: mol_type = genomic DNA
acctgtgaggaggttggcactgaaa

SEQ ID NO: 707 - DNA, 25 bp, Homo sapiens
  source 1..25: mol_type = genomic DNA
ggacggctcagacgtggcctcctgc

SEQ ID NO: 708 - DNA, 25 bp, Homo sapiens
  source 1..25: mol_type = genomic DNA
tgcttggacggctcagacgtggcct

SEQ ID NO: 709 - DNA, 25 bp, Homo sapiens
  source 1..25: mol_type = genomic DNA
cccctgacctcgctccccgaacccc

SEQ ID NO: 710 - DNA, 25 bp, Homo sapiens
  source 1..25: mol_type = genomic DNA
cccctgacctcgctccccgaacccc

SEQ ID NO: 711 - DNA, 25 bp, Homo sapiens
  source 1..25: mol_type = genomic DNA
tggggaactcccgacaagatcaaga

SEQ ID NO: 712 - DNA, 25 bp, Homo sapiens
  source 1..25: mol_type = genomic DNA
ggaactcccgacaagatcaagaaag

SEQ ID NO: 713 - DNA, 25 bp, Homo sapiens
  source 1..25: mol_type = genomic DNA
cttcgcatcttagctgggccaccag

SEQ ID NO: 714 - DNA, 25 bp, Homo sapiens
  source 1..25: mol_type = genomic DNA
cgcatcttagctgggccaccagctg

SEQ ID NO: 715 - DNA, 25 bp, Homo sapiens
  source 1..25: mol_type = genomic DNA
acttggccgtcgcacctccagaact

SEQ ID NO: 716 - DNA, 25 bp, Homo sapiens
  source 1..25: mol_type = genomic DNA
cagtccacttggccgtcgcacctcc

SEQ ID NO: 717 - DNA, 25 bp, Homo sapiens
  source 1..25: mol_type = genomic DNA
tgccatctattgatgggacccagac

SEQ ID NO: 718 - DNA, 25 bp, Homo sapiens
  source 1..25: mol_type = genomic DNA
caagaaatttgccatctattgatgg

SEQ ID NO: 719 - DNA, 25 bp, Homo sapiens
  source 1..25: mol_type = genomic DNA
gtgctcagtcgcgacataccgacag

SEQ ID NO: 720 - DNA, 25 bp, Homo sapiens
  source 1..25: mol_type = genomic DNA
agggtgctcagtcgcgacataccga

SEQ ID NO: 721 - DNA, 25 bp, Homo sapiens
  source 1..25: mol_type = genomic DNA
gactgcaaggacatgagcgttgaag

SEQ ID NO: 722 - DNA, 25 bp, Homo sapiens
  source 1..25: mol_type = genomic DNA
gactgcaaggacatgagcgttgaag

SEQ ID NO: 723 - DNA, 25 bp, Homo sapiens
  source 1..25: mol_type = genomic DNA
ggagggtggctacaagtgccagtgt

SEQ ID NO: 724 - DNA, 25 bp, Homo sapiens
  source 1..25: mol_type = genomic DNA
tgcgtgaacctggagggtggctaca

SEQ ID NO: 725 - DNA, 25 bp, Homo sapiens
  source 1..25: mol_type = genomic DNA
ctcacctgcagatcattctctggga

SEQ ID NO: 726 - DNA, 25 bp, Homo sapiens
  source 1..25: mol_type = genomic DNA
tgcagatcattctctgggacaggtc

SEQ ID NO: 727 - DNA, 25 bp, Homo sapiens
  source 1..25: mol_type = genomic DNA
ccacttgtgtgtctagatctcctca

SEQ ID NO: 728 - DNA, 25 bp, Homo sapiens
  source 1..25: mol_type = genomic DNA
ctcagtggccgcctctactgggttg

SEQ ID NO: 729 - DNA, 20 bp, synthetic construct
  misc_feature 1..20: note = source = /note="Description of Artificial Sequence: Synthetic oligonucleotide"
  source 1..20: mol_type = other DNA
tttcaccatgttggccaggt

SEQ ID NO: 730 - DNA, 20 bp, synthetic construct
  misc_feature 1..20: note = source = /note="Description of Artificial Sequence: Synthetic oligonucleotide"
  source 1..20: mol_type = other DNA
tactgggtttcaccatgttg

SEQ ID NO: 731 - DNA, 20 bp, synthetic construct
  misc_feature 1..20: note = source = /note="Description of Artificial Sequence: Synthetic oligonucleotide"
  source 1..20: mol_type = other DNA
agaaaagttagccgggcgtg

SEQ ID NO: 732 - DNA, 20 bp, synthetic construct
  misc_feature 1..20: note = source = /note="Description of Artificial Sequence: Synthetic oligonucleotide"
  source 1..20: mol_type = other DNA
acaggcgcgcgacaccacgc

SEQ ID NO: 733 - RNA, 159 nt, synthetic construct
  misc_feature 1..159: note = source = /note="Description of Artificial Sequence: Synthetic polynucleotide"
  source 1..159: mol_type = other RNA
tttcaccatgttggccaggtgccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 734 - RNA, 159 nt, synthetic construct
  misc_feature 1..159: note = source = /note="Description of Artificial Sequence: Synthetic polynucleotide"
  source 1..159: mol_type = other RNA
tactgggtttcaccatgttggccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 735 - RNA, 159 nt, synthetic construct
  misc_feature 1..159: note = source = /note="Description of Artificial Sequence: Synthetic polynucleotide"
  source 1..159: mol_type = other RNA
agaaaagttagccgggcgtggccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 736 - RNA, 159 nt, synthetic construct
  misc_feature 1..159: note = source = /note="Description of Artificial Sequence: Synthetic polynucleotide"
  source 1..159: mol_type = other RNA
acaggcgcgcgacaccacgcgccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 737 - DNA, 20 bp, synthetic construct
  misc_feature 1..20: note = source = /note="Description of Artificial Sequence: Synthetic oligonucleotide"
  source 1..20: mol_type = other DNA
tcctggagcctgtgataaaa

SEQ ID NO: 738 - DNA, 20 bp, synthetic construct
  misc_feature 1..20: note = source = /note="Description of Artificial Sequence: Synthetic oligonucleotide"
  source 1..20: mol_type = other DNA
gctaacagttgcttttatca

SEQ ID NO: 739 - DNA, 20 bp, synthetic construct
  misc_feature 1..20: note = source = /note="Description of Artificial Sequence: Synthetic oligonucleotide"
  source 1..20: mol_type = other DNA
taacagttgcttttatcaca

SEQ ID NO: 740 - RNA, 159 nt, synthetic construct
  misc_feature 1..159: note = source = /note="Description of Artificial Sequence: Synthetic polynucleotide"
  source 1..159: mol_type = other RNA
tcctggagcctgtgataaaagccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 741 - RNA, 159 nt, synthetic construct
  misc_feature 1..159: note = source = /note="Description of Artificial Sequence: Synthetic polynucleotide"
  source 1..159: mol_type = other RNA
gctaacagttgcttttatcagccataattcctctgtaaaacttaaagaaggtttatagagttattatggtaaggcaatatgccgtggcgttggggatcgcctatgtccggttttaccggatctccctaaaggtgactaactttggttagtcaccttttt

SEQ ID NO: 742 - RNA, 159 nt, synthetic construct
  misc_feature 1..159: note = source = /note="Description of Artificial Sequence: Synthetic polynucleotide"
  source 1..159: mol_type = other RNA
taacagttgcttttatcacagccataattcctctgtaaaacttaaagaaggtttatagagttattatggtaaggcaatatgccgtggcgttggggatcgcctatgtccggttttaccggatctccctaaaggtgactaactttggttagtcaccttttt

(19) United States
(12) Patent Application Publication
(10) Pub. No.: US 2026/0327601 A1
(43) Pub. Date: Oct. 22, 2026

(54) RNA-GUIDED NUCLEASES AND ACTIVE FRAGMENTS AND VARIANTS THEREOF AND METHODS OF USE

(71) Applicant: Life Edit Therapeutics, Inc., Durham, NC (US)

(72) Inventors: Tyson D. Bowen, Morrisville, NC (US); Alexandra Briner Crawley, Cary, NC (US); Tedd D. Elich, Durham, NC (US); Michael Coyle, Chapel Hill, NC (US)

(73) Assignee: Life Edit Therapeutics, Inc., Durham, NC (US)

(21) Appl. No.: 19/706,459

(22) Filed: Jun. 12, 2026

Related U.S. Application Data
(63) Continuation of application No. 17/634,723, filed on Aug. 11, 2020, now Pat. No. 12,680,096.
(60) Provisional application No. 62/885,483, filed on Aug. 12, 2019.
(60) Provisional application No. 62/901,875, filed on Sep. 18, 2019.
(60) Provisional application No. 63/030,088, filed on May 26, 2020.

(51) Int. Cl. C12N 15/10; C12N 9/22; C12N 15/79

(52) U.S. Cl. CPC C12N 15/102; C12N 9/22; C12N 15/79; C12N 2310/20

(57) ABSTRACT

Compositions and methods for binding to a target sequence of interest are provided. The compositions find use in cleaving or modifying a target sequence of interest, visualization of a target sequence of interest, and modifying the expression of a sequence of interest. Compositions comprise RNA-guided nuclease polypeptides, CRISPR RNAs, trans-activating CRISPR RNAs, guide RNAs, and nucleic acid molecules encoding the same. Vectors and host cells comprising the nucleic acid molecules are also provided. Further provided are CRISPR systems for binding a target sequence of interest, wherein the CRISPR system comprises an RNA-guided nuclease polypeptide and one or more guide RNAs. Methods and kits for detecting a target DNA sequence are also provided.

### CROSS-REFERENCE TO RELATED APPLICATIONS

This application is a continuation of U.S. application Ser. No. 17/634,723, filed Feb. 11, 2022, which is a national stage filing under 35 U.S.C. 371 of International Application No. PCT/US2020/045759, filed Aug. 11, 2020, and published by the International Bureau in English on Feb. 18, 2021, and which International Application claims the benefit of priority to U.S. Provisional Application Nos. 62/885,483, filed Aug. 12, 2019, 62/901,875, filed Sep. 18, 2019, and 63/030,088, filed May 26, 2020, each of which application is incorporated by reference herein in its entirety.

### FIELD OF THE INVENTION

The present invention relates to the field of molecular biology and gene editing.

### REFERENCE TO A SEQUENCE LISTING SUBMITTED ELECTRONICALLY AS AN XML FILE

The instant application contains a Sequence Listing which has been submitted in xml format via USPTO Patent Center and is hereby incorporated by reference in its entirety. Said xml copy, created on Jun. 11, 2026, is named L103438_1170USC1_00432_Seq_List and is 846,928 bytes in size.

### BACKGROUND OF THE INVENTION

Targeted genome editing or modification is rapidly becoming an important tool for basic and applied research. Initial methods involved engineering nucleases such as meganucleases, zinc finger fusion proteins or TALENs, requiring the generation of chimeric nucleases with engineered, programmable, sequence-specific DNA-binding domains specific for each particular target sequence. RNA-guided nucleases, such as the Clustered Regularly Interspaced Short Palindromic Repeats (CRISPR)-associated (Cas) proteins of the CRISPR-Cas bacterial system, allow for the targeting of specific sequences by complexing the nucleases with guide RNA that specifically hybridizes with a particular target sequence. Producing target-specific guide RNAs is less costly and more efficient than generating chimeric nucleases for each target sequence. Such RNA-guided nucleases can be used to edit genomes optionally through the introduction of a sequence-specific, double-stranded break that is repaired via error-prone non-homologous end-joining (NHEJ) to introduce a mutation at a specific genomic location. Alternatively, heterologous DNA may be introduced into the genomic site via homology-directed repair.

### BRIEF SUMMARY OF THE INVENTION

Compositions and methods for binding a target sequence of interest are provided. The compositions find use in cleaving or modifying a target sequence of interest, detection of a target sequence of interest, and modifying the expression of a sequence of interest. Compositions comprise RNA-guided nuclease (RGN) polypeptides, CRISPR RNAs (crRNAs), trans-activating CRISPR RNAs (tracrRNAs), guide RNAs (gRNAs), nucleic acid molecules encoding the same, and vectors and host cells comprising the nucleic acid molecules. Also provided are CRISPR systems for binding a target sequence of interest, wherein the CRISPR system comprises an RNA-guided nuclease polypeptide and one or more guide RNAs. Thus, methods disclosed herein are drawn to binding a target sequence of interest, and in some embodiments, cleaving or modifying the target sequence of interest. The target sequence of interest can be modified, for example, as a result of non-homologous end joining or homology-directed repair with an introduced donor sequence. Further provided are methods and kits for detecting a target DNA sequence of a DNA molecule using detector single-stranded DNA.

### DETAILED DESCRIPTION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended embodiments. Although specific terms are employed herein, they are used in a generic and descriptive sense only and not for purposes of limitation.

### I. Overview

RNA-guided nucleases (RGNs) allow for the targeted manipulation of a single site within a genome and are useful in the context of gene targeting for therapeutic and research applications. In a variety of organisms, including mammals, RNA-guided nucleases have been used for genome engineering by stimulating non-homologous end joining and homologous recombination, for example. The compositions and methods described herein are useful for creating single- or double-stranded breaks in polynucleotides, modifying polynucleotides, detecting a particular site within a polynucleotide, or modifying the expression of a particular gene.

The RNA-guided nucleases disclosed herein can alter gene expression by modifying a target sequence. In specific embodiments, the RNA-guided nucleases are directed to the target sequence by a guide RNA (gRNA) as part of a Clustered Regularly Interspaced Short Palindromic Repeats (CRISPR) RNA-guided nuclease system. The RGNs are considered “RNA-guided” because guide RNAs form a complex with the RNA-guided nucleases to direct the RNA-guided nuclease to bind to a target sequence and in some embodiments, introduce a single-stranded or double-stranded break at the target sequence. After the target sequence has been cleaved, the break can be repaired such that the DNA sequence of the target sequence is modified during the repair process. Thus, provided herein are methods for using the RNA-guided nucleases to modify a target sequence in the DNA of host cells. For example, RNA-guided nucleases can be used to modify a target sequence at a genomic locus of eukaryotic cells or prokaryotic cells.

### II. RNA-Guided Nucleases

Provided herein are RNA-guided nucleases. The term RNA-guided nuclease (RGN) refers to a polypeptide that binds to a particular target nucleotide sequence in a sequence-specific manner and is directed to the target nucleotide sequence by a guide RNA molecule that is complexed with the polypeptide and hybridizes with the target sequence. Although an RNA-guided nuclease can be capable of cleaving the target sequence upon binding, the term RNA-guided nuclease also encompasses nuclease-dead RNA-guided nucleases that are capable of binding to, but not cleaving, a target sequence. Cleavage of a target sequence by an RNA-guided nuclease can result in a single- or double-stranded break. RNA-guided nucleases only capable of cleaving a single strand of a double-stranded nucleic acid molecule are referred to herein as nickases.

The RNA-guided nucleases disclosed herein include the APG05733.1, APG06207.1, APG01647.1, APG08032.1, APG05712.1, APG01658.1, APG06498.1, APG09106.1, APG09882.1, APG02675.1, APG01405.1, APG06250.1, APG06877.1, APG09053.1, APG04293.1, APG01308.1, APG06646.1, APG09748, and APG07433.1 RNA-guided nucleases, the amino acid sequences of which are set forth, respectively, as SEQ ID NO: 1, 9, 16, 23, 30, 38, 46, 54, 61, 69, 75, 82, 89, 95, 103, 110, 117, 137 or 235, and active fragments or variants thereof that retain the ability to bind to a target nucleotide sequence in an RNA-guided sequence-specific manner. In some of these embodiments, the active fragment or variant of the APG05733.1, APG06207.1, APG01647.1, APG08032.1, APG05712.1, APG01658.1, APG06498.1, APG09106.1, APG09882.1, APG02675.1, APG01405.1, APG06250.1, APG06877.1, APG09053.1, APG04293.1, APG01308.1, APG06646.1, APG09748, or APG07433.1 RGN is capable of cleaving a single- or double-stranded target sequence. In some embodiments, an active variant of the APG05733.1, APG06207.1, APG01647.1, APG08032.1, APG05712.1, APG01658.1, APG06498.1, APG09106.1, APG09882.1, APG02675.1, APG01405.1, APG06250.1, APG06877.1, APG09053.1, APG04293.1, APG01308.1, APG06646.1, APG09748, or APG07433.1 RGN comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth as SEQ ID NO: 1, 9, 16, 23, 30, 38, 46, 54, 61, 69, 75, 82, 89, 95, 103, 110, 117, 137 or 235. In certain embodiments, an active fragment of the APG05733.1, APG06207.1, APG01647.1, APG08032.1, APG05712.1, APG01658.1, APG06498.1, APG09106.1, APG09882.1, APG02675.1, APG01405.1, APG06250.1, APG06877.1, APG09053.1, APG04293.1, APG01308.1, APG06646.1, APG09748, or APG07433.1 RGN comprises at least 50, 100, 150, 200, 250, 300, 350, 400, 450, 500, 550, 600, 650, 700, 750, 800, 850, 900, 950, 1000, 1050 or more contiguous amino acid residues of the amino acid sequence set forth as SEQ ID NO: 1, 9, 16, 23, 30, 38, 46, 54, 61, 69, 75, 82, 89, 95, 103, 110, 117, 137, or 235. RNA-guided nucleases provided herein can comprise at least one nuclease domain (e.g., DNase, RNase domain) and at least one RNA recognition and/or RNA binding domain to interact with guide RNAs. Further domains that can be found in RNA-guided nucleases provided herein include, but are not limited to: DNA binding domains, helicase domains, protein-protein interaction domains, and dimerization domains. In specific embodiments, the RNA-guided nucleases provided herein can comprise at least 70%, 75%, 80%, 85%, 90%, 91%, 92%, 93%, 94%, 95%, 96%, 97%, 98%, 99% to one or more of a DNA binding domains, helicase domains, protein-protein interaction domains, and dimerization domains.

A target nucleotide sequence is bound by an RNA-guided nuclease provided herein and hybridizes with the guide RNA associated with the RNA-guided nuclease. The target sequence can then be subsequently cleaved by the RNA-guided nuclease if the polypeptide possesses nuclease activity. The terms “cleave” or “cleavage” refer to the hydrolysis of at least one phosphodiester bond within the backbone of a target nucleotide sequence that can result in either single-stranded or double-stranded breaks within the target sequence. The presently disclosed RGNs can cleave nucleotides within a polynucleotide, functioning as an endonuclease or can be an exonuclease, removing successive nucleotides from the end (the 5′ and/or the 3′ end) of a polynucleotide. In other embodiments, the disclosed RGNs can cleave nucleotides of a target sequence within any position of a polynucleotide and thus function as both an endonuclease and exonuclease. The cleavage of a target polynucleotide by the presently disclosed RGNs can result in staggered breaks or blunt ends.

The presently disclosed RNA-guided nucleases can be wild-type sequences derived from bacterial or archaeal species. Alternatively, the RNA-guided nucleases can be variants or fragments of wild-type polypeptides. The wild-type RGN can be modified to alter nuclease activity or alter PAM specificity, for example. In some embodiments, the RNA-guided nuclease is not naturally-occurring.

In certain embodiments, the RNA-guided nuclease functions as a nickase, only cleaving a single strand of the target nucleotide sequence. Such RNA-guided nucleases have a single functioning nuclease domain. In some of these embodiments, additional nuclease domains have been mutated such that the nuclease activity is reduced or eliminated.

In other embodiments, the RNA-guided nuclease lacks nuclease activity altogether or exhibits reduced nuclease activity, and is referred to herein as nuclease-dead or nuclease inactive. Any method known in the art for introducing mutations into an amino acid sequence, such as PCR-mediated mutagenesis and site-directed mutagenesis, can be used for generating nickases or nuclease-dead RGNs. See, e.g., U.S. Publ. No. 2014/0068797 and U.S. Pat. No. 9,790,490; each of which is incorporated by reference in its entirety.

RNA-guided nucleases that lack nuclease activity can be used to deliver a fused polypeptide, polynucleotide, or small molecule payload to a particular genomic location. In some of these embodiments, the RGN polypeptide or guide RNA can be fused to a detectable label to allow for detection of a particular sequence. As a non-limiting example, a nuclease-dead RGN can be fused to a detectable label (e.g., fluorescent protein) and targeted to a particular sequence associated with a disease to allow for detection of the disease-associated sequence.

Alternatively, nuclease-dead RGNs can be targeted to particular genomic locations to alter the expression of a desired sequence. In some embodiments, the binding of a nuclease-dead RNA-guided nuclease to a target sequence results in the repression of expression of the target sequence or a gene under transcriptional control by the target sequence by interfering with the binding of RNA polymerase or transcription factors within the targeted genomic region. In other embodiments, the RGN (e.g., a nuclease-dead RGN) or its complexed guide RNA further comprises an expression modulator that, upon binding to a target sequence, serves to either repress or activate the expression of the target sequence or a gene under transcriptional control by the target sequence. In some of these embodiments, the expression modulator modulates the expression of the target sequence or regulated gene through epigenetic mechanisms.

In other embodiments, the nuclease-dead RGNs or a RGN with only nickase activity can be targeted to particular genomic locations to modify the sequence of a target polynucleotide through fusion to a base-editing polypeptide, for example a deaminase polypeptide or active variant or fragment thereof that deaminates a nucleotide base, resulting in conversion from one nucleotide base to another. The base-editing polypeptide can be fused to the RGN at its N-terminal or C-terminal end. Additionally, the base-editing polypeptide may be fused to the RGN via a peptide linker. A non-limiting example of a deaminase polypeptide that is useful for such compositions and methods include a cytidine deaminase or an adenosine deaminase (such as the adenosine deaminase base editor described in Gaudelli et al. (2017) Nature 551:464-471, U.S. Publ. Nos. 2017/0121693 and 2018/0073012, and International Publ. No. WO/2018/027078, or any of the deaminases disclosed in International Appl. No. PCT/US2019/068079, each of which is herein incorporated by reference in its entirety). Further, it is known in the art that certain fusion proteins between an RGN and a base-editing enzyme may also comprise at least one uracil stabilizing polypeptide that increases the mutation rate of a cytidine, deoxycytidine, or cytosine to a thymidine, deoxythymidine, or thymine in a nucleic acid molecule by a deaminase. Non-limiting examples of uracil stabilizing polypeptides include those disclosed in U.S. Provisional Appl. No. 63/052,175, filed Jul. 15, 2020, and a uracil glycosylase inhibitor (UGI) domain (SEQ ID NO: 261), which may increase base editing efficiency (U.S. Pat. No. 10,167,547, herein incorporated by reference). Therefore, a fusion protein may comprise an RGN described herein or a variant thereof, a deaminase, and optionally at least one uracil stabilizing polypeptides, such as UGI.

RNA-guided nucleases that are fused to a polypeptide or domain can be separated or joined by a linker. The term “linker,” as used herein, refers to a chemical group or a molecule linking two molecules or moieties, e.g., a binding domain and a cleavage domain of a nuclease. In some embodiments, a linker joins a gRNA binding domain of an RNA guided nuclease and a base-editing polypeptide, such as a deaminase. In some embodiments, a linker joins a nuclease-dead RGN and a deaminase. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The presently disclosed RNA-guided nucleases can comprise at least one nuclear localization signal (NLS) to enhance transport of the RGN to the nucleus of a cell. Nuclear localization signals are known in the art and generally comprise a stretch of basic amino acids (see, e.g., Lange et al., J. Biol. Chem. (2007) 282:5101-5105). In particular embodiments, the RGN comprises 2, 3, 4, 5, 6 or more nuclear localization signals. The nuclear localization signal(s) can be a heterologous NLS. Non-limiting examples of nuclear localization signals useful for the presently disclosed RGNs are the nuclear localization signals of SV40 Large T-antigen, nucleopasmin, and c-Myc (see, e.g., Ray et al. (2015) Bioconjug Chem 26 (6): 1004-7). In particular embodiments, the RGN comprises the NLS sequence set forth as SEQ ID NO: 125 or 127. The RGN can comprise one or more NLS sequences at its N-terminus, C-terminus, or both the N-terminus and C-terminus. For example, the RGN can comprise two NLS sequences at the N-terminal region and four NLS sequences at the C-terminal region.

Other localization signal sequences known in the art that localize polypeptides to particular subcellular location(s) can also be used to target the RGNs, including, but not limited to, plastid localization sequences, mitochondrial localization sequences, and dual-targeting signal sequences that target to both the plastid and mitochondria (see, e.g., Nassoury and Morse (2005) Biochim Biophys Acta 1743:5-19; Kunze and Berger (2015) Front Physiol dx.doi.org/10.3389/fphys.2015.00259; Herrmann and Neupert (2003) IUBMB Life 55:219-225; Soll (2002) Curr Opin Plant Biol 5:529-535; Carrie and Small (2013) Biochim Biophys Acta 1833:253-259; Carrie et al. (2009) FEBS J 276:1187-1195; Silva-Filho (2003) Curr Opin Plant Biol 6:589-595; Peeters and Small (2001) Biochim Biophys Acta 1541:54-63; Murcha et al. (2014) J Exp Bot 65:6301-6335; Mackenzie (2005) Trends Cell Biol 15:548-554; Glaser et al. (1998) Plant Mol Biol 38:311-338).

In certain embodiments, the presently disclosed RNA-guided nucleases comprise at least one cell-penetrating domain that facilitates cellular uptake of the RGN. Cell-penetrating domains are known in the art and generally comprise stretches of positively charged amino acid residues (i.e., polycationic cell-penetrating domains), alternating polar amino acid residues and non-polar amino acid residues (i.e., amphipathic cell-penetrating domains), or hydrophobic amino acid residues (i.e., hydrophobic cell-penetrating domains) (see, e.g., Milletti F. (2012) Drug Discov Today 17:850-860). A non-limiting example of a cell-penetrating domain is the trans-activating transcriptional activator (TAT) from the human immunodeficiency virus 1.

The nuclear localization signal, plastid localization signal, mitochondrial localization signal, dual-targeting localization signal, and/or cell-penetrating domain can be located at the amino-terminus (N-terminus), the carboxyl-terminus (C-terminus), or in an internal location of the RNA-guided nuclease.

The presently disclosed RGNs can be fused to an effector domain, such as a cleavage domain, a deaminase domain, or an expression modulator domain, either directly or indirectly via a linker peptide. Such a domain can be located at the N-terminus, the C-terminus, or an internal location of the RNA-guided nuclease. In some of these embodiments, the RGN component of the fusion protein is a nuclease-dead RGN.

In some embodiments, the RGN fusion protein comprises a cleavage domain, which is any domain that is capable of cleaving a polynucleotide (i.e., RNA, DNA, or RNA/DNA hybrid) and includes, but is not limited to, restriction endonucleases and homing endonucleases, such as Type IIS endonucleases (e.g., FokI) (see, e.g., Belfort et al. (1997) Nucleic Acids Res. 25:3379-3388; Linn et al. (eds.) Nucleases, Cold Spring Harbor Laboratory Press, 1993).

In other embodiments, the RGN fusion protein comprises a deaminase domain that deaminates a nucleotide base, resulting in conversion from one nucleotide base to another, and includes, but is not limited to, a cytidine deaminase or an adenosine deaminase base editor (see, e.g., Gaudelli et al. (2017) Nature 551:464-471, U.S. Publ. Nos. 2017/0121693 and 2018/0073012, U.S. Pat. No. 9,840,699, and International Publ. No. WO/2018/027078).

In some embodiments, the effector domain of the RGN fusion protein can be an expression modulator domain, which is a domain that either serves to upregulate or downregulate transcription. The expression modulator domain can be an epigenetic modification domain, a transcriptional repressor domain or a transcriptional activation domain.

In some of these embodiments, the expression modulator of the RGN fusion protein comprises an epigenetic modification domain that covalently modifies DNA or histone proteins to alter histone structure and/or chromosomal structure without altering the DNA sequence, leading to changes in gene expression (i.e., upregulation or downregulation). Non-limiting examples of epigenetic modifications include acetylation or methylation of lysine residues, arginine methylation, serine and threonine phosphorylation, and lysine ubiquitination and sumoylation of histone proteins, and methylation and hydroxymethylation of cytosine residues in DNA. Non-limiting examples of epigenetic modification domains include histone acetyltransferase domains, histone deacetylase domains, histone methyltransferase domains, histone demethylase domains, DNA methyltransferase domains, and DNA demethylase domains.

In other embodiments, the expression modulator of the fusion protein comprises a transcriptional repressor domain, which interacts with transcriptional control elements and/or transcriptional regulatory proteins, such as RNA polymerases and transcription factors, to reduce or terminate transcription of at least one gene. Transcriptional repressor domains are known in the art and include, but are not limited to, Sp1-like repressors, IκB, and Krüppel associated box (KRAB) domains.

In yet other embodiments, the expression modulator of the fusion protein comprises a transcriptional activation domain, which interacts with transcriptional control elements and/or transcriptional regulatory proteins, such as RNA polymerases and transcription factors, to increase or activate transcription of at least one gene. Transcriptional activation domains are known in the art and include, but are not limited to, a herpes simplex virus VP16 activation domain and an NFAT activation domain.

The presently disclosed RGN polypeptides can comprise a detectable label or a purification tag. The detectable label or purification tag can be located at the N-terminus, the C-terminus, or an internal location of the RNA-guided nuclease, either directly or indirectly via a linker peptide. In some of these embodiments, the RGN component of the fusion protein is a nuclease-dead RGN. In other embodiments, the RGN component of the fusion protein is an RGN with nickase activity.

A detectable label is a molecule that can be visualized or otherwise observed. The detectable label may be fused to the RGN as a fusion protein (e.g., fluorescent protein) or may be a small molecule conjugated to the RGN polypeptide that can be detected visually or by other means. Detectable labels that can be fused to the presently disclosed RGNs as a fusion protein include any detectable protein domain, including but not limited to, a fluorescent protein or a protein domain that can be detected with a specific antibody. Non-limiting examples of fluorescent proteins include green fluorescent proteins (e.g., GFP, EGFP, ZsGreen1) and yellow fluorescent proteins (e.g., YFP, EYFP, ZsYellow1). Non-limiting examples of small molecule detectable labels include radioactive labels, such as 3H and 35S.

RGN polypeptides can also comprise a purification tag, which is any molecule that can be utilized to isolate a protein or fused protein from a mixture (e.g., biological sample, culture medium). Non-limiting examples of purification tags include biotin, myc, maltose binding protein (MBP), and glutathione-S-transferase (GST).

### II. Guide RNA

The present disclosure provides guide RNAs and polynucleotides encoding the same. The term “guide RNA” refers to a nucleotide sequence having sufficient complementarity with a target nucleotide sequence to hybridize with the target sequence and direct sequence-specific binding of an associated RNA-guided nuclease to the target nucleotide sequence. Thus, a RGN's respective guide RNA is one or more RNA molecules (generally, one or two), that can bind to the RGN and guide the RGN to bind to a particular target nucleotide sequence, and in those instances wherein the RGN has nickase or nuclease activity, also cleave the target nucleotide sequence. In general, a guide RNA comprises a CRISPR RNA (crRNA) and in some embodiments, a trans-activating CRISPR RNA (tracrRNA). Native guide RNAs that comprise both a crRNA and a tracrRNA generally comprise two separate RNA molecules that hybridize to each other through the repeat sequence of the crRNA and the anti-repeat sequence of the tracrRNA.

Native direct repeat sequences within a CRISPR array generally range in length from 28 to 37 base pairs, although the length can vary between about 23 bp to about 55 bp. Spacer sequences within a CRISPR array generally range from about 32 to about 38 bp in length, although the length can be between about 21 bp to about 72 bp. Each CRISPR array generally comprises less than 50 units of the CRISPR repeat-spacer sequence. The CRISPRs are transcribed as part of a long transcript termed the primary CRISPR transcript, which comprises much of the CRISPR array. The primary CRISPR transcript is cleaved by Cas proteins to produce crRNAs or in some cases, to produce pre-crRNAs that are further processed by additional Cas proteins into mature crRNAs. Mature crRNAs comprise a spacer sequence and a CRISPR repeat sequence. In some embodiments in which pre-crRNAs are processed into mature (or processed) crRNAs, maturation involves the removal of about one to about six or more 5′, 3′, or 5′ and 3′ nucleotides. For the purposes of genome editing or targeting a particular target nucleotide sequence of interest, these nucleotides that are removed during maturation of the pre-crRNA molecule are not necessary for generating or designing a guide RNA.

A CRISPR RNA (crRNA) comprises a spacer sequence and a CRISPR repeat sequence. The “spacer sequence” is the nucleotide sequence that directly hybridizes with the target nucleotide sequence of interest. The spacer sequence is engineered to be fully or partially complementary with the target sequence of interest. In various embodiments, the spacer sequence can comprise from about 8 nucleotides to about 30 nucleotides, or more. For example, the spacer sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the spacer sequence is about 10 to about 26 nucleotides in length, or about 12 to about 30 nucleotides in length. In particular embodiments, the spacer sequence is about 30 nucleotides in length. In some embodiments, the degree of complementarity between a spacer sequence and its corresponding target sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In particular embodiments, the spacer sequence is free of secondary structure, which can be predicted using any suitable polynucleotide folding algorithm known in the art, including but not limited to mFold (see, e.g., Zuker and Stiegler (1981) Nucleic Acids Res. 9:133-148) and RNAfold (see, e.g., Gruber et al. (2008) Cell 106 (1): 23-24).

The CRISPR RNA repeat sequence comprises a nucleotide sequence that comprises a region with sufficient complementarity to hybridize to a tracrRNA. In various embodiments, the CRISPR RNA repeat sequence can comprise from about 8 nucleotides to about 30 nucleotides, or more. For example, the CRISPR repeat sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the CRISPR repeat sequence is about 21 nucleotides in length. In some embodiments, the degree of complementarity between a CRISPR repeat sequence and its corresponding tracrRNA sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In particular embodiments, the CRISPR repeat sequence comprises the nucleotide sequence of SEQ ID NO: 2, 10, 17, 24, 31, 39, 47, 55, 62, 70, 76, 83, 90, 96, 104, 111, 118, 240, 273, or 287, or an active variant or fragment thereof that when comprised within a guide RNA, is capable of directing the sequence-specific binding of an associated RNA-guided nuclease provided herein to a target sequence of interest. In certain embodiments, an active CRISPR repeat sequence variant of a wild-type sequence comprises a nucleotide sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the nucleotide sequence set forth as SEQ ID NO: 2, 10, 17, 24, 31, 39, 47, 55, 62, 70, 76, 83, 90, 96, 104, 111, 118, 240, 273 or 287. In certain embodiments, an active CRISPR repeat sequence fragment of a wild-type sequence comprises at least 5, 6, 7, 8, 9, 10, 11, 12, 13, 14, 15, 16, 17, 18, 19, or 20 contiguous nucleotides of the nucleotide sequence set forth as SEQ ID NO: 2, 10, 17, 24, 31, 39, 47, 55, 62, 70, 76, 83, 90, 96, 104, 111, 118, 240, 273, or 287.

In certain embodiments, the crRNA is not naturally-occurring. In some of these embodiments, the specific CRISPR repeat sequence is not linked to the engineered spacer sequence in nature and the CRISPR repeat sequence is considered heterologous to the spacer sequence. In certain embodiments, the spacer sequence is an engineered sequence that is not naturally occurring.

A trans-activating CRISPR RNA or tracrRNA molecule comprises a nucleotide sequence comprising a region that has sufficient complementarity to hybridize to a CRISPR repeat sequence of a crRNA, which is referred to herein as the anti-repeat region. In some embodiments, the tracrRNA molecule further comprises a region with secondary structure (e.g., stem-loop) or forms secondary structure upon hybridizing with its corresponding crRNA. In particular embodiments, the region of the tracrRNA that is fully or partially complementary to a CRISPR repeat sequence is at the 5′ end of the molecule and the 3′ end of the tracrRNA comprises secondary structure. This region of secondary structure generally comprises several hairpin structures, including the nexus hairpin, which is found adjacent to the anti-repeat sequence. The nexus hairpin often has a conserved nucleotide sequence in the base of the hairpin stem, with the motif UNANNA, CNANNG, CNANNU, UNANNG, UNANNC, or CNANNU (SEQ ID NOs: 8, 37, 45, 53, 68, and 102, respectively) found in many nexus hairpins in tracrRNAs. There are often terminal hairpins at the 3′ end of the tracrRNA that can vary in structure and number, but often comprise a GC-rich Rho-independent transcriptional terminator hairpin followed by a string of U's at the 3′ end. See, for example, Briner et al. (2014) Molecular Cell 56:333-339, Briner and Barrangou (2016) Cold Spring Harb Protoc; doi: 10.1101/pdb.top090902, and U.S. Publication No. 2017/0275648, each of which is herein incorporated by reference in its entirety.

In various embodiments, the anti-repeat region of the tracrRNA that is fully or partially complementary to the CRISPR repeat sequence comprises from about 8 nucleotides to about 30 nucleotides, or more. For example, the region of base pairing between the tracrRNA anti-repeat sequence and the CRISPR repeat sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In particular embodiments, the anti-repeat region of the tracrRNA that is fully or partially complementary to a CRISPR repeat sequence is about 20 nucleotides in length. In some embodiments, the degree of complementarity between a CRISPR repeat sequence and its corresponding tracrRNA anti-repeat sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more.

In various embodiments, the entire tracrRNA can comprise from about 60 nucleotides to more than about 140 nucleotides. For example, the tracrRNA can be about 60, about 65, about 70, about 75, about 80, about 85, about 90, about 95, about 100, about 105, about 110, about 115, about 120, about 125, about 130, about 135, about 140, or more nucleotides in length. In particular embodiments, the tracrRNA is about 80 to about 90 nucleotides in length, including about 80, about 81, about 82, about 83, about 84, about 85, about 86, about 87, about 88, about 89, and about 90 nucleotides in length. In certain embodiments, the tracrRNA is about 85 nucleotides in length.

In particular embodiments, the tracrRNA comprises the nucleotide sequence of SEQ ID NO: 3, 11, 18, 25, 32, 40, 48, 56, 63, 71, 77, 84, 91, 97, 105, 112, or 119, or an active variant or fragment thereof that when comprised within a guide RNA is capable of directing the sequence-specific binding of an associated RNA-guided nuclease provided herein to a target sequence of interest. In certain embodiments, an active tracrRNA sequence variant of a wild-type sequence comprises a nucleotide sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the nucleotide sequence set forth as SEQ ID NO: 3, 11, 18, 25, 32, 40, 48, 56, 63, 71, 77, 84, 91, 97, 105, 112, 119, 241, 274, or 286. In certain embodiments, an active tracrRNA sequence fragment of a wild-type sequence comprises at least 5, 10, 15, 20, 25, 30, 35, 40, 45, 50, 55, 60, 65, 70, 75, 80, or more contiguous nucleotides of the nucleotide sequence set forth as SEQ ID NO: 3, 11, 18, 25, 32, 40, 48, 56, 63, 71, 77, 84, 91, 97, 105, 112, 119, 241, 274, or 286.

Two polynucleotide sequences can be considered to be substantially complementary when the two sequences hybridize to each other under stringent conditions. Likewise, an RGN is considered to bind to a particular target sequence within a sequence-specific manner if the guide RNA bound to the RGN binds to the target sequence under stringent conditions. By “stringent conditions” or “stringent hybridization conditions” is intended conditions under which the two polynucleotide sequences will hybridize to each other to a detectably greater degree than to other sequences (e.g., at least 2-fold over background). Stringent conditions are sequence-dependent and will be different in different circumstances. Typically, stringent conditions will be those in which the salt concentration is less than about 1.5 M Na ion, typically about 0.01 to 1.0 M Na ion concentration (or other salts) at pH 7.0 to 8.3, and the temperature is at least about 30 °C for short sequences (e.g., 10 to 50 nucleotides) and at least about 60 °C for long sequences (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37 °C, and a wash in 1× to 2×SSC (20×SSC=3.0 M NaCl/0.3 M trisodium citrate) at 50 to 55 °C. Exemplary moderate stringency conditions include hybridization in 40 to 45% formamide, 1.0 M NaCl, 1% SDS at 37 °C, and a wash in 0.5× to 1×SSC at 55 to 60 °C. Exemplary high stringency conditions include hybridization in 50% formamide, 1 M NaCl, 1% SDS at 37 °C, and a wash in 0.1×SSC at 60 to 65 °C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The Tm is the temperature (under defined ionic strength and pH) at which 50% of a complementary target sequence hybridizes to a perfectly matched sequence. For DNA-DNA hybrids, the Tm can be approximated from the equation of Meinkoth and Wahl (1984) Anal. Biochem. 138:267-284: Tm=81.5 °C+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Generally, stringent conditions are selected to be about 5 °C lower than the thermal melting point (Tm) for the specific sequence and its complement at a defined ionic strength and pH. However, severely stringent conditions can utilize a hybridization and/or wash at 1, 2, 3, or 4 °C lower than the thermal melting point (Tm); moderately stringent conditions can utilize a hybridization and/or wash at 6, 7, 8, 9, or 10 °C lower than the thermal melting point (Tm); low stringency conditions can utilize a hybridization and/or wash at 11, 12, 13, 14, 15, or 20 °C lower than the thermal melting point (Tm). Using the equation, hybridization and wash compositions, and desired Tm, those of ordinary skill will understand that variations in the stringency of hybridization and/or wash solutions are inherently described. An extensive guide to the hybridization of nucleic acids is found in Tijssen (1993) Laboratory Techniques in Biochemistry and Molecular Biology-Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, New York).

The guide RNA can be a single guide RNA or a dual-guide RNA system. A single guide RNA comprises the crRNA and tracrRNA on a single molecule of RNA, whereas a dual-guide RNA system comprises a crRNA and a tracrRNA present on two distinct RNA molecules, hybridized to one another through at least a portion of the CRISPR repeat sequence of the crRNA and at least a portion of the tracrRNA, which may be fully or partially complementary to the CRISPR repeat sequence of the crRNA. In some of those embodiments wherein the guide RNA is a single guide RNA, the crRNA and tracrRNA are separated by a linker nucleotide sequence. In general, the linker nucleotide sequence is one that does not include complementary bases in order to avoid the formation of secondary structure within or comprising nucleotides of the linker nucleotide sequence. In some embodiments, the linker nucleotide sequence between the crRNA and tracrRNA is at least 3, at least 4, at least 5, at least 6, at least 7, at least 8, at least 9, at least 10, at least 11, at least 12, or more nucleotides in length. In particular embodiments, the linker nucleotide sequence of a single guide RNA is at least 4 nucleotides in length. In certain embodiments, the linker nucleotide sequence is the nucleotide sequence set forth as SEQ ID NO: 123.

The single guide RNA or dual-guide RNA can be synthesized chemically or via in vitro transcription. Assays for determining sequence-specific binding between a RGN and a guide RNA are known in the art and include, but are not limited to, in vitro binding assays between an expressed RGN and the guide RNA, which can be tagged with a detectable label (e.g., biotin) and used in a pull-down detection assay in which the guide RNA: RGN complex is captured via the detectable label (e.g., with streptavidin beads). A control guide RNA with an unrelated sequence or structure to the guide RNA can be used as a negative control for non-specific binding of the RGN to RNA. In certain embodiments, the guide RNA is SEQ ID NO: 4, 12, 19, 26, 33, 41, 49, 57, 64, 72, 78, 85, 92, 98, 106, 113, or 120, wherein the spacer sequence can be any sequence and is indicated as a poly-N sequence.

In certain embodiments, the guide RNA can be introduced into a target cell, organelle, or embryo as an RNA molecule. The guide RNA can be transcribed in vitro or chemically synthesized. In other embodiments, a nucleotide sequence encoding the guide RNA is introduced into the cell, organelle, or embryo. In some of these embodiments, the nucleotide sequence encoding the guide RNA is operably linked to a promoter (e.g., an RNA polymerase III promoter). The promoter can be a native promoter or heterologous to the guide RNA-encoding nucleotide sequence.

In various embodiments, the guide RNA can be introduced into a target cell, organelle, or embryo as a ribonucleoprotein complex, as described herein, wherein the guide RNA is bound to an RNA-guided nuclease polypeptide.

The guide RNA directs an associated RNA-guided nuclease to a particular target nucleotide sequence of interest through hybridization of the guide RNA to the target nucleotide sequence. A target nucleotide sequence can comprise DNA, RNA, or a combination of both and can be single-stranded or double-stranded. A target nucleotide sequence can be genomic DNA (i.e., chromosomal DNA), plasmid DNA, or an RNA molecule (e.g., messenger RNA, ribosomal RNA, transfer RNA, micro RNA, small interfering RNA). The target nucleotide sequence can be bound (and in some embodiments, cleaved) by an RNA-guided nuclease in vitro or in a cell. The chromosomal sequence targeted by the RGN can be a nuclear, plastid or mitochondrial chromosomal sequence. In some embodiments, the target nucleotide sequence is unique in the target genome.

The target nucleotide sequence is adjacent to a protospacer adjacent motif (PAM). A protospacer adjacent motif is generally within about 1 to about 10 nucleotides from the target nucleotide sequence, including about 1, about 2, about 3, about 4, about 5, about 6, about 7, about 8, about 9, or about 10 nucleotides from the target nucleotide sequence. The PAM can be 5′ or 3′ of the target sequence. In some embodiments, the PAM is 3′ of the target sequence for the presently disclosed RGNs. Generally, the PAM is a consensus sequence of about 3-4 nucleotides, but in particular embodiments, can be 2, 3, 4, 5, 6, 7, 8, 9, or more nucleotides in length. In various embodiments, the PAM sequence recognized by the presently disclosed RGNs comprises the consensus sequence set forth as SEQ ID NO: 7, 15, 22, 29, 36, 44, 52, 60, 67, 81, 88, 101, 109, or 116.

In particular embodiments, an RNA-guided nuclease having SEQ ID NO: 1, 9, 16, 23, 30, 38, 46, 54, 61, 69, 75, 82, 89, 95, 103, 110, or 117 or an active variant or fragment thereof binds respectively a target nucleotide sequence adjacent to a PAM sequence set forth as SEQ ID NO: 7, 15, 22, 29, 36, 44, 52, 60, 67, 81, 88, 101, 109, or 116. In some embodiments, an RNA-guided nuclease having SEQ ID NO: 54 or 137 or an active variant or fragment thereof binds a target nucleotide sequence adjacent to a PAM sequence set forth as SEQ ID NO: 147. In some of these embodiments, the RGN binds to a guide sequence comprising a CRISPR repeat sequence set forth in SEQ ID NO: 2, 10, 17, 24, 31, 39, 47, 55, 62, 70, 76, 83, 90, 96, 104, 111, or 118, respectively, or an active variant or fragment thereof, and a tracrRNA sequence set forth in SEQ ID NO: 3, 11, 18, 25, 32, 40, 48, 56, 63, 71, 77, 84, 91, 97, 105, 112, or 119, respectively, or an active variant or fragment thereof. The RGN systems are described further in Example 1 and Tables 1 and 2 of the present specification.

It is well-known in the art that PAM sequence specificity for a given nuclease enzyme is affected by enzyme concentration (see, e.g., Karvelis et al. (2015) Genome Biol 16:253), which may be modified by altering the promoter used to express the RGN, or the amount of ribonucleoprotein complex delivered to the cell, organelle, or embryo.

Upon recognizing its corresponding PAM sequence, the RGN can cleave the target nucleotide sequence at a specific cleavage site. As used herein, a cleavage site is made up of the two particular nucleotides within a target nucleotide sequence between which the nucleotide sequence is cleaved by an RGN. The cleavage site can comprise the 1^{st} and 2^{nd}, 2^{nd} and 3^{rd}, 3^{rd} and 4^{th}, 4^{th} and 5^{th}, 5^{th} and 6^{th}, 7^{th} and 8^{th}, or 8^{th} and 9^{th} nucleotides from the PAM in either the 5′ or 3′ direction. In some embodiments, the cleavage site may be over 10, 11, 12, 13, 14, 15, 16, 17, 18, 19, or 20 nucleotides from the PAM in either the 5′ or 3′ direction. In some embodiments, the cleavage site is 4 nucleotides away from the PAM. In other embodiments, the cleavage site is at least 15 nucleotides away from the PAM. As RGNs can cleave a target nucleotide sequence resulting in staggered ends, in some embodiments, the cleavage site is defined based on the distance of the two nucleotides from the PAM on the positive (+) strand of the polynucleotide and the distance of the two nucleotides from the PAM on the negative (−) strand of the polynucleotide.

The PAM is regarded as a hallmark of the RNA-guided nucleases of Type II CRISPR systems (Szczelkun et al., PNAS, 111:9798-9803, 2014; Sternberg et al., Nature 507:62-67, 2014). Interestingly, although APG06646.1 and APG04293.1 function as RNA-guided nucleases and possess many of the same domains as Type II CRISPR Cas9 nucleases, they each lack the typical PAM-Interacting domain (PID; Interpro: IPR032237; Pfam: PF16595). Accordingly, APG06646.1 and APG04293.1 also do not possess the typical PAM requirement, which is a motif of 2-5 nucleotides as described above. Instead, these proteins have unique DNA recognition domains at their C-termini (residues 821-1092 of APG06646.1 (full length sequence is SEQ ID NO: 117); residues 1064-1401 of APG04293.1 (full-length sequence is SEQ ID NO: 103). These unique DNA recognition domains enable the nucleases to cleave at a genomic target site based on a single-nucleotide motif in the vicinity of the genomic target sequence (SEQ ID NO: 109; see Table 2).

APG04293.1 also possesses a unique signature domain of 133 amino acid residues proximal to its N-terminus (residues 144-276). The function of this domain is not known in either Type II CRISPR Cas9 nucleases or generally in the art.

### III. Nucleotides Encoding RNA-Guided Nucleases, CRISPR RNA, and/or tracrRNA

The present disclosure provides polynucleotides comprising the presently disclosed CRISPR RNAs, tracrRNAs, and/or sgRNAs and polynucleotides comprising a nucleotide sequence encoding the presently disclosed RNA-guided nucleases, CRISPR RNAs, tracrRNAs, and/or sgRNAs. Presently disclosed polynucleotides include those comprising or encoding a CRISPR repeat sequence comprising the nucleotide sequence of SEQ ID NO: 2, 10, 17, 24, 31, 39, 47, 55, 62, 70, 76, 83, 90, 96, 104, 111, 118, 240, 273, or 287, or an active variant or fragment thereof that when comprised within a guide RNA is capable of directing the sequence-specific binding of an associated RNA-guided nuclease to a target sequence of interest. Also disclosed are polynucleotides comprising or encoding a tracrRNA comprising the nucleotide sequence of SEQ ID NO: 3, 11, 18, 25, 32, 40, 48, 56, 63, 71, 77, 84, 91, 97, 105, 112, 119, 241, 274, or 286, or an active variant or fragment thereof that when comprised within a guide RNA is capable of directing the sequence-specific binding of an associated RNA-guided nuclease to a target sequence of interest. Polynucleotides are also provided that encode an RNA-guided nuclease comprising the amino acid sequence set forth as SEQ ID NO: 1, 9, 16, 23, 30, 38, 46, 54, 61, 69, 75, 82, 89, 95, 103, 110, 117, 137, or 235, and active fragments or variants thereof that retain the ability to bind to a target nucleotide sequence in an RNA-guided sequence-specific manner.

The use of the term “polynucleotide” is not intended to limit the present disclosure to polynucleotides comprising DNA. Those of ordinary skill in the art will recognize that polynucleotides can comprise ribonucleotides (RNA) and combinations of ribonucleotides and deoxyribonucleotides. Such deoxyribonucleotides and ribonucleotides include both naturally occurring molecules and synthetic analogues. These include peptide nucleic acids (PNAs), PNA-DNA chimers, locked nucleic acids (LNAs), and phosphothiorate linked sequences. The polynucleotides disclosed herein also encompass all forms of sequences including, but not limited to, single-stranded forms, double-stranded forms, DNA-RNA hybrids, triplex structures, stem-and-loop structures, and the like.

The nucleic acid molecules encoding RGNs can be codon optimized for expression in an organism of interest. A “codon-optimized” coding sequence is a polynucleotide coding sequence having its frequency of codon usage designed to mimic the frequency of preferred codon usage or transcription conditions of a particular host cell. Expression in the particular host cell or organism is enhanced as a result of the alteration of one or more codons at the nucleic acid level such that the translated amino acid sequence is not changed. Nucleic acid molecules can be codon optimized, either wholly or in part. Codon tables and other references providing preference information for a wide range of organisms are available in the art (see, e.g., Campbell and Gowri (1990) Plant Physiol. 92:1-11 for a discussion of plant-preferred codon usage). Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Polynucleotides encoding the RGNs, crRNAs, tracrRNAs, and/or sgRNAs provided herein can be provided in expression cassettes for in vitro expression or expression in a cell, organelle, embryo, or organism of interest. The cassette will include 5′ and 3′ regulatory sequences operably linked to a polynucleotide encoding an RGN, crRNA, tracrRNAs, and/or sgRNAs provided herein that allows for expression of the polynucleotide. The cassette may additionally contain at least one additional gene or genetic element to be cotransformed into the organism. Where additional genes or elements are included, the components are operably linked. The term “operably linked” is intended to mean a functional linkage between two or more elements. For example, an operable linkage between a promoter and a coding region of interest (e.g., region coding for an RGN, crRNA, tracrRNAs, and/or sgRNAs) is a functional link that allows for expression of the coding region of interest. Operably linked elements may be contiguous or non-contiguous. When used to refer to the joining of two protein coding regions, by operably linked is intended that the coding regions are in the same reading frame. Alternatively, the additional gene(s) or element(s) can be provided on multiple expression cassettes. For example, the nucleotide sequence encoding a presently disclosed RGN can be present on one expression cassette, whereas the nucleotide sequence encoding a crRNA, tracrRNA, or complete guide RNA can be on a separate expression cassette. Such an expression cassette is provided with a plurality of restriction sites and/or recombination sites for insertion of the polynucleotides to be under the transcriptional regulation of the regulatory regions. The expression cassette may additionally contain a selectable marker gene.

The expression cassette will include in the 5′-3′ direction of transcription, a transcriptional (and, in some embodiments, translational) initiation region (i.e., a promoter), an RGN-, crRNA-, tracrRNA- and/or sgRNA-encoding polynucleotide of the invention, and a transcriptional (and in some embodiments, translational) termination region (i.e., termination region) functional in the organism of interest. The promoters of the invention are capable of directing or driving expression of a coding sequence in a host cell. The regulatory regions (e.g., promoters, transcriptional regulatory regions, and translational termination regions) may be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention. As used herein, a chimeric gene comprises a coding sequence operably linked to a transcription initiation region that is heterologous to the coding sequence.

Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Additional regulatory signals include, but are not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, inducible, growth stage-specific, cell type-specific, tissue-preferred, tissue-specific, or other promoters for expression in the organism of interest. See, for example, promoters set forth in WO 99/43838 and in U.S. Pat. Nos. 8,575,425; 7,790,846; 8,147,856; 8,586832; 7,772,369; 7,534,939; 6,072,050; 5,659,026; 5,608,149; 5,608,144; 5,604,121; 5,569,597; 5,466,785; 5,399,680; 5,268,463; 5,608,142; and 6,177,611; herein incorporated by reference.

For expression in plants, constitutive promoters also include CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); and MAS (Velten et al. (1984) EMBO J. 3:2723-2730).

Examples of inducible promoters are the Adh1 promoter which is inducible by hypoxia or cold stress, the Hsp70 promoter which is inducible by heat stress, the PPDK promoter and the pepcarboxylase promoter which are both inducible by light. Also useful are promoters which are chemically inducible, such as the In2-2 promoter which is safener induced (U.S. Pat. No. 5,364,780), the Axigl promoter which is auxin induced and tapetum specific but also active in callus (PCT US01/22169), the steroid-responsive promoters (see, for example, the ERE promoter which is estrogen induced, and the glucocorticoid-inducible promoter in Schena et al. (1991) Proc. Natl. Acad. Sci. USA 88:10421-10425 and McNellis et al. (1998) Plant J. 14 (2): 247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-specific or tissue-preferred promoters can be utilized to target expression of an expression construct within a particular tissue. In certain embodiments, the tissue-specific or tissue-preferred promoters are active in plant tissue. Examples of promoters under developmental control in plants include promoters that initiate transcription preferentially in certain tissues, such as leaves, roots, fruit, seeds, or flowers. A “tissue specific” promoter is a promoter that initiates transcription only in certain tissues. Unlike constitutive expression of genes, tissue-specific expression is the result of several interacting levels of gene regulation. As such, promoters from homologous or closely related plant species can be preferable to use to achieve efficient and reliable expression of transgenes in particular tissues. In some embodiments, the expression comprises a tissue-preferred promoter. A “tissue preferred” promoter is a promoter that initiates transcription preferentially, but not necessarily entirely or solely in certain tissues.

In some embodiments, the nucleic acid molecules encoding a RGN, crRNA, and/or tracrRNA comprise a cell type-specific promoter. A “cell type specific” promoter is a promoter that primarily drives expression in certain cell types in one or more organs. Some examples of plant cells in which cell type specific promoters functional in plants may be primarily active include, for example, BETL cells, vascular cells in roots, leaves, stalk cells, and stem cells. The nucleic acid molecules can also include cell type preferred promoters. A “cell type preferred” promoter is a promoter that primarily drives expression mostly, but not necessarily entirely or solely in certain cell types in one or more organs. Some examples of plant cells in which cell type preferred promoters functional in plants may be preferentially active include, for example, BETL cells, vascular cells in roots, leaves, stalk cells, and stem cells.

The nucleic acid sequences encoding the RGNs, crRNAs, tracrRNAs, and/or sgRNAs can be operably linked to a promoter sequence that is recognized by a phage RNA polymerase for example, for in vitro mRNA synthesis. In such embodiments, the in vitro-transcribed RNA can be purified for use in the methods described herein. For example, the promoter sequence can be a T7, T3, or SP6 promoter sequence or a variation of a T7, T3, or SP6 promoter sequence. In such embodiments, the expressed protein and/or RNAs can be purified for use in the methods of genome modification described herein.

In certain embodiments, the polynucleotide encoding the RGN, crRNA, tracrRNA, and/or sgRNA also can be linked to a polyadenylation signal (e.g., SV40 polyA signal and other signals functional in plants) and/or at least one transcriptional termination sequence. Additionally, the sequence encoding the RGN also can be linked to sequence(s) encoding at least one nuclear localization signal, at least one cell-penetrating domain, and/or at least one signal peptide capable of trafficking proteins to particular subcellular locations, as described elsewhere herein.

The polynucleotide encoding the RGN, crRNA, tracrRNA, and/or sgRNA can be present in a vector or multiple vectors. A “vector” refers to a polynucleotide composition for transferring, delivering, or introducing a nucleic acid into a host cell. Suitable vectors include plasmid vectors, phagemids, cosmids, artificial/mini-chromosomes, transposons, and viral vectors (e.g., lentiviral vectors, adeno-associated viral vectors, baculoviral vector). The vector can comprise additional expression control sequences (e.g., enhancer sequences, Kozak sequences, polyadenylation sequences, transcriptional termination sequences), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001.

The vector can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D).

In some embodiments, the expression cassette or vector comprising the sequence encoding the RGN polypeptide can further comprise a sequence encoding a crRNA and/or a tracrRNA, or the crRNA and tracrRNA combined to create a guide RNA. The sequence(s) encoding the crRNA and/or tracrRNA can be operably linked to at least one transcriptional control sequence for expression of the crRNA and/or tracrRNA in the organism or host cell of interest. For example, the polynucleotide encoding the crRNA and/or tracrRNA can be operably linked to a promoter sequence that is recognized by RNA polymerase III (Pol III). Examples of suitable Pol III promoters include, but are not limited to, mammalian U6, U3, H1, and 7SL RNA promoters and rice U6 and U3 promoters.

As indicated, expression constructs comprising nucleotide sequences encoding the RGNs, crRNA, tracrRNA, and/or sgRNA can be used to transform organisms of interest. Methods for transformation involve introducing a nucleotide construct into an organism of interest. By “introducing” is intended to introduce the nucleotide construct to the host cell in such a manner that the construct gains access to the interior of the host cell. The methods of the invention do not require a particular method for introducing a nucleotide construct to a host organism, only that the nucleotide construct gains access to the interior of at least one cell of the host organism. The host cell can be a eukaryotic or prokaryotic cell. In particular embodiments, the eukaryotic host cell is a plant cell, a mammalian cell, or an insect cell. Methods for introducing nucleotide constructs into plants and other host cells are known in the art including, but not limited to, stable transformation methods, transient transformation methods, and virus-mediated methods.

The methods result in a transformed organism, such as a plant, including whole plants, as well as plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells, pollen).

“Transgenic organisms” or “transformed organisms” or “stably transformed” organisms or cells or tissues refers to organisms that have incorporated or integrated a polynucleotide encoding a RGN, crRNA, and/or tracrRNA of the invention. It is recognized that other exogenous or endogenous nucleic acid sequences or DNA fragments may also be incorporated into the host cell. Agrobacterium- and biolistic-mediated transformation remain the two predominantly employed approaches for transformation of plant cells. However, transformation of a host cell may be performed by infection, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAE dextran procedure, and viral mediated, liposome mediated and the like. Viral-mediated introduction of a polynucleotide encoding an RGN, crRNA, and/or tracrRNA includes retroviral, lentiviral, adenoviral, and adeno-associated viral mediated introduction and expression, as well as the use of Caulimoviruses, Geminiviruses, and RNA plant viruses.

Transformation protocols as well as protocols for introducing polypeptides or polynucleotide sequences into plants may vary depending on the type of host cell (e.g., monocot or dicot plant cell) targeted for transformation. Methods for transformation are known in the art and include those set forth in U.S. Pat. Nos. 8,575,425; 7,692,068; 8,802,934; 7,541,517; each of which is herein incorporated by reference. See, also, Rakoczy-Trojanowska, M. (2002) Cell Mol Biol Lett. 7:849-858; Jones et al. (2005) Plant Methods 1:5; Rivera et al. (2012) Physics of Life Reviews 9:308-345; Bartlett et al. (2008) Plant Methods 4:1-12; Bates, G. W. (1999) Methods in Molecular Biology 111:359-366; Binns and Thomashow (1988) Annual Reviews in Microbiology 42:575-606; Christou, P. (1992) The Plant Journal 2:275-281; Christou, P. (1995) Euphytica 85:13-27; Tzfira et al. (2004) TRENDS in Genetics 20:375-383; Yao et al. (2006) Journal of Experimental Botany 57:3737-3746; Zupan and Zambryski (1995) Plant Physiology 107:1041-1047; Jones et al. (2005) Plant Methods 1:5;

Transformation may result in stable or transient incorporation of the nucleic acid into the cell. “Stable transformation” is intended to mean that the nucleotide construct introduced into a host cell integrates into the genome of the host cell and is capable of being inherited by the progeny thereof. “Transient transformation” is intended to mean that a polynucleotide is introduced into the host cell and does not integrate into the genome of the host cell.

Methods for transformation of chloroplasts are known in the art. See, for example, Svab et al. (1990) Proc. Nail. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

The cells that have been transformed may be grown into a transgenic organism, such as a plant, in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

Alternatively, cells that have been transformed may be introduced into an organism. These cells could have originated from the organism, wherein the cells are transformed in an ex vivo approach.

The sequences provided herein may be used for transformation of any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers.

Vegetables include, but are not limited to, tomatoes, lettuce, green beans, lima beans, peas, and members of the genus Curcumins such as cucumber, cantaloupe, and musk melon. Ornamentals include, but are not limited to, azalea, hydrangea, hibiscus, roses, tulips, daffodils, petunias, carnation, poinsettia, and chrysanthemum. Preferably, plants of the present invention are crop plants (for example, maize, sorghum, wheat, sunflower, tomato, crucifers, peppers, potato, cotton, rice, soybean, sugarbeet, sugarcane, tobacco, barley, oilseed rape, etc.).

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides. Further provided is a processed plant product or byproduct that retains the sequences disclosed herein, including for example, soymeal.

The polynucleotides encoding the RGNs, crRNAs, and/or tracrRNAs can also be used to transform any prokaryotic species, including but not limited to, archaea and bacteria (e.g., Bacillus sp., Klebsiella sp. Streptomyces sp., Rhizobium sp., Escherichia sp., Pseudomonas sp., Salmonella sp., Shigella sp., Vibrio sp., Yersinia sp., Mycoplasma sp., Agrobacterium, Lactobacillus sp.).

The polynucleotides encoding the RGNs, crRNAs, and/or tracrRNAs can be used to transform any eukaryotic species, including but not limited to animals (e.g., mammals, insects, fish, birds, and reptiles), fungi, amoeba, algae, and yeast.

Conventional viral and non-viral based gene transfer methods can be used to introduce nucleic acids in mammalian cells or target tissues. Such methods can be used to administer nucleic acids encoding components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6 (10): 1149-1154 (1988); Vigne, Restorative Neurology and Neuroscience 8:35-36 (1995); Kremer & Perricaudet, British Medical Bulletin 51 (1): 31-44 (1995); Haddada et al., in Current Topics in Microbiology and Immunology, Doerfler and Bohm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration). The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids takes advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Viral. 66:2731-2739 (1992); Johann et al., J. Viral. 66:1635-1640 (1992); Sommnerfelt et al., Viral. 176:58-59 (1990); Wilson et al., J. Viral. 63:2374-2378 (1989); Miller et al., 1. Viral. 65:2220-2224 (1991); PCT/US94/05700).

In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Katin, Human Gene Therapy 5:793-801 (1994); Muzyczka, 1. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., 1. Viral. 63:03822-3828 (1989). Packaging cells are typically used to form virus particles that are capable of infecting a host cell. Such cells include 293 cells, which package adenovirus, and ψJ2 cells or PA317 cells, which package retrovirus.

Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences.

The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLaS3, Huhl, Huh4, Huh7, HUVEC, HASMC, HEKn, HEKa, MiaPaCell, Panel, PC-3, TFl, CTLL-2, CIR, Rat6, CVI, RPTE, AIO, T24, 182, A375, ARH-77, Calul, SW480, SW620, SKOV3, SK-UT, CaCo2, P388DI, SEM-K2, WEHI-231, HB56, TIB55, lurkat, 145.01, LRMB, Bcl-1, BC-3, IC21, DLD2, Raw264.7, NRK, NRK-52E, MRC5, MEF, Hep G2, HeLa B, HeLa T4. COS, COS-1, COS-6, COS-M6A, BS-C-1 monkey kidney epithelial, BALB/3T3 mouse embryo fibroblast, 3T3 Swiss, 3T3-LI, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-I cells, BEAS-2B, bEnd.3, BHK-21, BR 293, BxPC3, C3H-10T1/2, C6/36, Cal-27, CHO, CHO-7, CHO—IR, CHO-KI, CHO-K2, CHO-T, CHO Dhfr−/−, COR-L23, COR-L23/CPR, COR-L235010, CORL23/R23, COS-7, COV-434, CML TI, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, lurkat, IY cells, K562 cells, Ku812, KCL22, KGI, KYOI, LNCap, Ma-Mel 1-48, MC-38, MCF-7, MCF-10A, MDA-MB-231, MDA-MB-468, MDA-MB-435, MDCKII, MDCKII, MOR/0.2R, MONO-MAC 6, MTD-IA, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THPI cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

In some embodiments, one or more vectors described herein are used to produce a non-human transgenic animal or transgenic plant. In some embodiments, the transgenic animal is a mammal, such as a mouse, rat, or rabbit.

### IV. Variants and Fragments of Polypeptides and Polynucleotides

The present disclosure provides active variants and fragments of a naturally-occurring (i.e., wild-type) RNA-guided nuclease, the amino acid sequence of which is set forth as SEQ ID NO: 1, 9, 16, 23, 30, 38, 46, 54, 61, 69, 75, 82, 89, 95, 103, 110, 117, 137, or 235, as well as active variants and fragments of naturally-occurring CRISPR repeats, such as the sequence set forth as SEQ ID NO: 2, 10, 17, 24, 31, 39, 47, 55, 62, 70, 76, 83, 90, 96, 104, 111, 118, 240, 273, or 287, and active variant and fragments of naturally-occurring tracrRNAs, such as the sequence set forth as SEQ ID NO: 3, 11, 18, 25, 32, 40, 48, 56, 63, 71, 77, 84, 91, 97, 105, 112, 119, 241, 274, or 286, and polynucleotides encoding the same.

While the activity of a variant or fragment may be altered compared to the polynucleotide or polypeptide of interest, the variant and fragment should retain the functionality of the polynucleotide or polypeptide of interest. For example, a variant or fragment may have increased activity, decreased activity, different spectrum of activity or any other alteration in activity when compared to the polynucleotide or polypeptide of interest.

Fragments and variants of naturally-occurring RGN polypeptides, such as those disclosed herein, will retain sequence-specific, RNA-guided DNA-binding activity. In particular embodiments, fragments and variants of naturally-occurring RGN polypeptides, such as those disclosed herein, will retain nuclease activity (single-stranded or double-stranded).

Fragments and variants of naturally-occurring CRISPR repeats, such as those disclosed herein, will retain the ability, when part of a guide RNA (comprising a tracrRNA), to bind to and guide an RNA-guided nuclease (complexed with the guide RNA) to a target nucleotide sequence in a sequence-specific manner.

Fragments and variants of naturally-occurring tracrRNAs, such as those disclosed herein, will retain the ability, when part of a guide RNA (comprising a CRISPR RNA), to guide an RNA-guided nuclease (complexed with the guide RNA) to a target nucleotide sequence in a sequence-specific manner.

The term “fragment” refers to a portion of a polynucleotide or polypeptide sequence of the invention. “Fragments” or “biologically active portions” include polynucleotides comprising a sufficient number of contiguous nucleotides to retain the biological activity (i.e., binding to and directing an RGN in a sequence-specific manner to a target nucleotide sequence when comprised within a guideRNA). “Fragments” or “biologically active portions” include polypeptides comprising a sufficient number of contiguous amino acid residues to retain the biological activity (i.e., binding to a target nucleotide sequence in a sequence-specific manner when complexed with a guide RNA). Fragments of the RGN proteins include those that are shorter than the full-length sequences due to the use of an alternate downstream start site. A biologically active portion of an RGN protein can be a polypeptide that comprises, for example, 10, 25, 50, 100, 150, 200, 250, 300, 350, 400, 450, 500, 550, 600, 650, 700, 750, 800, 850, 900, 950, 1000, 1050 or more contiguous amino acid residues of SEQ ID NO: 1, 9, 16, 23, 30, 38, 46, 54, 61, 69, 75, 82, 89, 95, 103, 110, 117, 137, or 235. Such biologically active portions can be prepared by recombinant techniques and evaluated for sequence-specific, RNA-guided DNA-binding activity. A biologically active fragment of a CRISPR repeat sequence can comprise at least 8 contiguous amino acids of SEQ ID NO: 2, 10, 17, 24, 31, 39, 47, 55, 62, 70, 76, 83, 90, 96, 104, 111, 118, 240, 273, or 287. A biologically active portion of a CRISPR repeat sequence can be a polynucleotide that comprises, for example, 8, 9, 10, 11, 12, 13, 14, 15, 16, 17, 18, 19, or 20 contiguous nucleotides of SEQ ID NO: 2, 10, 17, 24, 31, 39, 47, 55, 62, 70, 76, 83, 90, 96, 104, 111, 118, 240, 273, or 287. A biologically active portion of a tracrRNA can be a polynucleotide that comprises, for example, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80 or more contiguous nucleotides of SEQ ID NO: 3, 11, 18, 25, 32, 40, 48, 56, 63, 71, 77, 84, 91, 97, 105, 112, 119, 241, 274, or 286.

In general, “variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” or “wild type” polynucleotide or polypeptide comprises a naturally occurring nucleotide sequence or amino acid sequence, respectively. For polynucleotides, conservative variants include those sequences that, because of the degeneracy of the genetic code, encode the native amino acid sequence of the gene of interest. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode the polypeptide or the polynucleotide of interest. Generally, variants of a particular polynucleotide disclosed herein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide disclosed herein (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides disclosed herein is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

In particular embodiments, the presently disclosed polynucleotides encode an RNA-guided nuclease polypeptide comprising an amino acid sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to an amino acid sequence of SEQ ID NO: 1, 9, 16, 23, 30, 38, 46, 54, 61, 69, 75, 82, 89, 95, 103, 110, 117, 137, or 235.

A biologically active variant of an RGN polypeptide of the invention may differ by as few as about 1-15 amino acid residues, as few as about 1-10, such as about 6-10, as few as 5, as few as 4, as few as 3, as few as 2, or as few as 1 amino acid residue. In specific embodiments, the polypeptides can comprise an N-terminal or a C-terminal truncation, which can comprise at least a deletion of 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1050 amino acids or more from either the N or C terminus of the polypeptide.

In certain embodiments, the presently disclosed polynucleotides comprise or encode a CRISPR repeat comprising a nucleotide sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to the nucleotide sequence set forth as SEQ ID NO: 2, 10, 17, 24, 31, 39, 47, 55, 62, 70, 76, 83, 90, 96, 104, 111, 118, 240, 273, or 287.

The presently disclosed polynucleotides can comprise or encode a tracrRNA comprising a nucleotide sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to the nucleotide sequence set forth as SEQ ID NO: 3, 11, 18, 25, 32, 40, 48, 56, 63, 71, 77, 84, 91, 97, 105, 112, 119, 241, 274, or 286.

Biologically active variants of a CRISPR repeat or tracrRNA of the invention may differ by as few as about 1-15 nucleotides, as few as about 1-10, such as about 6-10, as few as 5, as few as 4, as few as 3, as few as 2, or as few as 1 nucleotide. In specific embodiments, the polynucleotides can comprise a 5′ or 3′ truncation, which can comprise at least a deletion of 10, 15, 20, 25, 30, 35, 40, 45, 50, 55, 60, 65, 70, 75, 80 nucleotides or more from either the 5′ or 3′ end of the polynucleotide.

It is recognized that modifications may be made to the RGN polypeptides, CRISPR repeats, and tracrRNAs provided herein creating variant proteins and polynucleotides. Changes designed by man may be introduced through the application of site-directed mutagenesis techniques. Alternatively, native, as yet-unknown or as yet unidentified polynucleotides and/or polypeptides structurally and/or functionally-related to the sequences disclosed herein may also be identified that fall within the scope of the present invention. Conservative amino acid substitutions may be made in nonconserved regions that do not alter the function of the RGN proteins. Alternatively, modifications may be made that improve the activity of the RGN.

Variant polynucleotides and proteins also encompass sequences and proteins derived from a mutagenic and recombinogenic procedure such as DNA shuffling. With such a procedure, one or more different RGN proteins disclosed herein (e.g., SEQ ID NO: 1, 9, 16, 23, 30, 38, 46, 54, 61, 69, 75, 82, 89, 95, 103, 110, 117, 137, or 235) is manipulated to create a new RGN protein possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the RGN sequences provided herein and other known RGN genes to obtain a new gene coding for a protein with an improved property of interest, such as an increased Kₘ in the case of an enzyme. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458. A “shuffled” nucleic acid is a nucleic acid produced by a shuffling procedure such as any shuffling procedure set forth herein. Shuffled nucleic acids are produced by recombining (physically or virtually) two or more nucleic acids (or character strings), for example in an artificial, and optionally recursive, fashion. Generally, one or more screening steps are used in shuffling processes to identify nucleic acids of interest; this screening step can be performed before or after any recombination step. In some (but not all) shuffling embodiments, it is desirable to perform multiple rounds of recombination prior to selection to increase the diversity of the pool to be screened. The overall process of recombination and selection are optionally repeated recursively. Depending on context, shuffling can refer to an overall process of recombination and selection, or, alternately, can simply refer to the recombinational portions of the overall process.

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. Amino acid substitution matrices and their use in quantifying the similarity between two sequences are well-known in the art and described, e.g., in Dayhoff et al. (1978) “A model of evolutionary change in proteins.” In “Atlas of Protein Sequence and Structure,” Vol. 5, Suppl. 3 (ed. M. O. Dayhoff), pp. 345-352. Natl. Biomed. Res. Found., Washington, D.C. and Henikoff et al. (1992) Proc. Natl. Acad. Sci. USA 89:10915-10919. The BLOSUM62 matrix is often used as a default scoring substitution matrix in sequence alignment protocols. The gap existence penalty is imposed for the introduction of a single amino acid gap in one of the aligned sequences, and the gap extension penalty is imposed for each additional empty amino acid position inserted into an already opened gap. The alignment is defined by the amino acids positions of each sequence at which the alignment begins and ends, and optionally by the insertion of a gap or multiple gaps in one or both sequences, so as to arrive at the highest possible score. While optimal alignment and scoring can be accomplished manually, the process is facilitated by the use of a computer-implemented alignment algorithm, e.g., gapped BLAST 2.0, described in Altschul et al. (1997) Nucleic Acids Res. 25:3389-3402, and made available to the public at the National Center for Biotechnology Information Website (www.ncbi.nlm.nih.gov). Optimal alignments, including multiple alignments, can be prepared using, e.g., PSI-BLAST, available through www.ncbi.nlm.nih.gov and described by Altschul et al. (1997) Nucleic Acids Res. 25:3389-3402.

With respect to an amino acid sequence that is optimally aligned with a reference sequence, an amino acid residue “corresponds to” the position in the reference sequence with which the residue is paired in the alignment. The “position” is denoted by a number that sequentially identifies each amino acid in the reference sequence based on its position relative to the N-terminus. Owing to deletions, insertion, truncations, fusions, etc., that must be taken into account when determining an optimal alignment, in general the amino acid residue number in a test sequence as determined by simply counting from the N-terminal will not necessarily be the same as the number of its corresponding position in the reference sequence. For example, in a case where there is a deletion in an aligned test sequence, there will be no amino acid that corresponds to a position in the reference sequence at the site of deletion. Where there is an insertion in an aligned reference sequence, that insertion will not correspond to any amino acid position in the reference sequence. In the case of truncations or fusions there can be stretches of amino acids in either the reference or aligned sequence that do not correspond to any amino acid in the corresponding sequence.

### V. Antibodies

Antibodies to the RGN polypeptides or ribonucleoproteins comprising the RGN polypeptides of the present invention, including those having the amino acid sequence set forth as SEQ ID NO: 1, 9, 16, 23, 30, 38, 46, 54, 61, 69, 75, 82, 89, 95, 103, 110, or 117 or active variants or fragments thereof, are also encompassed. Methods for producing antibodies are well known in the art (see, for example, Harlow and Lane (1988) Antibodies: A Laboratory Manual, Cold Spring Harbor Laboratory, Cold Spring Harbor, N.Y.; and U.S. Pat. No. 4,196,265). These antibodies can be used in kits for the detection and isolation of RGN polypeptides or ribonucleoproteins. Thus, this disclosure provides kits comprising antibodies that specifically bind to the polypeptides or ribonucleoproteins described herein, including, for example, polypeptides having the sequence of SEQ ID NO: 1, 9, 16, 23, 30, 38, 46, 54, 61, 69, 75, 82, 89, 95, 103, 110, or 117.

### VI. Systems and Ribonucleoprotein Complexes for Binding a Target Sequence of Interest and Methods of Making the Same

The present disclosure provides a system for binding a target sequence of interest, wherein the system comprises at least one guide RNA or a nucleotide sequence encoding the same, and at least one RNA-guided nuclease or a nucleotide sequence encoding the same. The guide RNA hybridizes to the target sequence of interest and also forms a complex with the RGN polypeptide, thereby directing the RGN polypeptide to bind to the target sequence. In some of these embodiments, the RGN comprises an amino acid sequence of SEQ ID NO: 1, 9, 16, 23, 30, 38, 46, 54, 61, 69, 75, 82, 89, 95, 103, 110, 117, 137, or 235, or an active variant or fragment thereof. In various embodiments, the guide RNA comprises a CRISPR repeat sequence comprising the nucleotide sequence of SEQ ID NO: 2, 10, 17, 24, 31, 39, 47, 55, 62, 70, 76, 83, 90, 96, 104, 111, 118, 240, 273, or 287, or an active variant or fragment thereof. In particular embodiments, the guide RNA comprises a tracrRNA comprising a nucleotide sequence of SEQ ID NO: 3, 11, 18, 25, 32, 40, 48, 56, 63, 71, 77, 84, 91, 97, 105, 112, 119, 241, 274, or 286, or an active variant or fragment thereof. The guide RNA of the system can be a single guide RNA or a dual-guide RNA. In particular embodiments, the system comprises a RNA-guided nuclease that is heterologous to the guideRNA, wherein the RGN and guideRNA are not naturally complexed in nature.

The system for binding a target sequence of interest provided herein can be a ribonucleoprotein complex, which is at least one molecule of an RNA bound to at least one protein. The ribonucleoprotein complexes provided herein comprise at least one guide RNA as the RNA component and an RNA-guided nuclease as the protein component. Such ribonucleoprotein complexes can be purified from a cell or organism that naturally expresses an RGN polypeptide and has been engineered to express a particular guide RNA that is specific for a target sequence of interest. Alternatively, the ribonucleoprotein complex can be purified from a cell or organism that has been transformed with polynucleotides that encode an RGN polypeptide and a guide RNA and cultured under conditions to allow for the expression of the RGN polypeptide and guide RNA. Thus, methods are provided for making an RGN polypeptide or an RGN ribonucleoprotein complex. Such methods comprise culturing a cell comprising a nucleotide sequence encoding an RGN polypeptide, and in some embodiments a nucleotide sequence encoding a guide RNA, under conditions in which the RGN polypeptide (and in some embodiments, the guide RNA) is expressed. The RGN polypeptide or RGN ribonucleoprotein can then be purified from a lysate of the cultured cells.

Methods for purifying an RGN polypeptide or RGN ribonucleoprotein complex from a lysate of a biological sample are known in the art (e.g., size exclusion and/or affinity chromatography, 2D-PAGE, HPLC, reversed-phase chromatography, immunoprecipitation). In particular methods, the RGN polypeptide is recombinantly produced and comprises a purification tag to aid in its purification, including but not limited to, glutathione-S-transferase (GST), chitin binding protein (CBP), maltose binding protein, thioredoxin (TRX), poly (NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, 10×His, biotin carboxyl carrier protein (BCCP), and calmodulin. Generally, the tagged RGN polypeptide or RGN ribonucleoprotein complex is purified using immobilized metal affinity chromatography. It will be appreciated that other similar methods known in the art may be used, including other forms of chromatography or for example immunoprecipitation, either alone or in combination.

An “isolated” or “purified” polypeptide, or biologically active portion thereof, is substantially or essentially free from components that normally accompany or interact with the polypeptide as found in its naturally occurring environment. Thus, an isolated or purified polypeptide is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

Particular methods provided herein for binding and/or cleaving a target sequence of interest involve the use of an in vitro assembled RGN ribonucleoprotein complex. In vitro assembly of an RGN ribonucleoprotein complex can be performed using any method known in the art in which an RGN polypeptide is contacted with a guide RNA under conditions to allow for binding of the RGN polypeptide to the guide RNA. As used herein, “contact”, contacting”, “contacted,” refer to placing the components of a desired reaction together under conditions suitable for carrying out the desired reaction. The RGN polypeptide can be purified from a biological sample, cell lysate, or culture medium, produced via in vitro translation, or chemically synthesized. The guide RNA can be purified from a biological sample, cell lysate, or culture medium, transcribed in vitro, or chemically synthesized. The RGN polypeptide and guide RNA can be brought into contact in solution (e.g., buffered saline solution) to allow for in vitro assembly of the RGN ribonucleoprotein complex.

### VII. Methods of Binding, Cleaving, or Modifying a Target Sequence

The present disclosure provides methods for binding, cleaving, and/or modifying a target nucleotide sequence of interest. The methods include delivering a system comprising at least one guide RNA or a polynucleotide encoding the same, and at least one RGN polypeptide or a polynucleotide encoding the same to the target sequence or a cell, organelle, or embryo comprising the target sequence. In some of these embodiments, the RGN comprises the amino acid sequence of SEQ ID NO: 1, 9, 16, 23, 30, 38, 46, 54, 61, 69, 75, 82, 89, 95, 103, 110, 117, 137, or 235, or an active variant or fragment thereof. In various embodiments, the guide RNA comprises a CRISPR repeat sequence comprising the nucleotide sequence of SEQ ID NO: 2, 10, 17, 24, 31, 39, 47, 55, 62, 70, 76, 83, 90, 96, 104, 111, 118, 240, 273, or 287, or an active variant or fragment thereof. In particular embodiments, the guide RNA comprises a tracrRNA comprising the nucleotide sequence of SEQ ID NO: 3, 11, 18, 25, 32, 40, 48, 56, 63, 71, 77, 84, 91, 97, 105, 112, 119, 241, 274, or 286, or an active variant or fragment thereof. The guide RNA of the system can be a single guide RNA or a dual-guide RNA. The RGN of the system may be nuclease dead RGN, have nickase activity, or may be a fusion polypeptide. In some embodiments, the fusion polypeptide comprises a base-editing polypeptide, for example a cytidine deaminase or an adenosine deaminase. In other embodiments, the RGN fusion protein comprises a reverse transcriptase. In other embodiments, the RGN fusion protein comprises a polypeptide that recruits members of a functional nucleic acid repair complex, such as a member of the nucleotide excision repair (NER) or transcription coupled-nucleotide excision repair (TC-NER) pathway (Wei et al., 2015, PNAS USA 112 (27): E3495-504; Troelstra et al., 1992, Cell 71:939-953; Marnef et al., 2017, J Mol Biol 429 (9): 1277-1288), as described in U.S. Provisional Application No. 62/966,203, which was filed on Jan. 27, 2020, and is incorporated by reference in its entirety. In some embodiments, the RGN fusion protein comprises CSB (van den Boom et al., 2004, J Cell Biol 166 (1): 27-36; van Gool et al., 1997, EMBO J 16 (19): 5955-65; an example of which is set forth as SEQ ID NO: 268), which is a member of the TC-NER (nucleotide excision repair) pathway and functions in the recruitment of other members. In further embodiments, the RGNABP fusion protein comprises an active domain of CSB, such as the acidic domain of CSB which comprises amino acid residues 356-394 of SEQ ID NO: 268 (Teng et al., 2018, Nat Commun 9 (1): 4115).

In particular embodiments, the RGN and/or guide RNA is heterologous to the cell, organelle, or embryo to which the RGN and/or guide RNA (or polynucleotide(s) encoding at least one of the RGN and guide RNA) are introduced.

In those embodiments wherein the method comprises delivering a polynucleotide encoding a guide RNA and/or an RGN polypeptide, the cell or embryo can then be cultured under conditions in which the guide RNA and/or RGN polypeptide are expressed. In various embodiments, the method comprises contacting a target sequence with an RGN ribonucleoprotein complex. The RGN ribonucleoprotein complex may comprise an RGN that is nuclease dead or has nickase activity. In some embodiments, the RGN of the ribonucleoprotein complex is a fusion polypeptide comprising a base-editing polypeptide. In certain embodiments, the method comprises introducing into a cell, organelle, or embryo comprising a target sequence an RGN ribonucleoprotein complex. The RGN ribonucleoprotein complex can be one that has been purified from a biological sample, recombinantly produced and subsequently purified, or in vitro-assembled as described herein. In those embodiments wherein the RGN ribonucleoprotein complex that is contacted with the target sequence or a cell organelle, or embryo has been assembled in vitro, the method can further comprise the in vitro assembly of the complex prior to contact with the target sequence, cell, organelle, or embryo.

A purified or in vitro assembled RGN ribonucleoprotein complex can be introduced into a cell, organelle, or embryo using any method known in the art, including, but not limited to electroporation. Alternatively, an RGN polypeptide and/or polynucleotide encoding or comprising the guide RNA can be introduced into a cell, organelle, or embryo using any method known in the art (e.g., electroporation).

Upon delivery to or contact with the target sequence or cell, organelle, or embryo comprising the target sequence, the guide RNA directs the RGN to bind to the target sequence in a sequence-specific manner. In those embodiments wherein the RGN has nuclease activity, the RGN polypeptide cleaves the target sequence of interest upon binding. The target sequence can subsequently be modified via endogenous repair mechanisms, such as non-homologous end joining, or homology-directed repair with a provided donor polynucleotide.

Methods to measure binding of an RGN polypeptide to a target sequence are known in the art and include chromatin immunoprecipitation assays, gel mobility shift assays, DNA pull-down assays, reporter assays, microplate capture and detection assays. Likewise, methods to measure cleavage or modification of a target sequence are known in the art and include in vitro or in vivo cleavage assays wherein cleavage is confirmed using PCR, sequencing, or gel electrophoresis, with or without the attachment of an appropriate label (e.g., radioisotope, fluorescent substance) to the target sequence to facilitate detection of degradation products. Alternatively, the nicking triggered exponential amplification reaction (NTEXPAR) assay can be used (see, e.g., Zhang et al. (2016) Chem. Sci. 7:4951-4957). In vivo cleavage can be evaluated using the Surveyor assay (Guschin et al. (2010) Methods Mol Biol 649:247-256).

In some embodiments, the methods involve the use of a single type of RGN complexed with more than one guide RNA. The more than one guide RNA can target different regions of a single gene or can target multiple genes.

In those embodiments wherein a donor polynucleotide is not provided, a double-stranded break introduced by an RGN polypeptide can be repaired by a non-homologous end-joining (NHEJ) repair process. Due to the error-prone nature of NHEJ, repair of the double-stranded break can result in a modification to the target sequence. As used herein, a “modification” in reference to a nucleic acid molecule refers to a change in the nucleotide sequence of the nucleic acid molecule, which can be a deletion, insertion, or substitution of one or more nucleotides, or a combination thereof. Modification of the target sequence can result in the expression of an altered protein product or inactivation of a coding sequence.

In those embodiments wherein a donor polynucleotide is present, the donor sequence in the donor polynucleotide can be integrated into or exchanged with the target nucleotide sequence during the course of repair of the introduced double-stranded break, resulting in the introduction of the exogenous donor sequence. A donor polynucleotide thus comprises a donor sequence that is desired to be introduced into a target sequence of interest. In some embodiments, the donor sequence alters the original target nucleotide sequence such that the newly integrated donor sequence will not be recognized and cleaved by the RGN. Integration of the donor sequence can be enhanced by the inclusion within the donor polynucleotide of flanking sequences that have substantial sequence identity with the sequences flanking the target nucleotide sequence, allowing for a homology-directed repair process. In those embodiments wherein the RGN polypeptide introduces double-stranded staggered breaks, the donor polynucleotide can comprise a donor sequence flanked by compatible overhangs, allowing for direct ligation of the donor sequence to the cleaved target nucleotide sequence comprising overhangs by a non-homologous repair process during repair of the double-stranded break.

In those embodiments wherein the method involves the use of an RGN that is a nickase (i.e., is only able to cleave a single strand of a double-stranded polynucleotide), the method can comprise introducing two RGN nickases that target identical or overlapping target sequences and cleave different strands of the polynucleotide. For example, an RGN nickase that only cleaves the positive (+) strand of a double-stranded polynucleotide can be introduced along with a second RGN nickase that only cleaves the negative (−) strand of a double-stranded polynucleotide.

In various embodiments, a method is provided for binding a target nucleotide sequence and detecting the target sequence, wherein the method comprises introducing into a cell, organelle, or embryo at least one guide RNA or a polynucleotide encoding the same, and at least one RGN polypeptide or a polynucleotide encoding the same, expressing the guide RNA and/or RGN polypeptide (if coding sequences are introduced), wherein the RGN polypeptide is a nuclease-dead RGN and further comprises a detectable label, and the method further comprises detecting the detectable label. The detectable label may be fused to the RGN as a fusion protein (e.g., fluorescent protein) or may be a small molecule conjugated to or incorporated within the RGN polypeptide that can be detected visually or by other means.

Also provided herein are methods for modulating the expression of a target sequence or a gene of interest under the regulation of a target sequence. The methods comprise introducing into a cell, organelle, or embryo at least one guide RNA or a polynucleotide encoding the same, and at least one RGN polypeptide or a polynucleotide encoding the same, expressing the guide RNA and/or RGN polypeptide (if coding sequences are introduced), wherein the RGN polypeptide is a nuclease-dead RGN. In some of these embodiments, the nuclease-dead RGN is a fusion protein comprising an expression modulator domain (i.e., epigenetic modification domain, transcriptional activation domain or a transcriptional repressor domain) as described herein.

The present disclosure also provides methods for binding and/or modifying a target nucleotide sequence of interest. The methods include delivering a system comprising at least one guide RNA or a polynucleotide encoding the same, and at least one fusion polypeptide comprises an RGN of the invention and a base-editing polypeptide, for example a cytidine deaminase or an adenosine deaminase, or a polynucleotide encoding the fusion polypeptide, to the target sequence or a cell, organelle, or embryo comprising the target sequence.

One of ordinary skill in the art will appreciate that any of the presently disclosed methods can be used to target a single target sequence or multiple target sequences. Thus, methods comprise the use of a single RGN polypeptide in combination with multiple, distinct guide RNAs, which can target multiple, distinct sequences within a single gene and/or multiple genes. Also encompassed herein are methods wherein multiple, distinct guide RNAs are introduced in combination with multiple, distinct RGN polypeptides. These guide RNAs and guide RNA/RGN polypeptide systems can target multiple, distinct sequences within a single gene and/or multiple genes.

In one aspect, the invention provides kits containing any one or more of the elements disclosed in the above methods and compositions. In some embodiments, the kit comprises a vector system and instructions for using the kit. In some embodiments, the vector system comprises (a) a first regulatory element operably linked to a DNA sequence encoding the crRNA sequence and one or more insertion sites for inserting a guide sequence upstream of the encoded crRNA sequence, wherein when expressed, the guide sequence directs sequence-specific binding of a CRISPR complex to a target sequence in a eukaryotic cell, wherein the CRISPR complex comprises a CRISPR enzyme complexed with the guide RNA polynucleotide; and/or (b) a second regulatory element operably linked to an enzyme coding sequence encoding said CRISPR enzyme comprising a nuclear localization sequence. Elements may be provided individually or in combinations, and may be provided in any suitable container, such as a vial, a bottle, or a tube.

In some embodiments, the kit includes instructions in one or more languages. In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10.

In some embodiments, the kit comprises one or more oligonucleotides corresponding to a guide sequence for insertion into a vector so as to operably link the guide sequence and a regulatory element. In some embodiments, the kit comprises a homologous recombination template polynucleotide. In one aspect, the invention provides methods for using one or more elements of a CRISPR system. The CRISPR complex of the invention provides an effective means for modifying a target polynucleotide. The CRISPR complex of the invention has a wide variety of utility including modifying (e.g., deleting, inserting, translocating, inactivating, activating, base editing) a target polynucleotide in a multiplicity of cell types. As such the CRISPR complex of the invention has a broad spectrum of applications in, e.g., gene therapy, drug screening, disease diagnosis, and prognosis. An exemplary CRISPR complex comprises a CRISPR enzyme complexed with a guide sequence hybridized to a target sequence within the target polynucleotide.

### VIII. Target Polynucleotides

In one aspect, the invention provides for methods of modifying a target polynucleotide in a eukaryotic cell, which may be in vivo, ex vivo or in vitro. In some embodiments, the method comprises sampling a cell or population of cells from a human or non-human animal or plant (including microalgae) and modifying the cell or cells. Culturing may occur at any stage ex vivo. The cell or cells may even be re-introduced into the non-human animal or plant (including micro-algae).

Using natural variability, plant breeders combine most useful genes for desirable qualities, such as yield, quality, uniformity, hardiness, and resistance against pests. These desirable qualities also include growth, day length preferences, temperature requirements, initiation date of floral or reproductive development, fatty acid content, insect resistance, disease resistance, nematode resistance, fungal resistance, herbicide resistance, tolerance to various environmental factors including drought, heat, wet, cold, wind, and adverse soil conditions including high salinity The sources of these useful genes include native or foreign varieties, heirloom varieties, wild plant relatives, and induced mutations, e.g., treating plant material with mutagenic agents. Using the present invention, plant breeders are provided with a new tool to induce mutations. Accordingly, one skilled in the art can analyze the genome for sources of useful genes, and in varieties having desired characteristics or traits employ the present invention to induce the rise of useful genes, with more precision than previous mutagenic agents and hence accelerate and improve plant breeding programs.

The target polynucleotide of an RGN system can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA). Without wishing to be bound by theory, it is believed that the target sequence should be associated with a PAM (protospacer adjacent motif); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence).

The target polynucleotide of a CRISPR complex may include a number of disease-associated genes and polynucleotides as well as signaling biochemical pathway-associated genes and polynucleotides. Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease (e.g., a causal mutation). The transcribed or translated products may be known or unknown, and further may be at a normal or abnormal level. Examples of disease-associated genes and polynucleotides are available from McKusick-Nathans Institute of Genetic Medicine, Johns Hopkins University (Baltimore, Md.) and National Center for Biotechnology Information, National Library of Medicine (Bethesda, Md.), available on the World Wide Web.

Although CRISPR systems are particularly useful for their relative ease in targeting to genomic sequences of interest, there still remains an issue of what the RGN can do to address a causal mutation. One approach is to produce a fusion protein between an RGN (preferably an inactive or nickase variant of the RGN) and a base-editing enzyme or the active domain of a base editing enzyme, such as a cytidine deaminase or an adenosine deaminase base editor (U.S. Pat. No. 9,840,699, herein incorporated by reference). In some embodiments, the methods comprise contacting a DNA molecule with (a) a fusion protein comprising an RGN of the invention and a base-editing polypeptide such as a deaminase; and (b) a gRNA targeting the fusion protein of (a) to a target nucleotide sequence of the DNA strand; wherein the DNA molecule is contacted with the fusion protein and the gRNA in an amount effective and under conditions suitable for the deamination of a nucleotide base. In some embodiments, the target DNA sequence comprises a sequence associated with a disease or disorder, and wherein the deamination of the nucleotide base results in a sequence that is not associated with a disease or disorder. In some embodiments, the target DNA sequence resides in an allele of a crop plant, wherein the particular allele of the trait of interest results in a plant of lesser agronomic value. The deamination of the nucleotide base results in an allele that improves the trait and increases the agronomic value of the plant.

In some embodiments, the DNA sequence comprises a T→C or A→G point mutation associated with a disease or disorder, and wherein the deamination of the mutant C or G base results in a sequence that is not associated with a disease or disorder. In some embodiments, the deamination corrects a point mutation in the sequence associated with the disease or disorder.

In some embodiments, the sequence associated with the disease or disorder encodes a protein, and wherein the deamination introduces a stop codon into the sequence associated with the disease or disorder, resulting in a truncation of the encoded protein. In some embodiments, the contacting is performed in vivo in a subject susceptible to having, having, or diagnosed with the disease or disorder. In some embodiments, the disease or disorder is a disease associated with a point mutation, or a single-base mutation, in the genome. In some embodiments, the disease is a genetic disease, a cancer, a metabolic disease, or a lysosomal storage disease.

### Modifying Causal Mutations Using Base-Editing

An example of a genetically inherited disease which could be corrected using an approach that relies on an RGN-base editor fusion protein of the invention is Hurler Syndrome. Hurler Syndrome, also known as MPS-1, is the result of a deficiency of α-L-iduronidase (IDUA) resulting in a lysosomal storage disease characterized at the molecular level by the accumulation of dermatan sulfate and heparan sulfate in lysosomes. This disease is generally an inherited genetic disorder caused by mutations in the IDUA gene encoding α-L-iduronidase. Common IDUA mutations are W402X and Q70X, both nonsense mutations resulting in premature termination of translation. Such mutations are well addressed by precise genome editing (PGE) approaches, since reversion of a single nucleotide, for example by a base-editing approach, would restore the wild-type coding sequence and result in protein expression controlled by the endogenous regulatory mechanisms of the genetic locus. Additionally, since heterozygotes are known to be asymptomatic, a PGE therapy that targets one of these mutations would be useful to a large proportion of patients with this disease, as only one of the mutated alleles needs to be corrected (Bunge et al. (1994) Hum. Mol. Genet. 3 (6): 861-866, herein incorporated by reference).

Current treatments for Hurler Syndrome include enzyme replacement therapy and bone marrow transplants (Vellodi et al. (1997) Arch. Dis. Child. 76 (2): 92-99; Peters et al. (1998) Blood 91 (7): 2601-2608, herein incorporated by reference). While enzyme replacement therapy has had a dramatic effect on the survival and quality of life of Hurler Syndrome patients, this approach requires costly and time-consuming weekly infusions. Additional approaches include the delivery of the IDUA gene on an expression vector or the insertion of the gene into a highly expressed locus such as that of serum albumin (U.S. Pat. No. 9,956,247, herein incorporated by reference). However, these approaches do not restore the original IDUA locus to the correct coding sequence. A genome-editing strategy would have a number of advantages, most notably that regulation of gene expression would be controlled by the natural mechanisms present in healthy individuals. Additionally, using base editing does not necessitate causing a double stranded DNA breaks, which could lead to large chromosomal rearrangements, cell death, or oncogenecity by the disruption of tumor suppression mechanisms. A general strategy may be directed toward using RGN-base editor fusion proteins of the invention to target and correct certain disease-causing mutations in the human genome. It will be appreciated that similar approaches to target diseases that can be corrected by base-editing may also be pursued. It will be further appreciated that similar approaches to target disease-causing mutations in other species, particularly common household pets or livestock, can also be deployed using the RGNs of the invention. Common household pets and livestock include dogs, cats, horses, pigs, cows, sheep, chickens, donkeys, snakes, ferrets, and fish including salmon and shrimp.

### Modifying Causal Mutations by Targeted Deletion

RGNs of the invention could also be useful in human therapeutic approaches where the causal mutation is more complicated. For example, some diseases such as Friedreich's Ataxia and Huntington's Disease are the result of a significant increase in repeats of a three nucleotide motif at a particular region of a gene, which affects the ability of the expressed protein to function or to be expressed. Friedreich's Ataxia (FRDA) is an autosomal recessive disease resulting in progressive degeneration of nervous tissue in the spinal cord. Reduced levels of the frataxin (FXN) protein in the mitochondria cause oxidative damages and iron deficiencies at the cellular level. The reduced FXN expression has been linked to a GAA triplet expansion within the intron 1 of the somatic and germline FXN gene. In FRDA patients, the GAA repeat frequently consists of more than 70, sometimes even more than 1000 (most commonly 600-900) triplets, whereas unaffected individuals have about 40 repeats or less (Pandolfo et al. (2012) Handbook of Clinical Neurology 103:275-294; Campuzano et al. (1996) Science 271:1423-1427; Pandolfo (2002) Adv. Exp. Med. Biol. 516:99-118; all herein incorporated by reference).

The expansion of the trinucleotide repeat sequence causing Friedreich's Ataxia (FRDA) occurs in a defined genetic locus within the FXN gene, referred to as the FRDA instability region. RNA guided nucleases (RGNs) may be used for excising the instability region in FRDA patient cells. This approach requires 1) an RGN and guide RNA sequence that can be programmed to target the allele in the human genome; and 2) a delivery approach for the RGN and guide sequence. Many nucleases used for genome editing, such as the commonly used Cas9 nuclease from S. pyogenes (SpCas9), are too large to be packaged into adeno-associated viral (AAV) vectors, especially when considering the length of the SpCas9 gene and the guide RNA in addition to other genetic elements required for functional expression cassettes. This makes an approach using SpCas9 more difficult.

Certain RNA guided nucleases of the invention are well suited for packaging into an AAV vector along with a guide RNA. Packing two guide RNAs would likely require a second vector, but this approach still compares favorably to what would be required of a larger nuclease such as SpCas9, which may require splitting the protein sequence between two vectors. The present invention encompasses a strategy using RGNs of the invention in which a region of genomic instability is removed. Such a strategy is applicable to other diseases and disorders which have a similar genetic basis, such as Huntington's Disease. Similar strategies using RGNs of the invention may also be applicable to similar diseases and disorders in non-human animals of agronomic or economic importance, including dogs, cats, horses, pigs, cows, sheep, chickens, donkeys, snakes, ferrets, and fish including salmon and shrimp.

### Modifying Causal Mutations by Targeted Mutagenesis

RGNs of the invention could also be to introduce disruptive mutations that may result in a beneficial effect. Genetic defects in the genes encoding hemoglobin, particularly the beta globin chain (the HBB gene), can be responsible for a number of diseases known as hemoglobinopathies, including sickle cell anemia and thalassemias.

In adult humans, hemoglobin is a heterotetramer comprising two alpha (α)-like globin chains and two beta (β)-like globin chains and 4 heme groups. In adults the α2β2 tetramer is referred to as Hemoglobin A (HbA) or adult hemoglobin. Typically, the alpha and beta globin chains are synthesized in an approximate 1:1 ratio and this ratio seems to be critical in terms of hemoglobin and red blood cell (RBC) stabilization. In a developing fetus, a different form of hemoglobin, fetal hemoglobin (HbF), is produced which has a higher binding affinity for oxygen than Hemoglobin A such that oxygen can be delivered to the baby's system via the mother's blood stream. Fetal hemoglobin also contains two a globin chains, but in place of the adult β-globin chains, it has two fetal gamma (γ)-globin chains (i.e., fetal hemoglobin is α2γ2). The regulation of the switch from production of gamma- to beta-globin is quite complex, and primarily involves a down-regulation of gamma globin transcription with a simultaneous up-regulation of beta globin transcription. At approximately 30 weeks of gestation, the synthesis of gamma globin in the fetus starts to drop while the production of beta globin increases. By approximately 10 months of age, the newborn's hemoglobin is nearly all α2β2 although some HbF persists into adulthood (approximately 1-3% of total hemoglobin). In the majority of patients with hemoglobinopathies, the genes encoding gamma globin remain present, but expression is relatively low due to normal gene repression occurring around parturition as described above.

Sickle cell disease is caused by a V6E mutation in the β globin gene (HBB) (a GAG to GTG at the DNA level), where the resultant hemoglobin is referred to as “hemoglobinS” or “HbS.” Under lower oxygen conditions, HbS molecules aggregate and form fibrous precipitates. These aggregates cause the abnormality or ‘sickling’ of the RBCs, resulting in a loss of flexibility of the cells. The sickling RBCs are no longer able to squeeze into the capillary beds and can result in vaso-occlusive crisis in sickle cell patients. In addition, sickled RBCs are more fragile than normal RBCs, and tend towards hemolysis, eventually leading to anemia in the patient.

Treatment and management of sickle cell patients is a life-long proposition involving antibiotic treatment, pain management and transfusions during acute episodes. One approach is the use of hydroxyurea, which exerts its effects in part by increasing the production of gamma globin. Long term side effects of chronic hydroxyurea therapy are still unknown, however, and treatment gives unwanted side effects and can have variable efficacy from patient to patient. Despite an increase in the efficacy of sickle cell treatments, the life expectancy of patients is still only in the mid to late 50's and the associated morbidities of the disease have a profound impact on a patient's quality of life.

Thalassemias (alpha thalassemias and beta thalassemia) are also diseases relating to hemoglobin and typically involve a reduced expression of globin chains. This can occur through mutations in the regulatory regions of the genes or from a mutation in a globin coding sequence that results in reduced expression or reduced levels or functional globin protein. Treatment of thalassemias usually involves blood transfusions and iron chelation therapy. Bone marrow transplants are also being used for treatment of people with severe thalassemias if an appropriate donor can be identified, but this procedure can have significant risks.

One approach that has been proposed for the treatment of both SCD and beta thalassemias is to increase the expression of gamma globin so that HbF functionally replaces the aberrant adult hemoglobin. As mentioned above, treatment of SCD patients with hydroxyurea is thought to be successful in part due to its effect on increasing gamma globin expression (DeSimone (1982) Proc Nat'l Acad Sci USA 79 (14): 4428-31; Ley, et al., (1982) N. Engl. J. Medicine, 307:1469-1475; Ley, et al., (1983) Blood 62:370-380; Constantoulakis et al., (1988) Blood 72 (6): 1961-1967, all herein incorporated by reference). Increasing the expression of HbF involves identification of genes whose products play a role in the regulation of gamma globin expression. One such gene is BCL11A. BCL11A encodes a zinc finger protein that expressed in adult erythroid precursor cells, and down-regulation of its expression leads to an increase in gamma globin expression (Sankaran et at (2008) Science 322:1839, herein incorporated by reference). Use of an inhibitory RNA targeted to the BCL11A gene has been proposed (e.g., U.S. Patent Publication 2011/0182867, herein incorporated by reference) but this technology has several potential drawbacks, including that complete knock down may not be achieved, delivery of such RNAs may be problematic, and the RNAs must be present continuously, requiring multiple treatments for life.

RGNs of the invention may be used to target the BCL11A enhancer region to disrupt expression of BCL11A, thereby increasing gamma globin expression. This targeted disruption can be achieved by non-homologous end joining (NHEJ), whereby an RGN of the invention targets to a particular sequence within the BCL11A enhancer region, makes a double-stranded break, and the cell's machinery repairs the break, typically simultaneously introducing deleterious mutations. Similar to what is described for other disease targets, RGNs of the invention may have advantages over other known RGNs due to their relatively small size, which enables packaging expression cassettes for the RGN and its guide RNA into a single AAV vector for in vivo delivery. Similar strategies using RGNs of the invention may also be applicable to similar diseases and disorders in both humans and in non-human animals of agronomic or economic importance.

### IX. Cells Comprising a Polynucleotide Genetic Modification

Provided herein are cells and organisms comprising a target sequence of interest that has been modified using a process mediated by an RGN, crRNA, and/or tracrRNA as described herein. In some of these embodiments, the RGN comprises the amino acid sequence of SEQ ID NO: 1, 9, 16, 23, 30, 38, 46, 54, 61, 69, 75, 82, 89, 95, 103, 110, 117, 137, or 235, or an active variant or fragment thereof. In various embodiments, the guide RNA comprises a CRISPR repeat sequence comprising the nucleotide sequence of SEQ ID NO: 2, 10, 17, 24, 31, 39, 47, 55, 62, 70, 76, 83, 90, 96, 104, 111, 118, 240, 273, or 287, or an active variant or fragment thereof. In particular embodiments, the guide RNA comprises a tracrRNA comprising the nucleotide sequence of SEQ ID NO: 3, 11, 18, 25, 32, 40, 48, 56, 63, 71, 77, 84, 91, 97, 105, 112, 119, 241, 274, or 286, or an active variant or fragment thereof. The guide RNA of the system can be a single guide RNA or a dual-guide RNA.

The modified cells can be eukaryotic (e.g., mammalian, plant, insect cell) or prokaryotic. Also provided are organelles and embryos comprising at least one nucleotide sequence that has been modified by a process utilizing an RGN, crRNA, and/or tracrRNA as described herein. The genetically modified cells, organisms, organelles, and embryos can be heterozygous or homozygous for the modified nucleotide sequence.

The chromosomal modification of the cell, organism, organelle, or embryo can result in altered expression (up-regulation or down-regulation), inactivation, or the expression of an altered protein product or an integrated sequence. In those instances wherein the chromosomal modification results in either the inactivation of a gene or the expression of a non-functional protein product, the genetically modified cell, organism, organelle, or embryo is referred to as a “knock out”. The knock out phenotype can be the result of a deletion mutation (i.e., deletion of at least one nucleotide), an insertion mutation (i.e., insertion of at least one nucleotide), or a nonsense mutation (i.e., substitution of at least one nucleotide such that a stop codon is introduced).

Alternatively, the chromosomal modification of a cell, organism, organelle, or embryo can produce a “knock in”, which results from the chromosomal integration of a nucleotide sequence that encodes a protein. In some of these embodiments, the coding sequence is integrated into the chromosome such that the chromosomal sequence encoding the wild-type protein is inactivated, but the exogenously introduced protein is expressed.

In other embodiments, the chromosomal modification results in the production of a variant protein product. The expressed variant protein product can have at least one amino acid substitution and/or the addition or deletion of at least one amino acid. The variant protein product encoded by the altered chromosomal sequence can exhibit modified characteristics or activities when compared to the wild-type protein, including but not limited to altered enzymatic activity or substrate specificity.

In yet other embodiments, the chromosomal modification can result in an altered expression pattern of a protein. As a non-limiting example, chromosomal alterations in the regulatory regions controlling the expression of a protein product can result in the overexpression or downregulation of the protein product or an altered tissue or temporal expression pattern.

### X. Kits and Methods for Detecting Target DNA or Single-Stranded DNA

Some RGNs (e.g., APG09106.1 and APG09748, set forth as SEQ ID NOs: 54 and 137) can promiscuously cleave non-targeted single-stranded DNA (ssDNA) once activated by detection of a target DNA. Thus, provided herein are compositions and methods for detecting a target DNA (double-stranded or single-stranded) in a sample. In some embodiments, the desired target may exist as RNA, such as the genome or part of a genome of an RNA virus, such as for example a coronavirus. In some embodiments, the coronavirus may be a SARS-like coronavirus. In further embodiments, the coronavirus may be SARS-COV-2, SARS-COV, or a bat SARS-like coronavirus such as bat-SL-CoVZC45 (accession MG772933). In embodiments where the target exists as RNA, the target may be reverse-transcribed into a DNA molecule which can be effectively targeted by the RGN. Reverse-transcription may be followed by an amplification step, such as RT-PCR methods known in the art, which involve thermocycling, or may be by isothermal methods such as RT-LAMP (reverse transcription loop-mediated isothermal amplification) (Notomi et al., Nucleic Acids Res 28: E63, (2000)).

These compositions and methods involve the use of a detector ssDNA that does not hybridize with the guideRNA and is a non-target ssDNA. In some embodiments, the detector ssDNA comprises a detectable label that provides a detectable signal after cleavage of the detector ssDNA. A non-limiting example is a detector ssDNA that comprises a fluorophore/quencher pair wherein the fluorophore does not fluoresce when the detector ssDNA is whole (i.e., uncleaved) as its signal is suppressed by the presence of the quencher in close proximity. Cleavage of the detector ssDNA results in removal of the quencher and the fluorescent label can then be detected. Non-limiting examples of fluorescent labels or dyes include Cy5, fluorescein (e.g., FAM, 6 FAM, 5 (6) FAM, FITC), Cy3, Alexa Fluor® dyes, and Texas Red. Non-limiting examples of quenchers include Iowa BlackRFQ, Iowa Black® RQ, a Qxl quencher, an ATTO quencher, and a QSY dye. In some embodiments, the detector ssDNA comprises a second quencher, such as an internal quencher like ZEN™, TAO™, and Black Hole Quencher®, which can lower background and increase signal detection.

In other embodiments, the detector ssDNA comprises a detectable label that provides a detectable signal before cleavage of the detector ssDNA and cleavage of the ssDNA inhibits or prevents detection of the signal. A non-limiting example of such a scenario is a detector ssDNA that comprises a fluorescence resonance energy transfer (FRET) pair. FRET is a process by which radiationless transfer of energy occurs from an excited state of a first (donor) fluorophore to a second (acceptor) fluorophore in close proximity. The emission spectrum of the donor fluorophore overlaps with the excitation spectrum of the acceptor fluorophore. Thus, the acceptor fluorophore will fluoresce when the detector ssDNA is whole (i.e., uncleaved) and the acceptor fluorophore will no longer fluoresce when the detector ssDNA is cleaved because the donor and acceptor fluorophore will no longer be in close proximity to one another. FRET donor and acceptor fluorophores are known in the art and include, but are not limited to cyan fluorescent protein (CFP)/green fluorescent protein (GFP), Cy3/Cy5, and GFP/yellow fluorescent protein (YFP).

In some embodiments, the detector ssDNA has a length of from about 2 nucleotides to about 30 nucleotides, including but not limited to about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 nucleotides, about 26 nucleotides, about 27 nucleotides, about 28 nucleotides, about 29 nucleotides, and about 30 nucleotides.

Methods of detecting a target DNA of a DNA molecule comprise contacting a sample with an RGN, a guide RNA capable of hybridizing with the RGN and a target DNA sequence in a DNA molecule, and a detector single-stranded DNA (ssDNA) that does not hybridize with the guide RNA, followed by measuring a detectable signal produced by cleavage of the ssDNA by the RGN, thereby detecting the target DNA sequence of the DNA molecule. In some embodiments, the method can comprise a step of amplification of the nucleic acid molecules within a sample, either before or simultaneously with contact with the RGN and guideRNA. In some of these embodiments, specific sequences to which the guide RNA will hybridize can be amplified in order to increase sensitivity of a detection method.

The sample in which a target DNA can be detected using these compositions and methods comprising a detector ssDNA include any sample comprising or believed to comprise a nucleic acid (e.g., DNA or RNA molecule). The sample can be derived from any source including a synthetic combination of purified nucleic acids or a biological sample such as respiratory swab (e.g., nasopharyngeal swab) extracts, a cell lysate, a patient sample, cells, tissues, saliva, blood, serum, plasma, urine, aspirate, biopsy samples, cerebral spinal fluid, or organism (e.g., bacteria, virus).

The contacting of the sample with the RGN, guide RNA, and detector ssDNA can include contacting in vitro, ex vivo, or in vivo. In some embodiments, the detector ssDNA and/or the RGN and/or guide RNA is immobilized on for example, a lateral flow device, wherein the sample contacts the immobilized detector ssDNA and/or RGN and/or guide RNA. In some embodiments, antibodies against antigen moieties on the detector ssDNA are immobilized on, for example, a lateral flow device in a manner that allows differentiation of cleaved detector ssDNA from intact detector ssDNA.

In some embodiments, the methods can further comprise determining the amount of the target DNA present in the sample. The measurement of the detectable signal in the test sample can be compared to a reference measurement (e.g., a measurement of a reference sample or series thereof comprising a known amount of target DNA).

Non-limiting examples of applications of the compositions and methods include single-nucleotide polymorphism (SNP) detection, cancer screening, detection of a bacterial infection, detection of antibiotic resistance, and detection of a viral infection.

The detectable signal produced by cleavage of the ssDNA by the RGN can be measured using any suitable method known in the art including but not limited to measuring fluorescent signal, a visual analysis of bands on a gel, a colorimetric change, and the presence or absence of an electrical signal.

The present invention provides kits for detecting a target DNA of a DNA molecule in a sample, wherein the kit comprises an RGN polypeptide, a guide RNA capable of hybridizing with the RGN and a target DNA sequence in a DNA molecule, and a detector ssDNA that does not hybridize with the guide RNA.

Also provided herein are methods of cleaving single-stranded DNAs by contacting a population of nucleic acids, wherein the population comprises a target DNA sequence of a DNA molecule and a plurality of non-target ssDNAs with an RGN and a guide RNA capable of hybridizing with the RGN and the target DNA sequence.

The article “a” and “an” are used herein to refer to one or more than one (i.e., to at least one) of the grammatical object of the article. By way of example, “a polypeptide” means one or more polypeptides.

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended embodiments.

Non-limiting embodiments include:

1. A nucleic acid molecule comprising a polynucleotide encoding an RNA-guided nuclease (RGN) polypeptide, wherein said polynucleotide comprises a nucleotide sequence encoding an RGN polypeptide comprising an amino acid sequence having at least 95% sequence identity to SEQ ID NO: 1, 9, 16, 23, 30, 38, 46, 54, 61, 69, 75, 82, 89, 95, 103, 110, or 117;
wherein said RGN polypeptide is capable of binding a target DNA sequence of a DNA molecule in an RNA-guided sequence specific manner when bound to a guide RNA (gRNA) capable of hybridizing to said target DNA sequence, and
wherein said polynucleotide encoding an RGN polypeptide is operably linked to a promoter heterologous to said polynucleotide.

2 The nucleic acid molecule of embodiment 1, wherein said RGN polypeptide is capable of cleaving said target DNA sequence upon binding.

3. The nucleic acid molecule of embodiment 2, wherein said RGN polypeptide is capable of generating a double-stranded break.

4. The nucleic acid molecule of embodiment 2, wherein said RGN polypeptide is capable of generating a single-stranded break.

5. The nucleic acid molecule of embodiment 2, wherein said RGN polypeptide is nuclease inactive.

6 The nucleic acid molecule of any one of embodiments 1-5, wherein the RGN polypeptide is operably fused to a base-editing polypeptide.

7. The nucleic acid molecule of embodiment 6, wherein the base-editing polypeptide is a deaminase.

8 The nucleic acid molecule of embodiment 7, wherein the deaminase is a cytidine deaminase or an adenosine deaminase.

9. The nucleic acid molecule of any one of embodiments 1-8, wherein the RGN polypeptide comprises one or more nuclear localization signals.

10. The nucleic acid molecule of any one of embodiments 1-9, wherein the RGN polypeptide is codon optimized for expression in a eukaryotic cell.

11. The nucleic acid molecule of any one of embodiments 1-10, wherein said target DNA sequence is located adjacent to a protospacer adjacent motif (PAM).

12. A vector comprising the nucleic acid molecule of any one of embodiments 1-11.

13. The vector of embodiment 12, further comprising at least one nucleotide sequence encoding said gRNA capable of hybridizing to said target DNA sequence.

14. The vector of embodiment 13, wherein the guide RNA comprises a CRISPR RNA comprising a CRISPR repeat sequence having at least 95% sequence identity to SEQ ID NO: 2, 10, 17, 24, 31, 39, 47, 55, 62, 70, 76, 83, 90, 96, 104, 111, or 118.

15. The vector of embodiment 13 or 14, wherein said gRNA comprises a tracrRNA.

16. The vector of embodiment 15, wherein the tracrRNA has at least 95% sequence identity to SEQ ID NO: 3, 11, 18, 25, 32, 40, 48, 56, 63, 71, 77, 84, 91, 97, 105, 112, or 119.

17. The vector of embodiment 15 or 16, where said gRNA is a single guide RNA.

18. The vector of embodiment 15 or 16, wherein said gRNA is a dual-guide RNA.

19. A cell comprising the nucleic acid molecule of any one of embodiments 1-11 or the vector of any one of embodiments 12-18.

20. A method for making an RGN polypeptide comprising culturing the cell of embodiment 18 under conditions in which the RGN polypeptide is expressed.

21. A method for making an RGN polypeptide comprising introducing into a cell a heterologous nucleic acid molecule comprising a nucleotide sequence encoding an RNA-guided nuclease (RGN) polypeptide comprising an amino acid sequence having at least 95% sequence identity to SEQ ID NO: 1, 9, 16, 23, 30, 38, 46, 54, 61, 69, 75, 82, 89, 95, 103, 110, or 117;
wherein said RGN polypeptide is capable of binding a target DNA sequence of a DNA molecule in an RNA-guided sequence specific manner when bound to a guide RNA (gRNA) capable of hybridizing to said target DNA sequence;
and culturing said cell under conditions in which the RGN polypeptide is expressed.

22. The method of embodiment 20 or 21, further comprising purifying said RGN polypeptide.

23. The method of embodiment 20 or 21, wherein said cell further expresses one or more guide RNAs that binds to said RGN polypeptide to form an RGN ribonucleoprotein complex.

24. The method of embodiment 23, further comprising purifying said RGN ribonucleoprotein complex.

25. A nucleic acid molecule comprising a polynucleotide encoding a CRISPR RNA (crRNA), wherein said crRNA comprises a spacer sequence and a CRISPR repeat sequence, wherein said CRISPR repeat sequence comprises a nucleotide sequence having at least 95% sequence identity to SEQ ID NO: 2, 10, 17, 24, 31, 39, 47, 55, 62, 70, 76, 83, 90, 96, 104, 111, or 118;
wherein a guide RNA comprising:
   a) said crRNA; and optionally,
   b) a trans-activating CRISPR RNA (tracrRNA) capable of hybridizing to said CRISPR repeat sequence of said crRNA;
is capable of hybridizing to a target DNA sequence of a DNA molecule in a sequence specific manner through the spacer sequence of said crRNA when said guide RNA is bound to an RNA-guided nuclease (RGN) polypeptide, and
wherein said polynucleotide encoding a crRNA is operably linked to a promoter heterologous to said polynucleotide.

26. A vector comprising the nucleic acid molecule of embodiment 25.

27. The vector of embodiment 26, wherein said vector further comprises a polynucleotide encoding said tracrRNA.

28. The vector of embodiment 27, wherein said tracrRNA comprises a nucleotide sequence having at least 95% sequence identity to SEQ ID NO: 3, 11, 18, 25, 32, 40, 48, 56, 63, 71, 77, 84, 91, 97, 105, 112, or 119.

29. The vector of embodiment 27 or 28, wherein said polynucleotide encoding said crRNA and said polynucleotide encoding said tracrRNA are operably linked to the same promoter and are encoded as a single guide RNA.

30. The vector of embodiment 27 or 28, wherein said polynucleotide encoding said crRNA and said polynucleotide encoding said tracrRNA are operably linked to separate promoters.

31. The vector of any one of embodiments 26-30, wherein said vector further comprises a polynucleotide encoding said RGN polypeptide, wherein said RGN polypeptide comprises an amino acid sequence having at least 95% sequence identity to SEQ ID NO: 1, 9, 16, 23, 30, 38, 46, 54, 61, 69, 75, 82, 89, 95, 103, 110, or 117.

32. A nucleic acid molecule comprising a polynucleotide encoding a trans-activating CRISPR RNA (tracrRNA) comprising a nucleotide sequence having at least 95% sequence identity to SEQ ID NO: 3, 11, 18, 25, 32, 40, 48, 56, 63, 71, 77, 84, 91, 97, 105, 112, or 119;
wherein a guide RNA comprising:
   a) said tracrRNA; and
   b) a crRNA comprising a spacer sequence and a CRISPR repeat sequence, wherein said tracrRNA is capable of hybridizing with said CRISPR repeat sequence of said crRNA;
is capable of hybridizing to a target DNA sequence of a DNA molecule in a sequence specific manner through the spacer sequence of said crRNA when said guide RNA is bound to an RNA-guided nuclease (RGN) polypeptide, and
wherein said polynucleotide encoding a tracrRNA is operably linked to a promoter heterologous to said polynucleotide.

33. A vector comprising the nucleic acid molecule of embodiment 32.

34. The vector of embodiment 33, wherein said vector further comprises a polynucleotide encoding said crRNA.

35. The vector of embodiment 34, wherein the CRISPR repeat sequence of said crRNA comprises a nucleotide sequence having at least 95% sequence identity to SEQ ID NO: 2, 10, 17, 24, 31, 39, 47, 55, 62, 70, 76, 83, 90, 96, 104, 111, or 118.

36. The vector of embodiment 34 or 35, wherein said polynucleotide encoding said crRNA and said polynucleotide encoding said tracrRNA are operably linked to the same promoter and are encoded as a single guide RNA. 37. The vector of embodiment 34 or 35, wherein said polynucleotide encoding said crRNA and said polynucleotide encoding said tracrRNA are operably linked to separate promoters. 38. The vector of any one of embodiments 33-37, wherein said vector further comprises a polynucleotide encoding said RGN polypeptide, wherein said RGN polypeptide comprises an amino acid sequence having at least 95% sequence identity to SEQ ID NO: 1, 9, 16, 23, 30, 38, 46, 54, 61, 69, 75, 82, 89, 95, 103, 110 or 117.

39. A system for binding a target DNA sequence of a DNA molecule, said system comprising:
a) one or more guide RNAs capable of hybridizing to said target DNA sequence or one or more polynucleotides comprising nucleotide sequences encoding the one or more guide RNAs (gRNAs); and
b) an RNA-guided nuclease (RGN) polypeptide comprising an amino acid sequence having at least 95% sequence identity to SEQ ID NO: 1, 9, 16, 23, 30, 38, 46, 54, 61, 69, 75, 82, 89, 95, 103, 110, or 117; or a polynucleotide comprising a nucleotide sequence encoding the RGN polypeptide;
wherein said nucleotide sequences encoding the one or more guide RNAs and encoding the RGN polypeptide are each operably linked to a promoter heterologous to said nucleotide sequence; and
wherein the one or more guide RNAs are capable of forming a complex with the RGN polypeptide, in order to direct said RGN polypeptide to bind to said target DNA sequence of the DNA molecule.

40. The system of embodiment 39, wherein said gRNA comprises a CRISPR repeat sequence comprising a nucleotide sequence having at least 95% sequence identity to SEQ ID NO: 2, 10, 17, 24, 31, 39, 47, 55, 62, 70, 76, 83, 90, 96, 104, 111, or 118.

41. The system of embodiment 39 or 40, wherein said gRNA comprises a tracrRNA.

42. The system of embodiment 41, wherein said tracrRNA comprises a nucleotide sequence having at least 95% sequence identity to SEQ ID NO: 3, 11, 18, 25, 32, 40, 48, 56, 63, 71, 77, 84, 91, 97, 105, 112, or 119.

43. The system of embodiment 41 or 42, wherein said gRNA is a single guide RNA (sgRNA).

44. The system of embodiment 41 or 42, wherein said gRNA is a dual-guide RNA.

45. The system of any one of embodiments 39-44, wherein said target DNA sequence is located adjacent to a protospacer adjacent motif (PAM).

46. The system of any one of embodiments 39-45, wherein the target DNA sequence is within a cell.

47. The system of embodiment 46, wherein the cell is a eukaryotic cell.

48. The system of embodiment 47, wherein the eukaryotic cell is a plant cell.

49. The system of embodiment 47, wherein the eukaryotic cell is a mammalian cell.

50. The system of embodiment 47, wherein the eukaryotic cell is an insect cell.

51. The system of embodiment 46, wherein the cell is a prokaryotic cell.

52. The system of any one of embodiments 39-51, wherein when transcribed the one or more guide RNAs is capable of hybridizing to the target DNA sequence and the guide RNA is capable of forming a complex with the RGN polypeptide to direct cleavage of the target DNA sequence.

53. The system of embodiment 52, wherein said RGN polypeptide is capable of generating a double-stranded break.

54. The system of embodiment 52, wherein said RGN polypeptide is capable of generating a single-stranded break.

55. The system of embodiment 52, wherein said RGN polypeptide is nuclease inactive.

56. The system of any one of embodiments 39-55, wherein the RGN polypeptide is operably linked to a base-editing polypeptide.

57. The system of embodiment 56, wherein the base-editing polypeptide is a deaminase.

58. The system of embodiment 57, wherein the deaminase is a cytidine deaminase or an adenosine deaminase.

59. The system of any one of embodiments 39-58, wherein the RGN polypeptide comprises one or more nuclear localization signals.

60. The system of any one of embodiments 39-59, wherein the RGN polypeptide is codon optimized for expression in a eukaryotic cell.

61. The system of any one of embodiments 39-60, wherein polynucleotides comprising the nucleotide sequences encoding the one or more guide RNAs and the polynucleotide comprising the nucleotide sequence encoding an RGN polypeptide are located on one vector.

62. The system of any one of embodiments 39-61, wherein said system further comprises one or more donor polynucleotides or one or more nucleotide sequences encoding the one or more donor polynucleotides.

63. A method for binding a target DNA sequence of a DNA molecule comprising delivering a system according to any one of embodiments 39-62, to said target DNA sequence or a cell comprising the target DNA sequence.

64. The method of embodiment 63, wherein said RGN polypeptide or said guide RNA further comprises a detectable label, thereby allowing for detection of said target DNA sequence.

65. The method of embodiment 63, wherein said guide RNA or said RGN polypeptide further comprises an expression modulator, thereby modulating expression of said target DNA sequence or a gene under transcriptional control by said target DNA sequence.

66. A method for cleaving or modifying a target DNA sequence of a DNA molecule comprising delivering a system according to any one of embodiments 39-62, to said target DNA sequence or a cell comprising the DNA molecule and cleavage or modification of said target DNA sequence occurs.

67. The method of embodiment 66, wherein said modified target DNA sequence comprises insertion of heterologous DNA into the target DNA sequence.

68. The method of embodiment 66, wherein said modified target DNA sequence comprises deletion of at least one nucleotide from the target DNA sequence.

69. The method of embodiment 66, wherein said modified target DNA sequence comprises mutation of at least one nucleotide in the target DNA sequence.

70. A method for binding a target DNA sequence of a DNA molecule, said method comprising:
a) assembling an RNA-guided nuclease (RGN) ribonucleotide complex in vitro by combining:
   i) one or more guide RNAs capable of hybridizing to the target DNA sequence; and
   ii) an RGN polypeptide comprising an amino acid having at least 95% sequence identity to SEQ ID NO: 1, 9, 16, 23, 30, 38, 46, 54, 61, 69, 75, 82, 89, 95, 103, 110, or 117
   under conditions suitable for formation of the RGN ribonucleotide complex; and
b) contacting said target DNA sequence or a cell comprising said target DNA sequence with the in vitro-assembled RGN ribonucleotide complex;
wherein the one or more guide RNAs hybridize to the target DNA sequence, thereby directing said RGN polypeptide to bind to said target DNA sequence.

71. The method of embodiment 70, wherein said RGN polypeptide or said guide RNA further comprises a detectable label, thereby allowing for detection of said target DNA sequence.

72. The method of embodiment 70, wherein said guide RNA or said RGN polypeptide further comprises an expression modulator, thereby allowing for the modulation of expression of said target DNA sequence.

73. A method for cleaving and/or modifying a target DNA sequence of a DNA molecule, comprising contacting the DNA molecule with:
a) an RNA-guided nuclease (RGN) polypeptide, wherein said RGN comprises an amino acid having at least 95% sequence identity to SEQ ID NO: 1, 9, 16, 23, 30, 38, 46, 54, 61, 69, 75, 82, 89, 95, 103, 110, or 117; and
b) one or more guide RNAs capable of targeting the RGN of (a) to the target DNA sequence;
wherein the one or more guide RNAs hybridize to the target DNA sequence, thereby directing said RGN polypeptide to bind to said target DNA sequence and cleavage and/or modification of said target DNA sequence occurs.

74. The method of embodiment 73, wherein the cleavage by said RGN polypeptide generates a double-stranded break.

The method of embodiment 73, wherein the cleavage by said RGN polypeptide generates a 75. single-stranded break.

76. The method of embodiment 73, wherein said RGN polypeptide is nuclease inactive.

77. The method of any one of embodiments 73-76, wherein said RGN polypeptide is operably linked to a base-editing polypeptide.

78. The method of embodiment 77, wherein said base-editing polypeptide comprises a deaminase.

79. The method of embodiment 78, wherein said deaminase is a cytidine deaminase or an adenosine deaminase.

80. The method of any one of embodiments 73-79, wherein said modified target DNA sequence comprises insertion of heterologous DNA into the target DNA sequence.

81. The method of any one of embodiments 73-79, wherein said modified target DNA sequence comprises deletion of at least one nucleotide from the target DNA sequence.

82. The method of any one of embodiments 73-79, wherein said modified target DNA sequence comprises mutation of at least one nucleotide in the target DNA sequence.

83. The method of any one of embodiments 70-82, wherein said gRNA comprises a CRISPR repeat sequence comprising a nucleotide sequence having at least 95% sequence identity to SEQ ID NO: 2, 10, 17, 24, 31, 39, 47, 55, 62, 70, 76, 83, 90, 96, 104, 111, or 118.

84. The method of any one of embodiments 70-83, wherein said gRNA comprises a tracrRNA.

85. The method of embodiment 84, wherein said tracrRNA comprises a nucleotide sequence having at least 95% sequence identity to SEQ ID NO: 3, 11, 18, 25, 32, 40, 48, 56, 63, 71, 77, 84, 91, 97, 105, 112, or 119.

86. The method of embodiment 84 or 85, wherein said gRNA is a single guide RNA (sgRNA).

87. The method of embodiment 84 or 85, wherein said gRNA is a dual-guide RNA.

88. The method of any one of embodiments 63-87, wherein said target DNA sequence is located adjacent to a protospacer adjacent motif (PAM).

89. The method of any one of embodiments 63-88, wherein the target DNA sequence is within a cell.

90. The method of embodiment 89, wherein the cell is a eukaryotic cell.

91. The method of embodiment 90, wherein the eukaryotic cell is a plant cell.

92. The method of embodiment 90, wherein the eukaryotic cell is a mammalian cell.

93. The method of embodiment 90, wherein the eukaryotic cell is an insect cell.

94. The method of embodiment 89, wherein the cell is a prokaryotic cell.

95. The method of any one of embodiments 89-94, further comprising culturing the cell under conditions in which the RGN polypeptide is expressed and cleaves the target DNA sequence to produce a DNA molecule comprising a modified DNA sequence; and selecting a cell comprising said modified target DNA sequence.

96. A cell comprising a modified target DNA sequence according to the method of embodiment 95.

97. The cell of embodiment 96, wherein the cell is a eukaryotic cell.

98. The cell of embodiment 97, wherein the eukaryotic cell is a plant cell.

99. A plant comprising the cell of embodiment 98.

100. A seed comprising the cell of embodiment 98.

101. The cell of embodiment 97, wherein the eukaryotic cell is a mammalian cell.

102. The cell of embodiment 98, wherein the eukaryotic cell is an insect cell.

103. The cell of embodiment 96, wherein the cell is a prokaryotic cell.

104. A method for producing a genetically modified cell with a correction in a causal mutation for a genetically inherited disease, the method comprising introducing into the cell:
a) an RNA-guided nuclease (RGN) polypeptide, wherein the RGN polypeptide comprises an amino acid sequence having at least 95% sequence identity to SEQ ID NO: 1, 9, 16, 23, 30, 38, 46, 54, 61, 69, 75, 82, 89, 95, 103, 110, or 117; or a polynucleotide encoding said RGN polypeptide, wherein said polynucleotide encoding the RGN polypeptide is operably linked to a promoter to enable expression of the RGN polypeptide in the cell; and
b) a guide RNA (gRNA), wherein the gRNA comprises a CRISPR repeat sequence comprising a nucleotide sequence having at least 95% sequence identity to SEQ ID NO: 2, 10, 17, 24, 31, 39, 47, 55, 62, 70, 76, 83, 90, 96, 104, 111, or 118, or a polynucleotide encoding said gRNA, wherein said polynucleotide encoding the gRNA is operably linked to a promoter to enable expression of the gRNA in the cell
whereby the RGN and gRNA target to the genomic location of the causal mutation and modify the genomic sequence to remove the causal mutation.

105. The method of embodiment 104, wherein the RGN is operably linked to a base-editing polypeptide.

106. The method of embodiment 105, wherein the base-editing polypeptide is a deaminase.

107. The method of embodiment 106, wherein the deaminase is a cytidine deaminase or an adenosine deaminase.

108. The method of any one of embodiments 104-107, wherein the cell is an animal cell.

109. The method of any one of embodiments 104-107, wherein the cell is a mammalian cell.

110. The method of embodiment 108, wherein the cell is derived from a dog, cat, mouse, rat, rabbit, horse, cow, pig, or human.

111. The method of embodiment 108, wherein the genetically inherited disease is caused by a single nucleotide polymorphism.

112. The method of embodiment 111, wherein the genetically inherited disease is Hurler Syndrome.

113. The method of embodiment 112, wherein the gRNA further comprises a spacer sequence that targets a region proximal to the causal single nucleotide polymorphism.

114. A method for producing a genetically modified cell with a deletion in a disease-causing genomic region of instability, the method comprising introducing into the cell:
a) an RNA-guided nuclease (RGN) polypeptide, wherein the RGN polypeptide comprises an amino acid sequence having at least 95% sequence identity to SEQ ID NO: 1, 9, 16, 23, 30, 38, 46, 54, 61, 69, 75, 82, 89, 95, 103, 110, or 117; or a polynucleotide encoding said RGN polypeptide, wherein said polynucleotide encoding the RGN polypeptide is operably linked to a promoter to enable expression of the RGN polypeptide in the cell; and
b) a guide RNA (gRNA), wherein the gRNA comprises a CRISPR repeat sequence comprising a nucleotide sequence having at least 95% sequence identity to SEQ ID NO: 2, 10, 17, 24, 31, 39, 47, 55, 62, 70, 76, 83, 90, 96, 104, 111, or 118, or a polynucleotide encoding said gRNA, wherein said polynucleotide encoding the gRNA is operably linked to a promoter to enable expression of the gRNA in the cell, and further wherein the gRNA comprises a spacer sequence that targets the 5′ flank of the genomic region of instability; and
c) a second guide RNA (gRNA), wherein the gRNA comprises a CRISPR repeat sequence comprising a nucleotide sequence having at least 95% sequence identity to SEQ ID NO: 2, 10, 17, 24, 31, 39, 47, 55, 62, 70, 76, 83, 90, 96, 104, 111, or 118, or a polynucleotide encoding said gRNA, wherein said polynucleotide encoding the gRNA is operably linked to a promoter to enable expression of the second gRNA in the cell, and further wherein said second gRNA comprises a spacer sequence that targets the 3′ flank of the genomic region of instability;
whereby the RGN and the two gRNAs target to the genomic region of instability and at least a portion of the genomic region of instability is removed.

115. The method of embodiment 114, wherein the cell is an animal cell.

116. The method of embodiment 114, wherein the cell is a mammalian cell.

117. The method of embodiment 115, wherein the cell is derived from a dog, cat, mouse, rat, rabbit, horse, cow, pig, or human.

118. The method of embodiment 115, wherein the genetically inherited disease is Friedrich's

Ataxia or Huntington's Disease.

119. The method of embodiment 118, wherein the first gRNA further comprises a spacer sequence that targets a region within or proximal to the genomic region of instability.

120. The method of embodiment 118, wherein the second gRNA further comprises a spacer sequence that targets a region within or proximal to genomic region of instability.

121. A method for producing a genetically modified mammalian hematopoietic progenitor cell having decreased BCL11A mRNA and protein expression, the method comprising introducing into an isolated human hematopoietic progenitor cell:
a) an RNA-guided nuclease (RGN) polypeptide, wherein the RGN polypeptide comprises an amino acid sequence having at least 95% sequence identity to SEQ ID NO: 1, 9, 16, 23, 30, 38, 46, 54, 61, 69, 75, 82, 89, 95, 103, 110, or 117; or a polynucleotide encoding said RGN polypeptide, wherein said polynucleotide encoding the RGN polypeptide is operably linked to a promoter to enable expression of the RGN polypeptide in the cell; and
b) a guide RNA (gRNA), wherein the gRNA comprises a CRISPR repeat sequence comprising a nucleotide sequence having at least 95% sequence identity to SEQ ID NO: 2, 10, 17, 24, 31, 39, 47, 55, 62, 70, 76, 83, 90, 96, 104, 111, or 118, or a polynucleotide encoding said gRNA, wherein said polynucleotide encoding the gRNA is operably linked to a promoter to enable expression of the gRNA in the cell,
whereby the RGN and gRNA are expressed in the cell and cleave at the BCL11A enhancer region, resulting in genetic modification of the human hematopoietic progenitor cell and reducing the mRNA and/or protein expression of BCL11A.

122. The method of embodiment 121, wherein the gRNA further comprises a spacer sequence that targets a region within or proximal to the BCL11A enhancer region.

123. The method of any one of embodiments 104-122, wherein the guide RNA comprises a tracrRNA comprising a nucleotide sequence having at least 95% sequence identity to SEQ ID NO: 3, 11, 18, 25, 32, 40, 48, 56, 63, 71, 77, 84, 91, 97, 105, 112, or 119.

124. A system for binding a target DNA sequence of a DNA molecule, said system comprising:
a) one or more guide RNAs capable of hybridizing to said target DNA sequence or one or more polynucleotides comprising one or more nucleotide sequences encoding the one or more guide RNAs (gRNAs); and
b) an RNA-guided nuclease (RGN) polypeptide comprising an amino acid sequence having at least 95% sequence identity to SEQ ID NO: 1, 9, 16, 23, 30, 38, 46, 54, 61, 69, 75, 82, 89, 95, 103, 110, or 117;
wherein the one or more guide RNAs are capable of hybridizing to the target DNA sequence, and
wherein the one or more guide RNAs are capable of forming a complex with the RGN polypeptide, in order to direct said RGN polypeptide to bind to said target DNA sequence of the DNA molecule.

125. The system of embodiment 124, wherein said RGN polypeptide is nuclease inactive or capable of functioning as a nickase.

126. The system of embodiment 124 or 125, wherein said RGN polypeptide is operably fused to a base-editing polypeptide.

127. The system of embodiment 126, wherein the base-editing polypeptide is a deaminase.

128. The system of embodiment 127, wherein the deaminase is a cytidine deaminase or an adenosine deaminase.

129. A method of detecting a target DNA sequence of a DNA molecule in a sample, the method comprising:
a) contacting the sample with:
   i) an RNA-guided nuclease (RGN) polypeptide comprising an amino acid sequence having at least 95% sequence identity to SEQ ID NO: 1, 9, 16, 23, 30, 38, 46, 54, 61, 69, 75, 82, 89, 95, 103, 110, 117, or 137, wherein said RGN polypeptide is capable of binding said target DNA sequence of a DNA molecule in an RNA-guided sequence specific manner when bound to a guide RNA capable of hybridizing to said target DNA sequence;
   ii) said guide RNA; and
   iii) a detector single-stranded DNA (ssDNA) that does not hybridize with the guide RNA; and
b) measuring a detectable signal produced by cleavage of the detector ssDNA by the RGN, thereby detecting the target DNA.

130. The method of embodiment 129, wherein said sample comprises DNA molecules from a cell lysate.

131. The method of embodiment 129, wherein said sample comprises cells.

132. The method of embodiment 131, wherein said cells are eukaryotic cells.

133. The method of embodiment 129, wherein the DNA molecule comprising the target DNA sequence is produced by reverse-transcription of an RNA template molecule present in a sample comprising RNA.

134 The method of embodiment 133, wherein the RNA template molecule is an RNA virus.

135. The method of embodiment 134, wherein the RNA virus is a coronavirus.

136. The method of embodiment 135, wherein the coronavirus is a bat SARS-like coronavirus, SARS-COV, or SARS-COV-2.

137. The method of any one of embodiments 133-136, wherein the sample comprising RNA is derived from a sample comprising cells.

138. The method of any one of embodiments 129-137, wherein said detector ssDNA comprises a fluorophore/quencher pair.

139. The method of any one of embodiments 129-137, wherein said detector ssDNA comprises a fluorescence resonance energy transfer (FRET) pair.

140. The method of any one of embodiments 129-139, wherein said guide RNA comprises a CRISPR repeat sequence comprising a nucleotide sequence having at least 95% sequence identity to SEQ ID NO: 2, 10, 17, 24, 31, 39, 47, 55, 62, 70, 76, 83, 90, 96, 104, 111, 118, or 273.

141. The method of any one of embodiments 129-140, wherein said guide RNA comprises a tracrRNA.

142. The method of embodiment 141, wherein said tracrRNA comprises a nucleotide sequence having at least 95% sequence identity to SEQ ID NO: 3, 11, 18, 25, 32, 40, 48, 56, 63, 71, 77, 84, 91, 97, 105, 112, 119, or 274.

143. The method of embodiment 141 or 142, wherein said guide RNA is a single guide RNA.

144 The method of embodiment 141 or 142, wherein said guide RNA is a dual-guide RNA.

145. The method of any one of embodiments 129-144, wherein said method further comprises amplifying nucleic acids in the sample prior to or together with the contacting of step a).

146. The method of embodiment 145, wherein the amplification is of a DNA molecule produced by reverse transcription of an RNA molecule.

147. A kit for detecting a target DNA sequence of a DNA molecule in a sample, the kit comprising:
a) an RNA-guided nuclease (RGN) polypeptide comprising an amino acid sequence having at least 95% sequence identity to SEQ ID NO: 1, 9, 16, 23, 30, 38, 46, 54, 61, 69, 75, 82, 89, 95, 103, 110, 117, or 137, wherein said RGN polypeptide is capable of binding said target DNA sequence of a DNA molecule in an RNA-guided sequence specific manner when bound to a guide RNA capable of hybridizing to said target DNA sequence;
b) said guide RNA; and
c) a detector single-stranded DNA (ssDNA) that does not hybridize with the guide RNA.

148. The kit of embodiment 147, wherein said detector ssDNA comprises a fluorophore/quencher pair

149. The kit of embodiment 147, wherein said detector ssDNA comprises a fluorescence resonance energy transfer (FRET) pair.

150. The kit of any one of embodiments 147-149, wherein said guide RNA comprises a CRISPR repeat sequence comprising a nucleotide sequence having at least 95% sequence identity to SEQ ID NO: 2, 10, 17, 24, 31, 39, 47, 55, 62, 70, 76, 83, 90, 96, 104, 111, 118, or 273.

151. The kit of any one of embodiments 147-150, wherein said guide RNA comprises a tracrRNA.

152. The kit of embodiment 151, wherein said tracrRNA comprises a nucleotide sequence having at least 95% sequence identity to SEQ ID NO: 3, 11, 18, 25, 32, 40, 48, 56, 63, 71, 77, 84, 91, 97, 105, 112, 119, or 274.

153. The kit of embodiment 151 or 152, wherein said guide RNA is a single guide RNA.

154. The kit of embodiment 151 or 152, wherein said guide RNA is a dual-guide RNA.

155. A method of cleaving single-stranded DNAs, the method comprising contacting a population of nucleic acids, wherein said population comprises a DNA molecule comprising a target DNA sequence and a plurality of non-target ssDNAs with:
a) an RNA-guided nuclease (RGN) polypeptide comprising an amino acid sequence having at least 95% sequence identity to SEQ ID NO: 1, 9, 16, 23, 30, 38, 46, 54, 61, 69, 75, 82, 89, 95, 103, 110, 117, or 137, wherein said RGN polypeptide is capable of binding said target DNA sequence in an RNA-guided sequence specific manner when bound to a guide RNA capable of hybridizing to said target DNA sequence; and
b) said guide RNA;
wherein the RGN polypeptide cleaves non-target ssDNAs of said plurality.

156. The method of embodiment 155, wherein said population of nucleic acids are within a cell lysate.

157. The method of embodiment 155 or 156, wherein said guide RNA comprises a CRISPR repeat sequence comprising a nucleotide sequence having at least 95% sequence identity to SEQ ID NO: 2, 10, 17, 24, 31, 39, 47, 55, 62, 70, 76, 83, 90, 96, 104, 111, 118, or 273.

158. The method of any one of embodiments 155-157, wherein said guide RNA comprises a tracrRNA.

159. The method of embodiment 158, wherein said tracrRNA comprises a nucleotide sequence having at least 95% sequence identity to SEQ ID NO: 3, 11, 18, 25, 32, 40, 48, 56, 63, 71, 77, 84, 91, 97, 105, 112, 119, or 274.

160. The method of embodiment 158 or 159, wherein said guide RNA is a single guide RNA.

161. The method of embodiment 158 or 159, wherein said guide RNA is a dual-guide RNA.

162. A nucleic acid molecule comprising a polynucleotide encoding a CRISPR RNA (crRNA), wherein said crRNA comprises a spacer sequence and a CRISPR repeat sequence, wherein said CRISPR repeat sequence comprises a nucleotide sequence having at least 95% sequence identity to SEQ ID NO: 240;
wherein a guide RNA comprising:
   a) said crRNA; and optionally
   b) a trans-activating CRISPR RNA (tracrRNA) capable of hybridizing to said CRISPR repeat sequence of said crRNA;
is capable of hybridizing to a target DNA sequence of a DNA molecule in a sequence specific manner through the spacer sequence of said crRNA when said guide RNA is bound to an RNA-guided nuclease (RGN) polypeptide, and
wherein said polynucleotide encoding a crRNA is operably linked to a promoter heterologous to said polynucleotide.

163. A vector comprising the nucleic acid molecule of embodiment 162.

164. The vector of embodiment 163, wherein said vector further comprises a polynucleotide encoding said tracrRNA.

165. The vector of embodiment 164, wherein said tracrRNA comprises a nucleotide sequence having at least 95% sequence identity to SEQ ID NO: 241.

166. The vector of embodiment 164 or 165, wherein said polynucleotide encoding said crRNA and said polynucleotide encoding said tracrRNA are operably linked to the same promoter and are encoded as a single guide RNA.

167. The vector of embodiment 164 or 165, wherein said polynucleotide encoding said crRNA and said polynucleotide encoding said tracrRNA are operably linked to separate promoters.

168. The vector of any one of embodiments 163-167, wherein said vector further comprises a polynucleotide encoding said RGN polypeptide, wherein said RGN polypeptide comprises an amino acid sequence having at least 95% sequence identity to SEQ ID NO: 235.

169. A nucleic acid molecule comprising a polynucleotide encoding a trans-activating CRISPR RNA (tracrRNA) comprising a nucleotide sequence having at least 95% sequence identity to SEQ ID NO: 241;
wherein a guide RNA comprising:
   a) said tracrRNA; and
   b) a crRNA comprising a spacer sequence and a CRISPR repeat sequence, wherein said tracrRNA is capable of hybridizing with said CRISPR repeat sequence of said crRNA;
is capable of hybridizing to a target DNA sequence of a DNA molecule in a sequence specific manner through the spacer sequence of said crRNA when said guide RNA is bound to an RNA-guided nuclease (RGN) polypeptide, and
wherein said polynucleotide encoding a tracrRNA is operably linked to a promoter heterologous to said polynucleotide.

170. A vector comprising the nucleic acid molecule of embodiment 169.

171. The vector of embodiment 170, wherein said vector further comprises a polynucleotide encoding said crRNA.

172. The vector of embodiment 171, wherein the CRISPR repeat sequence of said crRNA comprises a nucleotide sequence having at least 95% sequence identity to SEQ ID NO: 240.

173. The vector of embodiment 171 or 172, wherein said polynucleotide encoding said crRNA and said polynucleotide encoding said tracrRNA are operably linked to the same promoter and are encoded as a single guide RNA.

174. The vector of embodiment 171 or 172, wherein said polynucleotide encoding said crRNA and said polynucleotide encoding said tracrRNA are operably linked to separate promoters.

175. The vector of any one of embodiments 170-174, wherein said vector further comprises a polynucleotide encoding said RGN polypeptide, wherein said RGN polypeptide comprises an amino acid sequence having at least 95% sequence identity to SEQ ID NO: 235.

The following examples are offered by way of illustration and not by way of limitation.

### EXAMPLES

### Example 1. Identification of RNA-Guided Nucleases

Seventeen distinct CRISPR-associated RNA-guided nucleases (RGN's) were identified and are described in Table 1 below. Table 1 provides the name of each RGN, its amino acid sequence, the source from which it was derived, and processed crRNA and tracrRNA sequences (see Example 2 for methods of identification). Table 1 further provides a generic single guide RNA (sgRNA) sequence, where the poly-N indicates the location of the spacer sequence which determines the nucleic acid target sequence of the sgRNA. RGN systems APG05733.1, APG06207.1, APG01647.1, APG08032.1, APG02675.1, APG01405.1, APG06250.1, APG04293.1, and APG01308.1) had the conserved sequence in the base of the hairpin stem of the tracrRNA of UNANNA (SEQ ID NO: 8). For APG05712.1, the sequence in the same location is CNANNG (SEQ ID NO: 37). For APG01658.1, the sequence in the same location is CNANNU (SEQ ID NO: 45). For RGN systems APG06498.1 and APG06877.1, the conserved sequence in the base of the hairpin stem of the tracrRNA is UNANNG (SEQ ID NO: 53). For APG09882.1 and APG06646.1, the sequence in the same location is UNANNC (SEQ ID NO: 68). For APG09053.1, the sequence in the same location is CNANNU (SEQ ID NO: 102).

TABLE 1
Summary of SEQ IDs and CRISPR associated systems

| | | | crRNA | | |
|---|---|---|---|---|---|
| | | | repeat | | |
| | SEQ | | seq | tracrRNA | sgRNA |
| | ID | | (SEQ ID | (SEQ ID | (SEQ ID |
| RGN ID | NO | Source | NO) | NO) | NO) |
| APG05733.1 | 1 | Bacillus sp. | 2 | 3 | 4 |
| APG06207.1 | 9 | Chryseobacterium | 10 | 11 | 12 |
| | | sp. | | | |
| APG01647.1 | 16 | Sphingobacterium | 17 | 18 | 19 |
| | | sp. | | | |
| APG08032.1 | 23 | Chryseobacterium | 24 | 25 | 26 |
| | | sp. | | | |
| APG05712.1 | 30 | Brucella sp. | 31 | 32 | 33 |
| APG01658.1 | 38 | Staphylococcus sp. | 39 | 40 | 41 |
| APG06498.1 | 46 | Bacillus sp. | 47 | 48 | 49 |
| APG09106.1 | 54 | Brevibacillus sp. | 55 | 56 | 57 |
| APG09882.1 | 61 | Enterococcus sp. | 62 | 63 | 64 |
| APG02675.1 | 69 | Sphingobacterium | 70 | 71 | 72 |
| | | sp. | | | |
| APG01405.1 | 75 | Chryseobacterium | 76 | 77 | 78 |
| | | sp. | | | |
| APG06250.1 | 82 | Chryseobacterium | 83 | 84 | 85 |
| | | sp. | | | |
| APG06877.1 | 89 | Bacillus sp. | 90 | 91 | 92 |
| APG09053.1 | 95 | Rhizobium sp. | 96 | 97 | 98 |
| APG04293.1 | 103 | Myroides sp. | 104 | 105 | 106 |
| APG01308.1 | 110 | Chryseobacterium | 111 | 112 | 113 |
| | | sp. | | | |
| APG06646.1 | 117 | Bacillus sp. | 118 | 119 | 120 |

### Example 2: Guide RNA Identification and sgRNA Construction

Cultures of bacteria that natively express the RNA-guided nuclease system under investigation were grown to mid-log phase (OD600 of ~0.600), pelleted, and flash frozen. RNA was isolated from the pellets using a mirVANA miRNA Isolation Kit (Life Technologies, Carlsbad, CA), and sequencing libraries were prepared from the isolated RNA using an NEBNext Small RNA Library Prep kit (NEB, Beverly, MA). The library prep was fractionated on a 6% polyacrylamide gel to capture the RNA species less than 200 nt to detect crRNAs and tracrRNAs, respectively. Deep sequencing (75 bp paired-end) was performed on a Next Seq 500 (High Output kit) by a service provider (MoGene, St. Louis, MO). Reads were quality trimmed using Cutadapt and mapped to reference genomes using Bowtie2. A custom RNAseq pipeline was written in python to detect the crRNA and tracrRNA transcripts. Processed crRNA boundaries were determined by sequence coverage of the native repeat spacer array. The anti-repeat portion of the tracrRNA was identified using permissive BLASTn parameters. RNA sequencing depth confirmed the boundaries of the processed tracrRNA by identifying the transcript containing the anti-repeat. Manual curation of RNAs was performed using secondary structure prediction by NUPACK, an RNA folding software. sgRNA cassettes were prepared by DNA synthesis and were generally designed as follows (5′->3′): 20-30 bp spacer sequence, operably linked at its 3′ end to the processed repeat portion of the crRNA, operably linked to a 4 bp noncomplementary linker (AAAG; SEQ ID NO: 123), operably linked at its 3′ end to the processed tracrRNA. Other 4 bp noncomplementary linkers may also be used.

For in vitro assays, sgRNAs were synthesized by in vitro transcription of the sgRNA cassettes with a GeneArt™ Precision gRNA Synthesis Kit (ThermoFisher). Processed crRNA and tracrRNA sequences for each of the RGN polypeptides are identified and are set forth in Table 1. See below for the sgRNAs constructed for PAM libraries 1 and 2.

### Example 3: Determination of PAM Requirements for Each RGN

PAM requirements for each RGN were determined using a PAM depletion assay essentially adapted from Kleinstiver et al. (2015) Nature 523:481-485 and Zetsche et al. (2015) Cell 163:759-771. Briefly, two plasmid libraries (L1 and L2) were generated in a pUC18 backbone (ampR), with each containing a distinct 30 bp protospacer (target) sequence flanked by 8 random nucleotides (i.e., the PAM region). The target sequence and flanking PAM region of library 1 and library 2 for each RGN are set forth in Table 2.

The libraries were separately electroporated into E. coli BL21 (DE3) cells harboring pRSF-1b expression vectors containing an RGN of the invention (codon optimized for E. coli) along with a cognate sgRNA containing a spacer sequence corresponding to the protospacer in L1 or L2. Sufficient library plasmid was used in the transformation reaction to obtain >10⁶ CFU. Both the RGN and sgRNA in the pRSF-1b backbone were under the control of T7 promoters. The transformation reaction was allowed to recover for 1 hr after which it was diluted into LB media containing carbenicillin and kanamycin and grown overnight. The following day the mixture was diluted into self-inducing Overnight Express™ Instant TB Medium (Millipore Sigma) to allow expression of the RGN and sgRNA, and grown for an additional 4 h or 20 h after which the cells were spun down and plasmid DNA was isolated with a Mini-prep kit (Qiagen, Germantown, MD). In the presence of the appropriate sgRNA, plasmids containing a PAM that is recognizable by the RGN will be cleaved resulting in their removal from the population. Plasmids containing PAMs that are not recognizable by the RGN, or that are transformed into bacteria not containing an appropriate sgRNA, will survive and replicate. The PAM and protospacer regions of uncleaved plasmids were PCR-amplified and prepared for sequencing following published protocols (16s-metagenomic library prep guide 15044223B, Illumina, San Diego, CA). Deep sequencing (75 bp single end reads) was performed on a MiSeq (Illumina) by a service provider (MoGene, St. Louis, MO). Typically, 1-4M reads were obtained per amplicon. PAM regions were extracted, counted, and normalized to total reads for each sample. PAMs that lead to plasmid cleavage were identified by being underrepresented when compared to controls (i.e., when the library is transformed into E. coli containing the RGN but lacking an appropriate sgRNA). To represent PAM requirements for a novel RGN, the depletion ratios (frequency in sample/frequency in control) for all sequences in the region in question were converted to enrichment values with α-log base 2 transformation. Sufficient PAMs were defined as those with enrichment values >2.3 (which corresponds to depletion ratios <~ 0.2). PAMs above this threshold in both libraries were collected and used to generate web logos, which for example can be generated using a web-based service on the internet known as “weblogo”. PAM sequences were identified and reported when there was a consistent pattern in the top enriched PAMs. A consensus PAM (having an enrichment factor (EF)>2.3) for each RGN is provided in Table 2. The PAM orientation is also indicated in Table 2. As noted elsewhere in this application, APG06646.1 and APG04293.1 nucleases do not possess PAM-interacting domains. The results in Table 2 show that they also do not possess the typical PAM requirement, which is 2-5 nucleotides. APG06646.1 and APG04293.1 were shown to have single nucleotide requirements for cleavage.

TABLE 2
PAM or PAM-like determination

| | sgRNA L1 | sgRNA L2 | PAM | |
|---|---|---|---|---|
| | (SEQ ID | (SEQ ID | (SEQ | |
| RGN ID | NO) | NO) | ID NO) | PAM orientation |
| APG05733.1 | 5 | 6 | 7 | 5′-target-PAM-3′ |
| APG06207.1 | 13 | 14 | 15 | 5′-target-PAM-3′ |
| APG01647.1 | 20 | 21 | 22 | 5′-target-PAM-3′ |
| APG08032.1 | 27 | 28 | 29 | 5′-target-PAM-3′ |
| APG05712.1 | 34 | 35 | 36 | 5′-target-PAM-3′ |
| APG01658.1 | 42 | 43 | 44 | 5′-target-PAM-3′ |
| APG06498.1 | 50 | 51 | 52 | 5′-target-PAM-3′ |
| APG09106.1 | 58 | 59 | 60 | 5′-PAM-target-3′ |
| APG09882.1 | 65 | 66 | 67 | 5′-target-PAM-3′ |
| APG02675.1 | 73 | 74 | 15 | 5′-target-PAM-3′ |
| APG01405.1 | 79 | 80 | 81 | 5′-target-PAM-3′ |
| APG06250.1 | 86 | 87 | 88 | 5′-target-PAM-3′ |
| APG06877.1 | 93 | 94 | 7 | 5′-target-PAM-3′ |
| APG09053.1 | 99 | 100 | 101 | 5′-target-PAM-3′ |
| APG04293.1 | 107 | 108 | 109 | 5′-target-PAM-3′ |
| APG01308.1 | 114 | 115 | 116 | 5′-target-PAM-3′ |
| APG06646.1 | 121 | 122 | 109 | 5′-target-PAM-3′ |

### Example 4: Engineering the Guide RNA to Increase Nuclease Activity

### 4.1 RGNs APG09748 and APG09106.1

For RGNs APG09748 (set forth as SEQ ID NO: 137, and APG09748 crRNA repeat sequence, tracrRNA sequence, and generic sgRNA sequence are set forth as SEQ ID NOs: 273, 274, and 275, respectively; all sequences are described in International Appl. No. PCT/US2019/068079, which is incorporated by reference in its entirety) and APG09106.1, which have very high sequence identity and have the same PAM, RNA folding predictions were used to determine regions in the guide RNA that can be altered to optimize nuclease activity. The stability of the crRNA: tracrRNA base pairing in the repeat:antirepeat region was increased by shortening the repeat:antirepeat region, adding G-C base pairs, and removing G-U wobble pairs. “Optimized” guide variants were tested and compared to the wild-type gRNA using the RGN APG09748 in in vitro cleavage assays.

To produce RGNs for RNP formation, expression plasmids containing an RGN fused to a C-terminal His6 (SEQ ID NO: 276) or His10 (SEQ ID NO: 277) tag were constructed and transformed into BL21 (DE3) strains of E. coli. Expression was performed using Magic Media (Thermo Fisher) supplemented with 50 μg/mL kanamycin. After lysis and clarification, the protein was purified by immobilized metal affinity chromatography and quantified using the Qubit protein quantitation kit (Thermo Fisher) or by UV-vis using a calculated extinction coefficient.

Ribonucleoprotein (RNP) was prepared by incubating the purified RGN with sgRNA at a ~2:1 ratio for 20 min at room temperature. For in vitro cleavage reactions, RNPs were incubated with plasmids or linear dsDNA containing the targeted protospacer flanked by a preferred PAM sequence for >30 min at room temperature. Two target nucleic acid sequences within the TRAC locus, TRAC11 (SEQ ID NO: 278) and TRAC14 (SEQ ID NO: 279), were tested. gRNAs were assayed both for targeted activity with the correct target nucleic acid sequence (for example, the gRNA has TRAC11 spacer sequence and the assayed target is TRAC11) and without the correct target nucleic acid sequence (for example, the gRNA has TRAC11 spacer sequence and the assayed target is TRAC14). Activity determined by plasmid cleavage is assessed by agarose gel electrophoresis. Results are shown in Table 3. Guide variants are listed as SEQ ID NOs: 280-283, and are provided with spacer sequences. These guide sequences use a noncomplementary nucleotide linker of AAAA (SEQ ID NO: 284). The optimized gRNA (SEQ ID NO: 285; poly-N indicates location of spacer sequence), with increased repeat:antirepeat binding, has optimized tracrRNA (SEQ ID NO: 286) and optimized crRNA (SEQ ID NO: 287) components. The optimized guide variant was able to cleave two loci where previously no cleavage was detected using the wild-type guide RNA. Through optimization of hybridization in the repeat:antirepeat region, in vitro cleavage of APG09748 increased from 0% cleavage to 100% cleavage for multiple targets in the TRAC locus.

TABLE 3
Editing efficiency of APG09748 with engineered guide variants

| gRNA variant | Guide | Assayed | Gel 1 - 2 μL load | | Gel 2 - 1 μL load | |
|---|---|---|---|---|---|---|
| (SEQ ID NO.) | Design | Target | % intact | % cleaved | % intact | % cleaved |
| 280 | Optimized | TRAC11 | 68 | 32 | 57 | 43 |
| 280 | Optimized | TRAC14 | 100 | 0 | 100 | 0 |
| 281 | Optimized | TRAC11 | 100 | 0 | 100 | 0 |
| 281 | Optimized | TRAC14 | 70 | 30 | 69 | 31 |
| 282 | WT | TRAC11 | 100 | 0 | 100 | 0 |
| 282 | WT | TRAC14 | 100 | 0 | 100 | 0 |
| 283 | WT | TRAC11 | 100 | 0 | 100 | 0 |
| 283 | WT | TRAC14 | 100 | 0 | 100 | 0 |
| None | | TRAC11 | 100 | 0 | 100 | 0 |
| None | | TRAC14 | 100 | 0 | 100 | 0 |

Additional optimized gRNA variants were designed and assayed. Further, different lengths of spacer sequence were also tested to determine how spacer length might affect cleavage efficiency. The sgRNA outside of the spacer sequence is referred to as the “backbone” in this assay. In Table 4, these are denoted as “WT” (SEQ ID NO: 288, the wild type sequence), and the three optimized sgRNAs: VI (SEQ ID NO: 289), V2 (SEQ ID NO: 290) and V3 (SEQ ID NO: 291). All of these sequences have a poly-N to indicate the location of the spacer sequence. Guides were expressed as sgRNAs by in vitro transcription (IVT). Compared to the wild-type sgRNA backbone, VI is 87.8% identical, V2 is 92.4% identical, and V3 is 85.5% identical. Synthetic tracrRNA:crRNA duplexes (“synthetic”) representing dual-guide RNAs but otherwise similar to the wild type and optimized sgRNAs recited above were also produced and tested.

For this set of assays, RGN APG09106.1 was used; otherwise, methods for in vitro cleavage reactions were similar to what is described above. The targeted nucleic acid sequences were Target 1 (SEQ ID NO: 292) and Target 2 (SEQ ID NO: 293). The results are shown in Table 4.

TABLE 4
Editing efficiency of APG09106.1 with engineered
guide variants

| | | | | Spacer | |
|---|---|---|---|---|---|
| RNA | | Spacer | | SEQ ID | Cleavage |
| Source | Target | Length | Backbone | NO. | % |
| Synthetic | 2 | 18 | WT | 294 | 12.3 |
| Synthetic | 1 | 20 | WT | 295 | 0 |
| Synthetic | 2 | 20 | WT | 296 | 55.0 |
| Synthetic | 1 | 25 | WT | 297 | 0 |
| Synthetic | 2 | 25 | WT | 298 | 61.4 |
| IVT | 2 | 25 | VI | 299 | 1.1 |
| IVT | 2 | 25 | V2 | 300 | 0.9 |
| IVT | 2 | 25 | V3 | 301 | 0.7 |
| IVT | 2 | 20 | V3 | 302 | 21.0 |
| IVT | 1 | 25 | V3 | 303 | 2.0 |

### 4.2 RGN APG07433.1

RNA folding predictions were used to determine regions in the guide RNA for APG07433.1 (set forth as SEQ ID NO: 235 and described in U.S. Appl. Publ. No. 2019/0367949 and International Appl. Publ. No. WO 2019/236566, each of which is herein incorporated by reference in its entirety) that can be altered to optimize nuclease activity and shorten the guide RNA for packaging into viral vectors. Two areas were identified as potential locations to make alterations: the repeat:antirepeat pairing between the crRNA and tracrRNA region and the terminal hairpins in the tracrRNA. The repeat:antirepeat region was truncated to 7 (APG07433.1-7 bp, SEQ ID NOs: 238 and 239), 13 (APG07433.1-13 bp, SEQ ID NOs: 250 and 251), and 15 base pairs in length (APG07433.1-15 bp, SEQ ID NOs: 242 and 243). Additionally, a fourth variant was tested that altered the sequence of the repeat:antirepeat region and reduced the pairing to 11 basepairs in length (APG07433.1-11 bp-syn, SEQ ID NOs: 240 and 241), with an introduction of a smaller RNA bulge compared to the wildtype guide. Truncations were also made to the terminal hairpins in the tracrRNA, including a reduction of the native sequence to 40 (APG07433.1-40ntTHP, SEQ ID NOs: 254 and 255) and 42 (APG07433.1-42ntTHP, SEQ ID NOs: 244 and 245) nucleotides in length. Altered stem-loops were designed to shorten these to 35 (APG07433.1-35ntTHP-syn, SEQ ID NOs: 248 and 249) and 39 (APG07433.1-39ntTHP-syn, SEQ ID NOs: 246 and 247) nucleotides as well. One guide combined the 13-basepair shortened repeat:antirepeat region and the 42-nucleotide shortened terminal hairpin (APG07433.1-13bp42ntTH, SEQ ID NOs: 252 and 253). The effects of spacer length on cleavage was also tested. Spacer lengths of 25 (APG07433.1-native [25], SEQ ID NOs: 236 and 237; spacer sequence is set forth as SEQ ID NO: 271) and 18 (APG07433.1-native [18], SEQ ID NOs: 256 and 257; spacer sequence is set forth as SEQ ID NO: 272) nucleotides on the wild type guide RNA backbone were also tested.

The following dual guide RNAs were prepared by annealing crRNAs and tracrRNAs by preparing a solution containing 20 UM crRNA and 10 μM tracrRNA in annealing buffer (Synthego), then heating to 78 °C for 10 min and then at 37 °C for 30 min. RNPs were formed by incubation with purified APG07433.1 protein at 0.5 UM and the dual guide RNA at 1 μM in phosphate buffered saline for 20 minutes. The guide RNAs used in this experiment are shown in Table 5.

TABLE 5
Dual guide RNAs

| | crRNA | tracrRNA |
|---|---|---|
| Dual gRNA Name | SEQ ID NO | SEQ ID NO |
| APG07433.1-native[25] | 236 | 237 |
| APG07433.1-7bp | 238 | 239 |
| APG07433.1-11bp-syn | 240 | 241 |
| APG07433.1-15bp | 242 | 243 |
| APG07433.1-42ntTHP | 244 | 245 |
| APG07433.1-39ntTHP-syn | 246 | 247 |
| APG07433.1-35ntTHP-syn | 248 | 249 |
| APG07433.1-13bp | 250 | 251 |
| APG07433.1-13bp42ntTH | 252 | 253 |
| APG07433.1-40ntTHP | 254 | 255 |
| APG07433.1-native[18] | 256 | 257 |
| APG07433.1cr-13bp tr-35ntTHP | 258 | 259 |

These RNPs were incubated with a PCR product containing the appropriate target sequence (SEQ ID NO: 260) produced by amplification using FAM and Cy3 labeled primers, to facilitate accurate and sensitive quantitation of cleavage product. The reactions contained 250 nM RNP, produced as above, and 150 nM of the FAM and Cy3 labeled PCR product in 1× Cutsmart buffer (New England Biolabs). The reaction was allowed to proceed for 15 min at 37 °C and was terminated by adding RNase A to 0.1 mg/mL and EDTA to 45 mM. The quenched reaction was heated at 50 °C for 30 min and 95 °C for 5 minutes. The samples were then analyzed using native acrylamide gel electrophoresis on a 5% TBE gel (Bio-rad). These were imaged on a ChemiDoc MP imager. Each of the two dyes could be used for quantitation, and each sample was performed in duplicate. The results of this experiment are shown in Table 6.

TABLE 6
Cleavage results

| | % Cleaved | | | | |
|---|---|---|---|---|---|
| | Cy3 Channel | | FAM channel | | Overall |
| RNP | Rep 1 | Rep 2 | Rep 1 | Rep 2 | average |
| Nuclease only | 0 | 0 | 0 | 0 | 0 |
| (no guide) | | | | | |
| APG07433.1-native | 66.41 | 61.16 | 68.81 | 56.85 | 63.3 |
| [25] | | | | | |
| APG07433.1-7bp | 0 | 3.38 | 2.83 | 0 | 1.6 |
| APG07433.1-11bp-syn | 66.33 | 76.38 | 66.73 | 68 | 69.4 |
| APG07433.1-15bp | 50.7 | 65.61 | 54.62 | 59.95 | 57.7 |
| APG07433.1-42ntTHP | 44.81 | 50.59 | 48.84 | 51.77 | 49.0 |
| APG07433.1-39ntTHP- | 19.16 | 32.88 | 24.91 | 33.43 | 27.6 |
| syn | | | | | |
| APG07433.1-35ntTHP- | 26.77 | 38.61 | 32.09 | 40.2 | 34.4 |
| syn | | | | | |
| APG07433.1-13bp | 44.36 | 33.03 | 42.93 | 35.22 | 38.9 |
| APG07433.1- | 35.91 | 36.74 | 33.84 | 39.2 | 36.4 |
| 13bp42ntTHP | | | | | |
| APG07433.1-40ntTHP | 44.52 | 57.14 | 48.34 | 57.84 | 52.0 |
| APG07433.1-native | 11.1 | 9.76 | 12.93 | 13.24 | 11.8 |
| [18] | | | | | |
| APG07433.1cr-13bp | 10.66 | 12.25 | 12.65 | 13.54 | 12.3 |
| tr-35ntTHP | | | | | |

This analysis demonstrates that the native backbone outperforms most of the shortened variants, and a variant containing a shortened target sequence (APG07433.1-native [18], SEQ ID NOs: 256 and 257). Of the shortened backbone sequences, that with the highest level of cleavage is the sequence named APG07433.1-11 bp-syn, which comprises a 25 nt targeting sequence (spacer; SEQ ID NO: 271), SEQ ID NO: 240 for the crRNA and SEQ ID NO: 241 as the tracrRNA. This guide variant included an altered repeat:antirepeat stem loop with an engineered bulge in the stem.

### Example 5: Demonstration of Gene Editing Activity in Mammalian Cells

RGN expression cassettes were produced and introduced into vectors for mammalian expression. RGNs APG09748, APG09106.1, APG05712.1, APG01658.1, APG05733.1, APG06498.1, APG06646.1, APG09882.1, APG01405.1, and APG01308.1 were each codon-optimized for mammalian expression (SEQ ID NOs: 304, 305, and 411-418, respectively), and the expressed proteins were operably fused at the N-terminal end to an SV40 nuclear localization sequence (NLS; SEQ ID NO: 125) and to 3×FLAG tags (SEQ ID NO: 126), and operably fused at the C-terminal end to nucleoplasmin NLS sequences (SEQ ID NO: 127). Two copies of the NLS sequence were used, operably fused in tandem. Each expression cassette was under control of a cytomegalovirus (CMV) promoter (SEQ ID NO: 306). It is known in the art that the CMV transcription enhancer (SEQ ID NO: 307) may also be included in constructs comprising the CMV promoter. Guide RNA expression constructs encoding a single gRNA each under the control of a human RNA polymerase III U6 promoter (SEQ ID NO: 308) were produced and introduced into an expression vector. Guides targeted regions of selected genes, including RelA, AurkB, GAPDH, LINC01509, HBB, CFTR, HPRT1, TRA, EMX1, and VEGFA, as shown in Table 7. For RNA-guided nuclease APG09106.1, specific residues were mutated to increase nuclease activity of the protein, specifically the T849 residue of APG09106 was mutated to arginine (SEQ ID NO: 309). This point mutation increased editing rates in mammalian cells.

The constructs described above were introduced into mammalian cells. One day prior to transfection, 1×10⁵ HEK293T cells (Sigma) were plated in 24-well dishes in Dulbecco's modified Eagle medium (DMEM) plus 10% (vol/vol) fetal bovine serum (Gibco) and 1% Penicillin-Streptomycin (Gibco). The next day when the cells were at 50-60% confluency, 500 ng of a RGN expression plasmid plus 500 ng of a single gRNA expression plasmid were co-transfected using 1.5 μL of Lipofectamine 3000 (Thermo Scientific) per well, following the manufacturer's instructions. After 48 hours of growth, total genomic DNA was harvested using a genomic DNA isolation kit (Machery-Nagel) according to the manufacturer's instructions.

The total genomic DNA was then analyzed to determine the rate of editing in the targeted gene. Oligonucleotides were produced to be used for PCR amplification and subsequent analysis of the amplified genomic target site (SEQ ID NOs: 310 and 311). All PCR reactions were performed using 10 μL of 2× Master Mix Phusion High-Fidelity DNA polymerase (Thermo Scientific) in a 20 μL reaction including 0.5 μM of each primer. Large genomic regions encompassing each target gene were first amplified using PCR #1 primers (SEQ ID NOs: 310 and 311), using a program of: 98 °C, 1 min; 30 cycles of [98 °C, 10 sec; 62 °C, 15 sec; 72 °C, 5 min]; 72 °C, 5 min; 12 °C, forever.

One microliter of this PCR reaction was then further amplified using primers specific for each guide (PCR #2 primers; SEQ ID NOs: 365-370), using a program of: 98 °C, 1 min; 35 cycles of [98 °C, 10 sec; 67 °C, 15 sec; 72 °C, 30 sec]; 72 °C, 5 min; 12 °C, forever. Primers for PCR #2 include Nextera Read 1 and Read 2 Transposase Adapter overhang sequences for Illumina sequencing.

Following the second PCR amplification, DNA was cleaned using a PCR cleanup kit (Zymo) according to the manufacturer's instructions and eluted in water. 200-500 ng of purified PCR #2 product was combined with 2 μL of 10×NEB Buffer 2 and water in a 20 μL reaction and annealed to form heteroduplex DNA using a program of: 95 °C, 5 min; 95-85 °C, cooled at a rate of 2 °C/see; 85-25 °C, cooled at a rate of 0.1 °C/sec.; 12 °C, forever. Following annealing, 5 μL of DNA was removed as a no enzyme control, and 1 μL of T7 Endonuclease I (NEB) was added and the reaction incubated at 37 °C for 1 hr. After incubation, 5× FlashGel loading dye (Lonza) was added and 5 μL of each reaction and controls were analyzed by a 2.2% agarose FlashGel (Lonza) using gel electrophoresis. Following visualization of the gel, the percentage of non-homologous end joining (NHEJ) was determined using the following equation: % NHEJ events=100×[1-(1-fraction cleaved)(½)], where (fraction cleaved) is defined as: (density of digested products)/(density of digested products+undigested parental band).

For some samples, SURVEYOR® was used to analyze the results following expression in mammalian cells. Cells were incubated at 37 °C for 72 h post-transfection before genomic DNA extraction. Genomic DNA was extracted using the QuickExtract DNA Extraction Solution (Epicentre) following the manufacturer's protocol. The genomic region flanking the RGN target site was PCR amplified, and products were purified using QiaQuick Spin Column (Qiagen) following the manufacturer's protocol. 200-500 ng total of the purified PCR products were mixed with 1 μl 10× Taq DNA Polymerase PCR buffer (Enzymatics) and ultrapure water to a final volume of 10 μl, and subjected to a re-annealing process to enable heteroduplex formation: 95 °C for 10 min, 95 °C to 85 °C ramping at −2 °C/s, 85 °C to 25 °C at −0.25 °C/s, and 25 °C hold for 1 min.

After reannealing, products were treated with SURVEYOR® nuclease and SURVEYOR® enhancer S (Integrated DNA Technologies) following the manufacturer's recommended protocol and analyzed on 4-20% Novex TBE polyacrylamide gels (Life Technologies). Gels were stained with SYBR Gold DNA stain (Life Technologies) for 10 min and imaged with a Gel Doc gel imaging system (Bio-rad). Quantification was based on relative band intensities. Indel percentage was determined by the formula, 100×(1−(1−(b+c)/(a+b+c))½), where a is the integrated intensity of the undigested PCR product, and b and c are the integrated intensities of each cleavage product.

Additionally, products from PCR #2 containing Illumina overhang sequences underwent library preparation following the Illumina 16S Metagenomic Sequencing Library protocol. Deep sequencing was performed on an Illumina Mi-Seq platform by a service provider (MOGene). Typically, 200,000 of 250 bp paired-end reads (2×100,000 reads) are generated per amplicon. The reads were analyzed using CRISPResso (Pinello, et al. 2016 Nature Biotech, 34:695-697) to calculate the rates of editing. Output alignments were hand-curated to confirm insertion and deletion sites as well as identify microhomology sites at the recombination sites. The overall rates of editing, as well as the deletion rate and insertion rate in each sample, are shown in Table 7. All experiments were performed in human cells. The “target” is the targeted sequence within the gene target. For each target sequence, the guide RNA comprised the complementary RNA spacer sequence and the appropriate sgRNA depending on the RGN used. A selected breakdown of experiments by guide RNA is shown in Tables 8.1 and 8.2.

TABLE 7
Overall rates of editing for gene targets

| | | | | Overall | | |
|---|---|---|---|---|---|---|
| | Gene | Guide RNA | Target | Editing | Deletion | Insertion |
| RGN | Target | (SEQ ID NO.) | (SEQ ID NO.) | Rate | Rate | Rate |
| APG09106.1 | AurkB | 316 | 314 | 0.55% | 100% | |
| APG09106.1 | AurkB | 312 | 315 | 0.60% | 54% | 46% |
| APG09106.1 | AurkB | 316 | 314 | 2.97% | 98% | 2.00% |
| T849R | | | | | | |
| APG09106.1 | AurkB | 312 | 315 | 2.36% | 100% | |
| T849R | | | | | | |
| APG05712.1 | RelA | 419 | 482 | 0.36% | 72.33% | 27.67% |
| APG05712.1 | AurkB | 420 | 483 | 1.39% | 25.94% | 75.12% |
| APG05712.1 | RelA | 421 | 484 | 1.04% | 32.10% | 70.33% |
| APG05712.1 | RelA | 422 | 485 | 0.28% | 64.13% | 35.87% |
| APG01658.1 | RelA | 423 | 486 | 10.03% | 70.66% | 29.33% |
| APG01658.1 | GAPDH | 424 | 487 | 5.60% | 76.96% | 25.97% |
| APG01658.1 | LINC01509 | 425 | 488 | 9.87% | 75.10% | 26.61% |
| APG01658.1 | HBB | 426 | 489 | 0.42% | 63.86% | 36.13% |
| APG01658.1 | CFTR | 427 | 490 | 6.06% | 75.42% | 27.86% |
| APG01658.1 | AurkB | 428 | 491 | 13.07% | 88.02% | 13.14% |
| APG05733.1 | HPRT1 | 429 | 492 | 0.36% | | 100% |
| APG05733.1 | AurkB | 430 | 493 | 5.01% | 86.40% | 13.60% |
| APG05733.1 | RelA | 431 | 494 | 0.76% | | 100% |
| APG05733.1 | HPRT1 | 432 | 495 | 0.17% | 38.42% | 61.58% |
| APG05733.1 | RelA | 433 | 496 | 0.96% | 77.52% | 22.48% |
| APG06498.1 | TRA | 434 | 497 | 35.35% | 92.25% | 8.29% |
| APG06498.1 | TRA | 435 | 498 | 21.04% | 84.87% | 16.34% |
| APG06498.1 | TRA | 436 | 499 | 10.16% | 89.44% | 10.69% |
| APG06498.1 | TRA | 437 | 500 | 13.21% | 87.24% | 15.00% |
| APG06498.1 | TRA | 438 | 501 | 13.56% | 86.73% | 13.66% |
| APG06498.1 | TRA | 439 | 502 | 9.62% | 95.85% | 19.90% |
| APG06498.1 | TRA | 440 | 503 | 1.51% | 75.89% | 24% |
| APG06498.1 | TRA | 441 | 504 | 2.63% | 88.81% | 11.76% |
| APG06498.1 | TRA | 442 | 505 | 4.27% | 52.09% | 47.90% |
| APG06646.1 | TRA | 443 | 497 | 0.33% | 0% | 100% |
| APG06646.1 | VEGFA | 444 | 506 | 4.52% | 40.83% | 63.79% |
| APG06646.1 | AurkB | 445 | 507 | 0.66% | 69.38% | 30.62% |
| APG06646.1 | AurkB | 446 | 508 | 6.55% | 79.97% | 20.37% |
| APG06646.1 | TRA | 447 | 509 | 2.47% | 73.40% | 40.81% |
| APG06646.1 | TRA | 448 | 510 | 0.14% | 34.46% | 65.55% |
| APG06646.1 | TRA | 449 | 511 | 0.61% | 0% | 100% |
| APG06646.1 | AurkB | 450 | 493 | 7.94% | 67.95% | 32.40% |
| APG06646.1 | TRA | 451 | 512 | 7.03% | 86.95% | 14.03% |
| APG06646.1 | TRA | 452 | 513 | 0.50% | 21.80% | 78.10% |
| APG06646.1 | TRA | 453 | 514 | 3.53% | 86.42% | 16.28% |
| APG06646.1 | RelA | 454 | 515 | 6.22% | 80.39% | 45.34% |
| APG06646.1 | TRA | 455 | 516 | 0.38% | 0% | 100% |
| APG06646.1 | TRA | 456 | 517 | 0.34% | 66.76% | 48.22% |
| APG06646.1 | RelA | 457 | 518 | 3.78% | 75.25% | 67.90% |
| APG06646.1 | TRA | 458 | 519 | 0.42% | 69.61% | 30.39% |
| APG06646.1 | TRA | 459 | 520 | 0.08% | 100.00% | 0.00% |
| APG06646.1 | AurkB | 460 | 521 | 1.09% | 30.18% | 69.81% |
| APG06646.1 | AurkB | 461 | 522 | 0.28% | 86.60% | 21.78% |
| APG06646.1 | AurkB | 462 | 523 | 10.92% | 78.87% | 22.05% |
| APG06646.1 | TRA | 463 | 504 | 0.08% | 0% | 100% |
| APG06646.1 | TRA | 464 | 505 | 2.84% | 38.26% | 61.47% |
| APG06646.1 | TRA | 465 | 524 | 0.43% | 71.67% | 28.33% |
| APG06646.1 | TRA | 466 | 525 | 0.03% | 0.00% | 100.00% |
| APG06646.1 | TRA | 467 | 526 | 0.08% | 100% | 0% |
| APG09882.1 | RelA | 468 | 482 | 0.32% | 100.00% | 0.00% |
| APG09882.1 | EMX1 | 469 | 527 | 12.84% | 82.46% | 17.79% |
| APG09882.1 | VEGFA | 470 | 528 | 14.76% | 92.56% | 7.77% |
| APG09882.1 | TRA | 471 | 529 | 14.66% | 94.19% | 7.33% |
| APG09882.1 | TRA | 472 | 530 | 7.84% | 95.07% | 5.75% |
| APG09882.1 | VEGFA | 473 | 531 | 24.45% | 88.96% | 11.57% |
| APG09882.1 | TRA | 474 | 532 | 14.43% | 90.24% | 10.96% |
| APG01405.1 | RelA | 475 | 482 | 0.06% | 0% | 100% |
| APG01405.1 | AurkB | 476 | 533 | 6.81% | 80.58% | 20.27% |
| APG01405.1 | AurkB | 477 | 534 | 0.54% | 33.83% | 66.16% |
| APG01405.1 | HPRT1 | 478 | 535 | 1.07% | 42.14% | 57.87% |
| APG01405.1 | RelA | 479 | 484 | 0.55% | 2.07% | 97.93% |
| APG01308.1 | HPRT1 | 480 | 536 | 1.19% | 80.57% | 19.43% |
| APG01308.1 | AurkB | 481 | 537 | 0.90% | 94.97% | 6.46% |

Specific insertions and deletions for respective guides are shown in Tables 8.1 and 8.2. In these tables, the target sequence is identified by bold upper case letters. The 8mer PAM regions are double underlined, with the main recognized nucleotides in bold. Insertions are identified by lowercase letters. Deletions are indicated with dashes ( - - - ). The INDEL location is calculated from the PAM proximal edge of the target sequence, with the edge being location 0. The location is positive (+) if the location is on the target side of the edge; the location is negative (−) if the location is on the PAM side of the edge.

TABLE 8.1
Specific insertions and deletions for Guide 831 using RGN APG09106.1

| | # | % | % of | | INDEL | |
|---|---|---|---|---|---|---|
| Edited target sequence | Reads | Reads | INDELs | Type | Location | Size |
| GTCTGATTGCCTGTCGTTGCCCCTCCCAGATCAT | 92294 | 99.40 | | | | |
| GGAGGAGTTGGCAGA (wild type; SEQ ID | | | | | | |
| NO: 316) | | | | | | |
| GTCTGATTGCCTGTCGTTGCCCCTCCCA------ | 263 | 0.28 | 54.22 | Deletion | +19 | 8 |
| --AGGAGTTGGCAGA (SEQ ID NO: 317) | | | | | | |
| GTCTGATTGCCTGTCGTTGCCCctaagtgtatta | 222 | 0.24 | 45.77 | Insertion | +13 | 20 |
| agcattgtctcagagattttGGAGGAGTTGGCAG | | | | | | |
| A (SEQ ID NO: 318) | | | | | | |

TABLE 8.2
Specific insertions and deletions for Guide 831 using APG09106.1 T849R

| | # | % | % of | | INDEL | |
|---|---|---|---|---|---|---|
| Edited target sequence | Reads | Reads | INDELs | Type | Location | Size |
| GTCTGATTGCCTGTCGTTGCCCCTCCCAGATCAT | 189881 | 97.64 | | | | |
| GGAGGAGTTGGCAGA (wild type; SEQ ID | | | | | | |
| NO: 316) | | | | | | |
| GTCTGATTGCCCTGTCGTTGCCCC---------- | 602 | 0.309 | 13.129 | Deletion | +14 | 10 |
| TGGAGGAGTTGGCAGA (SEQ ID NO: 319) | | | | | | |
| GTCTGATTGCCTGTCGTTGCCCCTCCCAGATC- | 394 | 0.202 | 8.593 | Deletion | +23 | 2 |
| GGAGGAGTTGGCAGA (SEQ ID NO: 320) | | | | | | |
| GTCTGATTGCCTGTCGTTGCCCCTCCCAGAT--- | 399 | 0.205 | 8.702 | Deletion | +22 | 5 |
| --AGGAGTTGGCAGA (SEQ ID NO: 321) | | | | | | |
| GTCTGATTGCCTGTCGTTGCCCaTC-------- | 379 | 0.194 | 8.266 | Deletion & | +16 | 10 |
| TG GGAGTTGGCAGA (SEQ ID NO: 322) | | | | Mutation | | |
| GTCTGATTGCCTGTCGTTGCCCCTC-------- | 350 | 0.179 | 7.633 | Deletion | +16 | 8 |
| TGGAGGAGTTGGCAGA (SEQ ID NO: 323) | | | | | | |
| GTCTGAT-------------------------- | 309 | 0.158 | 6.739 | Deletion | −1 | 26 |
| TGGAGGAGTTGGCAGA (SEQ ID NO: 324) | | | | | | |
| GTCTGATTGCCTGTCGTTGCCCCTC--------- | 280 | 0.143 | 6.106 | Deletion | +16 | 9 |
| GGAGGAGTTGGCAGA (SEQ ID NO: 325) | | | | | | |
| GTCTGATTGCCTGTCGTTGCCCCTCC------- | 274 | 0.140 | 5.976 | Deletion & | +17 | 7 |
| aGGAGGAGTTGGCAGA (SEQ ID NO: 326) | | | | Mutation | | |
| GTCTGATTGCCTGTCGTTGCCC------------ | 251 | 0.129 | 5.474 | Deletion | +13 | 15 |
| GGAGTTGGCAGA (SEQ ID NO: 327) | | | | | | |
| GTCTGATTGCCTGTCGTTGCCC------- | 250 | 0.128 | 5.452 | Deletion | +13 | 7 |
| ATCATGGAGGAGTTGGCAGA (SEQ ID NO: | | | | | | |
| 328) | | | | | | |
| GTCTGATTGCCTGTCGTTGCCCCTC------ | 231 | 0.118 | 5.038 | Deletion | +16 | 6 |
| CATGGAGGAGTTGGCAGA (SEQ ID NO: | | | | | | |
| 329) | | | | | | |
| GTCTGATTGCCTGTCGTTGCCCCTCCCA------ | 218 | 0.112 | 4.754 | Deletion | +19 | 30 |
| ------------------------GTACT (SEQ | | | | | | |
| ID NO: 330) | | | | | | |
| GTCTGATTGCCTGTCGTTGCCCC----- | 206 | 0.105 | 4.492 | Deletion & | +14 | 5 |
| aATCtTGGAGGAGTTGGCAGA (SEQ ID NO: | | | | Mutation | | |
| 331) | | | | | | |
| GTCTGATTGCCTGTCGTTGCCC-------- | 162 | 0.083 | 3.533 | Deletion & | +13 | 8 |
| TgggATGGAGGAGTTGGCAGA (SEQ ID NO: | | | | Mutation | | |
| 332) | | | | | | |
| GTCTGATTGCCTGTCGTTGCCCCTC--------- | 158 | 0.081 | 3.446 | Deletion | +16 | 14 |
| -----AGTTGGCAGA (SEQ ID NO: 333) | | | | | | |
| GTCTGATTGCCTGTCGTTGCCCC------- | 122 | 0.062 | 2.660 | Deletion | +14 | 7 |
| TCATGGAGGAGTTGGCAGA (SEQ ID NO: | | | | | | |
| 334) | | | | | | |

### Example 6: Demonstration of Gene Editing Activity in Plant Cells

RNA-guided nuclease activity of an RGN of the invention is demonstrated in plant cells using protocols adapted from Li, et al., 2013 (Nat. Biotech. 31:688-691). Briefly, a plant codon optimized version of an RGN of the invention (SEQ ID NO: 1, 9, 16, 23, 30, 38, 46, 54, 61, 69, 75, 82, 89, 95, 103, 110, or 117) operably linked to a nucleic acid sequence encoding for an N-terminal SV40 nuclear localization signal are cloned behind the strong constitutive 35S promoter in a transient transformation vector. sgRNAs targeting one or more sites in the plant PDS gene that flank an appropriate PAM sequence are cloned behind a plant U6 promoter in a second transient expression vector. The expression vectors are introduced into Nicotiana benthamiana mesophyll protoplasts using PEG-mediated transformation. The transformed protoplasts are incubated in the dark for up to 36 hr. Genomic DNA is isolated from the protoplasts using a DNeasy Plant Mini Kit (Qiagen). The genomic region flanking the RGN target site is PCR amplified, and products are purified using QiaQuick Spin Column (Qiagen) following the manufacturer's protocol. 200-500 ng total of the purified PCR products are mixed with 1 μl 10× Taq DNA Polymerase PCR buffer

(Enzymatics) and ultrapure water to a final volume of 10 μl, and subjected to a re-annealing process to enable heteroduplex formation: 95 °C for 10 min, 95 °C to 85 °C ramping at −2 °C/s, 85 °C to 25 °C at −0.25 °C/s, and 25 °C hold for 1 min.

After reannealing, products are treated with SURVEYOR nuclease and SURVEYOR enhancer S (Integrated DNA Technologies) following the manufacturer's recommended protocol and analyzed on 4-20% Novex TBE polyacrylamide gels (Life Technologies). Gels are stained with SYBR Gold DNA stain (Life Technologies) for 10 min and imaged with a Gel Doc gel imaging system (Bio-rad). Quantification is based on relative band intensities. Indel percentage is determined by the formula, 100× (1−(1−(b+c)/(a+b+c))½), where a is the integrated intensity of the undigested PCR product, and b and c are the integrated intensities of each cleavage product.

### Example 7: Identification of Disease Targets

A database of clinical variants was obtained from NCBI Clin Var database, which is available through the world wide web at the NCBI Clin Var website. Pathogenic Single Nucleotide Polymorphisms (SNPs) were identified from this list. Using the genomic locus information, CRISPR targets in the region overlapping and surrounding each SNP were identified. A selection of SNPs that can be corrected using base editing in combination with an RGN system comprising APG09748 or APG09106.1 to target the causal mutation (“Casl Mut.”) is listed in Table 9.1. A selection of SNPs that can be corrected using base editing in combination with an RGN system comprising APG06646.1 or APG01658.1 to target the causal mutation (“Casl Mut.”) is listed in Table 9.2. In both Table 9.1 and 9.2, only one alias of each disease is listed. The “RS #” corresponds to the RS accession number through the SNP database at the NCBI website. The Chromosome Accession number provides accession reference information found through the NCBI website. Tables 9.1 and 9.2 also provide genomic target sequence information suitable for RGN systems APG09748 or APG09106.1 (in Table 9.1), or APG06646.1 or APG01658.1 (in Table 9.2) for each disease. The target sequence information also provides protospacer sequence for the production of the necessary sgRNA for the corresponding RGN of the invention.

TABLE 9.1
Disease Targets for APG09748 and APG09106.1

| | | | | | Target |
|---|---|---|---|---|---|
| | | Casl | | Gene | (SEQ |
| Disease | RS# | Mut. | Chromosome Accession | Symbol | ID NO.) |
| Stargardt disease 1 | 1800728 | A > G | NC_000001.10, NC_000001.11 | ABCA4 | 313 |
| Glycogen storage disease type 1A | 1801175 | C > T | NC_000017.10, NC_000017.11 | G6PC | 335 |
| Severe combined immunodeficiency | 3218716 | C > T | NC_000014.8, NC_000014.9 | MYH7 | 336 |
| disease | | | | | |
| Phenylketonuria | 5030858 | G > A | NC_000012.11, NC_000012.12 | PAH | 337 |
| Hyperphenylalaninemia | 5030860 | T > C | NC_000012.11, NC_000012.12 | PAH | 338 |
| Alpha-1-antitrypsin deficiency | 28929474 | C > T | NC_000014.8, NC_000014.9 | SERPINA1 | 339 |
| MECP2-Related Disorders | 28934906 | G > A | NC_000023.10, NC_000023.11 | MECP2 | 340 |
| Inclusion body myopathy 2 | 28937594 | A > G | NC_000009.11, NC_000009.12 | GNE | 341 |
| Von Willebrand disease | 41276738 | C > T | NC_000012.11, NC_000012.12 | VWF | 342 |
| Breast and/or ovarian cancer | 41293455 | G > A | NC_000017.10, NC_000017.11 | BRCA1 | 343 |
| MECP2-Related Disorders | 61750240 | G > A | NC_000023.10, NC_000023.11 | MECP2 | 344 |
| MEFV-Related Disorder | 61752717 | T > C | NC_000016.9, NC_000016.10 | MEFV | 345 |
| Breast and/or ovarian cancer | 62625307 | G > A | NC_000017.10, NC_000017.11 | BRCA1 | 346 |
| Breast and/or ovarian cancer | 62625308 | G > A | NC_000017.10, NC_000017.11 | BRCA1 | 347 |
| Hereditary cancer-predisposing syndrome | 63749795 | C > T | NC_000003.11 , NC_000003.12 | MLH1 | 348 |
| Hereditary cancer-predisposing syndrome | 63749849 | C > T | NC_000002.11, NC_000002.12 | MSH2 | 349 |
| Hereditary cancer-predisposing syndrome | 63750636 | C > T | NC_000002.11, NC_000002.12 | MSH2 | 350 |
| Carnitine palmitoyltransferase II | 74315294 | C > T | NC_000001.10, NC_000001.11 | CPT2 | 351 |
| deficiency | | | | | |
| Cystic fibrosis | 74597325 | C > T | NC_000007.13, NC_000007.14 | CFTR | 352 |
| Deficiency of | 75391579 | A > G | NC_000009.11, NC_000009.12 | GALT | 353 |
| UDPglucose-hexose-1-phosphate | | | | | |
| uridylyltransferase | | | | | |
| Cystic fibrosis | 75527207 | G > A | NC_000007.13, NC_000007.14 | CFTR | 354 |
| Amyloidogenic transthyretin amyloidosis | 76992529 | G > A | NC_000018.9, NC_000018.10 | TTR | 355 |
| Cystic fibrosis | 77010898 | G > A | NC_000007.13, NC_000007.14 | CFTR | 356 |
| Metachromatic leukodystrophy | 80338815 | C > T | NC_000022.10, NC_000022.11 | ARSA | 357 |
| Cowden syndrome 3 | 80338844 | C > T | NC_000011.9, NC_000011.10 | SDHD | 358 |
| Smith-Lemli-Opitz syndrome | 80338853 | G > A | NC_000011.9, NC_000011.10 | DHCR7 | 359 |
| Breast and/or ovarian cancer | 80356962 | C > T | NC_000017.10, NC_000017.11 | BRCA1 | 360 |
| Breast and/or ovarian cancer | 80357123 | G > A | NC_000017.10, NC_000017.11 | BRCA1 | 361 |
| Inborn genetic diseases | 80358259 | A > G | NC_000018.9, NC_000018.10 | NPC1 | 362 |
| Breast and/or ovarian cancer | 80359212 | C > T | NC_000013.10, NC_000013.11 | BRCA2 | 363 |
| Fanconi anemia | 104886457 | G > A | NC_000009.11, NC_000009.12 | FANCC | 364 |
| SLC26A2-Related Disorders | 104893915 | C > T | NC_000005.9, NC_000005.10 | SLC26A2 | 365 |
| Cardiomyopathy | 104894368 | C > T | NC_000012.11, NC_000012.12 | MYL2 | 366 |
| Deafness, X-linked | 104894396 | C > T | NC_000013.10, NC_000013.11 | GJB2 | 367 |
| Inborn genetic diseases | 104894635 | C > T | NC_000017.10, NC_000017.11 | SGSH | 368 |
| Familial Mediterranean fever | 104895097 | C > T | NC_000016.9, NC_000016.10 | MEFV | 369 |
| Familial dysautonomia | 111033171 | A > G | NC_000009.11, NC_000009.12 | ELP1 | 370 |
| Shwachman syndrome | 113993993 | A > G | NC_000007.13, NC_000007.14 | SBDS | 371 |
| RYR1-Related Disorders | 118192172 | C > T | NC_000019.9, NC_000019.10 | RYR1 | 372 |
| Ceroid lipofuscinosis neuronal 2 | 119455955 | G > A | NC_000011.9, NC_000011.10 | TPP1 | 373 |
| Medium-chain acyl-coenzyme A | 121434274 | G > A | NC_000001.10, NC_000001.11 | ACADM | 374 |
| dehydrogenase deficiency | | | | | |
| Primary hyperoxaluria | 121908529 | G > A | NC_000002.11, NC_000002.12 | AGXT | 375 |
| Cardiomyopathy | 121908987 | C > T | NC_000007.13, NC_000007.14 | PRKAG2 | 376 |
| Cowden syndrome | 121909219 | C > T | NC_000010.10, NC_000010.11 | PTEN | 377 |
| Cardiomyopathy | 121913628 | C > T | NC_000014.8, NC_000014.9 | MYH7 | 378 |
| Inborn genetic diseases | 121918243 | G > A | NC_000001.10, NC_000001.11 | MMACHC | 379 |
| PTRN11-related disorder | 121918457 | C > T | NC_000012.11, NC_000012.12 | PTPN11 | 380 |
| Juvenile myelomonocytic leukemia | 121918462 | C > T | NC_000012.11, NC_000012.12 | PTPN11 | 381 |
| Juvenile myelomonocytic leukemia | 121918466 | A > G | NC_000012.11, NC_000012.12 | PTPN11 | 382 |
| Mucopolysaccharidosis type I | 121965020 | C > T | NC_000004.11, NC_000004.12 | IDUA | 383 |
| Ceroid lipofuscinosis neuronal 1 | 137852700 | G > A | NC_000001.10, NC_000001.11 | PPT1 | 384 |
| CHEK2-Related Cancer Susceptibility | 137853007 | G > A | NC_000022.10, NC_000022.11 | CHEK2 | 385 |
| Colorectal cancer | 137854568 | C > T | NC_000005.9, NC_000005.10 | APC | 386 |
| Familial hypercholesterolemia | 137929307 | G > A | NC_000019.9, NC_000019.10 | LDLR | 387 |
| Cardio-facio-cutaneous syndrome | 180177035 | T > C | NC_000007.13, NC_000007.14 | BRAF | 388 |
| Familial cancer of breast | 180177083 | G > A | NC_000016.10, NC_000016.9 | PALB2 | 389 |
| MYBPC3-Related Disorders | 200411226 | C > T | NC_000011.9, NC_000011.10 | MYBPC3 | 390 |
| RYR1-Related Disorders | 200563280 | C > T | NC_000019.9, NC_000019.10 | RYR1 | 391 |
| MYBPC3-Related Disorders | 387907267 | G > A | NC_000011.9, NC_000011.10 | MYBPC3 | 392 |
| Desmoid disease, hereditary | 397515734 | C > T | NC_000005.9, NC_000005.10 | APC | 393 |
| Marfan Syndrome/Loeys-Dietz | 397515757 | C > T | NC_000015.9, NC_000015.10 | FBN1 | 394 |
| Syndrome/Familial Thoracic Aortic | | | | | |
| Aneurysms and Dissections | | | | | |
| Immunodeficiency 14 | 397518423 | G > A | NC_000001.10, NC_000001.11 | PIK3CD | 395 |
| Inborn genetic diseases | 398123009 | C > T | NC_000011.9, NC_000011.10 | PACS1 | 396 |
| B lymphoblastic leukemia lymphoma, | 529008617 | G > A | NC_000001.10, NC_000001.11 | MUTYH | 397 |
| no ICD-O subtype | | | | | |
| Familial cancer of breast | 587780021 | G > A | NC_000002.11, NC_000002.12 | BARD1 | 398 |
| Familial hypercholesterolemia | 746118995 | C > T | NC_000019.9, NC_000019.10 | LDLR | 399 |
| Familial hypercholesterolemia | 769370816 | G > A | NC_000019.10, NC_000019.9 | LDLR | 400 |

TABLE 9.2
Disease Targets for APG6646.1 or APG01658.1

| | | | Casl | | Gene | Target |
|---|---|---|---|---|---|---|
| Disease | RGN | RS# | Mut. | Chromosome Accession | Symbol | (SEQ ID NO.) |
| Stargardt disease 1 | APG01658.1 | 1800553 | C > T | NC_000001.10, NC_000001.11 | ABCA4 | 538 |
| Stargardt disease 1 | APG06646.1 | 1800553 | C > T | NC_000001.10, NC_000001.11 | ABCA4 | 539 |
| Hereditary cancer-predisposing syndrome | APG01658.1 | 5030804 | A > G | NC_000003.11, NC_000003.12 | VHL | 540 |
| Hereditary cancer-predisposing syndrome | APG06646.1 | 5030804 | A > G | NC_000003.11, NC_000003.12 | VHL | 541 |
| Phenylketonuria | APG01658.1 | 5030851 | G > A | NC_000012.11, NC_000012.12 | PAH | 542 |
| Phenylketonuria | APG06646.1 | 5030851 | G > A | NC_000012.11, NC_000012.12 | PAH | 543 |
| Melanoma and myeloma | APG01658.1 | 11547328 | G > A | NC_000012.11, NC_000012.12 | CDK4 | 544 |
| Melanoma and myeloma | APG06646.1 | 11547328 | G > A | NC_000012.11, NC_000012.12 | CDK4 | 545 |
| Familial hypercholesterolemia | APG01658.1 | 11547917 | C > T | NC_000019.9, NC_000019.10 | LDLR | 546 |
| Familial hypercholesterolemia | APG06646.1 | 11547917 | C > T | NC_000019.9, NC_000019.10 | LDLR | 547 |
| Charcot-Marie-Tooth disease | APG01658.1 | 61444459 | G > A | NC_000001.10, NC_000001.11 | LMNA | 548 |
| Charcot-Marie-Tooth disease | APG06646.1 | 61444459 | G > A | NC_000001.10, NC_000001.11 | LMNA | 549 |
| Angelman syndrome | APG01658.1 | 61751362 | G > A | NC_000023.10, NC_000023.11 | MECP2 | 550 |
| Angelman syndrome | APG06646.1 | 61751362 | G > A | NC_000023.10, NC_000023.11 | MECP2 | 551 |
| Phenylketonuria | APG01658.1 | 62514895 | C > T | NC_000012.11, NC_000012.12 | PAH | 552 |
| Phenylketonuria | APG06646.1 | 62514895 | C > T | NC_000012.11, NC_000012.12 | PAH | 553 |
| Phenylketonuria | APG01658.1 | 62516152 | C > T | NC_000012.11, NC_000012.12 | PAH | 554 |
| Phenylketonuria | APG06646.1 | 62516152 | C > T | NC_000012.11, NC_000012.12 | PAH | 555 |
| Lynch syndrome | APG01658.1 | 63749795 | C > T | NC_000003.11, NC_000003.12 | MLH1 | 556 |
| Lynch syndrome | APG06646.1 | 63749795 | C > T | NC_000003.11, NC_000003.12 | MLH1 | 557 |
| Hereditary cancer | APG01658.1 | 63749843 | C > T | NC_000002.11, NC_000002.12 | MSH6 | 558 |
| Hereditary cancer | APG06646.1 | 63749843 | C > T | NC_000002.11, NC_000002.12 | MSH6 | 559 |
| Hereditary cancer-predisposing syndrome | APG01658.1 | 63749849 | C > T | NC_000002.11, NC_000002.12 | MSH2 | 560 |
| Hereditary cancer-predisposing syndrome | APG06646.1 | 63749849 | C > T | NC_000002.11, NC_000002.12 | MSH2 | 561 |
| Hereditary cancer-predisposing syndrome | APG01658.1 | 63750508 | C > T | NC_000002.11, NC_000002.12 | MSH2 | 562 |
| Hereditary cancer-predisposing syndrome | APG06646.1 | 63750508 | C > T | NC_000002.11, NC_000002.12 | MSH2 | 563 |
| Hereditary cancer-predisposing syndrome | APG01658.1 | 63750636 | C > T | NC_000002.11, NC_000002.12 | MSH2 | 564 |
| Hereditary cancer-predisposing syndrome | APG06646.1 | 63750636 | C > T | NC_000002.11, NC_000002.12 | MSH2 | 565 |
| Hereditary cancer-predisposing syndrome | APG06646.1 | 63750871 | G > A | NC_000007.13, NC_000007.14 | PMS2 | 566 |
| Lynch syndrome | APG01658.1 | 63751194 | C > T | NC_000003.11, NC_000003.12 | MLH1 | 567 |
| Lynch syndrome | APG06646.1 | 63751194 | C > T | NC_000003.11, NC_000003.12 | MLH1 | 568 |
| Lynch syndrome | APG01658.1 | 63751711 | G > A | NC_000003.11, NC_000003.12 | MLH1 | 569 |
| Lynch syndrome | APG06646.1 | 63751711 | G > A | NC_000003.11, NC_000003.12 | MLH1 | 570 |
| Cystic fibrosis | APG01658.1 | 75039782 | C > T | NC_000007.13, NC_000007.14 | CFTR | 571 |
| Cystic fibrosis | APG06646.1 | 75039782 | C > T | NC_000007.13, NC_000007.14 | CFTR | 572 |
| Deafness | APG01658.1 | 76434661 | C > T | NC_000013.10, NC_000013.11 | GJB2 | 573 |
| Deafness | APG06646.1 | 76434661 | C > T | NC_000013.10, NC_000013.11 | GJB2 | 574 |
| Cystic fibrosis | APG01658.1 | 76713772 | G > A | NC_000007.13, NC_000007.14 | CFTR | 575 |
| Cystic fibrosis | APG06646.1 | 76713772 | G > A | NC_000007.13, NC_000007.14 | CFTR | 576 |
| Cardiomyopathy | APG01658.1 | 76992529 | G > A | NC_000018.9, NC_000018.10 | TTR | 577 |
| Cardiomyopathy | APG06646.1 | 76992529 | G > A | NC_000018.9, NC_000018.10 | TTR | 578 |
| Cystic fibrosis | APG01658.1 | 77010898 | G > A | NC_000007.13, NC_000007.14 | CFTR | 579 |
| Cystic fibrosis | APG06646.1 | 77010898 | G > A | NC_000007.13, NC_000007.14 | CFTR | 580 |
| Cystic fibrosis | APG01658.1 | 80224560 | G > A | NC_000007.13, NC_000007.14 | CFTR | 581 |
| Cystic fibrosis | APG06646.1 | 80224560 | G > A | NC_000007.13, NC_000007.14 | CFTR | 582 |
| Cowden syndrome | APG01658.1 | 80338844 | C > T | NC_000011.9, NC_000011.10 | SDHD | 583 |
| Cowden syndrome | APG06646.1 | 80338844 | C > T | NC_000011.9, NC_000011.10 | SDHD | 584 |
| SLC26A4-Related Disorders | APG01658.1 | 80338849 | G > A | NC_000007.13, NC_000007.14 | SLC26A4 | 585 |
| SLC26A4-Related Disorders | APG06646.1 | 80338849 | G > A | NC_000007.13, NC_000007.14 | SLC26A4 | 586 |
| Breast-ovarian cancer | APG01658.1 | 80356885 | C > T | NC_000017.10, NC_000017.11 | BRCA1 | 587 |
| Breast-ovarian cancer | APG06646.1 | 80356885 | C > T | NC_000017.10, NC_000017.11 | BRCA1 | 588 |
| Breast-ovarian cancer | APG01658.1 | 80358557 | C > T | NC_000013.10, NC_000013.11 | BRCA2 | 589 |
| Breast-ovarian cancer | APG06646.1 | 80358557 | C > T | NC_000013.10, NC_000013.11 | BRCA2 | 590 |
| Breast-ovarian cancer | APG01658.1 | 80358650 | G > A | NC_000013.10, NC_000013.11 | BRCA2 | 591 |
| Breast-ovarian cancer | APG06646.1 | 80358650 | G > A | NC_000013.10, NC_000013.11 | BRCA2 | 592 |
| Breast-ovarian cancer | APG01658.1 | 80358659 | C > T | NC_000013.10, NC_000013.11 | BRCA2 | 593 |
| Breast-ovarian cancer | APG06646.1 | 80358659 | C > T | NC_000013.10, NC_000013.11 | BRCA2 | 594 |
| Breast-ovarian cancer | APG06646.1 | 80358663 | C > T | NC_000013.10, NC_000013.11 | BRCA2 | 595 |
| Breast-ovarian cancer | APG01658.1 | 80358871 | G > A | NC_000013.10, NC_000013.11 | BRCA2 | 596 |
| Breast-ovarian cancer | APG06646.1 | 80358871 | G > A | NC_000013.10, NC_000013.11 | BRCA2 | 597 |
| Breast-ovarian cancer | APG01658.1 | 80358920 | C > T | NC_000013.10, NC_000013.11 | BRCA2 | 598 |
| Breast-ovarian cancer | APG06646.1 | 80358920 | C > T | NC_000013.10, NC_000013.11 | BRCA2 | 599 |
| Breast-ovarian cancer | APG01658.1 | 80358972 | C > T | NC_000013.10, NC_000013.11 | BRCA2 | 600 |
| Breast-ovarian cancer | APG06646.1 | 80358972 | C > T | NC_000013.10, NC_000013.11 | BRCA2 | 601 |
| Breast-ovarian cancer | APG01658.1 | 80358981 | C > T | NC_000013.10, NC_000013.11 | BRCA2 | 602 |
| Breast-ovarian cancer | APG06646.1 | 80358981 | C > T | NC_000013.10, NC_000013.11 | BRCA2 | 603 |
| Breast-ovarian cancer | APG01658.1 | 80359003 | G > A | NC_000013.10, NC_000013.11 | BRCA2 | 604 |
| Breast-ovarian cancer | APG06646.1 | 80359003 | G > A | NC_000013.10, NC_000013.11 | BRCA2 | 605 |
| Breast-ovarian cancer | APG01658.1 | 80359004 | G > A | NC_000013.10, NC_000013.11 | BRCA2 | 606 |
| Breast-ovarian cancer | APG06646.1 | 80359004 | G > A | NC_000013.10, NC_000013.11 | BRCA2 | 607 |
| Breast-ovarian cancer | APG01658.1 | 80359013 | G > A | NC_000013.10, NC_000013.11 | BRCA2 | 608 |
| Breast-ovarian cancer | APG06646.1 | 80359013 | G > A | NC_000013.10, NC_000013.11 | BRCA2 | 609 |
| Breast-ovarian cancer | APG01658.1 | 80359027 | G > A | NC_000013.10, NC_000013.11 | BRCA2 | 610 |
| Breast-ovarian cancer | APG06646.1 | 80359027 | G > A | NC_000013.10, NC_000013.11 | BRCA2 | 611 |
| Breast-ovarian cancer | APG01658.1 | 80359071 | G > A | NC_000013.10, NC_000013.11 | BRCA2 | 612 |
| Breast-ovarian cancer | APG06646.1 | 80359071 | G > A | NC_000013.10, NC_000013.11 | BRCA2 | 613 |
| Breast-ovarian cancer | APG01658.1 | 80359115 | C > T | NC_000013.10, NC_000013.11 | BRCA2 | 614 |
| Breast-ovarian cancer | APG06646.1 | 80359115 | C > T | NC_000013.10, NC_000013.11 | BRCA2 | 615 |
| Breast-ovarian cancer | APG01658.1 | 80359140 | C > T | NC_000013.10, NC_000013.11 | BRCA2 | 616 |
| Breast-ovarian cancer | APG06646.1 | 80359140 | C > T | NC_000013.10, NC_000013.11 | BRCA2 | 617 |
| Breast-ovarian cancer | APG06646.1 | 80359159 | C > T | NC_000013.10, NC_000013.11 | BRCA2 | 618 |
| Breast-ovarian cancer | APG01658.1 | 80359180 | C > T | NC_000013.10, NC_000013.11 | BRCA2 | 619 |
| Breast-ovarian cancer | APG06646.1 | 80359180 | C > T | NC_000013.10, NC_000013.11 | BRCA2 | 620 |
| Breast-ovarian cancer | APG01658.1 | 81002846 | G > A | NC_000013.10, NC_000013.11 | BRCA2 | 621 |
| Breast-ovarian cancer | APG06646.1 | 81002846 | G > A | NC_000013.10, NC_000013.11 | BRCA2 | 622 |
| Breast-ovarian cancer | APG06646.1 | 81002862 | A > G | NC_000013.10, NC_000013.11 | BRCA2 | 623 |
| Fanconi anemia | APG01658.1 | 104886457 | G > A | NC_000009.11, NC_000009.12 | FANCC | 624 |
| Fanconi anemia | APG06646.1 | 104886457 | G > A | NC_000009.11, NC_000009.12 | FANCC | 625 |
| Achondrogenesis | APG01658.1 | 104893915 | C > T | NC_000005.9, NC_000005.10 | SLC26A2 | 626 |
| Achondrogenesis | APG06646.1 | 104893915 | C > T | NC_000005.9, NC_000005.10 | SLC26A2 | 627 |
| RYR1-Related Disorders | APG01658.1 | 118192172 | C > T | NC_000019.9, NC_000019.10 | RYR1 | 628 |
| RYR1-Related Disorders | APG06646.1 | 118192172 | C > T | NC_000019.9, NC_000019.10 | RYR1 | 629 |
| CHEK2-Related Cancer Susceptibility | APG01658.1 | 137853007 | G > A | NC_000022.10, NC_000022.11 | CHEK2 | 630 |
| CHEK2-Related Cancer Susceptibility | APG06646.1 | 137853007 | G > A | NC_000022.10, NC_000022.11 | CHEK2 | 631 |
| Neurofibromatosis | APG06646.1 | 137854550 | A > G | NC_000017.10, NC_000017.11 | NF1 | 632 |
| Neurofibromatosis | APG01658.1 | 137854556 | G > A | NC_000017.10, NC_000017.11 | NF1 | 633 |
| Neurofibromatosis | APG06646.1 | 137854556 | G > A | NC_000017.10, NC_000017.11 | NF1 | 634 |
| Myopathy | APG01658.1 | 193922390 | C > T | NC_000014.8, NC_000014.9 | MYH7 | 635 |
| Myopathy | APG06646.1 | 193922390 | C > T | NC_000014.8, NC_000014.9 | MYH7 | 636 |
| Long QT syndrome | APG01658.1 | 199473428 | C > T | NC_000007.13, NC_000007.14 | KCNH2 | 637 |
| Long QT syndrome | APG06646.1 | 199473428 | C > T | NC_000007.13, NC_000007.14 | KCNH2 | 638 |
| Phenylketonuria | APG01658.1 | 199475575 | G > A | NC_000012.11, NC_000012.12 | PAH | 639 |
| Phenylketonuria | APG06646.1 | 199475575 | G > A | NC_000012.11, NC_000012.12 | PAH | 640 |
| Phenylketonuria | APG01658.1 | 199475679 | C > T | NC_000012.11, NC_000012.12 | PAH | 641 |
| Phenylketonuria | APG06646.1 | 199475679 | C > T | NC_000012.11, NC_000012.12 | PAH | 642 |
| MYBPC3-Related Disorders | APG01658.1 | 200411226 | C > T | NC_000011.9, NC_000011.10 | MYBPC3 | 643 |
| MYBPC3-Related Disorders | APG06646.1 | 200411226 | C > T | NC_000011.9, NC_000011.10 | MYBPC3 | 644 |
| Myopathy | APG06646.1 | 267606908 | T > C | NC_000014.8, NC_000014.9 | MYH7 | 645 |
| Cardiomyopathy | APG01658.1 | 267607003 | C > T | NC_000010.10, NC_000010.11 | RBM20 | 646 |
| Cardiomyopathy | APG06646.1 | 267607003 | C > T | NC_000010.10, NC_000010.11 | RBM20 | 647 |
| Noonan syndrome | APG01658.1 | 267607048 | A > G | NC_000010.10, NC_000010.11 | SHOC2 | 648 |
| Noonan syndrome | APG06646.1 | 267607048 | A > G | NC_000010.10, NC_000010.11 | SHOC2 | 649 |
| Familial hypercholesterolemia | APG01658.1 | 267607213 | G > A | NC_000019.9, NC_000019.10 | LDLR | 650 |
| Familial hypercholesterolemia | APG06646.1 | 267607213 | G > A | NC_000019.9, NC_000019.10 | LDLR | 651 |
| Lynch syndrome | APG01658.1 | 267607789 | G > A | NC_000003.11, NC_000003.12 | MLH1 | 652 |
| Lynch syndrome | APG06646.1 | 267607789 | G > A | NC_000003.11, NC_000003.12 | MLH1 | 653 |
| Lynch syndrome | APG01658.1 | 267607845 | G > A | NC_000003.11, NC_000003.12 | MLH1 | 654 |
| Lynch syndrome | APG06646.1 | 267607845 | G > A | NC_000003.11, NC_000003.12 | MLH1 | 655 |
| Hereditary cancer-predisposing syndrome | APG01658.1 | 267608098 | A > G | NC_000002.11, NC_000002.12 | MSH6 | 656 |
| Hereditary cancer-predisposing syndrome | APG06646.1 | 267608098 | A > G | NC_000002.11, NC_000002.12 | MSH6 | 657 |
| MYBPC3-Related Disorders | APG01658.1 | 387906397 | A > G | NC_000011.9, NC_000011.10 | MYBPC3 | 658 |
| MYBPC3-Related Disorders | APG06646.1 | 387906397 | A > G | NC_000011.9, NC_000011.10 | MYBPC3 | 659 |
| Breast-ovarian cancer | APG01658.1 | 387906843 | G > A | NC_000017.10, NC_000017.11 | RAD51D | 660 |
| Breast-ovarian cancer | APG06646.1 | 387906843 | G > A | NC_000017.10, NC_000017.11 | RAD51D | 661 |
| MYBPC3-Related Disorders | APG01658.1 | 387907267 | G > A | NC_000011.9, NC_000011.10 | MYBPC3 | 662 |
| MYBPC3-Related Disorders | APG06646.1 | 387907267 | G > A | NC_000011.9, NC_000011.10 | MYBPC3 | 663 |
| Breast-ovarian cancer | APG01658.1 | 397507389 | G > A | NC_000013.10, NC_000013.11 | BRCA2 | 664 |
| Breast-ovarian cancer | APG06646.1 | 397507389 | G > A | NC_000013.10, NC_000013.11 | BRCA2 | 665 |
| Breast-ovarian cancer | APG01658.1 | 397507404 | G > A | NC_000013.10, NC_000013.11 | BRCA2 | 666 |
| Breast-ovarian cancer | APG06646.1 | 397507404 | G > A | NC_000013.10, NC_000013.11 | BRCA2 | 667 |
| Leukemia | APG01658.1 | 397507545 | G > A | NC_000012.11, NC_000012.12 | PTPN11 | 668 |
| Leukemia | APG06646.1 | 397507545 | G > A | NC_000012.11, NC_000012.12 | PTPN11 | 669 |
| Leukemia | APG01658.1 | 397507547 | A > G | NC_000012.11, NC_000012.12 | PTPN11 | 670 |
| Leukemia | APG06646.1 | 397507547 | A > G | NC_000012.11, NC_000012.12 | PTPN11 | 671 |
| Breast-ovarian cancer | APG01658.1 | 397507922 | G > A | NC_000013.10, NC_000013.11 | BRCA2 | 672 |
| Breast-ovarian cancer | APG06646.1 | 397507922 | G > A | NC_000013.10, NC_000013.11 | BRCA2 | 673 |
| Marfan syndrome | APG01658.1 | 397515757 | C > T | NC_000015.9, NC_000015.10 | FBN1 | 674 |
| Marfan syndrome | APG06646.1 | 397515757 | C > T | NC_000015.9, NC_000015.10 | FBN1 | 675 |
| MYBPC3-Related Disorders | APG01658.1 | 397516074 | C > T | NC_000011.9, NC_000011.10 | MYBPC3 | 676 |
| MYBPC3-Related Disorders | APG06646.1 | 397516074 | C > T | NC_000011.9, NC_000011.10 | MYBPC3 | 677 |
| Cardiomyopathy | APG01658.1 | 397516354 | C > T | NC_000019.9, NC_000019.10 | TNNI3 | 678 |
| Cardiomyopathy | APG06646.1 | 397516354 | C > T | NC_000019.9, NC_000019.10 | TNNI3 | 679 |
| Immunodeficiency | APG01658.1 | 397518423 | G > A | NC_000001.10, NC_000001.11 | PIK3CD | 680 |
| Immunodeficiency | APG06646.1 | 397518423 | G > A | NC_000001.10, NC_000001.11 | PIK3CD | 681 |
| B lymphoblastic leukemia lymphoma | APG01658.1 | 529008617 | G > A | NC_000001.10, NC_000001.11 | MUTYH | 682 |
| B lymphoblastic leukemia lymphoma | APG06646.1 | 529008617 | G > A | NC_000001.10, NC_000001.11 | MUTYH | 683 |
| Familial hypercholesterolemia | APG01658.1 | 570942190 | C > T | NC_000019.9, NC_000019.10 | LDLR | 684 |
| Familial hypercholesterolemia | APG06646.1 | 570942190 | C > T | NC_000019.9, NC_000019.10 | LDLR | 685 |
| Ataxia-telangiectasia syndrome | APG01658.1 | 587776551 | G > A | NC_000011.10, NC_000011.9 | ATM | 686 |
| Ataxia-telangiectasia syndrome | APG06646.1 | 587776551 | G > A | NC_000011.10, NC_000011.9 | ATM | 687 |
| Adenoid cystic carcinoma | APG01658.1 | 587778720 | C > T | NC_000017.10, NC_000017.11 | TP53 | 688 |
| Adenoid cystic carcinoma | APG06646.1 | 587778720 | C > T | NC_000017.10, NC_000017.11 | TP53 | 689 |
| Hereditary cancer-predisposing syndrome | APG01658.1 | 587779075 | C > T | NC_000002.11, NC_000002.12 | MSH2 | 690 |
| Hereditary cancer-predisposing syndrome | APG06646.1 | 587779075 | C > T | NC_000002.11, NC_000002.12 | MSH2 | 691 |
| Hereditary cancer-predisposing syndrome | APG06646.1 | 587779333 | T > C | NC_000007.13, NC_000007.14 | PMS2 | 692 |
| Ataxia-telangiectasia syndrome | APG01658.1 | 587779866 | A > G | NC_000011.10, NC_000011.9 | ATM | 693 |
| Ataxia-telangiectasia syndrome | APG06646.1 | 587779866 | A > G | NC_000011.10, NC_000011.9 | ATM | 694 |
| Breast cancer | APG01658.1 | 587780021 | G > A | NC_000002.11, NC_000002.12 | BARD1 | 695 |
| Breast cancer | APG06646.1 | 587780021 | G > A | NC_000002.11, NC_000002.12 | BARD1 | 696 |
| Breast-ovarian cancer | APG01658.1 | 587781629 | G > A | NC_000013.10, NC_000013.11 | BRCA2 | 697 |
| Breast-ovarian cancer | APG06646.1 | 587781629 | G > A | NC_000013.10, NC_000013.11 | BRCA2 | 698 |
| Hereditary cancer-predisposing syndrome | APG01658.1 | 587782144 | C > T | NC_000017.10, NC_000017.11 | TP53 | 699 |
| Hereditary cancer-predisposing syndrome | APG06646.1 | 587782144 | C > T | NC_000017.10, NC_000017.11 | TP53 | 700 |
| Marfan syndrome | APG01658.1 | 727503054 | A > G | NC_000015.9, NC_000015.10 | FBN1 | 701 |
| Marfan syndrome | APG06646.1 | 727503054 | A > G | NC_000015.9, NC_000015.10 | FBN1 | 702 |
| Noonan syndrome | APG01658.1 | 727503110 | T > C | NC_000012.11, NC_000012.12 | KRAS | 703 |
| Noonan syndrome | APG06646.1 | 727503110 | T > C | NC_000012.11, NC_000012.12 | KRAS | 704 |
| Familial hypercholesterolemia | APG01658.1 | 746118995 | C > T | NC_000019.9, NC_000019.10 | LDLR | 705 |
| Familial hypercholesterolemia | APG06646.1 | 746118995 | C > T | NC_000019.9, NC_000019.10 | LDLR | 706 |
| Familial hypercholesterolemia | APG01658.1 | 748944640 | G > A | NC_000019.9, NC_000019.10 | LDLR | 707 |
| Familial hypercholesterolemia | APG06646.1 | 748944640 | G > A | NC_000019.9, NC_000019.10 | LDLR | 708 |
| Familial hypercholesterolemia | APG01658.1 | 765696008 | G > A | NC_000019.10, NC_000019.9 | LDLR | 709 |
| Familial hypercholesterolemia | APG06646.1 | 765696008 | G > A | NC_000019.10, NC_000019.9 | LDLR | 710 |
| Familial hypercholesterolemia | APG01658.1 | 769370816 | G > A | NC_000019.10, NC_000019.9 | LDLR | 711 |
| Familial hypercholesterolemia | APG06646.1 | 769370816 | G > A | NC_000019.10, NC_000019.9 | LDLR | 712 |
| Familial hypercholesterolemia | APG01658.1 | 769737896 | C > T | NC_000019.10, NC_000019.9 | LDLR | 713 |
| Familial hypercholesterolemia | APG06646.1 | 769737896 | C > T | NC_000019.10, NC_000019.9 | LDLR | 714 |
| Familial hypercholesterolemia | APG01658.1 | 771019366 | A > G | NC_000019.10, NC_000019.9 | LDLR | 715 |
| Familial hypercholesterolemia | APG06646.1 | 771019366 | A > G | NC_000019.10, NC_000019.9 | LDLR | 716 |
| Paragangliomas | APG01658.1 | 772551056 | C > T | NC_000001.11, NC_000001.10 | SDHB | 717 |
| Paragangliomas | APG06646.1 | 772551056 | C > T | NC_000001.11, NC_000001.10 | SDHB | 718 |
| Hereditary cancer | APG01658.1 | 786201042 | C > T | NC_000002.12, NC_000002.11 | MSH6 | 719 |
| Hereditary cancer | APG06646.1 | 786201042 | C > T | NC_000002.12, NC_000002.11 | MSH6 | 720 |
| Familial hypercholesterolemia | APG01658.1 | 875989907 | G > A | NC_000019.9, NC_000019.10 | LDLR | 721 |
| Familial hypercholesterolemia | APG06646.1 | 875989907 | G > A | NC_000019.9, NC_000019.10 | LDLR | 722 |
| Familial hypercholesterolemia | APG01658.1 | 879254797 | G > A | NC_000019.10, NC_000019.9 | LDLR | 723 |
| Familial hypercholesterolemia | APG06646.1 | 879254797 | G > A | NC_000019.10, NC_000019.9 | LDLR | 724 |
| Familial hypercholesterolemia | APG01658.1 | 879254871 | C > T | NC_000019.10, NC_000019.9 | LDLR | 725 |
| Familial hypercholesterolemia | APG06646.1 | 879254871 | C > T | NC_000019.10, NC_000019.9 | LDLR | 726 |
| Familial hypercholesterolemia | APG01658.1 | 879255000 | T > C | NC_000019.10, NC_000019.9 | LDLR | 727 |
| Familial hypercholesterolemia | APG06646.1 | 879255000 | T > C | NC_000019.10, NC_000019.9 | LDLR | 728 |

### Example 8: Targeting Mutations Responsible for Friedreich Ataxia

The expansion of the trinucleotide repeat sequence causing Friedreich's Ataxia (FRDA) occurs in a defined genetic locus within the FXN gene, referred to as the FRDA instability region. RNA guided nucleases (RGNs) may be used for excising the instability region in FRDA patient cells. This approach requires 1) an RGN and guide RNA sequence that can be programmed to target the allele in the human genome; and 2) a delivery approach for the RGN and guide sequence. Many nucleases used for genome editing, such as the commonly used Cas9 nuclease from S. pyogenes (SpCas9), are too large to be packaged into adeno-associated viral (AAV) vectors, especially when considering the length of the SpCas9 gene and the guide RNA in addition to other genetic elements required for functional expression cassettes. This makes a viable approach using SpCas9 unlikely.

The compact RNA guided nucleases of the invention, such as APG09748, APG09106.1, and APG06646.1, are uniquely well suited for the excision of the FRDA instability region. Each RGN has a PAM requirement that is in the vicinity of the FRDA instability region. Additionally, each of these RGNs can be packaged into an AAV vector along with a guide RNA. Packing two guide RNAs would likely require a second vector, but this approach still compares favorably to what would be required of a larger nuclease such as SpCas9, which would require splitting the protein sequence between two vectors.

Table 10 shows the location of genomic target sequences suitable for targeting APG09748, APG09106.1, or APG06646.1 to the 5′ and 3′ flanks of the FRDA instability region, as well as the sequence of the sgRNAs for the genomic targets. Once at the locus, the RGN would excise the FA instability region. Excision of the region can be verified with Illumina sequencing of the locus.

TABLE 10
Genomic target sequences for RGN systems

| | Genome | | | |
|---|---|---|---|---|
| | target | | | |
| Location | sequence for | sgRNA for | Genome | |
| relative | APG09748 | APG09748 | target | |
| to | or | or | sequence for | sgRNA for |
| FRDA | APG09106.1 | APG09106.1 | APG06646.1 | APG06646.1 |
| instability | (SEQ ID | (SEQ ID | (SEQ ID | (SEQ ID |
| region | NO.) | NO.) | NO.) | NO.) |
| 5′ | 401 | 405 | 729 | 733 |
| 5′ | 402 | 406 | 730 | 734 |
| 3′ | 403 | 407 | 731 | 735 |
| 3′ | 404 | 408 | 732 | 736 |

### Example 9: Targeting Mutations Responsible for Sickle Cell Diseases

Targeting sequences within the BCL11A enhancer region (SEQ ID NO: 220) may provide a mechanism for increasing fetal hemoglobulin (HbF) to either cure or alleviate the symptoms of sickle cell diseases. For example, genome wide association studies have identified a set of genetic variations at BCL11A that are associated with increased HbF levels. These variations are a collection of SNPs found in non-coding regions of BCL11A that function as a stage-specific, lineage-restricted enhancer region. Further investigation revealed that this BCL11A enhancer is required in erythroid cells for BCL11A expression (Bauer et al, (2013) Science 343:253-257, incorporated by reference herein). The enhancer region was found within intron 2 of the BCL11A gene, and three areas of DNasel hypersensitivity (often indicative of a chromatin state that is associated with regulatory potential) in intron 2 were identified. These three areas were identified as “+62”, “+58” and “+55” in accordance with the distance in kilobases from the transcription start site of BCL11A. These enhancer regions are roughly 350 (+55); 550 (+58); and 350 (+62) nucleotides in length (Bauer et al., 2013).

### Example 9.1: Identifying Preferred RGN Systems

Here is described a potential treatment for beta-hemoglobinopathies using an RGN system that disrupts BCL11A binding to its binding site within the HBB locus, which is the gene responsible for making beta-globin in adult hemoglobin. This approach uses NHEJ which is more efficient in mammalian cells. In addition, this approach uses a nuclease of sufficiently small size that can be packaged into a single AAV vector for in vivo delivery.

The GATA1 enhancer motif in the human BCL11A enhancer region (SEQ ID NO: 220) is an ideal target for disruption using RNA guided nucleases (RGNs) to reduce BCL11A expression with concurrent re-expression of HbF in adult human erythrocytes (Wu et al. (2019) Nat Med 387:2554). Several PAM sequences compatible with APG09748 or APG09106.1 are readily apparent at the genetic locus surrounding this GATA1 site. These nucleases have a PAM sequence of 5′-DTTN-3′ (SEQ ID NO: 60) and are compact in size, potentially allowing their delivery along with an appropriate guide RNA in a single AAV or adenoviral vector. In addition to its size, APG06646.1 has a minimal PAM requirement (SEQ ID NO: 109) which makes it well-suited for this approach. This delivery approach bestows multiple advantages relative to others, such as access to hematopoietic stem cells and a well-established safety profile and manufacturing techniques.

The commonly used Cas9 nuclease from S. pyogenes (SpyCas9) requires a PAM sequence of 5′-NGG-3′, (SEQ ID NO: 101) several of which are present near the GATA1 motif. However, the size of SpyCas9 prevents packaging into a single AAV or adenoviral vector and thus forgoes the aforementioned advantages of this approach. While a dual delivery strategy may be employed, it would add significant manufacturing complexity and cost. Additionally, dual viral vector delivery significantly decreases the efficiency of gene correction, since a successful edit in a given cell requires infection with both vectors. An expression cassette encoding a human codon optimized APG09748 (SEQ ID NO: 409), APG09106.1 (SEQ ID NO: 410), or APG06646.1 (SEQ ID NO: 415) is produced, similar to those described in Example 5. Expression cassettes which express guide RNAs for RGNs APG09748, APG09106.1, or APG06646.1 are also produced. These guide RNAs comprise: 1) a protospacer sequence that is complementary to either the non-coding or coding DNA strand within the BCL11A enhancer locus (the target sequence) and 2) an RNA sequence required for association of the guide RNA with the RGN. Because several potential PAM sequences for targeting by each RGN surround the BCL11A GATA1 enhancer motif, several potential guide RNA constructs are produced to determine the best protospacer sequence that produces robust cleavage and NHEJ mediated disruption of the BCL11A GATA1 enhancer sequence. The target genomic sequences in Table 11 are evaluated using the sgRNA provided in Table 11.

TABLE 11
Target Sequences for BCL11A GATA1 enhancer locus using
APG06646.1

| Target genomic | | | |
|---|---|---|---|
| sequence for | sgRNA for | Target genomic | |
| APG09748 or | APG09748 or | sequence for | sgRNA for |
| APG0916.1 | APG0916.1 | APG06646.1 | APG06646.1 |
| (SEQ ID NO.) | (SEQ ID NO.) | (SEQ ID NO.) | (SEQ ID NO.) |
| 221 | 224 | 737 | 740 |
| 222 | 269 | 738 | 741 |
| 223 | 270 | 739 | 742 |

To evaluate the efficiency with which APG09748, APG09106.1, or APG06646.1 generates insertions or deletions that disrupt the BCL11A enhancer region, human cell lines such as human embryonic kidney cells (HEK cells) are used. A DNA vector comprising an RGN expression cassette (for example, as described in Example 5) is produced. A separate vector comprising an expression cassette comprising a coding sequence for a guide RNA sequence of Table 11 is also produced. Such an expression cassette may further comprise a human RNA polymerase III U6 promoter (SEQ ID NO: 308), as described in Example 5. Alternatively, a single vector comprising expression cassettes of both the RGN and guide RNA may be used. The vector is introduced into HEK cells using standard techniques such as those described in Example 5, and the cells are cultured for 1-3 days. Following this culture period, genomic DNA is isolated and the frequency of insertions or deletions is determined by using T7 Endonuclease I digestion and/or direct DNA sequencing, as described in Example 5.

A region of DNA encompassing the target BCL11A region is amplified by PCR with primers containing Illumina Nextera XT overhang sequences. These PCR amplicons are either examined for NHEJ formation using T7 Endonuclease I digestion or undergo library preparation following the Illumina 16S Metagenomic Sequencing Library protocol or a similar Next Generation Sequencing (NGS) library preparation. Following deep sequencing, the reads generated are analyzed by CRISPResso to calculate rates of editing. Output alignments are hand-curated to confirm insertion and deletion sites. This analysis identifies the preferred RGN and the corresponding preferred guide RNA (sgRNA). The analysis may result in either APG09748, APG09106.1, or APG06646.1 being equally preferred or that one RGN is most preferred. Additionally, the analysis may determine there is more than one preferred guide RNA, or that all target genomic sequences in Table 17 are equally preferred.

### Example 9.2: Assay for Expression of Fetal Hemoglobin

In this example, APG09748, APG09106.1, or APG06646.1 generated insertions or deletions disrupting the BCL11A enhancer region are assayed for expression of fetal hemoglobin. Healthy human donor CD34⁺ hematopoietic stem cells (HSCs) are used. These HSCs are cultured and vector(s) comprising expression cassettes comprising the coding regions of the preferred RGN and the preferred sgRNA are introduced using methods similar to those described in Example 5. Alternatively, electroporation may be used. Following electroporation, these cells are differentiated in vitro into erythrocytes using established protocols (for example, Giarratana et al. (2004) Nat Biotechnology 23:69-74, herein incorporated by reference). The expression of HbF is then measured using western blotting with an anti-human HbF antibody or quantified via High Performance Liquid Chromatography (HPLC). It is expected that successful disruption of the BCL11A enhancer locus will lead to an increase in HbF production when compared to HSCs electroporated with only the RGN but no guide.

### Example 9.3: Assay for Decreased Sickle Cell Formation

In this example, APG09748, APG09106.1, or APG06646.1 generated insertions or deletions disrupting the BCL11A enhancer region are assayed for decreased sickle-cell formation. Donor CD34⁺ hematopoietic stem cells (HSCs) from patients afflicted with sickle cell disease are used. These HSCs are cultured and vector(s) comprising expression cassettes comprising the coding regions of preferred RGN and the preferred sgRNA are introduced using methods similar to those described in Example 5. Alternatively, electroporation may be used. Following electroporation, these cells are differentiated in vitro into erythrocytes using established protocols (Giarratana et al. (2004) Nat Biotechnology 23:69-74). The expression of HbF is then measured using western blotting with an anti-human HbF antibody or quantified via High Performance Liquid Chromatography (HPLC). It is expected that successful disruption of the BCL11A enhancer locus will lead to an increase in HbF production when compared to HSCs electroporated with only the RGN but no guide.

Sickle cell formation is induced in these differentiated erythrocytes by the addition of metabisulfite. The numbers of sickled vs normal erythrocytes are counted using a microscope. It is expected that the numbers of sickled cells are less in cells treated with APG09748, APG09106.1, or APG06646.1 plus sgRNAs than with cells untreated, or treated with RGNs alone.

### Example 9.4: Disease Treatment Validation in a Murine Model

To evaluate the efficacy of using APG09748, APG09106.1, or APG06646.1 disruption of the BCL11A locus, suitable humanized mouse models of sickle cell anemia are used. Expression cassettes encoding for the preferred RGN and for the preferred sgRNA are packaged into AAV vectors or adenovirus vectors. In particular, adenovirus type Ad5/35 is effective at targeting HSCs. A suitable mouse model containing a humanized HBB locus with sickle cell alleles is chosen such as B6; FVB-Tg (LCR-HBA2,LCR-HBB*E26K) 53Hhb/J or B6.Cg-Hbatm1Paz HbbtmlTow Tg (HBA-HBBs) 41Paz/HhbJ. These mice are treated with granulocyte colony-stimulating factor alone or in combination with plerixafor to mobilize HSCs into circulation. AAVs or adenoviruses carrying the RGN and guide plasmid are then injected intravenously, and the mice are allowed to recover for a week. Blood obtained from these mice is tested in an in vitro sickling assay using metabisulfite, and the mice are followed longitudinally to monitor mortality rates and hematopoietic function. It is expected that treatment with AAVs or adenoviruses carrying an RGN and guide RNA will reduce sickling, mortality, and improve hematopoietic function when compared to mice treated with viruses lacking both expression cassettes, or with viruses carrying the RGN expression cassette alone.

### Example 10: Base Editing Activity in Mammalian Cells

An expression cassette that produces a cytidine deaminase-RGN fusion protein was constructed as follows. The coding sequence for RGNs (APG06646.1 described herein and APG08290.1, which was described in U.S. Appl. Publ. No. 2019/0367949 and International Appl. Publ. No. WO 2019/236566, each of which is herein incorporated by reference in its entirety) was codon optimized for mammalian expression and mutated to function as a nickase (SEQ ID NOs: 128 and 262, respectively). This coding sequence was introduced into an expression cassette which produces a fusion protein comprising an NLS at its N-terminal end (SEQ ID NO: 125), operably linked at its C-terminal end to a 3×FLAG tag (SEQ ID NO: 126), operably linked at its C-terminal end to a cytidine deaminase (SEQ ID NOs: 129-132, all of which are disclosed in International Appl. No. PCT/US2019/068079, which is herein incorporated by reference in its entirety) operably linked on its C-terminal end to an amino acid linker (SEQ ID NO: 133), operably linked on its C-terminal end to the RGN nickase, operably linked at its C-terminal end to a second NLS (SEQ ID NO: 127). These expression cassettes were each introduced into a pTwist CMV vector (Twist Bioscience) capable of driving expression of the fusion protein in mammalian cells. Separate vectors were also produced that expressed guide RNAs under control of the human U6 promoter, in mammalian cells. These guide RNAs (SEQ ID NOs: 134-136 for nAPG06646.1 and SEQ ID NOs: 263-265 for nAPG08290.1) are capable of guiding the deaminase-nRGN fusion proteins, or the RGN itself, to a targeted genomic sequence for base editing or gene editing, respectively.

500 ng of cytidine deaminase-RGN expression plasmids or standard RGN expression plasmid, and 500 ng of the guide RNA expression plasmids, were co-transfected into HEK293 FT cells at 75-90% confluency in 24-well plates using Lipofectamine 2000 reagent (Life Technologies). Cells were then incubated at 37 °C for 72 h. Genomic DNA was then extracted using the NucleoSpin 96 Tissue (Macherey-Nagel) following the manufacturer's protocol. The genomic region flanking the guideRNA target site was PCR amplified, and products were purified using ZR-96 DNA Clean and Concentrator (Zymo Research) following the manufacturer's protocol. The purified PCR products were then sent for Next Generation Sequencing on Illumina MiSeq (2×250). Results were analyzed for indel formation or specific cytosine mutation.

Table 12 below shows cytidine base editing and indel formation of each construct and guide combination. Interestingly, when comparing the activity of the RGN APG06646.1 with that of cytidine deaminase-nAPG06646.1 using the same guide RNA, up to 20× higher cytidine base editing than gene editing was observed. These results demonstrate that an RGN that has low nuclease activity at a particular target site can still be an efficient base editor at that site. Furthermore, since indel formation in base editing applications is often an unwanted outcome, RGNs that have low nuclease activity at a site may be preferred for base editing applications.

TABLE 12
Cytidine base editing and gene editing of RGN and cytidine
deaminase-nRGN

| | | % | % |
|---|---|---|---|
| | | Total Reads | Total Reads |
| | | with Cytidine | with INDEL |
| RGN or Deaminase-RGN | Guide RNA | Base Editing | Formation |
| ARM05-nAPG06646.1 | SGN000775 | 8.02 | 0.82 |
| ARM06CTD-nAPG06646.1 | SGN000775 | 0 | 0 |
| ARM08-nAPG06646.1 | SGN000775 | 5.15 | 0.46 |
| ARM11-nAPG06646.1 | SGN000775 | 0.66 | 0 |
| APG06646.1 | SGN000775 | 0 | 0.47 |
| ARM05-nAPG06646.1 | SGN000777 | 2.5 | 0.08 |
| ARM06CTD-nAPG06646.1 | SGN000777 | 0.53 | 0 |
| ARM08-nAPG06646.1 | SGN000777 | 8.12 | 0.25 |
| ARM11-nAPG06646.1 | SGN000777 | 3.52 | 1.04 |
| APG06646.1 | SGN000777 | 0 | 1.44 |
| ARM05-nAPG06646.1 | SGN000781 | 13.51 | 0.27 |
| ARM06CTD-nAPG06646.1 | SGN000781 | 2.69 | 0.13 |
| ARM08-nAPG06646.1 | SGN000781 | 12.97 | 0.12 |
| ARM11-nAPG06646.1 | SGN000781 | 4.7 | 0.14 |
| APG06646.1 | SGN000781 | 0 | 0.65 |
| ARM05-nAPG08290.1 | SGN000143 | 1.35 | 7.04 |
| ARM06CTD-nAPG08290.1 | SGN000143 | 1.36 | 3.17 |
| ARM08-nAPG08290.1 | SGN000143 | 2.59 | 10.98 |
| ARM11-nAPG08290.1 | SGN000143 | 4.78 | 5.77 |
| APG08290.1 | SGN000143 | 0 | 6.21 |
| ARM05-nAPG08290.1 | SGN000169 | 4.26 | 12.87 |
| ARM06CTD-nAPG08290.1 | SGN000169 | 1.63 | 8.29 |
| ARM08-nAPG08290.1 | SGN000169 | 6.99 | 14.27 |
| ARM11-nAPG08290.1 | SGN000169 | 4.99 | 6.39 |
| APG08290.1 | SGN000169 | 0 | 11.85 |
| ARM05-nAPG08290.1 | SGN000173 | 3.26 | 5.51 |
| ARM06CTD-nAPG08290.1 | SGN000173 | 0.72 | 0.96 |
| ARM08-nAPG08290.1 | SGN000173 | 6.6 | 6.06 |
| ARM11-nAPG08290.1 | SGN000173 | 2.83 | 4.82 |
| APG08290.1 | SGN000173 | 0 | 2.58 |

### Example 11: Trans ssDNA Cleavage

### 11.1 Determining Assay Conditions for Trans DNA Cleavage

Purified APG09748 (set forth as SEQ ID NO: 137 and described in International Appl. No. PCT/US2019/068079, which is incorporated by reference in its entirety) was incubated with single guide RNA (sgRNA) in Cutsmart buffer (New England Biolabs B7204S) at a final concentration of either 50 nM nuclease and 100 nM sgRNA or 200 nM nuclease and 400 nM sgRNA for 10 min. These RNP solutions were added to solutions of ssDNA—a target or mismatched negative control ssDNA—at a final concentration of 10 nM and reporter probes at a final concentration of 250 nM in 1.5× Cutsmart buffer (New England Biolabs B7204S). The reporter probes (TB0125 and TB0089, set forth as SEQ ID NOs: 138 and 139, respectively) contain a fluorescent dye at the 5′ end (56-FAM for TB0125 and Cy5 for TB0089), a quencher at the 3′ end (3IABKFQ for TB0125 and 3IAbRQSp for TB0089), and optionally an internal quencher (the internal quencher ZEN is only present on TB0125). Cleavage of the reporter probe results in dequenching of the fluorescent dye and thus an increase in fluorescence signal. To monitor fluorescence intensity, 10 μl of each reaction was incubated in a Corning low volume 384-well microplate at 30 °C in a microplate reader (CLARIOstar Plus).

A number of conditions were scouted in order to determine suitable parameters for this assay. In order to determine if there are effects of quenched probe design or fluorophore characteristics, two such reporters were included as a mixture in each reaction. They were at the same concentration as each other in any given reaction. In all cases, the control or target ssDNA concentration (LE201 or LE205, set forth as SEQ ID NOs: 140 and 141, respectively) was 10 nM. The RNP names signify the nuclease and the target as indicated in Table 13 below.

TABLE 13
Ribonucleoprotein complexes

| RNP Name | Nuclease | sgRNA | Intended Target |
|---|---|---|---|
| APG09748.1 | APG09748 | 27sg.1 | LE201 |
| | | (SEQ ID NO: 144) | (SEQ ID NO: 140) |
| APG09748.2 | APG09748 | 27sg.2 | LE205 |
| | | (SEQ ID NO: 145) | (SEQ ID NO: 141) |

The results are shown in Table 14 below.

TABLE 14
Results of trans DNA cleavage assays

| | | | | Slope (RFU/min) | |
|---|---|---|---|---|---|
| | [RNP] | [reporters] | | Cy5 | FAM |
| RNP Name | (nM) | (nM) | Target | channel | channel |
| APG09748.1 | 25 nM | 0 | LE201 | −0.30 | −2.40 |
| APG09748.1 | 25 nM | 50 | LE201 | 420.00 | 46.20 |
| APG09748.1 | 25 nM | 250 | LE201 | 1040.40 | 47.40 |
| APG09748.1 | 25 nM | 500 | LE201 | 1090.20 | 57.00 |
| APG09748.1 | 25 nM | 750 | LE201 | 996.60 | 36.60 |
| APG09748.1 | 25 nM | 1000 | LE201 | 770.40 | 36.00 |
| APG09748.1 | 25 nM | 0 | LE205 | 0.60 | −1.80 |
| APG09748.1 | 25 nM | 50 | LE205 | 6.00 | −1.20 |
| APG09748.1 | 25 nM | 250 | LE205 | 16.80 | −3.00 |
| APG09748.1 | 25 nM | 500 | LE205 | 33.00 | 1.80 |
| APG09748.1 | 25 nM | 750 | LE205 | 18.00 | 3.00 |
| APG09748.1 | 25 nM | 1000 | LE205 | −21.60 | −51.60 |
| APG09748.2 | 25 nM | 0 | LE201 | −0.18 | −1.80 |
| APG09748.2 | 25 nM | 50 | LE201 | 4.20 | 1.20 |
| APG09748.2 | 25 nM | 250 | LE201 | 42.60 | 3.00 |
| APG09748.2 | 25 nM | 500 | LE201 | 70.80 | 10.20 |
| APG09748.2 | 25 nM | 750 | LE201 | 67.80 | 10.80 |
| APG09748.2 | 25 nM | 1000 | LE201 | 55.80 | 7.80 |
| APG09748.2 | 25 nM | 0 | LE205 | 0.06 | −2.40 |
| APG09748.2 | 25 nM | 50 | LE205 | 321.00 | 64.20 |
| APG09748.2 | 25 nM | 250 | LE205 | 669.60 | 63.00 |
| APG09748.2 | 25 nM | 500 | LE205 | 706.80 | 52.20 |
| APG09748.2 | 25 nM | 750 | LE205 | 627.00 | 55.20 |
| APG09748.2 | 25 nM | 1000 | LE205 | 534.60 | 46.80 |
| APG09748.1 | 100 nM | 0 | LE201 | −0.42 | −1.80 |
| APG09748.1 | 100 nM | 50 | LE201 | 1063.80 | 155.40 |
| APG09748.1 | 100 nM | 250 | LE201 | 2386.20 | 144.00 |
| APG09748.1 | 100 nM | 500 | LE201 | 3058.80 | 108.60 |
| APG09748.1 | 100 nM | 750 | LE201 | 2974.20 | 99.00 |
| APG09748.1 | 100 nM | 1000 | LE201 | 3282.00 | 113.40 |
| APG09748.1 | 100 nM | 0 | LE205 | 1.20 | −0.36 |
| APG09748.1 | 100 nM | 50 | LE205 | 6.60 | 0.18 |
| APG09748.1 | 100 nM | 250 | LE205 | 27.00 | 1.80 |
| APG09748.1 | 100 nM | 500 | LE205 | 52.80 | 12.60 |
| APG09748.1 | 100 nM | 750 | LE205 | 57.60 | 9.00 |
| APG09748.1 | 100 nM | 1000 | LE205 | 73.80 | 16.20 |
| APG09748.2 | 100 nM | 0 | LE201 | 0.06 | −0.48 |
| APG09748.2 | 100 nM | 50 | LE201 | 25.80 | 9.00 |
| APG09748.2 | 100 nM | 250 | LE201 | 198.00 | 15.00 |
| APG09748.2 | 100 nM | 500 | LE201 | 265.80 | 16.20 |
| APG09748.2 | 100 nM | 750 | LE201 | 277.20 | 19.20 |
| APG09748.2 | 100 nM | 1000 | LE201 | 258.00 | 7.80 |
| APG09748.2 | 100 nM | 0 | LE205 | 0.12 | 0.00 |
| APG09748.2 | 100 nM | 50 | LE205 | 844.80 | 257.40 |
| APG09748.2 | 100 nM | 250 | LE205 | 2091.60 | 270.60 |
| APG09748.2 | 100 nM | 500 | LE205 | 2551.80 | 196.80 |
| APG09748.2 | 100 nM | 750 | LE205 | 2490.60 | 186.00 |
| APG09748.2 | 100 nM | 1000 | LE205 | 2511.00 | 138.00 |

From this experiment, it was concluded that the 100 nM concentration of RNP results generally in a higher cleavage rate of the reporter probe than the 25 nM RNP concentration. In general, reporter cleavage rates are higher at higher concentrations of the reporter oligonucleotides up to 250 to 500 nM reporter concentration, with little benefit observed from further increase in reporter concentrations. Notably, for the TB0089 reporter (detected in the Cy5 channel), there are substantially higher levels of background activity that interfere with target differentiation, especially at reporter concentrations higher than 250 nM. Therefore, it was concluded that reporter concentrations higher than 250 nM would not be beneficial. Both probes seem suitable for observation of target recognition by the RNP.

### 11.2 APG09748 Trans DNA Cleavage and Effect of Purification on Non-Specific Activity

Purified APG09748 was incubated with single guide RNA (sgRNA) in 1× Cutsmart buffer (New England Biolabs B7204S) at a final concentration of 200 nM nuclease and 400 nM sgRNA for 10 min at 37 °C. These RNP solutions are then added to solutions of ssDNA—a target or mismatched negative control ssDNA—at a final concentration of 10 nM and a reporter probe (TP0003) at a final concentration of 250 nM in 1.5× Cutsmart buffer (New England Biolabs B7204S). The reporter probe contains a fluorescent dye at the 5′ end and a quencher at the 3′ end. Cleavage of the reporter probe results in dequenching of the fluorescent dye and thus an increase in fluorescence signal. To monitor fluorescence intensity, 10 μl of each reaction was incubated in a Corning low volume 384-well microplate at 37 °C in a microplate reader (CLARIOstar Plus).

Incubation with target sequences resulted in a substantial increase in fluorescence intensity as a function of time relative to the negative control. The rate of cleavage is summarized as the slope of the linear portion of the fluorescence vs. time function as shown in Table 15.

TABLE 15
Results of trans DNA cleavage assay

| ssDNA substrate sequence | Slope (RFU/min) |
|---|---|
| Mismatch (LE111; SEQ ID NO: 142) | 549 |
| Match (LE113; SEQ ID NO: 143) | 7724 |

These data show differentiation of the target sequence from the negative control by the RNPs that is clearly detectable using the cleaved reporter probe.

### 11.3 PAM Determination Using a Parallel Plasmid DNA Library

Oligonucleotides (LE00680 and LE00688 set forth as SEQ ID NOs: 266 and 267, respectively) containing a target sequence preceded by a 5 nt degenerate PAM sequence (NNNNN) were annealed and cloned into double digested pUC19 plasmid using NEBuilder HiFi DNA Assembly Master Mix (New England Biolabs). Each colony resulting from the transformation of this reaction corresponds to a clonal plasmid DNA sequence so that preparations of plasmid DNA from cultures deriving from single colonies are unique plasmid preparations sampled from the original library. Plasmid preparations were obtained from a sampling of 96 colonies. These preparations were individually subjected to Sanger sequencing to verify their PAM sequence.

Purified APG09748 was incubated with sgRNA (27sg.2) in 1× Cutsmart buffer (New England Biolabs B7204S) at a final concentration of 200 nM nuclease and 400 nM sgRNA for 20 minutes at room temperature.

These RNP solutions were added at a final concentration of 100 nM to solutions of plasmid DNA targets at a final concentration of 8.3 nM and TB0125 and TB0089 reporters (set forth as SEQ ID NOs: 138 and 139, respectively) at 250 nM and 50 nM respectively in 1.5× Cutsmart buffer (New England Biolabs B7204S). To monitor fluorescence intensity, 10 μl of each reaction was incubated in a Corning low volume 384-well microplate at 37 °C in a microplate reader (CLARIOstar Plus).

The plasmid concentration in a minority of samples was either below the target for concentration normalization or the volume of the solution was insufficient to deliver the intended target amount to the reaction well. These samples were not excluded from analysis and are thus expected to contribute to error through inconsistencies between duplicates. Samples which used plasmids that, upon evaluation by Sanger sequencing, were determined to have cross contamination (multiple traces apparent in the PAM region) or alterations in the target sequence were removed from the analysis and their results are not shown below. The results of the analysis are shown in Table 16 below in descending order by slope in the FAM channel.

TABLE 16
PAM sequences

| | | Slope (RFU/min) | |
|---|---|---|---|
| PAM | SEQ ID NO | Cy5 channel | FAM channel |
| ATATG | 147 | 111.52 | 1728.13 |
| ATATG | 147 | 102.42 | 1612.86 |
| TATTG | 148 | 137.15 | 846.12 |
| TATTG | 148 | 148.61 | 799.82 |
| CGTTC | 149 | 151.06 | 744.18 |
| CGTTC | 149 | 134.61 | 735.35 |
| TTTTT | 150 | 167.71 | 731.11 |
| TTTTT | 150 | 149.83 | 711.78 |
| CAATC | 151 | 120.84 | 675.71 |
| AATCT | 152 | 134.67 | 622.52 |
| AATCT | 152 | 134.1 | 620.2 |
| CAATC | 151 | 96.94 | 608.58 |
| AAATT | 153 | 152.6 | 607.24 |
| AAATT | 153 | 150.57 | 591.77 |
| CATCC | 154 | 97.34 | 588.7 |
| AGATA | 155 | 214.69 | 579.56 |
| TATTC | 156 | 124.22 | 570.81 |
| ATTTT | 157 | 134.72 | 551.91 |
| AGATA | 155 | 198.45 | 544.81 |
| TATTC | 156 | 108.57 | 538.18 |
| TCTTT | 158 | 141.66 | 532.87 |
| ATTTT | 157 | 107.9 | 526.75 |
| AGATC | 159 | 106.93 | 497.71 |
| AATAA | 160 | 78.94 | 484.67 |
| TCTTT | 158 | 117.19 | 484.13 |
| GATCC | 161 | 112.85 | 479.91 |
| GATCC | 161 | 105.04 | 474.94 |
| AGATC | 159 | 106.5 | 473.98 |
| ATTCA | 162 | 115.03 | 473.24 |
| AATAA | 160 | 77.43 | 465.75 |
| AAAAT | 163 | 78.3 | 458.23 |
| TTATC | 164 | 102.14 | 456.73 |
| TATCA | 165 | 106.45 | 448.78 |
| TCACT | 166 | 167.02 | 429.36 |
| CTTAT | 167 | 82.9 | 418.38 |
| TATTG | 148 | 116.08 | 417.94 |
| TCACT | 166 | 160.29 | 415.64 |
| AAAAT | 163 | 56.43 | 411.26 |
| TGATA | 168 | 89.02 | 406.14 |
| TGATA | 168 | 85.99 | 395.57 |
| TACTA | 169 | 90.61 | 391.06 |
| ATTTT | 157 | 113.18 | 382.59 |
| TACTA | 169 | 74.33 | 364.56 |
| ATTTT | 157 | 106.72 | 364.02 |
| TATTG | 148 | 93.95 | 341.36 |
| CTTAT | 167 | 76.12 | 336.8 |
| ATATA | 170 | 83.39 | 326.86 |
| ATATA | 170 | 72.98 | 318.2 |
| TATCA | 165 | 65.41 | 317.53 |
| GACTC | 171 | 56.56 | 302.2 |
| GCTCA | 172 | 22.1 | 293.68 |
| TAACC | 173 | 56.08 | 283.99 |
| CGTGT | 174 | 66.57 | 268.89 |
| TAACC | 173 | 56.55 | 265.45 |
| TTTCG | 175 | 59.55 | 262.91 |
| CGTGT | 174 | 50.71 | 262.37 |
| TTTCG | 175 | 61.42 | 261.65 |
| CAGTA | 176 | 51.81 | 240.09 |
| GCTCA | 172 | 19.08 | 228.08 |
| CAGTA | 176 | 51.09 | 220.25 |
| CCCTT | 177 | 17.83 | 136.69 |
| CCTAT | 178 | 40.4 | 125.81 |
| TCCCT | 179 | 39.93 | 124.6 |
| AGGTT | 180 | 40.96 | 117.18 |
| AGGTT | 180 | 40.81 | 114.79 |
| TAACG | 181 | 32.53 | 94.68 |
| AACAG | 182 | 8.19 | 91.72 |
| CCTAT | 178 | 34.51 | 87.57 |
| TGAAC | 183 | 10.22 | 86.38 |
| TAAGT | 184 | 19.35 | 85.12 |
| TCCCT | 179 | 26.67 | 83.02 |
| TAACG | 181 | 27.62 | 79.17 |
| TAACG | 181 | 28.89 | 78.55 |
| TAACG | 181 | 22.23 | 78.36 |
| CTGTA | 185 | 23.27 | 77.06 |
| AGGGG | 186 | 30.8 | 74.4 |
| GCTGT | 187 | 28.3 | 71.54 |
| ATCTA | 188 | 18.73 | 69.15 |
| GCTGT | 187 | 25.61 | 69.06 |
| TTATC | 164 | 22.8 | 68.13 |
| AACAG | 182 | 23.98 | 67.27 |
| TCAGG | 189 | 17.25 | 67.04 |
| ATGTC | 190 | 13.91 | 65.93 |
| TGCTA | 191 | 26.33 | 65.73 |
| TAAAA | 192 | 17.32 | 65.43 |
| ATCTA | 188 | 16.88 | 65.3 |
| CTGTA | 185 | 21.81 | 64.15 |
| GACTC | 171 | 24.05 | 64.02 |
| ATTCA | 162 | 25.04 | 63.87 |
| TGAAC | 183 | 24.53 | 63.64 |
| CCCTT | 177 | 21.35 | 61.95 |
| ATGTC | 190 | 12.99 | 60.6 |
| ATCAG | 193 | 22.32 | 59.63 |
| ATCAG | 193 | 18.55 | 56.58 |
| CGGGG | 194 | 27.86 | 56.28 |
| TAAAA | 192 | 17.72 | 56.03 |
| TTCTC | 195 | 25.7 | 55.83 |
| GTCAT | 196 | 23.07 | 55.76 |
| GCGTG | 197 | 17.36 | 55.12 |
| GCGTG | 197 | 22.02 | 55.02 |
| GCAAT | 198 | 15.47 | 54.91 |
| TTCTC | 199 | 21.89 | 54.89 |
| GTCAT | 196 | 17.22 | 54.34 |
| GCAAT | 198 | 17.36 | 53.32 |
| AGCAT | 200 | 22.47 | 52.23 |
| GTCAA | 201 | 14.14 | 52.12 |
| AGAGC | 202 | 19.62 | 51.79 |
| AGAGC | 202 | 15.99 | 50.37 |
| GTGGG | 203 | 22.38 | 50.1 |
| CTGAA | 204 | 21.24 | 49.45 |
| CTGAT | 205 | 12.05 | 49.32 |
| ATGAA | 206 | 17.93 | 49.15 |
| ACCTT | 207 | 19.66 | 49.12 |
| ACCTT | 207 | 22.74 | 49.12 |
| GTCAA | 201 | 13 | 48.21 |
| AACCA | 208 | 17.81 | 48.04 |
| AACCA | 208 | 15.75 | 47.87 |
| GTAAA | 209 | 16.3 | 47.85 |
| TAGGA | 210 | 18.68 | 47.73 |
| CCGAA | 211 | 20.24 | 47.58 |
| AGCAT | 200 | 22.95 | 47.52 |
| CGGGG | 194 | 20.94 | 47.41 |
| GTCAT | 196 | 15.17 | 47.24 |
| CTGAA | 204 | 20.4 | 46.36 |
| GTGGG | 203 | 19.98 | 46.25 |
| TACGA | 212 | 16.79 | 46.18 |
| TGGCT | 213 | 14.81 | 45.75 |
| GTCAT | 196 | 15.55 | 45.51 |
| ATGAA | 206 | 19.11 | 45.09 |
| TAGGA | 210 | 21.65 | 44.94 |
| TTCTC | 199 | 21.44 | 44.76 |
| CCAAG | 214 | 16.98 | 44.61 |
| GTAAA | 209 | 15.35 | 44.59 |
| TGCTT | 215 | 15.79 | 44.54 |
| CTAAA | 216 | 12.72 | 44.28 |
| TGCTA | 191 | 16 | 44.14 |
| TACGA | 212 | 16.07 | 44.06 |
| TCGAT | 217 | 22.28 | 43.96 |
| TTCTC | 199 | 15.94 | 42.55 |
| CTGAT | 205 | 16.13 | 42.27 |
| TCAGG | 189 | 11.38 | 41.9 |
| CCAAG | 214 | 11.28 | 41.45 |
| AGGGG | 186 | 16.04 | 41.44 |
| TCGAT | 217 | 18.68 | 40.5 |
| CCGAA | 211 | 15.39 | 39.09 |
| CTAAA | 216 | 10.72 | 30.37 |
| GTAAC | 218 | 4.36 | 9.51 |
| TGCTT | 215 | −0.32 | -0.7 |
| TAAGT | 184 | −0.57 | -1.3 |
| CATCC | 154 | 0.47 | -1.95 |
| TGGCT | 213 | −0.03 | -2.2 |
| GTAAC | 219 | −0.46 | -2.3 |

Sequences with the highest slope appear to comply with the predicted PAM (DTTN set forth as SEQ ID NO: 60) determined by the plasmid depletion assay previously described in International Appl. No. PCT/US2019/068079, which is incorporated by reference in its entirety. In particular, we see a strong preference for “T” in the position two nucleotides 5′ of the target. Surprisingly, the most active PAM site observed in this assay (ATATG, SEQ ID NO: 147) did not exactly match the consensus of DTTN (SEQ ID NO: 60), suggesting some level of flexibility in this recognition site.

11.4. Trans ssDNA Cleavage Activity in the Presence of PCR Amplified DNA

PCR amplified targets were generated from genomic DNA (TRAC and VEGF amplicons set forth as SEQ ID NOs: 225 and 226, respectively) using appropriate primers. The VEGF target (set forth as SEQ ID NO: 227) was PCR amplified from HEK293T cell genomic DNA by primers with sequences LE573 and LE578 (set forth as SEQ ID NOs: 228 and 229, respectively). The TRAC target (set forth as SEQ ID NO: 230) was PCR amplified by LE257 and LE258 (set forth as SEQ ID NOs: 231 and 232, respectively).

RNPs were formed by incubation of APG09106.1 nuclease described herein and sgRNA at 0.5 μM and 1 μM respectively in 1×NEBuffer 2 (New England Biolabs) and incubated at room temperature for 20 minutes.

TABLE 17
Ribonucleoprotein complexes

| RNP | Nuclease | Guide RNA | Intended target |
|---|---|---|---|
| APG09106.2 | APG09106.1 | 27sg.2 | LET 126 amplicon |
| | | (SEQ ID NO: 145) | Randomized PAM |
| | | | amplicon |
| APG09106.836 | APG09106.1 | 27sg.836 | VEGF amplicon |
| | | (SEQ ID NO: 233) | |
| APG09106.838 | APG09106.1 | 27sg.838 | TRAC amplicon |
| | | (SEQ ID NO: 234) | |

The cleavage reaction was performed in 1.5×NEBuffer 2 with 1.5 μM ssDNA oligonucleotide reporter with a 5′ TEX615 label and a 3′ Iowa Black FQ quencher and 100 nM of the respective PCR product. Cleavage of the reporter probe results in dequenching of the fluorescent dye and thus an increase in fluorescence signal. To monitor fluorescence intensity, 10 μl of each reaction was incubated in a Corning low volume 384-well microplate at 37 °C in a microplate reader (CLARIOstar Plus). The results of kinetic analysis are shown in Table 18.

TABLE 18
Results of trans DNA cleavage assay

| | Target Conc. | | Slope |
|---|---|---|---|
| RNP | (nM) | Target | (RFU/min) |
| 28.836 | 30 | TRAC amplicon | 1204 |
| 28.838 | 30 | TRAC amplicon | 8436 |
| 28.838 | 30 | VEGF amplicon | 766 |
| 28.836 | 30 | VEGF amplicon | 3484 |

These results indicate specific activation of trans ssDNA cleavage activity in the presence of PCR amplified DNA from various sources. The activity is dependent on the concentration of the PCR amplified substrate.

11.5 Use of ssDNA Cleavage as a Diagnostic

Due to the capability of these nucleases to generate an optically detectable signal in the presence of a target DNA sequence, they promise utility for implementation into diagnostic devices for the detection of genetic disease or agents of infectious disease, such as bacteria, viruses, or fungi.

A diagnostic procedure may include isolation or amplification of nucleic acids from a sample to be tested. It may also be suitable to use some samples without performing any isolation or purification of nucleic acids, as they may be present in the sample at high enough quantities to be detectable without amplification (such as PCR) or free of materials that interfere with detection or signal production.

RNPs formed as described in the other examples could then be exposed to the sample (or processed sample as described in the preceding paragraph) along with a reporter, such as the fluorophore and quencher modified ssDNA oligonucleotides used in previous examples, or some other sort of ssDNA substrate that produces a visible or otherwise easily detectable signal when cleaved. If using fluorophore-quencher conjugated DNA oligonucleotides (as in the previously described examples), these can be detected using a fluorimeter as described in previous examples. To simplify detection, an endpoint assay can be performed instead of the kinetic assays described above, meaning that the assays can be performed for a fixed time and read out at the end of this elapsed time, relative to positive and negative controls.

These reagents may also be integrated into a lateral flow testing device which allows for the detection of a given disease-causing agent or specific nucleic acid sequence (such as a diseased allele in an individual) with very little instrumentation. In this assay, the ssDNA reporter would be conjugated to multiple molecules suitable for antibody or affinity reagent capture, such as fluorescein, biotin, and/or digoxigenin.

That which is claimed:

1. An RNA-guided nuclease (RGN) polypeptide, wherein said RGN polypeptide is selected from the group consisting of:
a) an RGN polypeptide comprising an amino acid sequence having at least 95% sequence identity to SEQ ID NO: 117, 30, 75, 1, 9, 16, 23, 38, 46, 61, 69, 82, 89, 95, 103, or 110; and
b) an RGN polypeptide comprising the amino acid sequence set forth as SEQ ID NO: 54; and wherein said RGN polypeptide is operably fused to one or more nuclear localization signals heterologous to said RGN polypeptide.

2. The RGN polypeptide of claim 1, wherein said RGN polypeptide of a) is nuclease inactive or is capable of functioning as a nickase.

3. The RGN polypeptide of claim 1, wherein said RGN polypeptide comprises the amino acid sequence set forth as SEQ ID NO: 117, 30, 75, 1, 9, 16, 23, 38, 46, 61, 69, 82, 89, 95, 103, or 110.

4. A fusion polypeptide comprising:
(A) an RNA-guided nuclease (RGN) polypeptide selected from the group consisting of:
i) an RGN polypeptide comprising an amino acid sequence having at least 95% sequence identity to SEQ ID NO: 117, 30, 75, 1, 9, 16, 23, 38, 46, 61, 69, 82, 89, 95, 103, or 110; and
ii) an RGN polypeptide comprising the amino acid sequence set forth as SEQ ID NO: 54; and
(B) a heterologous polypeptide.

5. The fusion polypeptide of claim 4, wherein said RGN polypeptide comprises the amino acid sequence set forth as SEQ ID NO: 117, 30, 75, 1, 9, 16, 23, 38, 46, 61, 69, 82, 89, 95, 103, or 110.

6. The fusion polypeptide of claim 4, wherein said RGN polypeptide of (A) i) is nuclease inactive or is capable of functioning as a nickase.

7. The fusion polypeptide of claim 4, wherein the heterologous polypeptide is a base-editing polypeptide or an effector domain.

8. The fusion polypeptide of claim 7, wherein said base-editing polypeptide is a deaminase.

9. The fusion polypeptide of claim 7, wherein said base-editing polypeptide is operably fused at the N-terminus or at the C-terminus of said RGN polypeptide.

10. The fusion polypeptide of claim 7, wherein said effector domain comprises a cleavage domain, a deaminase domain, or an expression modulator domain.

11. The fusion polypeptide of claim 10, wherein said expression modulator domain comprises an epigenetic modification domain, a transcriptional repressor domain, or a transcriptional activation domain.

12. The fusion polypeptide of claim 7, wherein said effector domain is operably fused at the N-terminus or at the C-terminus of said RGN polypeptide.

13. The fusion polypeptide of claim 7, wherein said effector domain is operably fused at an internal location of said RGN polypeptide.

14. A method for cleaving and/or modifying a target DNA sequence, comprising delivering to the target DNA sequence:
a) a nucleic acid molecule comprising a polynucleotide encoding an RNA-guided nuclease (RGN) polypeptide, wherein said RGN polypeptide is selected from the group consisting of:
i) an RGN polypeptide comprising an amino acid sequence having at least 95% sequence identity to SEQ ID NO: 117, 30, 75, 1, 9, 16, 23, 38, 46, 61, 69, 82, 89, 95, 103, or 110; and
ii) an RGN polypeptide comprising the amino acid sequence set forth as SEQ ID NO: 54;
wherein the RGN polypeptide encoded by the polynucleotide is expressed; and
b) one or more guide RNAs capable of targeting the RGN of a) to the target DNA sequence; thereby cleaving and/or modifying said target DNA sequence.

15. The method of claim 14, wherein said polynucleotide encoding an RGN polypeptide is operably linked to a promoter heterologous to said polynucleotide.

16. The method of claim 14, wherein said RGN polypeptide comprises the amino acid sequence set forth as SEQ ID NO: 117, 30, 75, 1, 9, 16, 23, 38, 46, 61, 69, 82, 89, 95, 103, or 110.

17. The method of claim 14, wherein said RGN polypeptide of a) i) is nuclease inactive or is capable of functioning as a nickase.

18. The method of claim 14, wherein said RGN polypeptide is operably fused to a base-editing polypeptide or an effector domain.

19. The method of claim 14, wherein the target sequence is within a eukaryotic cell.

20. The method of claim 14, wherein said polynucleotide encoding an RGN polypeptide is codon optimized for expression in a eukaryotic cell.
